SEQUENCE LISTING (186 sequences)

SEQ ID NO: 1 - protein, 106 aa, synthetic construct
MHHHHHHEVDQQILLQQLKSDYRQILLSYFTTDKALKEKIDKFANAVFCANIPVPEIIEIHMELIDEFSKQLRLEGRGDEDLMDYRLTLEDILRSLIEDYLFAIFK

SEQ ID NO: 2 - protein, 170 aa, synthetic construct
MNIFEALRIDQGLRLKIYKNTEGYYTIGIGHLLTKSPSLNAAKSELDKAIGRNCNGVITKDEAEKLFNQDVRSAVEDILFNAKLSPVFDSLDAVRRAALIIMVFQMGETGVAGFTNSLRMLQQKRWDEAAVNLAKSRWYNQTPNRAKRVITTFRTGTWDAYKLEHHHHHH

SEQ ID NO: 3 - protein, 137 aa, synthetic construct
MDIPKITTFLMFNNQAERSVIEDTLFFEDSEIITMAKYGENGPGDPGTVQHSIFTLNGQVFMAIDANSGTELPISLFVTVKDTIEMERLFNGLKDEGAILMPKTNMPPYREFAWVQDKFGVSFQLALPELEHHHHHH

SEQ ID NO: 4 - protein, 135 aa, synthetic construct
HMDRDRFMDEFFEQVEEIRGFIDKIAENVEEVKRKHSAILASPNPDEKTKEELEELMSDIKKTANKVSSKLKSIEQSIEQEEGLNRSSADLRIRKTQHSTLSRKFRSVIEDFLFTQALYRERASGRILEHHHHHH

SEQ ID NO: 5 - protein, 158 aa, synthetic construct
MKSTEKKELSHFRLKLETYLNEHFPEMSGNNPFITARSDEALRSYIEDVLFGFSHPEAESMASEVLYQGLHFSRYDTLVSVLEREFEQELPSPLPERLAPILLKNKAIQSVFAKYDLTDDFEASPEYEHLYTELTGTIVLLIESNHLPTILEHHHHHH

SEQ ID NO: 6 - protein, 139 aa, synthetic construct
MLAKILVIDDESTILQNIKFLLEIDGNEVLTASSSRSGIEDFLFNANSIDVVITDMKMPKSSGMDILRAIKKITPHMAVIILTGHGDLDNAILAMKEGAFEYLRKPVTAQDLSIAINNAINRKKLLMENERLEHHHHHH

SEQ ID NO: 7 - protein, 131 aa, synthetic construct
MARRILVVAAEAPIARSVIEDLLFNGFQPVAAEDYDSAVNSLNEPWPDLILLNWMLPGGSGIQFIKHLKRESMTRDIPVVMLTARGEEEDRVRGLETGADDYITKPFSPKELVARIKAVMRRILEHHHHHH

SEQ ID NO: 8 - protein, 165 aa, synthetic construct
EGDDPTNLESALIGKPVPKFRLESLDNPGQFYQADVLTQGKPVLLNVWATWCPTCRAEHQYLNQLSAQGIRVVGMNYKDDRRSAIEWLLFLGNPYALSLFDGDGMLGLDLGVEGAAATFLIDGNGIIRYRHAGDLNPRVWEEEIKPLWEKYSKEAAQLEHHHHHH

SEQ ID NO: 9 - protein, 93 aa, synthetic construct
MRPKLYKVMLLNDDYTPREFVTVVLKAVFRMSEDTGRRVMMTAHRFGSAVVGVFTRDVADTKARSAIEDGLFAGFPLTFTTEPEELEHHHHHH

SEQ ID NO: 10 - protein, 106 aa, synthetic construct
MSLLRSYAEDFLFTLAQAKASLAEAPSQPLSQRNTTLKHVEQQQDELFDLLDQMDVEVNNSIGDASERATFKASLREWKKTIQSDIKRPLQSLVDSGDLEHHHHHH

SEQ ID NO: 11 - protein, 115 aa, synthetic construct
MADLRSNIEDLLFNLAQIEKADNAAQVKDALTKMRAAALDAQKATPPKLEDKSPDSPEMKDFRHGFDILVGQIDDALKLANEGKVKEAQAAAEQLKTTLKAYIAKYLLEHHHHHH

SEQ ID NO: 12 - protein, 155 aa, synthetic construct
MNLDDTLDVLNDLLQTSKDGEAGFHACAEDLRDPQLKAAMLEASRDAAAAADELERIVLELGGKPKDSTSFAGDLHRRWVDLKSLVTGKDEEAVLNECERGLAVAAARYAAALEKSLPAEIHQVIERQAQGVRSHIEDVLFLRASRALEHHHHHH

SEQ ID NO: 13 - protein, 119 aa, synthetic construct
MSTVAVTDATFEADVLKSSKPVLVDFWAEWSGPSKQIAPALEQLSEELADVVTIAKVNIEDSPTTPSRYGVRGIPTMMLFRDGQMTSMKVGAMPKRSILEWLLFAGAQAALLEHHHHHH

SEQ ID NO: 14 - protein, 136 aa, synthetic construct
MQEAANRSPPYAPNPYPVDEIIGGDSVQSIQRRLLGTNWNPSAHDMQMSRIQAEDLFLAKVSIIRKMAGLHPSGDWMGWGARALDNPRTATGEEDLARLRSMLEDLLFRNEQSATFWRLVERVRLRADLEHHHHHH

SEQ ID NO: 15 - protein, 115 aa, synthetic construct
MADLRSNWEDLLFNLAQIEKADNAAQVKDALTKMRAAALDAQKATPPKLEDKSPDSPEMKDFRHGFDILVGQIDDALKLANEGKVKEAQAAAEQLKTTRKAYIAKYLLEHHHHHH

SEQ ID NO: 16 - protein, 103 aa, synthetic construct
MHHHHHHGWSENLYFQGSTVRIEIRFTNMARFKVELDLYFFKHSLKELEKRTGSEIRIEIEERDGEVRVEVEIRNSHEEEVRQIIEEIERWVRKMGGELRVEK

SEQ ID NO: 17 - protein, 142 aa, synthetic construct
HMQSNAMTFAHEVVKSNVKNVKDRKGKEKQVLFNGLTTSKLRNLMEQVNRLYTIAFNSNEDQLAFKFELDLYFLKHSFYYEAGREKSVDEFLKKTLMFPIIDRVIKKESKKFFLDYCKYFEALVAYAKYYQKEDLEHHHHHH

SEQ ID NO: 18 - protein, 113 aa, synthetic construct
MHHHHHHLLGFYKQYKALSEYIDKKYKLSLNDLAVLDLTMKHATDEKVLMQSFLKTAMDELDLSRTKLLVSIRRLIEKERLSKVRSSKDERKIYIYLNFKDELDFYFLFEDVE

SEQ ID NO: 19 - protein, 113 aa, synthetic construct
MHHHHHHADLEDNWETLNDNLKVIANADNAAQVKDALTKMRAAALDAQKATPPKLEDKSPDSPEMKDFRHGFDILVGQIDDALKAANEGGVFKAELDAYFLHYTRNAYIQKYL

SEQ ID NO: 20 - protein, 143 aa, synthetic construct
MHHHHHHENLYFQGIPRITIHAFCARPETAALIEKAAADRRMSRAATIVRDGGLEAAVDYYQNQPTPSLVMVETLDGAQRLLHLLDSLAQVCDPGTKVVVVGQTNDFKLELDLYFRGVSAYLTQPLGPLQVIRAVGALYADPA

SEQ ID NO: 21 - protein, 107 aa, synthetic construct
MVTVEEEVYEFLKKKAKEEGTSVPAVIRKILKEYFGIEDRTRDYKRQDLEGSYIIVNGKKYYRINASLEFKNELDVYFELKSRGTTLNRFLKEMIMITVLEHHHHHH

SEQ ID NO: 22 - protein, 101 aa, synthetic construct
MTSTFDRVATIIAETADIPRETITPESHAIDDLGIDELDFLDIAFAIDKAFGISLPLFKWELDVYFGSATTEQYFVLKNLAARIDELVAAKGALEHHHHHH

SEQ ID NO: 23 - protein, 142 aa, synthetic construct
MRSKRVLVVEDNPDDIALIRRVLDRKDIHAQLEFVDNGAKALYQVQQAKYDLIILDIGLPIANGFEVMSAVRKPGANQHTPIVILTDNASFKRELDAYFAGASEAVDKSSNNVTDFYGRIYAIFSYWLTVNHAQLEHHHHHH

SEQ ID NO: 24 - protein, 102 aa, synthetic construct
MHHHHHHNTDELKQKYGRVYEIRIEGAEYENGEEAEFVFYFTRPSFKDELDFYFELHSKPDMAMKNLTFSAIVPEQEEELRQAAEEFPGLTFNTASRLMEIV

SEQ ID NO: 25 - protein, 128 aa, synthetic construct
MEFFDILEDVKEDHFEKLLEEAVEEVIDSGNELVRSPTPSNLKRYKNAIKEFLKLIEKKIYKLAGSFDMFKGELDLYFVVHAVNEKLMDLTEKIMKNEWQTINLAARIEEINGLILNLYRLEHHHHHH

SEQ ID NO: 26 - protein, 89 aa, synthetic construct
MHHHHHHGAESTAERSARFERDALAFAFKMELDAYFMTRNPADAEDLVQETYAKAYASFHQFREGTNLKAWLSEILTNTFSNSYRKKQR

SEQ ID NO: 27 - protein, 97 aa, synthetic construct
KDTEDEALMRTLLDHFDQYIKISKKISAETYAAVTDIEEPGRMADIVASHLPLDFKDELDIYFTADVKDRLNKVIDFINNEKEVLEIEKLEHHHHHH

SEQ ID NO: 28 - protein, 104 aa, synthetic construct
HMMTVAEDKTFQYIRQHHSNFSRIHVLEILPYLSALTFKDELDLYFTYHRWGNQDTLLELFTSLRSRNGWVHSLIGALRAAELSGLADEVARIYHSLEHHHHHH

SEQ ID NO: 29 - protein, 144 aa, synthetic construct
MSHKEPATLIKAIDGDTVKLMYKGQPMTFRLLLVDTPEVKHPKKGVEKYGPEASAFTKKMVENAKKIEVEFDKGQRTDQAGEGLAYIYADGKMVNAALVRQGLAKVAYVYKPNNAASFKLELDKYFAKHDKLNIWSLEHHHHHH

SEQ ID NO: 30 - protein, 149 aa, synthetic construct
MGGYAPFKVELDLYFMYPTAAGIAWSQDKAYYVADFVMNGFDTRVWFTPDAEWVMKQTDWETLDEVPAAVFNAFAASEFSDGVVQNVTWVQFPEWQPIVAIQVGKPNMQMKYQILFTPKGEVLRQQNITNAYNTLGASTFLLEHHHHHH

SEQ ID NO: 31 - protein, 166 aa, synthetic construct
MHHHHHHGGSLKITILCVGKGKALDFFKLELDFYFLGPYTKFDLIEVPDEKAPENMSDKEIEQVKEKEGQRILAKIKPQSTVITLEIQGKMLSSEGLAQELNQRMTQGQSDFVFVIGGSNGLHKDVLQRSNYALSFSKMTFPHIMMRVVLAEQVYRAFKIMRGSAS

SEQ ID NO: 32 - protein, 139 aa, synthetic construct
MNLDSFKEELDDYFNESYVAIMDSLFDKLLSKYEVKAPVPSPCFRNICKQMTKLHEKLFDVLPEEQTQMLFLRINASYKLHLKKQLSHLNVINDGGPQNGLVTADVAFYTGNLQALKGLKDLDLNMAEIWELEHHHHHH

SEQ ID NO: 33 - protein, 120 aa, synthetic construct
MHHHHHHGGSGSMSDIRKDLEERFDKIVEALKNSVDAAKASFRDAQFALDGFKLELDVYFIGSSFKEVRNYASEALSKINDLPITNDDKKLASNDVLKLVAEVWKKLEAIMADVYAWNTH

SEQ ID NO: 34 - protein, 104 aa, synthetic construct
MHHHHHHGGSAASAASSEHFEKLHEIFRGLLENLQGGPERLLGSAGSLASFKIELDIYFFQEANETLAEMEEELRYAPLTFRNPMMSKLRNYRKDLAKLHREVR

SEQ ID NO: 35 - protein, 130 aa, synthetic construct
TFVEKYEKQIKHFGMLTRWDDSQKYLSDNVHLVCEETANYLVIWCIDLEVEEKCALMSIVAHQTIVMQFILELAKSLKVDPRACFRQFFTKIKSDALDGFKLELDLYFFIEEVRGRAKLRIELEHHHHHH

SEQ ID NO: 36 - protein, 103 aa, synthetic construct
EQLSFDELTLINLSKVVTVNGHEVPMSIKAFEFLWYLASRENEVISESELAEKVIGDSSLSLFKYELDLYFLEKESFTTYSVTTVEGLGYKFERSLEHHHHHH

SEQ ID NO: 37 - protein, 114 aa, synthetic construct
MHHHHHHGGSEDPFFVIKGEIQKAVNSAQGALQRWTEALQGPSSAGLALFKVELDIYFLSEIEALLEDLDETISIVEANPRKFNLDATELSIRKAFITSTAQIVRDITDQAAAS

SEQ ID NO: 38 - protein, 107 aa, synthetic construct
MHHHHHHGGSSLLISYESDFKTTLEQAKASLAEAPSQPLSLASFKYELDIYFGDELFDLLDQMDVEVNNSIGDASERATYKAKLREWKKTIQSDIKRPLQSLVDSGD

SEQ ID NO: 39 - protein, 105 aa, synthetic construct
MHHHHHHGGSGAAAAALLARAAAARAELAALLAELAAAPARLAAATGALASFKIELDIYFIAAARATLAELEALLAELPPELAAPLRAELAARRAELAALAAEVE

SEQ ID NO: 40 - protein, 105 aa, synthetic construct
MHHHHHHGGSMEKEKELLEKLEKLKEELKKLMEELKKAPAELAAASGELASFKIELDIYFIKEAEETLKKIEELLKSLPEEIKEPLLKELEKKKKELEELKKQIE

SEQ ID NO: 41 - protein, 105 aa, synthetic construct
MHHHHHHGGSGEEEEELLEELEELKEELERKMEELKKAPKRLEEAEGELASFKIELDIYFIEEARRLLEEIEELLEELPEEVREPLLEELEERKKELEELEKKIE

SEQ ID NO: 42 - protein, 105 aa, synthetic construct
MHHHHHHGGSSAAAAARAARLAALLARLDALLAEIRAAPAALAELASGLASFKYELDIYFAEEAEATLAEIRALLAGAPPAVAAPLRAELAAREAELAALRARLE

SEQ ID NO: 43 - protein, 105 aa, synthetic construct
MHHHHHHGGSAKAAEEFLKELKKLKEELKKLLKEIKKAPEEKKKLKSELASFKYELDIYFGEEAEAKLKEIKKMLETAPEEIREPELEELKEIEKELKKLREKLE

SEQ ID NO: 44 - protein, 105 aa, synthetic construct
MHHHHHHGGSSMAMEEFLKELEKLKEEMKKLLEEMKKAPKKKEEIKSELASFKYELDIYFGKELKKTLEKIKELLKTAPKELAEPLLKELEKIEKEIEELKKKLE

SEQ ID NO: 45 - protein, 138 aa, synthetic construct
NLDSFKEELDDYFKEKIVKEFKKFCEETLSKYKVERPTPSKEIKEICAKIREIHEEKVNEWPEEEVRELMLKMWEEFKKELNKRLKELGVTNDGGEKYKIVKEDLDYLESTIRSLPGLENLDLNTESIWDLEHHHHHH

SEQ ID NO: 46 - protein, 138 aa, synthetic construct
NLDSFKEELDDYFKKKIVEEFDKFCEEKLSKYKVETPTPSPVFKEIAAKFKALHDELVGKWPEEDLRELVLELNKEFKKHLNKRLKELGVTNDGGEKYKIVKADLAYLESTIRSLPGLSDLDLNWEEVFKLEHHHHHH

SEQ ID NO: 47 - protein, 138 aa, synthetic construct
NLDSFKEELDDYFKEEIVKEFDEFCKKVLSKYKVEKPTPSKEFKEICSKIKEIHEKLKNVLPEEELKELMKKMNEVLKKHLNERLKELGVTDDGGEKYKIVKEDIDYIVDTIRSLEGLSDLDLNWEEIWKLEHHHHHH

SEQ ID NO: 48 - protein, 138 aa, synthetic construct
NLDSFKEELDDYFKEKIVEEFKKKCEELISKYVVKEPVPSPEIKELCKYIKELHEKIYNKYPEEFVQEIFKKIWDIFIEELSKRLKELGVTNDGGKKYKLVKKDIDYLVSVIKSLPGLSNLDLNWERIWNLEHHHHHH

SEQ ID NO: 49 - protein, 138 aa, synthetic construct
NLDSFKEELDDYFKEKIVKEFEKLCKELISKYEVKKPTPSPEIKKICEYLKKKHEELKDKYPEEFVKEIFKKMWEVFKKELSKQLKKLGVTNDGGEKYKIVKEDLNYLVDVIKSLEGLSDLDLNWEEIWNLEHHHHHH

SEQ ID NO: 50 - protein, 138 aa, synthetic construct
NLDSFKEELDDYFKEKIVAEFKKLCEELLSKYEVKSPVPSPEFKKIAEYLKKLHEELVDKWPLEYVRKLFLKIMKIFKEELKKQLDKLGVTNDGGEKYKQVKADVDYIISVVKSLPGLSDLDLNWEEIWNLEHHHHHH

SEQ ID NO: 51 - protein, 105 aa, synthetic construct
MHHHHHHGGSMKALEEFLKKLEELKKKLKALLEKIEKAPEEKEKEKSSLGKFKYELDLYFLKEAEKVLKEIKEMLKTAPEEIREPELEELKKIEEKIKELKKKLE

SEQ ID NO: 52 - protein, 105 aa, synthetic construct
MHHHHHHGGSAAAAEEFLAKLEELKKELKELLERIKKAPKELEEDKSELEKFKYELDLYFMEKAEETLEKIRKMLETAPEELREPLLEELKKIEKELKELKKKLN

SEQ ID NO: 53 - protein, 105 aa, synthetic construct
MHHHHHHGGSAEEEEAFLRELAALREERDALLERIKKAPEEKKEDKSELEKFKYELDLYFMEELEATLKRIKAMLATAPEELAKPLREELEKVEKEIKELKKELE

SEQ ID NO: 54 - protein, 105 aa, synthetic construct
MHHHHHHGGSSSHMKEFLEELEKLLKELEKLMKRIEKAPKEKEEEKSELEKFKYELDLYFMEEAKKTLKKIKELLATAPKEISEPLLKELKKIEEKLKKLEEELK

SEQ ID NO: 55 - protein, 105 aa, synthetic construct
MHHHHHHGGSSALTEEYIKRLEELKRELKEKLERIKKAPEEKEKEKSKLEEFKYELDIYFMKEAEKLLEEIKELLKTAPPELAEPLLKELEKIEKELEELKKKLY

SEQ ID NO: 56 - protein, 105 aa, synthetic construct
MHHHHHHGGSAAAAAARAAELAALRARLDALLERIEKAPEEKKKDDSELGKFKYELDLYFMEEAEATLAQIRALLATAPAELAAPALAELDAIEKKLAELRKKLK

SEQ ID NO: 57 - protein, 105 aa, synthetic construct
MHHHHHHGGSMADLEEFLKKLEELKEELNKLLERIKKAPEEKKKTKDELEKFKYELDLYFIEEANKVLKEIKKLLEKAPQEISEPMLEELKKIEKEIEELKKKLK

SEQ ID NO: 58 - protein, 107 aa, synthetic construct
MHHHHHHGGSSKLEELKKKFEETFKKAVETLKEAPKLPKELREFKIELDKYFAEKLKELLEKMKEEVEKEVKDKKKKEELLKEIEEKEKEVEEKIEKPLKKLIEESK

SEQ ID NO: 59 - protein, 107 aa, synthetic construct
MHHHHHHGGSSRLEELKKEFEETFEKAVKILKETPTLPPELAKFKIELDKYFAKKLEDLLKEMEEEVEEEVEDEEERERLLEEIKERRKEVEEKIVKPLKELIKKSK

SEQ ID NO: 60 - protein, 107 aa, synthetic construct
MHHHHHHGGSSRLEQLRKEYDETFQKAKETLAEAPNLPPELAKFKIELDKYFAQKLQELLKKMEEAVAEEVADEKERAALLEELAKRRQEVEEKIVKPLKELIEKSK

SEQ ID NO: 61 - protein, 108 aa, synthetic construct
MGSSHHHHHHGSSKDLQFGDLEIYEDKNSIKVKGKEIPMSEESFKKIYLLAKNPNVVLSEEELLKEVTGDKSLELFKFELDIYFLKKAGITEYEIEKVEGKGYKFKKK

SEQ ID NO: 62 - protein, 108 aa, synthetic construct
MGSSHHHHHHGSSPDLVYGGLEINKETKELYVNGKKIPMSEKSFKVLYVLAKRPNEVVSYEELREEVWGCCSLELFKFELDLYFLKEVGFDEIAVEYVPGKGYVFTKR

SEQ ID NO: 63 - protein, 108 aa, synthetic construct
MGSSHHHHHHGSSIDLKFGDLELEIERKKLFVNGKEIPMSEKSFLRIYLLAKRPNEVISLEELLKEVCGCCSLELFKFELDIYFLKEAGYTEYAIEYVPGKGFMFRKT

SEQ ID NO: 64 - protein, 108 aa, synthetic construct
MGSSHHHHHHGSSVDLKYGDLELKEKTKTLLVKGKEIPCSDESFKFLWLLAKNYNKVISLEELLEKVLGSRSLELFKYELDLYFLKEAGYTEYAIVKVPGKGYKFEKV

SEQ ID NO: 65 - protein, 108 aa, synthetic construct
MGSSHHHHHHGSSVDLQYGSLTLVEETKKIYVNGKEIPCSDKSFQLIYLLAKRPNEVISKEELAEEVTGDKSLELFKWELDVYFLKKAGYTEYAIIEVPGKGYKFAKV

SEQ ID NO: 66 - protein, 9 aa, synthetic construct
RSFIEDLLF

SEQ ID NO: 67 - protein, 9 aa, synthetic construct
RSAIEDGLF

SEQ ID NO: 68 - protein, 9 aa, synthetic construct
RSAIEDILF

SEQ ID NO: 69 - protein, 9 aa, synthetic construct
RSAIEDLLF

SEQ ID NO: 70 - protein, 9 aa, synthetic construct
RSGIEDFLF

SEQ ID NO: 71 - protein, 9 aa, synthetic construct
RSHIEDVLF

SEQ ID NO: 72 - protein, 9 aa, synthetic construct
RSIIEDLLF

SEQ ID NO: 73 - protein, 9 aa, synthetic construct
RSLIEDYLF

SEQ ID NO: 74 - protein, 9 aa, synthetic construct
RSQIEDLLF

SEQ ID NO: 75 - protein, 9 aa, synthetic construct
RSTIEDFLF

SEQ ID NO: 76 - protein, 9 aa, synthetic construct
RSVIEDTLF

SEQ ID NO: 77 - protein, 9 aa, synthetic construct
RSYIEDVLF

SEQ ID NO: 78 - protein, 9 aa, synthetic construct
RSYIEDWLF

SEQ ID NO: 79 - protein, 9 aa, synthetic construct
FKEELDKYF

SEQ ID NO: 80 - protein, 9 aa, synthetic construct
FKDELDKYF

SEQ ID NO: 81 - protein, 9 aa, synthetic construct
FKIELDKYF

SEQ ID NO: 82 - protein, 9 aa, synthetic construct
FKLELDKYF

SEQ ID NO: 83 - protein, 9 aa, synthetic construct
FKNELDKYF

SEQ ID NO: 84 - protein, 9 aa, synthetic construct
FKRELDKYF

SEQ ID NO: 85 - protein, 9 aa, synthetic construct
FKSELDKYF

SEQ ID NO: 86 - protein, 9 aa, synthetic construct
FKVELDKYF

SEQ ID NO: 87 - protein, 9 aa, synthetic construct
FKVELDLYF

SEQ ID NO: 88 - protein, 9 aa, synthetic construct
FKYELDKYF

SEQ ID NO: 89 - protein, 14 aa, synthetic construct
ELDSFKEELDKYFK

SEQ ID NO: 90 - protein, 14 aa, synthetic construct
ALASFKIELDIYFI

SEQ ID NO: 91 - protein, 14 aa, synthetic construct
ALDFFKLELDFYFL

SEQ ID NO: 92 - protein, 14 aa, synthetic construct
ALDGFKLELDLYFF

SEQ ID NO: 93 - protein, 14 aa, synthetic construct
ALDGFKLELDVYFI

SEQ ID NO: 94 - protein, 14 aa, synthetic construct
ELAKFKIELDKYFA

SEQ ID NO: 95 - protein, 14 aa, synthetic construct
ELASFKIELDIYFG

SEQ ID NO: 96 - protein, 14 aa, synthetic construct
ELASFKIELDIYFI

SEQ ID NO: 97 - protein, 14 aa, synthetic construct
ELEKFKYELDLYFI

SEQ ID NO: 98 - protein, 14 aa, synthetic construct
ELEKFKYELDLYFM

SEQ ID NO: 99 - protein, 14 aa, synthetic construct
ELGKFKYELDLYFM

SEQ ID NO: 100 - protein, 14 aa, synthetic construct
ELREFKIELDKYFA

SEQ ID NO: 101 - protein, 14 aa, synthetic construct
GLALFKVELDIYFL

SEQ ID NO: 102 - protein, 14 aa, synthetic construct
GLASFKIELDIYFI

SEQ ID NO: 103 - protein, 14 aa, synthetic construct
KLEEFKYELDIYFM

SEQ ID NO: 104 - protein, 14 aa, synthetic construct
NLDSFKEELDDYFK

SEQ ID NO: 105 - protein, 14 aa, synthetic construct
SLASFKIELDIYFF

SEQ ID NO: 106 - protein, 14 aa, synthetic construct
SLASFKYELDIYFG

SEQ ID NO: 107 - protein, 14 aa, synthetic construct
SLELFKFELDIYFL

SEQ ID NO: 108 - protein, 14 aa, synthetic construct
SLELFKFELDLYFL

SEQ ID NO: 109 - protein, 14 aa, synthetic construct
SLELFKWELDVYFL

SEQ ID NO: 110 - protein, 14 aa, synthetic construct
SLELFKYELDLYFL

SEQ ID NO: 111 - protein, 14 aa, synthetic construct
SLGKFKYELDLYFL

SEQ ID NO: 112 - protein, 14 aa, synthetic construct
SLSLFKYELDLYFL

SEQ ID NO: 113 - protein, 130 aa, synthetic construct
QVQLQQSGPGLVKPSETLSLTCTVSGGSISSSSYYWGWIRQPPGKGLEWIGSIYYSGSTYYNPSLKSRVTISVDTSKNQFSLKLSSVTAADTAVYYCARHGPLRFRLGYYDSSGYLFQHWGQGTLVTVSS

SEQ ID NO: 114 - protein, 125 aa, synthetic construct
QVQLVQSGGGLVQPGGSLRLSCATSGFNFRAFAMSWVRQAPGKGLEWAAVMSGTDDGTYYVESVKGRFTIFRDNSENTVYLQMNSLRADDSAIYYCAKGTLGHCSGVDCYYLDYWGRGTLVTVSS

SEQ ID NO: 115 - protein, 118 aa, synthetic construct
QVQLVQSGAEVKKPGASVRLSCKASGDTFTNEYVQWVRQAPGQGLEWMGLINPSGSGTAFARNFQGRVSMTRDTSTRTVYMDLTSLRYEDTAVYYCARMSRAGTFDLWGQGTMVTVSS

SEQ ID NO: 116 - protein, 124 aa, synthetic construct
EVQLVESGGGLVQPGGSLRLSCAASGFTFDNYAMGWVRQPPGKGLEWVSSFSGRGVSTYYADSVKGRFTVSRDSSKNTLFLQMNYLRVEDTAVYYCATYPYDILTGYYGAFDYWGQGALVTVSS

SEQ ID NO: 117 - protein, 131 aa, synthetic construct
QVQLQQWGAGLLKPSETLSLTCSFYGGSFSGYCWSWIRQSPGKGLEWIGEINHSGSTNYNPSLKTRVTILIDTSNNQFSLRLSSVTAADTAVYYCARERAIRRCTSTSCYRVGGVAGLDVWGQGTTVSVSS

SEQ ID NO: 118 - protein, 128 aa, synthetic construct
QVQLVQSGGGLAQPGESLRLSCAASGFTFSSYAMTWVRQAPGKGLEWVSSISGKGENIEYAESVKGRFTISRDNAKNTVDLEMNSLRAEDTATYFCAKHIYGAFIVVPNTLYDALDVWGQATKVTVSS

SEQ ID NO: 119 - protein, 125 aa, synthetic construct
QVQLQESGPGLVKPSETLSLTCAVSGGSVSSDTYYWSWIRQPPGKGLEWIGYIYNSGSTNYNPSLKSRVTISVDTSKSQFSLNLGSVTAADTAVYYCAREGAGTYPPKNDAFDIWGQGTLVTVSS

SEQ ID NO: 120 - protein, 120 aa, synthetic construct
QVQLQESGGGLVKPGGSLRLSCAASGFTFSSYSMNWVRQAPGKGLEWVSSISSSSSYIYYADSVKGRFTISRDNAKNSLYLQMNSLRAEDTAVYYCARGIIDYGDYFDYWGQGTLVTVSS

SEQ ID NO: 121 - protein, 118 aa, synthetic construct
EVQLVESGAEVKKPGASVRVSCKASGFTFTDHYMHWVRQAPGQGLEWMGLINPTGVNTFYAQNFRGRVTMTRDTSTKTDYLEVRSLTSQDTAMYYCARMSRYGAFDIWGQGTMVTVSL

SEQ ID NO: 122 - protein, 118 aa, synthetic construct
EVQLVESGAEVKKPGASVRLSCKASGDTFNNKYVHWVRQAPGQGLEWMGLINPSGSGTAFAQKFQGRVSMTRDTSTRTVYLGLTSLTYEDTAVYYCARMSRAGTFDIWDQGTMVTVSS

SEQ ID NO: 123 - protein, 118 aa, synthetic construct
QVQLVQSGAEVKKPGASVKLSCRASGYPITSHYMHWVRQAPGQGLEWMGIINPSGTGTSFARNFQGRVTMTRDTATRTVYMELSSLKSEDSAVYYCAGGTMGPLFDYWGQGTLVTVSS

SEQ ID NO: 124 - protein, 125 aa, synthetic construct
QVQLVESGGGVVQPGKSLKLSCAASGFTFSTYSMHWVRQAPGKGLEWVAIISYDGRNKYYANSVKGRFTISRDNSKNTLYLEINNLRPQDTAVYYCATDASNVEVPGKFAPLDKWGQGTLVTVSS

SEQ ID NO: 125 - protein, 122 aa, synthetic construct
QVQLVESGGGVVQPGRSLRLSCAASGFTLSSHGIHWVRQAPGKGLEWVAVTSFDGRNKKFGDSVKGRFTISRDNSKNTVYLQMNSLRTEDTAVYYCAKDWGDDYSRWYFDLWGRGTLVTVSS

SEQ ID NO: 126 - protein, 108 aa, synthetic construct
DIQLTQSPSTLSASVGDRVTITCRASQSISSWLAWYQQKPGKAPKLLIYDASSLESGVPSRFSGSGSGTEFTLTISSLQPDDFATYYCQQYNSYSRWTFGQGTKVEIK

SEQ ID NO: 127 - protein, 106 aa, synthetic construct
DIVLTQSPATLSLSPGERATLSCRASQSVGTYVAWYQHKVGQAPRLLIYDASTRATDIPARFSGSGSGTDFTLTITSLEPEDVAIYYCQQRVNLVTFGGGTKLEIK

SEQ ID NO: 128 - protein, 110 aa, synthetic construct
EIVLTQSPDTLSLSLGERATLFCRASETIDSRYLAWYQQKPGRAPRLLMSGTSVRATGIPDRFSGSGSGTDFTLTITRLEPEDFAVYYCQQYGSSPPRYTFGQGTKLAMK

SEQ ID NO: 129 - protein, 106 aa, synthetic construct
DIQLTQSPSTLSASVGDRVTITCRASQTIDTWLAWYQQKPGKAPKLLIYGASSLQSGVPSRFSASGSGTEFTLTISSLQPDDFAIYYCQQYKDYSTFGQGSKVEFM

SEQ ID NO: 130 - protein, 107 aa, synthetic construct
DIVMTQSPSSLSASVGDRVTITCQASRDIYKYLNWYQQKPGQAPKLLISDASNLETGVPSRFSGSGSGTDFTFTISSLQPEDIAAYYCQQYDNPLITFGQGTRLEIK

SEQ ID NO: 131 - protein, 108 aa, synthetic construct
EIVLTQSPGTLSLSPGERATLSCRASQSVSRNYLAWYQQKPGQAPRILVYDASNRAIGIPDRFSGSGSGTDFTLTISRLEPEDFAVYYCQQYGSSPGTFGQGTRLEIK

SEQ ID NO: 132 - protein, 113 aa, synthetic construct
EIVLTQSPDSLAVSLGERATINCKSSQSVLFSSDNKNYLAWYQQKPGQPPKLLIYWASTRESGVPDRFSGSGSGTDFTLTISSLQAEDVAVYYCQQYYNSPRTFGQGTKVEIK

SEQ ID NO: 133 - protein, 110 aa, synthetic construct
EIVLTQSPDTLSLSPGERATVSCRASQSVGAGYVAWYQQRPGQPPRLLIYGASVRATGIPDRFSGSGSGTDFSLTINRVEPEDFAVYYCQNYASSPPRYTFGQGTKLEIK

SEQ ID NO: 134 - protein, 110 aa, synthetic construct
EIVLTQSPGTLSLSPGERATLSCRASQSVGSGYLAWYQQKPGQAPRLLIYGASVRATGIPDRFSGSGSGTDFSLTINRVEPEDFAMYYCQNYGSSPPRYTFGQGTKLEIK

SEQ ID NO: 135 - protein, 110 aa, synthetic construct
EIVLTQSPGTLSLSPGDRATLSCRASENVGTSYLAWYQQKPGQAPRLLISGTSSRATGVPDRFSGSGSGRDFTLTISRLEPEDFAVYYCQQYGSSPPRYTFGQGTKLDMK

SEQ ID NO: 136 - protein, 110 aa, synthetic construct
EIVLTQSPGTLSLSPGERATLSCRASQSVRRNFLAWYQQKPGQAPRLLIYEASTRATGIPDRFSGSGSGTDFTLTISRLEPEDFAVYYCQQYDSSPPGVTFGPGTKVDIR

SEQ ID NO: 137 - protein, 108 aa, synthetic construct
EIVMTQSPATLSVSPGERATLSCRASQSVSSDLAWYQQKPGQAPRLLIYGATTRATGVPARFSGSGSGAEFTLTISSLQSGDFAVYYCQQYNDWPLITFGQGTRLEIK

SEQ ID NO: 138 - protein, 107 aa, synthetic construct
DIQMTQSPSSLSASVGDRVTITCQASHDITKYLNWYQQKPGRAPKLLIYDASNLKTGVPSRFSGSGSGTDFTFTITSLQPEDLATYYCQQSDVLPITFGQGTRLEIK

SEQ ID NO: 139 - protein, 111 aa, synthetic construct
GSSHHHHHHGSSDKNLVFGSLKINVKTKEVYVNGKKVPMSDESFLFLYILAKNYNKVISTKELLEKVYGPNAKGKLKLFKCELDLYFLEEAGYTEYKVEYVKGKGYKFVKN

SEQ ID NO: 140 - protein, 112 aa, synthetic construct
MGSSHHHHHHGSSCKDLVFGSLKLNCKTKELFVNGKKVPVSEESFKFLYILASNYNKVISEEELLKKVFGEDAKGKLKLFKCELDLYFLEEAGYTEYKIVYVPGKGYKFTKN

SEQ ID NO: 141 - protein, 112 aa, synthetic construct
MGSSHHHHHHGSSYKNLKFGKLELNEKTKEVLVNGKKVKISEESFKFLYLLAKHYNEVISEEELLKEVFGENAKGKLRLFKLELDLYFLKEAGYTEYEIEYVPGKGYKFVKN

SEQ ID NO: 142 - protein, 111 aa, synthetic construct
GSSHHHHHHGSSGKNLKFGNMYLDVKTKKLYVNGKEVKISEESFNFLYILAKNFNKVISYDELLKKVYGPNAKGRLKLFKCELDLYFLKEAGYTEYEIVEVPGKGYKFTKK

SEQ ID NO: 143 - protein, 111 aa, synthetic construct
GSSHHHHHHGSSSKNLTYGDLVLKEDTKEVYVKGKKVPVSEKSFKFLYILAKNYNKVISEEELLKKVYGENAKGKLKLFKCELDLYFLKEAGYTEYSIVYVKGKGYKFVKN

SEQ ID NO: 144 - protein, 111 aa, synthetic construct
GSSHHHHHHGSSYKNLKFGDLTLDVKTKKVYVNGKLVPVSEESFKFLYILASNYNKVISEEELLKKVYGPNAKGKLKLFKCELDLYFLKEAGYTEYKIVYVKGKGYKFEKT

SEQ ID NO: 145 - protein, 111 aa, synthetic construct
GSSHHHHHHGSSYKDLKFGDLVLNEKTKKVYVNGKEVPVSEKSYKFLYLLASNYNKVISYEELLKKVYGENAKGKLLLFKEELDLYFLKEAGYTEYEIVEVPGKGYKFEKN

SEQ ID NO: 146 - protein, 111 aa, synthetic construct
GSSHHHHHHGSSSKDLKYGDLVLKEDTKKVYVKGKEIPVSPESFKFLYILAKNYGKVVSEEELLREVFGPNAKGSLKLFKCELDLYFLEEAGYTENKVVYVPGKGYKFVKN

SEQ ID NO: 147 - protein, 111 aa, synthetic construct
GSSHHHHHHGSSNTDLTFGDLKLDTKTKKLYVKGKEVPVSEESFKFLYLLASNYNKVISEKELLEKVFGPKAKGKLLLFKCELDLYFLKEAGYTEYEIVYVPGKGYMFKKN

SEQ ID NO: 148 - protein, 111 aa, synthetic construct
GSSHHHHHHGSSDKDLVFGNLKLNKKTKKVYVNGKEVPVSEESFKFLYLLASNYGKVISYEELLKKVYGENAKGELKLFKCELDLYFLKEAGYTEYEIVRVPGKGYKFEKT

SEQ ID NO: 149 - protein, 122 aa, synthetic construct
GSSHHHHHHGSSGKIEEILKKLKEKFEELIEEAKEGKEEWLSRLPECKDSLCGFKGELDLYFLEGYKKEIEEYAKKAIEEVEKLPISEEEKEKAKEKIKQLVKEAKEKLEKLKEEWLELRKK

SEQ ID NO: 150 - protein, 122 aa, synthetic construct
GSSHHHHHHGSSMTIEEIIKKLEEEAKKIIEELKKHVEKTKKLLKENKGKLKEFKYELDFYFIEKAKKKLEELKKEALEEVEKLPISEEEKEKAKKKIEELYKKYLKEIEKIEKEIKKEKEK

SEQ ID NO: 151 - protein, 122 aa, synthetic construct
GSSHHHHHHGSSMSIEEILEELKKKYEEIIKKAKEHIEKTKKKLKEEKGSLKEFKYELDKYFLEESYKKLEELYKEALKKVEELPISEEEKKKAKEEIKKLYEEYKKKLEELEKELKEEKEK

SEQ ID NO: 152 - protein, 122 aa, synthetic construct
GSSHHHHHHGSSMTVEEILAELEKEYKKIIEELKKHIEESKKKLKKDKGSLPEFKYELDEYFIEEAKKKLEELTKKALEKIEKLPISEEEKKKAKEKVKKIYEEYKKKIEELKEELEKDKKK

SEQ ID NO: 153 - protein, 122 aa, synthetic construct
GSSHHHHHHGSSMSIEDILADFKAKSDAIIKSLKEHIENTLKLLEENKDSLPEFKFELDRYFIAQAKADLDALTAEALAQVDALPISEEQKAAAKAQIKALAAAAQQKIDELKEELEKYQKK

SEQ ID NO: 154 - protein, 122 aa, synthetic construct
GSSHHHHHHGSSASVEDILNTLKAALDKIINDLKEHYNTTKEILKKTKGKLPTFKYELDEYFIEEAKKQIEELTKEALKEIDQLPISEEEKKKAKEQVQKLADEYKKKIEAIKKELEKDKKK

SEQ ID NO: 155 - protein, 122 aa, synthetic construct
GSSHHHHHHGSSMTPEEILKELEKEFEKIIEDLRAFIEETKKLLKEKKGELPEFKFELDKYFIKKAKKQLEEKKKEALEKIEKLPISEEEKEKLKKEVEKKYEEALKKIEELEKEVEEFQKK

SEQ ID NO: 156 - protein, 122 aa, synthetic construct
GSSHHHHHHGSSMTVQDILDEFKKAAEKIIKDLKEHIESTKRLLQKDKGSLPDFKYELDLYFIKNAKKKLEEQTQKALKAVDELPISEEEKKQAKEQIKKLSEEYQAKIEELEAEVLEARKK

SEQ ID NO: 157 - protein, 122 aa, synthetic construct
GSSHHHHHHGSSKSIEEIKKELEKKYEEIIEKLKEHIKESKKLKEKEKGKLDSFKYELDEYFIKKAEEEIEKLTKEALKEIEKLPISEEEKEKAKKEIEKLKEEVKKKIEELKRKLEEEKKK

SEQ ID NO: 158 - protein, 122 aa, synthetic construct
GSSHHHHHHGSSMTGEEIIEELKKKYKEIIESLKKKFKETVEKLKKNKENLKDFKFELDRYFIEKAKKEIEKLTEEAKKKIKELPISEEEKKELIEKVDELKKEVLKEIEKIEKELEEYKEK

SEQ ID NO: 159 - protein, 14 aa, synthetic construct
KLKLFKCELDLYFL

SEQ ID NO: 160 - protein, 14 aa, synthetic construct
KLKLFKCELDLYFL

SEQ ID NO: 161 - protein, 14 aa, synthetic construct
KLRLFKLELDLYFL

SEQ ID NO: 162 - protein, 14 aa, synthetic construct
RLKLFKCELDLYFL

SEQ ID NO: 163 - protein, 14 aa, synthetic construct
KLKLFKCELDLYFL

SEQ ID NO: 164 - protein, 14 aa, synthetic construct
KLKLFKCELDLYFL

SEQ ID NO: 165 - protein, 14 aa, synthetic construct
KLLLFKEELDLYFL

SEQ ID NO: 166 - protein, 14 aa, synthetic construct
SLKLFKCELDLYFL

SEQ ID NO: 167 - protein, 14 aa, synthetic construct
KLLLFKCELDLYFL

SEQ ID NO: 168 - protein, 14 aa, synthetic construct
ELKLFKCELDLYFL

SEQ ID NO: 169 - protein, 14 aa, synthetic construct
SLCGFKGELDLYFL

SEQ ID NO: 170 - protein, 14 aa, synthetic construct
KLKEFKYELDFYFI

SEQ ID NO: 171 - protein, 14 aa, synthetic construct
SLKEFKYELDKYFL

SEQ ID NO: 172 - protein, 14 aa, synthetic construct
SLPEFKYELDEYFI

SEQ ID NO: 173 - protein, 14 aa, synthetic construct
SLPEFKFELDRYFI

SEQ ID NO: 174 - protein, 14 aa, synthetic construct
KLPTFKYELDEYFI

SEQ ID NO: 175 - protein, 14 aa, synthetic construct
ELPEFKFELDKYFI

SEQ ID NO: 176 - protein, 14 aa, synthetic construct
SLPDFKYELDLYFI

SEQ ID NO: 177 - protein, 14 aa, synthetic construct
KLDSFKYELDEYFI

SEQ ID NO: 178 - protein, 14 aa, synthetic construct
NLKDFKFELDRYFI

SEQ ID NO: 179 - protein, 9 aa, synthetic construct
  VARIANT 3: note = Any amino acid
  VARIANT 7: note = Any amino acid
RSXIEDXLF

SEQ ID NO: 180 - protein, 9 aa, synthetic construct
  VARIANT 3: note = Any amino acid
  VARIANT 7: note = Any amino acid
FKXELDXYF

SEQ ID NO: 181 - protein, 118 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARMSRYGAFDIWGQGIMVTVSS

SEQ ID NO: 182 - protein, 110 aa, synthetic construct
EIVLTQSPGTLSLSPGERATLSCRASQSVSSSYLAWYQQKPGQAFRLLIYGASSRATGIPDRFSGSGSGTDFTLTISRLEPEDFAVYYCQQYGSSPPRYTFGQGTKLEIK

SEQ ID NO: 183 - protein, 15 aa, Severe acute respiratory syndrome coronavirus 2
SFKEELDKYFKNHTS

SEQ ID NO: 184 - protein, 15 aa, Middle East respiratory syndrome-related coronavirus
DFQDELDEFFKNVST

SEQ ID NO: 185 - protein, 12 aa, synthetic construct
  VARIANT 2..3: note = Any amino acid
  VARIANT 6: note = Any amino acid
  VARIANT 10: note = Any amino acid
LXXFKXELDXYF

SEQ ID NO: 186 - protein, 6 aa, synthetic construct
HHHHHH

(19) United States
(12) Patent Application Publication
(10) Pub. No.: US 2024/0285753 A1
(43) Pub. Date: Aug. 29, 2024

(54) EPITOPE-SCAFFOLD IMMUNOGENS FOR PANCORONAVIRUS VACCINES

(71) Applicant: Duke University, Durham, NC (US)

(72) Inventors: Mihai Azoitei, Durham, NC (US); Daniel Marston, Durham, NC (US); Anyway Brenda Kapingidza, Durham, NC (US); Barton F. Haynes, Durham, NC (US)

(21) Appl. No.: 18/417,663

(22) Filed: Jan. 19, 2024

Related U.S. Application Data
(60) Provisional application No. 63/480,841, filed on Jan. 20, 2023.
(60) Provisional application No. 63/446,104, filed on Feb. 16, 2023.

(51) Int. Cl. A61K 39/215; A61K 39/00; A61P 37/04; C07K 14/005; C07K 14/195; C07K 14/21; C07K 14/245; C07K 14/28; C07K 14/31; C07K 14/395; C07K 14/405; C07K 14/415; C07K 14/47; C07K 16/10

(52) U.S. Cl. CPC A61K 39/215; A61P 37/04; C07K 14/005; C07K 14/195; C07K 14/21; C07K 14/245; C07K 14/28; C07K 14/31; C07K 14/395; C07K 14/405; C07K 14/415; C07K 14/47; C07K 16/1003; A61K 2039/575; C07K 2318/20

(57) ABSTRACT

Disclosed are immunogens for stimulating an immune response to coronaviruses. The immunogens can be non-receptor binding domain (RBD) epitopes. The immunogens can be grafted into a scaffold polypeptide to produce an epitope scaffold. Antibodies induced by the immunogens are disclosed.

This application claims the benefit of and priority to U.S. Provisional Application No. 63/480,841, filed on Jan. 20, 2023, and U.S. Provisional Application No. 63/446,104, filed on Feb. 16, 2023, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### GOVERNMENT INTERESTS

This invention was made with government support under Grant No. P01AI158571 and Grant No. R01AI155804, awarded by the National Institute of Allergy & Infectious Diseases (NIH/NIAID). The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

Aspects of the invention are drawn to computationally-designed protein immunogens that can selectively amplify immunity against the coronavirus antigens as potential candidates for pancoronavirus vaccines.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### BACKGROUND OF THE INVENTION

COVID-19 has been associated with significant morbidity and mortality, representing the largest public health crisis of the present century. Current SARS-CoV-2 vaccines have been unable to keep pace with the rapid evolution of highly divergent viral variants, posing a considerable challenge to mitigating the ongoing pandemic. There is a need to develop broadly protective pancoronavirus vaccines.

### SUMMARY OF THE INVENTION

Disclosed are immunogenic compositions comprising, consisting essentially of, or consisting of an epitope that can stimulate an immune response to an S2 region of a human coronavirus spike protein. The immune response stimulated by the epitope can react with a genus Alphacoronavirus or Betacoronavirus. In some embodiments, the immune response can react with a SARS-Cov-1 or SARS-Cov-2 coronavirus. The epitope can stimulate an immune response to a fusion protein epitope or an S2 stem helix epitope from these viruses. In some embodiments, the epitope is grafted or transplanted into a protein scaffold to form an epitope scaffold (ES). In some embodiments, the epitope scaffold is produced using side chain grafting or backbone grafting techniques. In some embodiments, a root-mean-square-deviation (RMSD) of the epitope scaffold compared to a corresponding protein scaffold that does not contain the grafted or transplanted epitope is less than about 12 Å. In some embodiments, the RMSD is less than about 1.0 Å. In some embodiments, the immunogenic composition does not contain a receptor binding domain (RBD) from a coronavirus spike protein.

Disclosed are immunogenic compositions comprising, consisting essentially of, or consisting of epitopes that can stimulate an immune response to a fusion peptide or S2 stem helix epitope from a human coronavirus spike protein. The epitopes can be grafted into a scaffold protein. In some embodiments, the scaffold protein can be between about 40 and 400 amino acids in length. In some embodiments, the immunogenic composition does not stimulate an immune response to a receptor binding domain (RBD) from a coronavirus spike protein.

Disclosed are amino acid epitopes grafted into a scaffold protein (to produce an epitope scaffold) that can be bound by antibodies stimulated by a CoV-2 spike protein. The amino acid epitopes can comprise, consist essentially of, or consist of epitopes that can be bound by antibodies that bind to a fusion peptide amino acid epitope or a S2 stem helix amino acid epitope from CoV-2.

Disclosed are epitope scaffolds comprising, consisting essentially of, or consisting of amino acid epitopes to which antibodies directed to a fusion peptide or S2 stem helix epitope from a human coronavirus spike protein can bind. Immunogenic compositions comprising, consisting essentially of, or consisting of one or more of the epitope scaffolds are disclosed. In some embodiments, the epitope scaffold and the immunogenic composition do not contain an epitope that can stimulate an immune response to a receptor binding domain (RBD) from a coronavirus spike protein.

Disclosed are epitope scaffolds having an amino acid sequence SEQ ID NOs: 1-65 and SEQ ID NOs: 139-158 or a peptide having 90% identify thereto.

Disclosed are methods for stimulating an immune response in an individual or subject by administering an epitope scaffold, or immunogenic composition containing the epitope scaffold, to an individual.

Disclosed are methods for making an epitope scaffold.

Disclosed are antibodies isolated from sera of subjects previously exposed to a betacoronavirus. The isolated antibodies described herein can bind to the epitopes and/or to the epitope scaffolds described herein. In some embodiments, the antibodies can react with a genus Alphacoronavirus or Betacoronavirus. In some embodiments, the antibodies can react with a SARS-Cov-1 or SARS-Cov-2 coronavirus. In some embodiments, the antibodies can react to a fusion protein epitope or an S2 stem helix epitope from these viruses.

Disclosed herein are nanoparticles displaying any of the epitope scaffolds an amino acid sequence SEQ ID NOs: 1-65 and SEQ ID NOs: 139-158 or a peptide having 90% identify thereto. In some embodiments, the nanoparticles described herein can elicit an immune response in a subject inoculated with the nanoparticle.

### BRIEF DESCRIPTION OF THE DRAWINGS

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1. illustrates example designs of epitope scaffolds that bind to broadly cross-reactive antibodies against the fusion peptide. (A) Left: Structure of the pre-fusion spike trimer (individual monomers: blue, violet and pale green; glycans: grey; PDBid:6xr8), with the RBD (green), fusion peptide (red) and stem helix (orange) domains highlighted. Middle: Zoom of spike monomer with fusion peptide highlighted (red) showing key residues engaged by antibodies. Right: Structure of fusion peptide (red) bound to DH1058 mAb (grey) (PDBid:7tow). (B) Example computational models of DH1058 (grey) bound to ESs FP-2, FP-10 and FP-15 (green) with the grafted epitope shown in red sticks. (C) Example binding affinities of FP ESs and the synthetic fusion peptide to DH1058 and DH1294 mAbs as determined by SPR. nt=not tested. (D) Example results of binding of epitope scaffolds (ES) to diverse FP-targeting mAbs by ELISA. (E) Example ELISA binding results of DH1058 mAb to synthetic fusion peptides and representative ESs that contain alanine mutations at epitope residues critical for antibody recognition. Numbers in brackets indicate the equivalent position of the mutated epitope residues on spike. (F) Example crystal structure of DH1058 mAb (grey) in complex with FP-15 (salmon) overlaid with the computational model of the ESs (green). Epitope residues are shown in sticks (model: red; crystal structure: salmon).

FIG. 2. illustrates an example design of epitope scaffolds that bind to S2P6-like antibodies against the stem helix. (A) An example conformation of the stem helix epitope (orange) on the pre-fusion trimer, bound by S2P6 and CC40.8mAbs (PDBids:7rnj and 7sjs), and displayed on representative epitope scaffolds S2hlx-4 and -7. Key epitope residues are labeled and shown in sticks. (B) Example binding affinities of S2hlx ESs to S2P6, DH1057.1 and DH1057UCA mAbs. nt=not tested. (C) Representative single-cycle binding SPR traces used to determine the dissociation constants in (B). (D) example effects on S2P6 and DH1057 mAbs binding to epitope mutations in synthesized stem helix peptides (1140-1164) and representative S2-hlx ESs. (E) Example crystal structure of the DH1057.1 mAb (grey) bound to ES S2hlx-7 (orange) with the epitope in pink sticks. Inset shows details of the epitope/mAb interaction, with epitope residue K1149 highlighted. (F) Example comparison of the S2hlx-7 conformation in the computational model (pink) and the crystal structure (blue), both overall and in the epitope region.

FIG. 3. illustrates example designs of epitope scaffolds that engage the two major classes of antibodies against the stem helix. (A) Example overview of the computational design process to graft the antibody-bound CC40.8 epitope onto candidate scaffolds. ESs were designed by transplanting the epitope backbone with Rosetta, validated with Alphafold2 and optimized for expression with ProteinMPNN. (B) Top: Example computational models of CC40.8 mAb (gray and white) bound to ESs (purple) from the S2hlx-Ex2, S2hlx-Ex4, and S2hlx-Ex6 families of designs. The epitope is highlighted in orange sticks. Residues designed by ProteinMPNN and that can differ between designs from the same family are shown in pink sticks. Bottom: Example overlay of the grafted epitope conformation (orange) and the parent scaffold structure it replaced (purple). (C) Sequence identity between S2hlx-Ex2 family of ESs designed by ProteinMPNN. (D) Example binding of S2hlx-Ex ESs to S2P6, CC40.8 and DH1057.1 mAbs. ESs are grouped by the parent backbone grafting design they are based on: S2hlx-Ex2 (upper), S2hlx-Ex4 (middle), and S2hlx-Ex6 (lower). (E) Example dissociation constants of S2hlx-Ex2 based ESs to S2P6, DH1057 and CC40.8 mAbs and their precursors. nb=not binding. (F) Example ELISA binding to CC40.8, S2P6 and DH1057 mAbs by ESs based on different parent scaffolds and that contain alanine mutations at indicated epitope residues, with their corresponding position on spike shown in brackets. (G) Left: example alignment of the S2hlx-Ex19 parent scaffold (cyan) with the crystal structure of the unbound epitope scaffold (pink, with the epitope in magenta sticks). Residues Tyr62 and Phe66 that stabilize the grafted epitope conformation are highlighted. Right: Comparison of the stem helix epitope conformation in the pre-fusion spike (grey), induced by CC40.8 binding (orange) and displayed on S2hlx-Ex19 (magenta).

FIG. 4. illustrates example reactivity of engineered epitope scaffolds with sera from individuals with pre-existing immune responses to SARS-CoV-2 spike. (A) Left: Example ELISA binding of sera or plasma from patients with pre-existing SARS-CoV-2 immunity acquired by vaccination (red), infection (green), or vaccination followed by infection (purple) to recombinant WA-2 spike and RBD or to synthetic peptides encoding the fusion peptide (residues 808-833) or the stem helix peptide (1140-1164). Middle and right: Example binding of the same samples in (A) to select stem helix peptide and fusion peptide epitope scaffolds respectively. (B) Example comparison between sera binding to the synthesized fusion peptide with FP-15 ES, and the parent scaffold from which FP-15 was derived and that lacks the grafted epitope (FP-15 PS). (C) Example comparison between sera binding to the synthesized stem helix peptide with S2hlx-Ex19 ES, and with versions of this design where the epitope is mutated to reduce binding to either CC40.8 class antibodies (L1145A), to both CC40.8 and S2P6 classes of antibodies (F1148A), or where the epitope is replaced with the sequence from the parent scaffold it replaced (PShlx). Single lines represent measurements for individual samples in the respective cohort. For all graphs *=p<0.05 and **=p<0.01. (D) Example bivariate plot of labeling of memory B cells from Patient 25 with S2hlx-Ex19 conjugated to VB15 (x-axis) and AF647 (y-axis). A third bait comprising S2hlx-Ex19 PShlx conjugated to BV421 was used to exclude scaffold-specific memory B cells. The overall frequency of stem helix epitope-specific cells was 0.033% of memory B cells. (E) Example gating of stem helix epitope-specific memory B cells from Patient 27, comprising 0.047% of total memory B cells.

FIG. 5. illustrates FP antibodies with broad recognition against diverse coronaviruses that target a conserved epitope on the fusion peptide domain of SARS-CoV-2. (A) DH1058 (grey). (B) VN01H1 (blue). (C) Ab Cov44-62 (green), and (D) Ab Cov44-79 (orange) bound to their epitope on FP (magenta). Epitopes are centered on residues 813-824 with key contacts made with virus residues R815, E819 and F823.

FIG. 6. illustrates an example workflow for side chain grafting design of fusion peptide epitope scaffolds. Using the crystal structure of antibody DH1058 (lightpurple and cyan) bound to the FP epitope (yellow), candidate scaffolds (magenta) were queried computationally to identify proteins with exposed backbone regions that closely matched the structure of the FP fragment 815-823 (<0.5 Å RMS). On proteins that have regions with high structural mimicry to the DH1058-bound epitope, the epitope sequence (yellow) replaced the native scaffold to generate an ES; additional mutations were introduced into the scaffold to accommodate the grafted epitope, to prevent clashes with the DH1058 mAb in the modeled antibody-ES complex, and to revert any unnecessary scaffold mutations introduced at the automated computational design stage.

FIG. 7. illustrates an initial binding screen of designed FP epitope scaffolds to DH1058 mAb. ELISA binding of DH1058 mAb to immobilized FP ESs and a synthetic FP peptide (SARS-CoV-2 residues 808-833).

FIG. 8. illustrates example Surface Plasmon Resonance binding curves to determine the dissociation constants of FP ESs for DH1058 and DH1294 mAbs. Acquired data is shown in black and the curve fit is in red. Data is representative of at least two independent experiments.

FIG. 9. illustrates example FP ES binding to diverse antibodies that target the fusion peptide. ELISA binding of mAbs DH1058, DH1294, VN01H1, Cov44-62, and Cov44-79 to FP ESs and to a synthetic FP peptide (SARS-CoV-2 residues 808-833).

FIG. 10. illustrates example S2hlx ESs binding to S2P6 and DH1057.1 mAbs, but not to CC40.8 mAb. ELISA binding of mAbs S2P6, DH1057.1 and CC40.8 to S2hlx ESs measured by ELISA. Binding was compared to that of synthetics S2 stem helix peptides derived from SARS-CoV2 (¹¹⁴⁷SFKEELDKYFKNHTS¹¹⁶¹; SEQ ID NO: 183) and MERS (¹²³⁰DFQDELDEFFKNVST¹²⁴⁴; SEQ ID NO: 184). The synthetic peptide did not contain residue L1145 (SARS-CoV-2 numbering) that is critical for epitope binding by CC40.8.

FIG. 11. illustrates example Surface Plasmon Resonance curves of S2hlx ESs binding to S2P6, S2P6iGL, DH1057.1 and DH1057UCA mAbs. Acquired data is shown in black and the curve fit is in red.

FIG. 12. illustrates example ELISA binding of S2P6iGL mAb to S2hlx ESs. ESs binding was compared to that of a synthetic S2 stem helix peptide derived from SARS-CoV-2 (residues 1140-1164).

FIG. 13. illustrates example binding of S2hlx ESs to target mAb is mediated by the grafted epitope residues. ELISA binding of S2P6 and DH1057 mAbs to S2hlx ESs and to the parent scaffolds (PS) that served as templates for epitope grafting.

FIG. 14 illustrates example recombinant expression and S2P6 mAb binding of S2hlx-Ex ES designs generated by backbone grafting. (A) Coomassie-stained SDS-PAGE gel showed limited expression of initial S2hlx-Ex ESs. (B) Binding of mAb S2P6 to immobilized S2hlx-Ex ESs measured by ELISA.

FIG. 15 illustrates that example tagged S2hlx-Ex ES designs show improved expression and antibody binding. (A) Coomassie stained SDS-PAGE gel showing the expression of the initial S2hlx-Ex4 design and of different versions fused to the tags highlighted in magenta. (B) Coomassie stained SDS-PAGE gel showing expression of Trx-tagged versions of S2hlx-Ex2, Ex4, and Ex6. * marks expected molecular weight bands. (C) Binding of S2P6 and CC40.8 mAbs to tagged S2hlx-Ex ESs measured by ELISA.

FIG. 16 illustrates example binding of ES designed on structural homologs of the S2hlx-Ex4 parent scaffold. (A) Example structures of the S2hlx-Ex4 ES and of two related designs, S2hlx-Ex7 and S2hlx-Ex8, based on structurally homologous parent scaffolds (PDBids: 2qyw, 1lvf, and 3onj). The grafted epitope is shown in red. (B) Example ELISA binding of antibodies S2P6, DH1057, and CC40.8 to tagged ESs from (A).

FIG. 17 illustrates example expression of S2hlx-Ex2 and related successful MPNN designs. Coomassie stained SDS-PAGE gel showing the expression of the initial S2hlx-Ex2 design and of different MPNN versions.

FIG. 18 illustrates example surface Plasmon Resonance curves of S2hlx-Ex2 derived ESs binding to S2P6, S2P6iGL, DH1057.1, DH1057 UCA, CC40.8 and CC40.8iGL mAbs. Binding kinetics of S2hlx-Ex2 derived ESs to S2P6, DH1057, and CC40.8 and their germline variants as measured by Surface Plasmon Resonance (SPR).

FIG. 19 illustrates example binding affinities of S2hlx-Ex4 and Ex6 derived ESs to S2P6, DH1057.1, and CC40.8 mAbs. Binding kinetics of S2hlx-Ex4 and Ex6 derived ESs to S2P6, DH1057.1, and CC40.8 as measured by Surface Plasmon Resonance (SPR).

FIG. 20 illustrates example binding of S2hlx-Ex-15, -Ex19, and synthetic stem helix peptide to mature antibodies and their inferred precursors by ELISA. Stem Helix Peptide data was generated with the peptide absorbed onto the plate, while in the Stem Helix Peptide measurements the peptide was captured on a surface pre-coated with streptavidin. The binding of S2hlx-Ex15 was not measured by ELISA to CC40.8iGL here, but the dissociation constant for this interaction (K_{D}=6.4 nM) was determined by SPR (FIG. 3).

FIG. 21 illustrates example reactivity of designed ESs to sera from individuals with pre-existing immunity to the SARS-CoV-2 spike. (A) Example binding of stem helix peptide and select S2hlx and S2hlx-Ex epitope scaffolds. (B) Example binding of stem helix peptide, S2hlx-Ex15 and S2hlx-Ex15 epitope mutants that reduce binding to either the CC40.8-class of mAbs (LA) or to both the CC40.8 and S2P6 classes of mAbs (FA). (C) Example binding to FP ESs and to the parent scaffolds they were derived from and that lack the grafted epitope. Lines represent individual measurements for each sample in the group.

FIG. 22 illustrates examples of isolation of stem helix epitope-specific memory B cells from vaccinated then infected subjects with stem helix scaffolds. (A) Example ELISA binding of sera from indicated study participants with pre-existing SARS-CoV-2 immunity acquired by vaccination followed by infection to recombinant WA-2 spike and RBD or to synthetic peptides encoding the fusion peptide (residues 808-833) or the stem helix (1140-1164). (B) Example comparison between sera binding to the synthesized stem helix peptide with S2hlx-Ex19 ES, and with versions of this design where the epitope is mutated to reduce binding to either CC40.8 class antibodies (L1145A), to both CC40.8 and S2P6 classes of antibodies (F1148A), or where the epitope is replaced with the sequence from the parent scaffold it replaced (PShlx). Single lines represent measurements for individual samples. (C) Example flow cytometric gating strategy for the isolation of memory B cells from PBMCs of subjects.

FIG. 23 provides design characteristics and initial experimental screening data for fusion peptide (FP) epitope scaffolds tested experimentally. Fusion peptide epitope scaffolds originate from diverse parent proteins. ESs were expressed recombinantly in E. Coli and tested for binding to FP mAbs by ELISA (+++ denotes high affinity; ++ moderate affinity; + low affinity). SC=side chain; BB=backbone; AF=AlphaFold2.

FIG. 24 provides design characteristics and initial experimental screening data for S2 helix peptide (S2hlx) epitope scaffolds tested experimentally. S2hlx epitope scaffolds originate from diverse parent proteins. ESs were expressed recombinantly in E. Coli and tested for binding to stem helix mAbs by ELISA (+++ denotes high affinity; ++ moderate affinity; + low affinity). SC=side chain; BB=backbone; AF=AlphaFold2.

FIG. 25 provides design characteristics and initial experimental screening data for S2 helix extended (S2hlx-Ex) epitope scaffolds tested experimentally. S2hlx-Ex epitope scaffolds originate from diverse parent proteins. ESs were expressed recombinantly in E. Coli and tested for binding to stem helix mAbs by ELISA (+++ denotes high affinity; ++ moderate affinity; + low affinity). SC=side chain; BB=backbone; AF=AlphaFold2.

FIG. 26 illustrates example design and production of epitope-scaffolds. (A) Major vaccine targets on the Spike protein, and close-up of S2 stem helix epitope highlighting residues that have been previously reported as important for antibody recognition. (B-C) Example computational models of representative epitope-scaffold designs displaying the epitopes of mAbs S2P6 and CC40.8. (D) Example SDS-PAGE analysis of selected immunogens expressed with various fusion tags (NT=no tag). The thioredoxin (Trx) tag resulted in the greatest improvement in protein yields relative to the untagged constructs. (E) Example expression of epitope-scaffolds optimized with ProteinMPNN, a machine learning tool that predicts different amino acid sequences that will fold into a desired structure, and comparison to CC2, their template design. Figures modified from Caitlin Harris and Mihai Azoitei.

FIG. 27 shows example binding and mutagenesis analysis by ELISA. We assessed binding of wild-type epitope-scaffolds (SP7, CC19), mutated epitope-scaffolds containing alanine substitutions at key epitope residues (SP7 3A2, CC19 LA, CC19 FA), and the unmodified parent scaffold proteins corresponding to these designs (SP7 WT, CC19 WT) against antibodies S2P6 (A), CC40.8 (B), and DH1057 (C). Both wild-type epitope-scaffolds demonstrated high affinity binding to their target antibodies, whereas introducing mutations completely abrogated or significantly reduced these interactions. Furthermore, neither unmodified parent scaffold showed any binding activity with these antibodies.

FIG. 28 provides example structural analysis by X-ray crystallography. (A) Example crystal structure of SP7 in complex with antibody DH1057 and comparison of grafted epitope to computational model. (B) Example crystal structure of CC19 and comparison of epitope presentation by CC19 to CC40.8-bound epitope and epitope conformation on native Spike. Figures modified from Caitlin Harris and Dr. Mihai Azoitei.

FIG. 29 illustrates example reactivity of designed ESs to sera from non-human primates vaccinated with two doses of SARS-CoV-2 mRNA Spike. (A) Example pre-immunization and post-boost sera binding to WA-2 Spike, RBD and synthetic Fusion Peptide and S2 stem helix peptides. (B) Left: Example pre-immunizations and post-boost sera binding to the synthetic Fusion Peptide, and to FP ESs. Right: Post-boost sera binding to engineered FP ESs and to the parent scaffolds they were derived from and that lack the grafted epitope. (C) Left: Example pre-immunizations and post-boost sera binding to the synthetic S2 stem helix peptide and to S2hlx ESs. Right: Post-boost sera binding to engineered S2hlx ESs and to the parent scaffolds they were derived from and that lack the grafted epitope. (D) Example pre-immunizations and post-boost sera binding to the synthetic S2 stem helix peptide and to S2hlx-Ex ESs. For all graphs * p<0.05.

FIG. 30 illustrates the isolation and characterization of epitope scaffold reactive antibodies from subjects with pre-existing SARS-CoV-2 immunity. (A) shows graphs depicting sera binding profile of subjects from which B cells were isolated. Binding was measured to the synthetic peptide epitope, the epitope scaffold used as the sorting probe, and the corresponding parent scaffold (PS) that lacks the epitope and was employed for negative selection. (B) shows representative FACS plots of memory B cells labeled with epitope scaffold probes. Cells from Subject 26 were labeled with S2hlx-Ex19 conjugated to VB15 (x-axis) and AF647 (y-axis). A third bait comprising S2hlx-Ex19 PShlx conjugated to BV421 was used to exclude scaffold-specific memory B cells. (C) shows a schematic depicting binding of the isolated antibodies to diverse CoV spikes. (D) shows a schematic illustrating neutralization of diverse pseudo-viruses by isolated stem helix antibodies. CoV-1=SARS-CoV-1. All other pseudo-viruses are the indicated variant of SARS-CoV-2. (E) shows graphs depicting the binding of isolated antibodies to cells infected with two different SARS-CoV-2 VOCs (D614G, top; BA.1, bottom). Antibodies in red have been previously described, those depicted in green were isolated here, while those in blue represent controls; DH1047 targets the RBD domain; DH1058 targets the spike815-823 peptide domain; anti-HIV antibody VRC01 was included as a negative control. (F) NK cell degranulation ability of mAbs from (E).

FIG. 31 shows graphs depicting example ELISA binding of isolated stem helix antibodies to SARS-CoV-2 VOC spikes.

FIG. 32 shows graphs depicting example ELISA binding of isolated stem helix antibodies to human betacoronavirus spikes.

FIG. 33 shows graphs depicting example ELISA binding of isolated stem helix antibodies to animal betacoronavirus spikes.

FIG. 34 shows graphs depicting example ELISA binding of isolated spike815-823 antibodies to human and animal coronavirus spikes.

FIG. 35 shows a schematic depicting binding of the unmutated common ancestor (UCA) of isolated stem helix antibodies DH1501.1, DH1501.2, and DH1501.3 to multiple human coronavirus spikes. (A) Shows example ELISA binding data. (B) Shows example sequence alignments between the UCA (SEQ ID NO: 181 for heavy chain and SEQ ID NO: 182 for light chain) and mature antibodies DH1501.1 (SEQ ID NO: 121 for heavy chain and SEQ ID NO: 134 for light chain, DH1501.2 (SEQ ID NO: 122 for heavy chain and SEQ ID NO: 135 for light chain) and DH1501.3 (SEQ ID NO: 115 for heavy chain and SEQ ID NO: 128 for light chain). See also FIGS. 46-47.

FIG. 36 illustrates the immunogenicity of stem helix epitope scaffolds. (A) Shows a schematic depicting the development and NSEM analysis of the spycatcher/spytag mi03 nanoparticles (NP) displaying 60 epitope scaffold copies. (B) Shows a schematic of an example study design to determine the immunogenicity of stem helix nanoparticles in BALB/c mice. (C) Shows graphs depicting example binding of sera from animals immunized with the different epitope scaffold regimens against the SARS-CoV-2 WA-2 spike at different study time points. WA-2 and XBB refer to the SARS-CoV-2 variants. CoV-1=SARS-CoV-1. GXP4L=Pangolin CoV GX-P4L. SHC014=Bat CoV RsSHC014. (D) Shows a schematic depicting binding of sera after the second boost against diverse CoV spikes. Values are calculated as the logarithm of the area under the curve for binding curves measured as in (C). (E) Shows a graph depicting example binding of S2P6 mAb and sera from animals immunized with Ex_mosaic to the WT stem helix epitope peptide and mutated variants. (F) Shows a schematic of a study design to determine ability of Ex_mosaic-NP to protect against a live coronavirus challenge. (G) Shows a graph depicting the percentage of weight loss observed in animals treated with the procedure described in (F). Data are presented as the mean+/−standard deviation. (H) Shows schematic depicting example lung congestion scores and plaque analysis of infected animals. For (G) and (H), adjuvant n=4, Spike mRNA x4 n=5, Spike mRNA x2+Ex_mosaic x2 n=6. Data points correspond to individual animals. F was calculated by one way ANOVA using Brown-Forsythe test and comparisons using Tukeys test. ns p=0.9999; *¹p=0.027; *²p=0.033; *³p=0.005 *⁴p=0.00001; *⁵p=0.00005. Source data are provided as a Source Data file.

FIG. 37 illustrates example characterization of epitope scaffold nanoparticles. (A) Shows a schematic depicting NSEM sample image and 2D classification of the Ex_mosaic NP. (B) Shows a schematic depicting ELISA binding of mi03 nanoparticles displaying the indicated epitope scaffolds to stem helix antibodies S2P6, DH1057 and CC40.8.

FIG. 38 illustrates example binding of sera from BALBc immunized mice with stem helix epitope scaffold nanoparticles to an epitope scaffold not used in the immunization and the RBD. (A) Shows graphs depicting sera binding to a stem helix epitope scaffold S2hlx-Ex54 at indicated time points. (B) Shows graphs depicting example binding of sera to the RBD at indicated time points. Data are presented as the mean.

FIG. 39 illustrates example stem helix epitope specific binding of sera from K18-ACE2 mice primed with spike mRNA and boosted with either the epitope scaffold mosaic NP or spike mRNA. (A) Shows graphs depicting example binding of sera, at indicated time points, to a stem helix epitope scaffold not used in the immunizations (S2hlx-Ex54). (B) Shows graphs depicting example binding of sera, at indicated time points, to a synthesized peptide encoding the stem helix epitope. Data are presented as the mean. Data points correspond to individual animals. F was calculated by two-way ANOVA using Geisser-Greenhouse correction and comparisons using Tukeys test. ns=not significant; * p=0.015.

FIG. 40 illustrates epitope scaffolds binding to diverse mature and germline antibodies that target the spike epitope. (A) Example ELISA binding of mAbs DH1058, DH1294, VN01H1, C77G12, VP12E7, Cov44-62, and Cov44-79 to FP ESs and to a synthetic peptide encompassing the spike epitope (SARS-CoV-2 spike residues 808-833). (B) Example ELISA binding of mAbs DH1058UCA, DH1294UCA, VP12E7iGL, Cov44-62iGL, and Cov44-79iGL to FP ESs and to a synthetic peptide encompassing the spike epitope (SARS-CoV-2 spike residues 808-833).

FIG. 41. Illustrates example surface Plasmon Resonance curves of Spike (WA-2) proteins binding to S2P6, DH1057.1 and CC40.8 mAbs.

FIG. 42. Illustrates example S2hlx-Ex2 based MPNN designs showing broad binding. Binding of S2P6, DH1057.1 and CC40.8 mAbs to MPNN designs based on S2hlx-Ex2 epitope scaffolds were measured by ELISA.

FIG. 43. illustrates example binding affinities of S2hlx-Ex4, Ex6, and Ex3 derived epitope scaffolds to S2P6, DH1057.1, and CC40.8 mAbs. Binding kinetics were measured by Surface Plasmon Resonance (SPR).

FIG. 44. Illustrates examples of epitope scaffolds binding to diverse mature and germline antibodies that target the stem helix epitope. (A) ELISA binding of mAbs S2P6, DH1057.1, CC40.8, Cov30-14, Cov44-26, and Cov89-22 to S2hlx-Ex ESs and to Spike (WA-2) protein. (B) ELISA binding of mAbs S2P6iGL, DH1057UCA, CC40.8iGL, Cov44-26UCA, and Cov89-22iGL to S2hlx-Ex ESs and to Spike (WA-2) protein.

FIG. 45. Illustrates examples binding specificity of S2hlx-Ex epitope scaffolds for representative epitopes against the stem helix epitope. Indicated spike mutations known to affect the binding of stem helix were introduced in the respective epitope scaffolds.

FIG. 46 shows amino acid sequences corresponding to the heavy chain variable regions of antibodies described herein isolated from humans pre-exposed to SARS-CoV-2 using epitope scaffolds described herein.

FIG. 47 shows amino acid sequences corresponding to the light chain variable regions of antibodies described herein isolated from humans pre-exposed to SARS-CoV-2 using epitope scaffolds described herein.

FIG. 48 shows amino acid sequences of protein scaffolds (PDBid) designed using the BB grafting+MPNN+AF strategy described herein.

FIG. 49 shows amino acid sequences of scaffold (PDBid) epitopes designed using the BB grafting+MPNN+AF strategy described herein.

FIG. 50 shows results of neutralization assays (in 293T/ACE2 cells) of stem helix antibodies. Samples were diluted to 0.5 mg/ml or 1.0 mg/ml in PBS and assayed 1:20; 5-fold. Values show the antibody concentration (ug/ml) at which relative luminescence units (RLUs) were reduced 50% or 80% compared to virus control wells (no test sample). Values in black bold type are the samples considered positive for neutralizing antibody activity in the sample based on the criterion of >3X background.

FIG. 51 shows results of a SARS-CoV-2 microneutralization assay. The mean effective concentration for a dilution series is the greatest dilution of serum observed to exhibit ≥50% virus neutralization. All experimental samples were assayed as duplicate dilution series. Diluted five-fold. >50 ug/ml denotes endpoint not determined, most likely due to no or very low neutralization activity. 70.71 ug/ml denotes the geometric mean of one neutralized well at 50 ug/ml and an assumed neutralized well at 100 ug/ml.

FIG. 52 shows results of neutralization assay in s93/ACE2_TMPRSS2 cells of stem helix antibodies. Samples were diluted to 1.0 mg/ml in PBS and assayed at 1:20; 5 fold. Virus: Spike-pseudotyped viruses.

FIG. 53 shows ID50 for different antibodies described herein.

### DETAILED DESCRIPTION OF THE INVENTION

The desire to mount an effective defense against emerging SARS-CoV-2 variants and other human-infecting coronaviruses has prompted interest to develop a broadly protective pancoronavirus vaccine. Current vaccines primarily contain the RBD of the coronavirus spike protein. However, the RBD can mutate to escape the primary immunity (e.g., antibody responses) produced by individuals.

Described herein are coronavirus vaccines containing immunogens that are less variable than RBD. In some embodiments, these coronavirus vaccines contain immunogens that can stimulate immune responses to epitopes that are conserved across diverse coronaviruses. These “pan-coronavirus” vaccines can broadly protect against both currently circulating and emerging coronaviruses. These vaccines can induce high antibody titers against viral targets that have a low propensity to acquiring mutations.

Several broadly cross-reactive antibodies against epitopes in the fusion peptide and in the S2 stem helix region of the spike protein implicate these regions as highly-conserved viral targets for inclusion in vaccines. However, antibodies against these targets can be weakly elicited by vaccines, possibly owing to the poor accessibility of the fusion peptide and stem helix on the native spike.

As disclosed herein, epitope-accessible immunogenic compositions comprising an epitope that stimulates an immune response to an S2 region (contains fusion peptide and S2 stem helix; RBD is part of the S1 region) of a human coronavirus spike protein have been developed. The immunogenic compositions were developed using computational modeling methods to design ES immunogens that display the fusion peptide and/or the S2 stem helix epitopes. These ES molecules were bound by both mature and germline versions of multiple known human antibodies with high affinity and specificity and showed potent engagement of pre-existing SARS-CoV-2 immunity, thus illustrating that these can preferentially elicit responses against the fusion peptide and the S2 stem helix by vaccination.

Disclosed herein are computational-design, production and experimental characterization of protein immunogens that can selectively amplify immunity against the fusion peptide and/or S2 stem helix, for a pancoronavirus vaccine. In some embodiments, protein scaffolds (also called scaffold polypeptides) were identified that can accommodate epitopes that can stimulate antibodies to a coronavirus fusion protein or S2 stem helix region. The epitopes were “grafted” or “transplanted” into the protein scaffolds. The resulting ES contain immune system-accessible epitopes. The epitopes are displayed in an arrangement such that an immune response (e.g., antibodies) stimulated by immunogenic compositions containing the ES molecules can broadly react with different coronaviruses (e.g., with coronaviruses having different RBD sequences).

Aspects of the invention are drawn to immunogenic compositions. In some embodiments, an immunogenic composition comprises an epitope that can stimulate an immune response specific for an S2 region of a human coronavirus spike protein. In some embodiments, the epitope from the S2 region of the human coronavirus spike protein can be a fusion peptide epitope, a S2 stem helix epitope, or a combination thereof. In some embodiments, the immunogenic composition can comprise an epitope grafted into (e.g., inserted into) a scaffold protein. In some embodiments, the immunogenic composition can comprise any one of the epitope scaffolds FP-1 to FP-15, S2hlx-1 to S2hlx-15, S2hlx-Ex1 to S2hlx-35, and S2hlx-Ex2-Trx, S2hlx-Ex4-Trx, S2hlx-Ex6-Trx, S2hlx-Ex7-Trx, S2hlx-Ex8-Trx, S2hlx-Ex4-MBP, S2hlx-Ex4-GST, S2hlx-Ex4-FH8 and S2hlx-Ex4-SUMO, as described herein.

In embodiments, the immunogenic composition can exclude an immunogen that stimulates an immune response to a RBD from the spike protein.

Aspects of the invention are drawn to antibodies that can bind to the epitopes and/or epitope scaffolds described herein. In some embodiments, the antibodies described herein that can bind to the epitopes and/or epitope scaffolds described herein can also bind to an S2 region of a coronavirus spike protein. In some embodiments, the antibodies described herein that can bind to the epitopes and/or epitope scaffolds described herein can also bind to an Alphacoronavirus or Betacoronavirus. In some embodiments, the antibodies described herein that can bind to the epitopes and/or epitope scaffolds described herein can also bind to a Betacoronavirus. In some embodiments, the antibodies described herein that can bind to the epitopes and/or epitope scaffolds described herein can also bind to a Sarbecovirus. In some embodiments, the antibodies described herein that can bind to the epitopes and/or epitope scaffolds described herein can also bind to SARSr-CoV or SARS-CoV.

### Definitions

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

The term “administering” can refer to introducing a substance into a subject. Any route of administration can be utilized including, for example, intranasal, topical, oral, parenteral, intravitreal, intraocular, ocular, subretinal, intrathecal, intravenous, subcutaneous, transcutaneous, intracutaneous, intracranial and the like administration. For example, “parenteral administration” can refer to administration via injection or infusion. Parenteral administration includes, but is not limited to, subcutaneous administration, intravenous administration, and intramuscular administration. For example, the inhibitor and/or degrader can be administered intranasally, by inhalation, intrapulmonarily, or by injection (e.g., intravenous or subcutaneous). Herein, administering can refer to introducing an epitope scaffold or composition thereof into a subject. In some embodiments, the purpose of the administration is prophylactic protection against coronavirus infection and/or symptoms of disease caused by coronavirus infection.

The term “simultaneous administration” can refer to a first agent and a second agent administered less than about 15 minutes apart, e.g., less than about 10, 5, or 1 minutes. When the first agent and the second agent are administered simultaneously, the first and second treatments can be in the same composition (e.g., a composition comprising both the first and second therapeutic agents) or separately (e.g., the first therapeutic agent is contained in one composition and the second treatment is contained in another composition).

The term “sequential administration” can refer to a first agent and a second agent administered to a subject greater than about 15 minutes apart, such as greater than about 20, 30, 40, 50, 60 minutes, or greater than 60 minutes apart. Either agent can be administered first. For example, the first agent and the second agent can be included in separate compositions, which can be included in the same or different packages or kits.

The terms “co-administration” or the like, as used herein, can refer to the administration of a first active agent and at least one additional active agent to a single subject, and is intended to include treatment regimens in which the compounds and/or agents are administered by the same or different route of administration, in the same or a different dosage form, and at the same or different time.

As used herein, the term “domain” can refer to a functional portion, segment or region of a protein or polypeptide. “Interaction domain” can refer to a portion, segment or region of a protein, polypeptide or protein fragment that is responsible for the physical affinity of that protein, protein fragment or isolated domain for another protein, protein fragment or isolated domain.

The term “in combination” can refer to the use of more than one therapies (e.g., one or more prophylactic and/or therapeutic agents). The use of the term “in combination” does not restrict the order in which therapies are administered to a subject with a disease or disorder, or the route of administration.

The term “epitope” can refer to a protein determinant capable of specific binding to an immunoglobulin, a scFv, a T-cell receptor and the like.

The term “grafting” can refer to combining an epitope with a protein scaffold. Generally, as described herein, computational methods can be used to identify protein scaffolds that have regions that have the same shape or conformation as a selected epitope (e.g., the scaffolds have regions that can display the epitope such that an immune response to a desired conformation of the epitope can be produced). “Grafting” of the epitope into the protein scaffold at this region (e.g., the epitope can replace the same shape/conformation segment of the protein scaffold) produces the epitope scaffold. “Transplanting” can used instead of grafting.

As used herein, the term “immunogen” and related terms “immunogenic” refer to molecules that have the ability to induce an immune response, including antibodies and/or cellular immune responses in an animal, eg, a mammal. Although an immunogen may be antigenic, an “antigen” need not necessarily be an “immunogen” because such molecules may not induce a sufficient immune response. In some examples, this may be because of the antigen's size, conformation and the like. In some embodiments, an immunogenic composition can contain one or more immunogens that can induce an immune response that can specifically recognize viruses that contain the immunogen or antigen.

As used herein, the terms “immunological binding,” and “immunological binding properties” can refer to non-covalent interactions of the type which occur between an immunoglobulin molecule and an antigen for which the immunoglobulin is specific. The strength, or affinity of immunological binding interactions can be expressed in terms of the equilibrium binding constant (K_{D}) of the interaction, wherein a smaller K_{D} value represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables the cancellation of all parameters not related to affinity, and is equal to the equilibrium binding constant, K_{D}.

The terms “prevent,” “preventing” and/or “prevention” can refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of diseases or disorders provided herein. The terms encompass the inhibition or reduction of a symptom of the particular disease. For example, one or more of the following effects can result from the administration of a therapy or a combination of therapies as described herein: (i) the inhibition of the development or onset of a viral infection and/or a symptom associated therewith; and (ii) the inhibition of the recurrence of a viral infection and/or a symptom associated therewith.

As used herein, “limiting” means to limit SARS-CoV-2 infection in subjects at higher risk of SARS-CoV-2 infection. Groups at particularly high risk include adults over the age of 85, individuals suffering from various chronic lung problems, Individuals suffering from heart disease, individuals suffering from diabetes and/or obesity, and individuals suffering from any type of immunodeficiency. In this method, the epitope scaffolds or immunogenic compositions containing the epitope scaffolds can be used as vaccines.

The term “in vivo” can refer to an event that takes place in a subject's body.

The term “in vitro” can refer to an event that takes places outside of a subject's body.

The term “ex vivo” can refer to outside a living subject. Examples of ex vivo cell populations include in vitro cell cultures and biological samples such as fluid or tissue samples from humans or animals. Such samples can be obtained by methods well known in the art. Exemplary biological fluid samples include blood, cerebrospinal fluid, urine, saliva. Exemplary tissue samples include tumors and biopsies thereof. In this context, the present compounds can be in numerous applications, both therapeutic and experimental.

The terms “manage,” “managing,” and “management,” in the context of the administration of a therapy to a subject, can refer to the beneficial effects that a subject derives from a therapy, which does not result in a cure of a viral infection. In embodiments, a subject is administered one or more therapies to manage a viral infection so as to prevent the progression or worsening of the viral infection.

The phrase “pharmaceutical composition” or a “pharmaceutical formulation” can refer to a composition or pharmaceutical composition suitable for administration to a subject, such as a mammal, especially a human and that can refer to the combination of an active agent(s), or ingredient with a pharmaceutically acceptable carrier or excipient, making the composition suitable for diagnostic, therapeutic, or preventive use in vitro, in vivo, or ex vivo. A “pharmaceutical composition” can be sterile and can be free of contaminants that can elicit an undesirable response within the subject (e.g., the compound(s) in the pharmaceutical composition is pharmaceutical grade). Pharmaceutical compositions can be designed for administration to subjects or patients in need thereof via a number of different routes of administration including oral, intranasal, topical, intravenous, buccal, rectal, parenteral, intraperitoneal, intradermal, intratracheal, intramuscular, subcutaneous, by stent-eluting devices, catheters-eluting devices, intravascular balloons, inhalational and the like. In some embodiments, an immunogenic composition is a type of pharmaceutical composition.

As used throughout the present application, the term “polypeptide” is used in its broadest sense to refer to a sequence of subunit amino acids. The polypeptides described herein may be chemically synthesized or recombinantly expressed. Polypeptide can encompass a singular “polypeptide” as well as plural “polypeptides,” and can refer to a molecule composed of monomers (amino acids) linearly linked by amide bonds (also known as peptide bonds). The term “polypeptide” can refer to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology but is not necessarily translated from a designated nucleic acid sequence. It can be generated in any manner, including by chemical synthesis.

“Receptor binding domain” or “RBD” as used herein can refer to a region, segment or domain within a polypeptide studding (or covering) the envelope of a virus that is associated with or mediates the binding of the virus to a host cell, in particular to a host cell receptor. Polypeptides studding the envelope of a virus are commonly referred to as spikes or spike proteins. SARS-CoV-2, for example, binds to the ACE2 receptor to gain entry into respiratory and digestive epithelial cells. The S1-domain of SARS-CoV-2 contains an RBD which enables it to bind to ACE2 and fuse into the membrane of epithelial cells.

“Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

The term “subject,” “patient” or “individual” can refer to any organism to which aspects of the invention can be administered, e.g., for experimental, diagnostic, prophylactic, and/or therapeutic purposes. For example, subjects to which compounds of the disclosure can be administered include animals, such as mammals. Non-limiting examples of mammals include primates, such as humans. For veterinary applications, a wide variety of subjects will be suitable, e.g., livestock such as cattle, sheep, goats, cows, swine, and the like; poultry such as chickens, ducks, geese, turkeys, and the like; and domesticated animals for example pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

The terms “therapies” and/or “therapy” can refer to any protocol(s), method(s), compositions, formulations, and/or agent(s) that can be used in the prevention, treatment, management, or amelioration of a viral infection or a symptom associated therewith. In embodiments, the terms “therapies” and “therapy” can refer to biological therapy, supportive therapy, and/or other therapies useful in treatment, management, prevention, or amelioration of a viral infection or a symptom associated therewith known to one of skill in the art.

The terms “therapeutic agent”, and “therapeutic agents” can refer to any agent(s) which can be used in the prevention, treatment and/or management of a viral infection or a symptom associated therewith.

The term “therapeutically effective amount” can refer to that amount of an embodiment of the composition or pharmaceutical composition being administered that will relieve to some extent one or more of the symptoms of the disease or condition being treated, and/or that amount that will prevent, to some extent, one or more of the symptoms of the condition or disease that the subject being treated has or is at risk of developing.

As used herein, “treat” or “treating” means accomplishing one or more of the following: (a) reducing SARS-CoV-2 titer in a subject; (b) limiting any increase of SARS-CoV-2 titer in a subject; (c) reducing the severity of SARS-CoV-2 symptoms; (d) limiting or preventing development of symptoms after infection; (e) inhibiting worsening of SARS-CoV-2 symptoms, (f) limiting or preventing recurrence of SARS-CoV-2 symptoms in subjects that were previously symptomatic for SARS-CoV-2 infection. In some embodiment methods, epitope scaffolds and/or immunogenic compositions thereof, can used as “therapeutic vaccines” to ameliorate the existing infection and/or provide prophylaxis against infection with additional SARS-CoV-2 virus. In some embodiment methods, the one or more compositions can be administered prophylactically to a subject that is not known to be infected but may be at risk of exposure to coronavirus.

The terms “treat,” “treatment,” and “treating” can refer to the management and care of a subject for the purpose of combating a condition, disease or disorder, such as a viral infection, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications.

A “variant” can refer to a virus having one or more mutations as compared to a known virus. A strain can be a genetic variant or subtype of a virus. The terms “strain”, “variant,”, and “isolate” may be used interchangeably. In certain embodiments, a variant has developed a “specific group of mutations” that causes the variant to behave differently than that of the strain it originated from.

The term “viral infection” can refer to the invasion by, multiplication and/or presence of a virus in a cell or a subject.

In one embodiment, a viral infection can be an “active” infection. An active infection can refer to one in which the virus is replicating in a cell or a subject. Active infections can be characterized by the spread of the virus to other cells, tissues, and/or organs, from the cells, tissues, and/or organs initially infected by the virus.

In embodiments, the viral infection can be a “latent” infection. A latent infection can refer to one in which the virus is not replicating. In some embodiments, an infection can refer to the pathological state resulting from the presence of the virus in a cell or a subject, or by the invasion of a cell or subject by the virus.

The term “protein scaffold” or “scaffold polypeptide” can refer to a molecule that can be a “framework” for or that can “host” an epitope. A protein scaffold containing a “grafted epitope” is called an epitope scaffold or ES.

The term “grafting” can refer to combining an epitope with a protein scaffold. Generally, as described herein, computational methods can be used to identify protein scaffolds that have regions that have the same shape or conformation as a selected epitope (e.g., the scaffolds have regions that can display the epitope such that an immune response to the epitope can be produced). “Grafting” of the epitope into the protein scaffold at this region (e.g., the epitope can replace the same shape/conformation segment of the protein scaffold) produces the epitope scaffold. “Transplanting” can used instead of grafting.

### Coronavirus

A “coronavirus” can refer to an enveloped virus with a positive-sense single-stranded RNA genome and a nucleocapsid of helical symmetry that constitutes the subfamily Orthocoronavirinae, in the family Coronaviridae. In some embodiments, the coronavirus is an animal or human coronavirus. In embodiments, the coronavirus can comprise a genus Alphacoronavirus or Betacoronavirus. In embodiments, the genus Betacoronavirus can comprise a subgenus Sarbecovirus. In embodiments, the subgenus Sarbecovirus can comprise a species severe acute respiratory syndrome-related coronavirus (SARSr-CoV or SARS-CoV).

“SARSr-CoV or SARS-CoV species contains a strain of coronavirus called “SARS-CoV-2” which causes COVID-19 (coronavirus disease 2019), the respiratory illness responsible for the ongoing COVID-19 pandemic. Symptoms of COVID-19 can include, but may not be limited to fever, chills, cough, shortness of breath, difficulty breathing, fatigue, muscle or body aches, headache, chest pain, lower respiratory tract infections, upper respiratory tract infections, bronchiolitis, pneumonia, listlessness, diminished appetite, recurrent wheezing, and the like. Other or additional symptoms can include rhinorrhea, sore throat, malaise, repeated shaking, diarrhea, loss of smell and/or taste, muscle pain, and the like.

In some embodiments, the subject with a coronavirus infection exhibits one or more symptoms associated with mild COVID-19, moderate COVID-19, mild-to-moderate COVID-19, severe COVID-19 (e.g., critical COVID-19), or exhibits no symptoms associated with COVID-19 (asymptomatic). It should be understood that in reference to the treatment of patients with different COVID-19 disease severity, “asymptomatic” infection refers to patients diagnosed with COVID-19 by a standardized assay that do not present with symptoms.

In embodiments, the species SARSr-CoV or SARS-CoV species contains a strain of coronavirus called “SARS-CoV-1.” In embodiments, SARS-CoV-causes severe acute respiratory syndrome (SARS).

Coronavirus infections can be fatal. Coronavirus infections can result in sequela (e.g., long covid).

### Spike Protein

“Spike protein,” “S protein” or “S” can refer to a membrane anchored class I fusion protein expressed on the coronavirus surface, which gives the characteristic shape to the coronavirus under the electronic microscope. Spike protein can be target of host immune responses elicited by infection or vaccination. Spike protein is comprised of two distinct functional subunits: an N-terminal, membrane distal subunit designated S1, and a C-terminal, membrane proximal subunit designated S2.

The S1 domain of the coronavirus spike contains the receptor binding domain (RBD). RBD binds to cell-surface receptors and attaches to cells prior to virus entry into cells. Current SARS-CoV-2 vaccines contain sequences from RBD. However, frequent mutations in RBD can result in reduced efficacy of antibodies produced by an immune response to a prior virus. In some examples, this can result in reduced efficacy of current SARS-CoV-2 vaccines.

For SARS-CoV-1 and SARS-CoV-2, the cellular receptor is “angiotensin converting enzyme-2” or “ACE2”. ACE2 is a transmembrane protein that has an extracellular domain, a transmembrane domain, and an intracellular domain. It can be expressed in cells of the nasal passages, lungs, arteries, heart, kidney and intestines, for example. The human version of the enzyme can be referred to as hACE2.

The S2 domain of the coronavirus spike contains the fusion machinery that undergoes large structural rearrangements to mediate fusion of the host and viral membranes. The S1 domain serves to stabilize the S2 subunit in the pre-fusion state and facilitates the attachment to ligands on host cells through the RBD. In embodiments, epitopes disclosed herein can stimulate immune responses (e.g., antibodies) to the S2 region of the human coronavirus spike protein, which can comprise a fusion protein, a S2 stem helix epitope, or a combination thereof. The fusion protein and S2 stem helix are conserved amino acid regions, not subject to the variability of RBD. Because they are conserved, these regions can be the basis for a “pan-coronavirus vaccine” that may stimulate protection against a variety of diverse coronaviruses and be more efficacious over time, due to their conservation (e.g., virus mutations in these regions are less likely to occur).

### Epitopes

As used herein, the term “epitope” can include any protein determinant capable of specific binding to an immunoglobulin, a scFv, or a T-cell receptor. The variable region, of an antibody for example, allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can have chemically active surface groupings of molecules such as amino acids or sugar side chains and can have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3).

In embodiments, an amino acid epitope for antibody binding from a SARS-CoV-2 spike protein grafted into a scaffold protein can stimulate immune responses against a fusion peptide amino acid epitope, a S2 stem helix amino acid epitope, or a combination thereof. The epitope can be grafted into a scaffold protein. Herein, this configuration of an epitope grafted into a scaffold protein is referred to as an epitope scaffold (ES). The epitope can be any described in the following section related to the fusion peptide, or in the subsequent section related to the S2 stem helix.

In some embodiments, epitopes can stimulate antibodies specific for a fusion protein, including amino acids from about 815-823 of a SARS-Cov-2 spike. In some embodiments, epitopes can stimulate antibodies specific a S2 stem helix, including amino acids from about 1148-1156 or from about 1144-1157 of a SARS-Cov-2 spike (e.g., S2 helix “extended”).

In some embodiments, epitopes can be between about 5 and about 15 amino acids in length.

Epitopes that Stimulate Immune Responses to the Fusion Peptide

The terms “fusion peptide,” “fusion protein,” and “FP” can be used interchangeably and can refer to a region in the S2 subunit of the SARS-CoV-2 spike which shows high conservation across diverse coronaviruses.

In embodiments, the epitopes, and epitope scaffolds made therefrom, can stimulate immune responses to, and/or can bind antibodies that bind to, fusion peptide epitopes of coronavirus. The fusion peptide epitopes can comprise amino acids from about 800 to about 850, from about 808 to about 833, from about 813 to about 824, from about 816 to about 823, or from about 815 to about 823 of a SARS-Cov-2 spike protein. In some embodiments, epitopes from the fusion peptide can include amino acids from about 815-823 of a SARS-Cov-2 spike.

In some embodiments, the epitopes disclosed herein can be amino acid sequences encompassed by the sequence RSX₁IEDX₂LF (SEQ ID NO: 179). In this amino acid sequence, R comprises arginine, S comprises serine, I comprises isoleucine, E comprises glutamic acid, D comprises aspartic acid, L comprises leucine, and F comprises phenylalanine. In embodiments of this sequence, X₁ and X₂ can be any amino acid. In some embodiments, X₁ is selected from the group consisting of A, Q, G, H, I, L, F, T, Y and V. In some embodiments, X₂ is selected from the group consisting of G, I, L, F, T, W, Y and V.

In some embodiments, the epitope can include the amino acid sequences RSFIEDLLF (SEQ ID NO: 66), RSAIEDGLF (SEQ ID NO: 67), RSAIEDILF (SEQ ID NO: 68), RSAIEDLLF (SEQ ID NO: 69), RSGIEDFLF (SEQ ID NO: 70), RSHIEDVLF (SEQ ID NO: 71), RSIIEDLLF (SEQ ID NO: 72), RSLIEDYLF (SEQ ID NO: 73), RSQIEDLLF (SEQ ID NO: 74), RSTIEDFLF (SEQ ID NO: 75), RSVIEDTLF (SEQ ID NO: 76), RSYIEDVLF (SEQ ID NO: 77), RSYIEDWLF (SEQ ID NO: 78), or an amino acid sequence 85, 96, 87, 88, 89, 90, 91, 92, 93, 94, 95, 96, 97, 98 or 99% identical thereto.

In embodiments, antibodies that are stimulated by and/or react with a coronavirus fusion peptide can cross-react with the epitopes disclosed herein. Non-limiting examples of antibodies that can bind to the epitopes can include DH1058, DH1294, VN01H1, Cov44-62, Cov44-79. In some embodiments, 2, 3, 4 or all 5 of these antibodies bind to epitope scaffolds disclosed herein that contain epitopes related to coronavirus fusion peptides.

Epitopes that Stimulate Immune Responses to the S2 Stem Helix

The term “S2 stem helix” can refer to a region in the S2 subunit of the SARS-CoV-2 spike which shows high conservation across diverse coronaviruses.

In embodiments, epitopes and epitope scaffolds made therefrom, can stimulate immune responses to, and/or can bind antibodies that bind to, the S2 stem helix of coronavirus. The S2 stem helix epitopes can comprise amino acids from about 1130 to about 1170, from about 1140 to about 1163, from about 1144 to about 1158, from about 1144 to about 1157, or to about 1148 to about 1156 of a SARS-Cov-2 spike protein.

In some embodiments, the epitopes disclosed herein can be amino acid sequences encompassed by the sequence FKX₁ELDX₂YF (SEQ ID NO: 180). In this amino acid sequence, R comprises arginine, S comprises serine, I comprises isoleucine, E comprises glutamic acid, D comprises aspartic acid, L comprises leucine, and F comprises phenylalanine. In embodiments of this sequence, X₁ and X₂ can be any amino acid. In some embodiments, X₁ is selected from the group consisting of A, Q, G, H, I, L, F, T, Y and V. In some embodiments, X₂ is selected from the group consisting of G, I, L, F, T, W, Y and V.

In some embodiments, the epitope can comprise the amino acid sequences FKEELDKYF (SEQ ID NO: 79), FKDELDKYF (SEQ ID NO: 80), FKIELDKYF (SEQ ID NO: 81), FKLELDKYF (SEQ ID NO: 82), FKNELDKYF (SEQ ID NO: 83), FKRELDKYF (SEQ ID NO: 84), FKSELDKYF (SEQ ID NO: 85), FKVELDKYF (SEQ ID NO: 86), FKVELDLYF (SEQ ID NO: 87), FKYELDKYF (SEQ ID NO: 88) or an amino acid sequence 85, 96, 87, 88, 89, 90, 91, 92, 93, 94, 95, 96, 97, 98 or 99% identical thereto.

In other embodiments, the epitope can comprise the amino acid sequences ELDSFKEELDKYFK (SEQ ID NO: 89), ALASFKIELDIYFI (SEQ ID NO: 90), ALDFFKLELDFYFL (SEQ ID NO: 91), ALDGFKLELDLYFF (SEQ ID NO: 92), ALDGFKLELDVYFI (SEQ ID NO: 93), ELAKFKIELDKYFA (SEQ ID NO: 94), ELASFKIELDIYFG (SEQ ID NO: 95), ELASFKIELDIYFI (SEQ ID NO: 96), ELEKFKYELDLYFI (SEQ ID NO: 97), ELEKFKYELDLYFM (SEQ ID NO: 98), ELGKFKYELDLYFM (SEQ ID NO: 99), ELREFKIELDKYFA (SEQ ID NO: 100), GLALFKVELDIYFL (SEQ ID NO: 101), GLASFKIELDIYFI (SEQ ID NO: 102), KLEEFKYELDIYFM (SEQ ID NO: 103), NLDSFKEELDDYFK (SEQ ID NO: 104), SLASFKIELDIYFF (SEQ ID NO: 105), SLASFKYELDIYFG (SEQ ID NO: 106), SLELFKFELDIYFL (SEQ ID NO: 107), SLELFKFELDLYFL (SEQ ID NO: 108), SLELFKWELDVYFL (SEQ ID NO: 109), SLELFKYELDLYFL (SEQ ID NO: 110), SLGKFKYELDLYFL (SEQ ID NO: 111), SLSLFKYELDLYFL (SEQ ID NO: 112) or an amino acid sequence 85, 96, 87, 88, 89, 90, 91, 92, 93, 94, 95, 96, 97, 98 or 99% identical thereto.

In some embodiments, the epitope can comprise the amino acid sequences KLKLFKCELDLYFL (SEQ ID NO: 159), KLKLFKCELDLYFL (SEQ ID NO: 160), KLRLFKLELDLYFL (SEQ ID NO:161), RLKLFKCELDLYFL (SEQ ID NO: 162), KLKLFKCELDLYFL (SEQ ID NO: 163), KLKLFKCELDLYFL (SEQ ID NO: 164), KLLLFKEELDLYFL (SEQ ID NO: 165), SLKLFKCELDLYFL (SEQ ID NO: 166), KLLLFKCELDLYFL (SEQ ID NO: 167), ELKLFKCELDLYFL (SEQ ID NO: 168), SLCGFKGELDLYFL (SEQ ID NO: 169), KLKEFKYELDFYFI (SEQ ID NO: 170), SLKEFKYELDKYFL (SEQ ID NO: 171), SLPEFKYELDEYFI (SEQ ID NO: 172), SLPEFKFELDRYFI (SEQ ID NO: 173), KLPTFKYELDEYFI (SEQ ID NO: 174), ELPEFKFELDKYFI (SEQ ID NO: 175), SLPDFKYELDLYFI (SEQ ID NO: 176), KLDSFKYELDEYFI (SEQ ID NO: 177), NLKDFKFELDRYFI (SEQ ID NO: 178) or an amino acid sequence 85, 96, 87, 88, 89, 90, 91, 92, 93, 94, 95, 96, 97, 98 or 99% identical thereto. See Table 1.

TABLE 1
Design strategy: BB grafting + MPNN + AF.

| | Parent | | SEQ |
|---|---|---|---|
| | scaffold | | ID |
| Design name | (PDBid) | Epitope seq. | NO |
| S2hlx-Ex36 | 4wqw | KLKLFKCELDLYFL | 159 |
| S2hlx-Ex37 | 4wqw | KLKLFKCELDLYFL | 160 |
| S2hlx-Ex38 | 4wqw | KLRLFKLELDLYFL | 161 |
| S2hlx-Ex39 | 4wqw | RLKLFKCELDLYFL | 162 |
| S2hlx-Ex40 | 4wqw | KLKLFKCELDLYFL | 163 |
| S2hlx-Ex41 | 4wqw | KLKLFKCELDLYFL | 164 |
| S2hlx-Ex42 | 4wqw | KLLLFKEELDLYFL | 165 |
| S2hlx-Ex43 | 4wqw | SLKLFKCELDLYFL | 166 |
| S2hlx-Ex44 | 4wqw | KLLLFKCELDLYFL | 167 |
| S2hlx-Ex45 | 4l8i | ELKLFKCELDLYFL | 168 |
| S2hlx-Ex46 | 4l8i | SLCGFKGELDLYFL | 169 |
| S2hlx-Ex47 | 4l8i | KLKEFKYELDFYFI | 170 |
| S2hlx-Ex48 | 4l8i | SLKEFKYELDKYFL | 171 |
| S2hlx-Ex49 | 4l8i | SLPEFKYELDEYFI | 172 |
| S2hlx-Ex50 | 4l8i | SLPEFKFELDRYFI | 173 |
| S2hlx-Ex51 | 4l8i | KLPTFKYELDEYFI | 174 |
| S2hlx-Ex52 | 4l8i | ELPEFKFELDKYFI | 175 |
| S2hlx-Ex53 | 4l8i | SLPDFKYELDLYFI | 176 |
| S2hlx-Ex54 | 4l8i | KLDSFKYELDEYFI | 177 |
| S2hlx-Ex55 | 4l8i | NLKDFKFELDRYFI | 178 |

In embodiments, antibodies that are stimulated by and/or react with a coronavirus S2 stem helix can cross-react with the epitopes disclosed herein. Non-limiting examples of antibodies that can bind to the epitopes can include S2P6, DH1057.1, S2P6iGL, DH1057UCA, CC40.8. In some embodiments, 2, 3, 4 or all 5 of these antibodies bind to epitope scaffolds disclosed herein that contain epitopes related to coronavirus S2 stem helices.

### Protein Scaffolds or Scaffold Polypeptides

Herein, scaffolds refer to proteins or parts of proteins that are used as a “framework” or “host” for the epitopes described herein. Protein scaffolds into which an epitope has been grafted or transplanted are referred to as epitope scaffolds. Epitope scaffold molecules are engineered by transplanting the structure of the antibody-bound epitopes from viral molecules into the protein scaffolds. Unlike peptide-based immunogens that can sample diverse epitope conformations, epitope scaffolds generally present only the antibody-bound conformation of the target epitope on their surface, which typically leads to higher antibody affinity and the elicitation of antibodies specific for the structure of the target epitope.

Computational methods are used to find an appropriate scaffold for a particular epitope (e.g., a scaffold that can present the chosen epitope in a conformation to which a selected antibody can bind, and in conformation that can stimulate an appropriate immune response). In some embodiments, the computational methods used to model and identify the scaffolds include “side-chain grafting” and “backbone grafting.” For example, in these methods, computational techniques can configurationally “match” a section of a scaffold protein with a chosen epitope (e.g., match can refer to the ability of a part of the scaffold to display a transplanted epitope in a desired configuration). In side-chain grafting, the shape of the entire length of the epitope is matched with a like region of a scaffold protein. In backbone grafting, the ends of an epitope are matched with a like region of a scaffold protein that can accommodate the epitope ends.

Practically, in both side-chain and backbone grafting, libraries of potential protein scaffolds are searched to identify a protein scaffold that is compatible with a chosen epitope (e.g., search identifies proteins with exposed regions that closely match the structure of the epitope). The epitope is then used to replace the like region of the protein scaffold (i.e., the epitope is “grafted” into the protein scaffold) to produce the ES. The root-mean-square-deviation (RMSD) of the epitope-grafted ES as compared to the scaffold protein not containing the epitope can be determined. Low RMSD values are preferred. Additional mutations can be introduced into non-epitope regions of the ES to accommodate the grafted epitope. These mutations can lower the RMSD.

In various embodiments, example protein scaffolds used to produce the epitope scaffolds disclosed herein can be a polypeptide from a Kai A protein from Anabaena sp PCC7120 (PDBid 1r5q), a T4 Lysozyme protein from Escherichia coli virus T4 (PDBid 1l23); a Conserved Hypothetical protein from Staphylococcus aureus (PDBid 1tsj), a Syntaxin-1A protein from Rattus norvegicus (PDBid lez3), a PG0816 protein from Porphyromonas gingivalis W83 (PDBid 2apl), a Response regulator receiver protein from Acetivibrio thermocellus ATCC 27405 (PDBid 3jte), a Phosphate Regulon protein from Escherichia coli (PDBid 2jb9), a CcmG protein from Escherichia coli (PDBid 2blk), a ClpS 2 protein from Agrobacterium fabrum str. C58 (PDBid 4yjm), a t-SNARE VTI1 polypeptide from Saccharomyces cerevisiae (PDBid 3onj), a Catenin Alpha-protein from Mus musculus (PDBid 5y04), a PA2169 protein from Pseudomonas aeruginosa PAO1 (PDBid 4etr), a Thioredoxin protein from Caulobacter vibrioides NA1000 (PDBid 6esx), a WA352 protein from Oryza sativa indica (PDBid 5zt3), a B562RIL protein from Escherichia coli (PDBid 5yo4), or a molecule 90, 91, 92, 93, 94, 95, 96, 97, 98 or 99% identical thereto. In some embodiments, these scaffold proteins can be used as a framework for the epitopes related to the fusion peptide. In some embodiments, these protein scaffolds can be used for epitopes to which antibodies raised against fusion peptide amino acids 815-823 from the S2 spike region cross-react.

In various embodiments, example protein scaffolds used to produce the epitope scaffolds disclosed herein can be a Ferredog-Diesel protein (synthetic construct) (PDBid 6nuk), a CRISPR complex subunit Csm2 protein from Staphylococcus epidermidis RP62A (PDBid 6nbu), a SarX protein from Staphylococcus aureus sp. NCTC 8325 (PDBid 5ywj), a B562RIL protein from Escherichia coli (PDBid 5ym7), a CB15 Pilus assembly protein CpaE from Caulobacter vibrioides (PDBid 4n0p), an AvtR protein from Acidianus filamentous virus 6 (PDBid 4hv0), an Acyl carrier RPA2022 protein from Rhodopseudomonas palustris (PDBid 31mo), a Response regulator protein from Hahella chejuensis KCTC 2396 (PDBid 3kht), a RBSTsP2171 protein from Escherichia coli (PDBid 3fgx), a TM_1646 protein from Thermotoga maritima MSB8 (PDBid 2p61), a Diheme Cytochrome C protein from Rhodovulum sulfidophilum (PDBid 1h31), an ATP-dependent protease La 1 from Bacillus subtilis subsp. subtilis str. 168 (PDBid 3m65), a Mitochondrial antiviral signaling protein from Equus caballus (PDBid 4o9f), a Staphylococcal nuclease mutant T44V protein from Staphylococcus aureus (PDBid 2eyf), a Putative periplasmic protein BACEGG_01429 protein from Bacteroides eggerthii DSM 20697 (PDBid 4hbr), or a molecule 90, 91, 92, 93, 94, 95, 96, 97, 98 or 99% identical thereto. In some embodiments, these scaffold proteins can be used as a framework for the epitopes related to the S2 helix peptide. In some embodiments, these protein scaffolds can be used for epitopes to which antibodies raised against S2 helix peptide amino acids 1148-1156 from the S2 spike region cross-react.

In various embodiments, example protein scaffolds uses to produce the epitope scaffolds disclosed herein can be a UPF0247 E protein from Staphylococcus aureus (PDBid lvh0), a VPS54 protein from Mus musculus (PDBid 3nlb), a FFL_005 protein (Synthetic RSV epitope scaffold) (PDBid 4l8i), a Vtilb Habc domain from Mus musculus (PDBid 2qyw), a CDC37 protein from Homo sapiens (PDBid 2w0g), a SaeR protein from Staphylococcus aureus (PDBid 4wqw), a syntaxin 6 protein from Rattus norvegicus (PDBid 1lvf), a Vtilb Habc domain from Saccharomyces cerevisiae (PDBid 3onj), or a molecule 90, 91, 92, 93, 94, 95, 96, 97, 98 or 99% identical thereto. In some embodiments, these scaffold proteins can be used as a framework for the epitopes related to the S2 helix. In some embodiments, these protein scaffolds can be used for epitopes to which antibodies raised against S2 helix peptide amino acids 1144-1157 from the S2 spike region (extended S2 helix epitope) cross-react.

### Epitope Scaffolds

“Epitope scaffolds” or ES can refer to new antigens/immunogens designed using computational methods in which linear epitopes are transplanted to scaffold proteins for structural stabilization and immune presentation. ES are proteins engineered by transplanting the structure of antibody-bound epitopes onto protein scaffolds. Unlike peptide-based immunogens that can sample diverse epitope conformations, epitope scaffolds generally present only the antibody-bound conformation of the target epitope on their surface, which typically leads to higher antibody affinity and elicitation of antibodies specific for the structure of the target epitope. Unlike peptide antigens that can usually adopt multiple conformations in solution, epitope scaffolds are engineered to preferentially stabilize a particular epitope conformation, usually the conformation of the epitope when it is bound to a neutralizing antibody. In embodiments, epitope scaffolds can serve as vaccine candidates or as reagents to map different sera specificities. In embodiments, epitope scaffolds can serve as a platform to test the ability of computational design to manipulate protein structure and function. In embodiments, the epitopes disclosed herein, related to the S2 region of the human coronavirus spike protein, can be grafted or transplanted into a scaffold polypeptide to produce an epitope scaffold.

Herein, epitope grafting is used to design fusion peptide and stem helix epitope scaffolds that interact with broadly cross-reactive antibodies against the fusion peptide or stem helix regions of the coronavirus spike protein. These epitope scaffolds can be used to induce these cross-reactive antibodies using immunization.

“Grafting” epitopes into a protein scaffold to produce epitope scaffolds utilizes scaffold proteins that can accommodate replacement of an exposed segment with a crystallized conformation of the target epitope. For each suitable scaffold identified by computationally searching through the protein crystal structures, an exposed segment is replaced by the target epitope and

Additional amino acids within the epitope can be changed. Within an epitope fragment, some amino acid residues point toward the antibody and make interactions with it, and some amino acid residues point away and are essential for antibody interactions. During the design process, epitope residues that interact with the antibody are not changed, while those that do not contact the antibody can be changed. Typically, the latter amino acid residues point towards the scaffold and can be changed to stabilize the epitope in the scaffold. In embodiments, antibody-interacting residues in the epitope are not changed, while epitope residues that do not interact with the antibody can be changed. In some embodiments, the amino acid changes can improve display of the epitope within the epitope scaffold.

In some embodiments, an amino acid tag can be added to the protein scaffold (e.g., a histidine tag) to improve purification of the epitope scaffold, for example. In some embodiments, amino acids can be trimmed from the beginning and/or end of the parent protein scaffold, if they are thought to be detrimental or not necessary for the construct, for example. In some embodiments, a few amino acids can be added at the N- and C-terminus of the parent protein scaffold due to cloning sites used in the construction process.

Grafting in this way is facilitated when the replaced segment in the scaffold protein and the inserted epitope have similar translation and rotation transformations between their N- and C-termini, and when the surrounding peptide backbone does not clash with the inserted epitope. Grafted epitope-scaffolds can mimic the epitope-antibody interaction. Grafting methods can be used to handle complex epitopes.

“Side-chain grafting computational method” or “side-chain grafting” can refer to a procedure in which side chains of a linear epitope are transplanted to backbone position in other proteins that have exposed backbone segments conformationally similar to the backbone of the antibody-bound epitope. In embodiments, the fusion peptide epitope is grafted into the scaffold polypeptide using the side chain grafting computational method.

In embodiments, the ES can comprise between about 2 and about 25, about 5 and about 20 or about 7 and about 17 amino acid changes compared to a corresponding scaffold polypeptide not comprising the fusion peptide epitope. In embodiments, ES can comprise between about between about 2 and about 25, about 5 and about 20 or about 7 and 17 amino acid changes compared to a corresponding scaffold polypeptide not comprising the S2 stem helix epitope.

In embodiments, a root-mean-square-deviation (RMSD) of the ES as compared to a corresponding scaffold polypeptide not comprising the fusion peptide epitope is less than about 1.0, 0.9, 0.8, 0.7, 0.6, 0.5, 0.4, 0.3, 0.2 or 0.1 Å. In embodiments, RMSD of the ES as compared to a corresponding scaffold polypeptide not comprising the S2 stem helix epitope is less than about 1.0, 0.9, 0.8, 0.7, 0.6, 0.5, 0.4, 0.3, 0.2 or 0.1 Å.

“Backbone grafting computational method” or “backbone grafting” can refer to the procedure in which the native backbone of a candidate scaffold is replaced with the desired backbone conformation of a functional motif. In embodiments, backbone grafting imposes the conformation of a given epitope onto a scaffold and integrates that epitope conformation into the scaffold through backbone remodeling and sequence design in regions flanking the epitope. In embodiments, the S2 stem helix epitope is grafted into the scaffold polypeptide using the backbone grafting computational method.

In embodiments, the ES can comprise between about 8 and 38, about 10 and 35 or about 13 and about 32 amino acid changes compared to a corresponding scaffold polypeptide not comprising the S2 stem helix epitope.

In embodiments, a RMSD of the ES as compared to a corresponding scaffold polypeptide not comprising the S2 stem helix epitope is less than about 12, 11, 10, 9, 8, 7, 6, 5, 4, 3, 2 or 1 Å.

In some embodiments, the epitope from the S2 region of the human coronavirus spike protein is grafted into a scaffold polypeptide to produce an ES. In some embodiments, the epitope scaffold or the scaffold polypeptide can be about 70, 80, 90, 100, 110, 120, 130, 140, 150, 160, 170, 180, 190, 200, 210, 220, 230, 240, 250, 260, 270, 280, 290 or 300 amino acids in length.

While currently approved coronavirus vaccines and multiple next-generation vaccine candidates are based on RBD epitopes, the epitope scaffolds described herein are focused on conserved fusion peptide and stem helix epitopes. Elicitation of antibodies against these immunogens can lead to a “pan-coronavirus vaccine.” In some embodiments, the fusion peptide epitope and/or the S2 stem helix epitope is/are grafted into a protein scaffold using a side chain grafting computational method or a backbone grafting computation method.

An antibody can specifically bind to an epitope from an S2 region of a human coronavirus spike protein transplanted into a protein scaffold to produce an epitope scaffold, such as the fusion peptide or S2 stem helix. In some embodiments, the antibody can bind when the equilibrium binding constant (K_{D}) is ≤1 mM, ≤10 μM, ≤1 μM, ≤10 nM, ≤1 nM, ≤100 μM, ≤10 μM, or ≤1 μM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about IE-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M.

In some embodiments, a dissociation constant (K_{D}) for binding of DH1058 to the immunogenic composition comprising the fusion protein epitope grafted into the scaffold protein is between about 10⁻¹ and about 10³ nanomolar (nM).

In some embodiments, a dissociation constant (K_{D}) for binding of DH1294 to the immunogenic composition comprising the fusion protein epitope grafted into the scaffold protein is between about 10⁻¹ and about 10³ nanomolar (nM).

In some embodiments, a dissociation constant (K_{D}) for binding of S2P6 to the immunogenic composition comprising the S2 stem helix epitope grafted into the scaffold protein is between about 10⁻¹ and 10² nanomolar (nM).

In some embodiments, a dissociation constant (K_{D}) for binding of DH1057 to the immunogenic composition comprising the S2 stem helix epitope grafted into the scaffold protein is between about 10 micromolar (μM) and 10 nM.

In some embodiments, a dissociation constant (K_{D}) for binding of S2P6, DH1057 or CC40.8 to the immunogenic composition comprising the S2 stem helix epitope grafted into the scaffold protein is between about 1 and 10² nanomolar (nM).

In some embodiments, the epitope scaffolds can be molecules having SEQ ID NOs: 1-65, or a molecule having 85, 86, 87, 88, 89, 90, 91, 92, 93, 94, 95, 96, 97, 98 or 99% identity thereto.

In some embodiments, the epitope scaffolds may contain mutations, (e.g., amino-acid substitutions). For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members. In some embodiments, after side chain grafting or backbone grafting of the epitope into the protein scaffold, mutations in the regions of the epitope scaffold, outside of the epitope region, can be made to better accommodate the epitope.

The epitope scaffolds s can be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In a further embodiment, the epitope scaffolds of any embodiment of the invention may further comprise a tag, such as a detectable moiety or therapeutic agent. The tag(s) can be linked to the polypeptide through covalent bonding, including, but not limited to, disulfide bonding, hydrogen bonding, electrostatic bonding, recombinant fusion and conformational bonding. Alternatively, the tag(s) can be linked to the epitope scaffold by means of one or more linking compounds. Techniques for conjugating tags to polypeptides are well known to the skilled artisan. Polypeptides comprising a detectable tag can be used, for example, as probes to isolate B cells that are specific for the epitope present in the polypeptide. However, they may also be used for other detection and/or analytical purposes. Any suitable detection tag can be used, including but not limited to enzymes, prosthetic groups, fluorescent materials, luminescent materials, bioluminescent materials, radioactive materials, positron emitting metals, and nonradioactive paramagnetic metal ions. The tag used will depend on the specific detection/analysis techniques and/or methods used Such as flow cytometric detection, scanning laser cytometric detection, fluorescent immunoassays, enzyme-linked immunosorbent assays (ELISAS), radioimmunoassays (RIAS), bioassays (e.g., neutralization assays), Western blotting applications, etc. When the polypeptides of the invention are used for flow cytometric detections, scanning laser cytometric detections, or fluorescent immunoassays, the tag may comprise, for example, a fluorophore. A wide variety of fluorophores useful for fluorescently labeling the polypeptides of the invention are known to the skilled artisan. When the polypeptides are used for in vivo diagnostic use, the tag can comprise, for example, magnetic resonance imaging (MRI) contrast agents, such as gadolinium diethylenetriaminepentaacetic acid, to ultrasound contrast agents or to X-ray contrast agents, or by radioisotopic labeling.

The epitope scaffolds of the invention can also comprise a tag, such as a linker (including but not limited to an amino acid linker Such as cysteine or lysine), for binding to a particle, such as a virus-like particle. As another example, the polypeptides of the invention can usefully be attached to the surface of a microtiter plate for ELISA. The polypeptides of the invention can be fused to marker sequences to facilitate purification, as described in the examples that follow. Examples include, but are not limited to, the hexa-histidine tag, the myc tag or the flag tag.

In another embodiment, a plurality of the epitope scaffolds may be complexed to a dendrimer. Dendrimers are three dimensional, highly ordered oligomeric and/or polymeric compounds typically formed on a core molecule or designated initiator by reiterative reaction sequences adding the oligomers and/or polymers and providing an outer surface. Suitable dendrimers include, but are not limited to, “star burst” dendrimers and various dendrimer polycations. Methods for the preparation and use of dendrimers are well known to those of skill in the art.

In another embodiment, the epitope scaffolds may be fused (via recombinant or chemical means) via their N-terminus, C-terminus, or both N- and C-termini, to an oligomerization domain. Any suitable oligomerization domain can be used. In one non-limiting embodiment, the polypeptides are fused to GCN4 variants that form trimers (hence trimers or hexamers of the fused polypeptide could be displayed). In another non-limiting embodiment, the polypeptides are fused to a fibritin foldon domain that forms trimers. In other non-limiting embodiments, the oligomerization domain could be any protein that assembles into particles, including but not limited to particles made from a (non-viral) lumazine synthase protein and particles made from (non-viral) ferritin or ferritin-like proteins.

In another embodiment, the epitope scaffolds may be chemically conjugated to liposomes. In one non-limiting embodiment, the liposomes contain a fraction of PEGylated lipid in which the PEG groups are functionalized to carry a reactive group, and the polypeptide is chemically linked to the reactive group on the PEG. In another non-limiting embodiment, additional immune-stimulating compounds are included within the liposomes, either within the lipid layers or within the interior. In another non-limiting embodiment, specific cell-targeting molecules are included on the surface of the liposome, including but not limited to molecules that bind to proteins on the surface of dendritic cells.

In another embodiment, a plurality (i.e., 2 or more; preferably at least 5, 10, 15, 20, 25, 50, 75, 90, or more copies) of the epitope scaffolds may be present in a virus-like particle (VLP), to further enhance presentation of the polypeptide to the immune system. As used herein, a “virus-like particle” refers to a structure that in at least one attribute resembles a virus but which has not been demonstrated to be infectious. Virus-like particles in accordance with the invention do not carry genetic information encoding for the proteins of the virus-like particles. In general, virus-like particles lack a viral genome and, therefore, are noninfectious. In addition, virus-like particles can often be produced in large quantities by heterologous expression and can be easily purified. In some embodiments, the VLP comprises viral proteins that may undergo spontaneous self-assembly, including but not limited to recombinant proteins of adeno associated viruses, rotavirus, recombinant proteins of norwalkvirus, recombinant proteins of alphavirus, recombinant proteins of foot and mouth disease virus, recombinant proteins of retrovirus, recombinant proteins of hepatitis B virus, recombinant proteins of tobacco mosaic virus, recombinant proteins of flock house virus, and recombinant proteins of human papilloma virus, and Qbeta bacteriophage particles.

These configurations of the epitope scaffolds can be used as vaccines or antigenic formulations for treating or limiting SARS-CoV-2 infection, as discussed herein. In some embodiments, the VLPs may further comprise other epitope scaffolds presenting other epitopes from SARS-CoV-2 proteins.

In another embodiment, the epitope scaffolds may be present on a non-natural core particle. Such as a synthetic polymer, a lipid micelle or a metal. Such core particles can be used for organizing a plurality of polypeptides of the invention for delivery to a subject, resulting in an enhanced immune response. By way of example, synthetic polymer or metal core particles can use a calixarene organic scaffold to which is attached a plurality of peptide loops in the creation of an antibody mimic, or nanocrystalline particles can be used as a viral decoy that are composed of a wide variety of inorganic materials, including metals or ceramics. Preferred metals in this embodiment include chromium, rubidium, iron, Zinc, selenium, nickel, gold, silver, platinum. Preferred ceramic materials in this embodiment include silicon dioxide, titanium dioxide, aluminum oxide, ruthenium oxide and tin oxide. The core particles of this embodiment may be made from organic materials including carbon (diamond). Preferred polymers include polystyrene, nylon and nitrocellulose. For this type of nanocrystalline particle, particles made from tin oxide, titanium dioxide or carbon (diamond) are particularly preferred. A lipid micelle may be prepared by any means known in the art. See U.S. Pat. No. 7,229,624 and references disclosed therein.

The epitope scaffolds of the invention can be used to generate antibodies that recognize the epitopes of the invention. The method comprises administering to a subject a polypeptide, VLP, or composition of the invention. Such antibodies can be used, for example, in SARS-CoV-2 research. A subject employed in this embodiment is one typically employed for antibody production, including but not limited to mammals, such as, rodents, rabbits, goats, sheep, etc. The antibodies generated can be either polyclonal or monoclonal antibodies. Polyclonal antibodies are raised by injecting (e.g. subcutaneous or intramuscular injection) antigenic polypeptides into a suitable animal (e.g., a mouse or a rabbit). The antibodies are then obtained from blood samples taken from the animal. The techniques used to produce polyclonal antibodies are extensively described in the literature. Polyclonal antibodies produced by the subjects can be further purified, for example, by binding to and elution from a matrix that is bound with the polypeptide against which the antibodies were raised. Those of skill in the art will know of various standard techniques for purification and/or concentration of polyclonal, as well as monoclonal, antibodies. Monoclonal antibodies can also be generated using techniques known in the art.

### Immunogenic Compositions

Immunogenic compositions contain the epitopes and/or epitope scaffolds described herein. In some embodiments, the immunogenic composition does not contain a receptor binding domain (RBD) from coronavirus (e.g., amino acids 319-542 of SARS-CoV-2 spike protein). In some embodiments, the immunogenic composition does contain an RBD domain from coronavirus.

### Antibodies

An “antibody” or “antigen-binding polypeptide” can refer to a polypeptide or a polypeptide complex that specifically recognizes and binds to an antigen, such as fusion peptide or S2 stem helix epitope. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides. Non-limiting examples of antibodies that bind to epitopes similar to fusion peptides can include DH1058, DH1294, VN01H1, Cov44-62 and Cov44-79. Non-limiting examples of antibodies that bind to epitopes similar to S2 stem helix epitopes can include S2P6, DH1057.1, S2P6iGL, DH1057UCA and CC40.8.

The terms “antibody fragment” or “antigen-binding fragment” can refer to a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)2, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” can refer to a fusion protein of the variable regions of the heavy (VH) and light chains (VL) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. In embodiments the regions are connected with a short linker peptide, such as a short linker peptide of about ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. Very large naive human scFv libraries have been and can be created to offer a large source of rearranged antibody genes against a plethora of target molecules. Smaller libraries can be constructed from individuals with infectious diseases in order to isolate disease-specific antibodies.

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG1, IgG2, IgG3 and IgG4 and others. The immunoglobulin subclasses (isotypes) e.g., IgG1, IgG2, IgG3, IgG4, IgG5, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (K, k). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions can adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined.

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself.

Antibodies may be polyclonal or monoclonal; xenogeneic, allogeneic, or syngeneic; or modified forms thereof (for example, humanized, chimeric, etc.). Antibodies may also be fully human. Preferably, antibodies of the invention bind specifically or substantially specifically to a component of cBAF complex. The terms “monoclonal antibodies” and “monoclonal antibody composition,” as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Disclosed herein are epitopes and/or epitope scaffolds that can be used as immunogens. In some embodiments, the epitopes and/or epitope scaffolds can elicit sera with broad betacoronavirus reactivity.

In some embodiments, the epitopes and/or epitope scaffolds can interact with B cells that can give rise to antibodies with broad activity against the spike peptide or the stem helix from subjects with preexisting betacoronaviurs (e.g., SARS-CoV-2) immunity. In some embodiments, the preexisting immunity of the subject can be elicited by previous vaccination against betacoronavirus (e.g., SARS-CoV-2). In some embodiments, the preexisting immunity of the subject can be elicited by previous exposure (e.g., infection) to betacoronavirus (e.g., SARS-CoV-2). In some embodiments, the preexisting immunity of the subject can be elicited by previous vaccination against betacoronavirus (e.g., SARS-CoV-2) and previous exposure (e.g., infection) to betacoronavirus (e.g., SARS-CoV-2). In some embodiments, the epitopes and/or epitope scaffolds can protect as “boosts” against live virus challenge in subjects.

The epitopes and/or epitope scaffolds described herein can be used to isolate antibodies from sera of subjects with preexisting immunity to a betacoronavirus. In some embodiments, the epitopes and/or epitope scaffolds described herein can be used to isolate antibodies from sera of subjects pre-exposed to a sarbecovirus. In some embodiments, the epitopes and/or epitope scaffolds described herein can be used to isolate antibodies from sera of subjects with preexisting immunity to a SARS-CoV-2. In some embodiments, the subject is a human.

In some embodiments, the isolated antibody can be a recombinant monoclonal antibody. identified B cells can give rise to antibodies with broad activity against Betacoronaviruses. In some embodiments, the epitopes and/or epitope scaffolds can interact with B cells that can give rise to antibodies with broad activity against the spike peptide or the stem helix from people with preexisting SARS-CoV-2 immunity. In some embodiments, the epitopes and/or epitope scaffolds can interact with B cells that can give rise to antibodies with broad activity against the Fusion Peptide from people with preexisting SARS-CoV-2 immunity.

In some embodiments, the epitope and/or epitope scaffold can stimulate immune responses to a Fusion Peptide. In some embodiments, the epitope and/or epitope scaffold can stimulate immune responses to an S2 Stem Helix. In some embodiments, the S2 Stem Helix epitope and/or epitope scaffold include spike peptide. In some embodiments, the Fusion Peptide epitope and/or epitope scaffold can include FP-10.

In some embodiments, the antibody can be isolated from B cells identified using positive selection for an epitope scaffold and negative selection for the parental version of said scaffold that lacks the epitope. In some embodiments, positive selection can isolate antibodies that can bind to an epitope scaffold and negative selection can isolate antibodies that cannot bind to the parental version of said epitope scaffold that lack said epitope. In some embodiments, the antibody can bind to an epitope scaffold (SEQ ID NOs: 1-65 and 139-158) carrying an epitope (SEQ ID NOs: 66-112 and 159-180). In some embodiments, the isolated antibody cannot bind to a parent scaffold that lacks the epitope (SEQ ID NOs: 1-65 and 139-158). In some embodiments, the isolated antibody can bind to an epitope scaffold (SEQ ID NOs: 1-65 and 159-158) carrying an epitope (SEQ ID NOs: 66-112 and 159-180), and it cannot bind to a corresponding parent scaffold that lacks the epitope (SEQ ID NOs: 1-65 and 159-158). In some embodiments, the isolated antibody can bind to an epitope scaffold S2hlx-Ex19, and it cannot bind to the corresponding parental protein scaffold that lacks the epitope, S2hlx-Ex19-PS. In some embodiments, the isolated antibody can bind to S2hlx-Ex19. In some embodiments, the recombinant antibody can bind to FP-10. In some embodiments, the recombinant antibody cannot bind to FP-10. In some embodiments, the recombinant antibody can bind to FP-10, and it cannot bind to the corresponding parental protein scaffold that lacks the epitope, FP-10-PS.

In some embodiments, the isolated antibody can include any of the heavy chain variable region amino acid sequences included in Table 2 and/or any of the light chain variable region amino acid sequences included in Table 3. In some embodiments, the isolated antibody that reacts to the epitopes and epitope scaffolds described herein can include DH1490, DH1491, DH1501.1, DH1501.2, DH1501.3, DH1495, DH1496, Dh1497, DH1498, DH1499, DH1506, DH1509, DH1508. In some embodiments, isolated antibodies DH1490, DH1491, DH1501.1, DH1501.2, DH1501.3, DH1495, DH1496, DH1497, DH1498, DH1499, and DH1506 can react to stem helix epitopes and/or scaffold epitopes. In some embodiments, isolated antibodies DH1509, DH1508 can react to fusion peptide epitopes and/or scaffold epitopes.

In some embodiments, isolated antibodies DH1490, DH1491, DH1501.1, DH1501.2, DH1501.3, DH1495, DH1496, Dh1497, DH1498, DH1499, DH1506 can react to an epitope scaffold (e.g., S2hlx-Ex19) and cannot react to the corresponding parental protein scaffold that lacks the epitope (e.g., S2hlx-Ex19-PS). In some embodiments, isolated antibodies DH1490, DH1491, DH1501.1, DH1501.2, DH1501.3, DH1495, DH1496, Dh1497, DH1498, DH1499, DH1506 can react to S2hlx-Ex19.

In some embodiments, isolated antibodies DH1509, DH1508 can react to fusion peptide epitopes and/or scaffold epitopes. In some embodiments, DH1509, DH1508 can react to FP-10, and they cannot react to the corresponding parental protein scaffold that lacks the epitope FP-10-PS. In some embodiments, DH1509, DH1508 can react to FP-10.

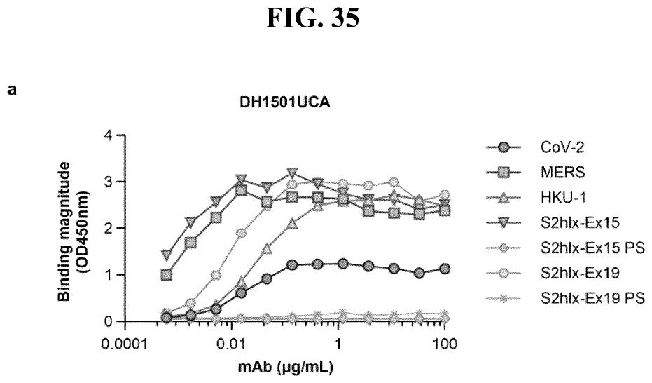

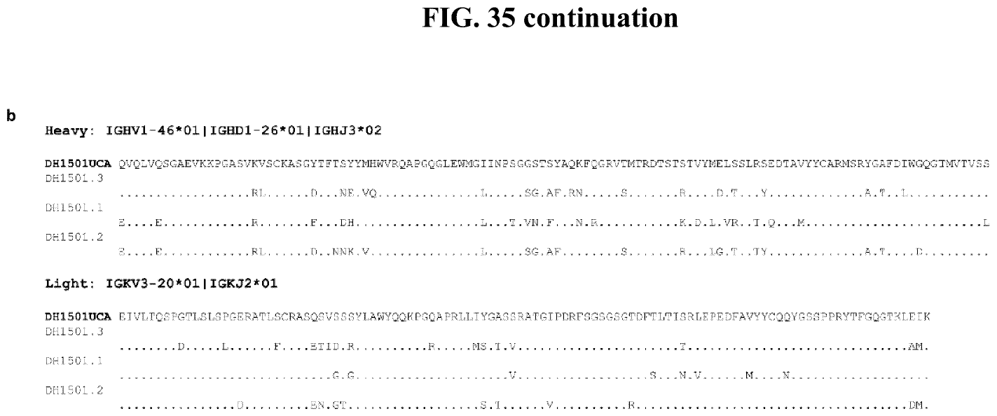

In some embodiments, DH1490, DH1491, DH1501.1, DH1501.2, DH1501.3, DH1495, DH1496, DH1497, DH1498, DH1499, DH1506, DH1509, DH1508 can react (e.g., bind) to spike proteins from betacoronaviruses. In some embodiments, DH1490, DH1491, DH1501.1, DH1501.2, DH1501.3, DH1495, DH1496, Dh1497, DH1498, DH1499, DH1506, DH1509, DH1508 can bind (e.g., bind) to spike proteins from SARS-CoV-2 VOCs including but not limited to WA-2, Delta, Ba1.1 and XBB; human betacoronavirus including but not limited to CoV-1, MERS, OC43 and HKU-1, 229E and NL63; and animal betacoronavirus including but not limited to GXP4L, SHC014 and RaTG13. In some embodiments, the inferred UCA (FIG. 35B and SEQ ID NOs: 181-182) of DH1501.1, DH1501.2 and DH1501.3 can bind to multiple spike proteins. In some embodiments, the inferred UCA of DH1501.1, DH1501.2, and DH1501.3 can bind to human coronavirus spike proteins CoV-2, MERS, and HKU-1.

In some embodiments, the isolated antibodies described herein (Table 2 and Table 3) can include unique VH/VL pairings not previously reported. In some embodiments, the epitopes and/or epitope scaffolds described herein can stimulate a clonally diverse response. In some embodiments, the antibodies described herein can neutralize SARS-COV-2 variants and SARS-CoV-1. In some embodiments, the antibodies described herein can neutralize D614G, B1.351(146R), B1.617.2, BA.1, BA2.75.2, BA.4/BA.5, BA.4.6, BF.7, BQ.1.1, XBB.1, XB.1.5 and CoV-1.

In some embodiments, the isolated antibodies described herein (Table 2 and Table 3) can mediate Fc effector functions. In some embodiments, the isolated antibodies described herein (Table 2 and Table 3) can bind to virus-infected cells. In some embodiments, the isolated antibodies described herein can bind to cells infected with either the D614G or the BA.1 variant of SARS-CoV-2 in the same range as the previously described stem helix antibodies CC40.8, S2P6 and DH1057.1. In some embodiments, the antibodies described herein can induce NK cell degranulation against D614, Ba.1 and BA.4/5, with potencies similar to those of known stem helix antibodies.

In some embodiments the antibody or antigen-binding fragment or variant thereof can comprise a heavy chain variable region HCVR including the amino acid sequence QVQLQQSGPGLVKPSETLSLTCTVSGGSISSSSYYWGWIRQPPGKGLEWIGSIYYSGS TYYNPSLKSRVTISVDTSKNQFSLKLSSVTAADTAVYYCARHGPLRFRLGYYDSSGY LFQHWGQGTLVTVSS (SEQ ID NO: 113) or a sequence that has at least 95% sequence identity thereto, and an LCVR region including the amino acid sequence DIQLTQSPSTLSASVGDRVTITCRASQSISSWLAWYQQKPGKAPKLLIYDASSLESGV PSRFSGSGSGTEFTLTISSLQPDDFATYYCQQYNSYSRWTFGQGTKVEIK (SEQ ID NO: 126) or a sequence that has at least 95% sequence identity thereto.

In some embodiments the antibody or antigen-binding fragment or variant thereof can comprise a heavy chain variable region HCVR including the amino acid sequence QVQLVQSGGGLVQPGGSLRLSCATSGFNFRAFAMSWVRQAPGKGLEWAAVMSGTD DGTYYVESVKGRFTIFRDNSENTVYLQMNSLRADDSAIYYCAKGTLGHCSGVDCYY LDYWGRGTLVTVSS (SEQ ID NO: 114) or a sequence that has at least 95% sequence identity thereto, and an LCVR region including the amino acid sequence DIVLTQSPATLSLSPGERATLSCRASQSVGTYVAWYQHKVGQAPRLLIYDASTRATDI PARFSGSGSGTDFTLTITSLEPEDVAIYYCQQRVNLVTFGGGTKLEIK (SEQ ID NO: 127) or a sequence that has at least 95% sequence identity thereto.

In some embodiments the antibody or antigen-binding fragment or variant thereof can comprise a heavy chain variable region HCVR including the amino acid sequence QVQLVQSGAEVKKPGASVRLSCKASGDTFTNEYVQWVRQAPGQGLEWMGLINPSG SGTAFARNFQGRVSMTRDTSTRTVYMDLTSLRYEDTAVYYCARMSRAGTFDLWGQ GTMVTVSS (SEQ ID NO: 115) or a sequence that has at least 95% sequence identity thereto, and an LCVR region including the amino acid sequence EIVLTQSPDTLSLSLGERATLFCRASETIDSRYLAWYQQKPGRAPRLLMSGTSVRATG IPDRFSGSGSGTDFTLTITRLEPEDFAVYYCQQYGSSPPRYTFGQGTKLAMK (SEQ ID NO: 128) or a sequence that has at least 95% sequence identity thereto.

In some embodiments the antibody or antigen-binding fragment or variant thereof can comprise a heavy chain variable region HCVR including the amino acid sequence EVQLVESGGGLVQPGGSLRLSCAASGFTFDNYAMGWVRQPPGKGLEWVSSFSGRG VSTYYADSVKGRFTVSRDSSKNTLFLQMNYLRVEDTAVYYCATYPYDILTGYYGAF DYWGQGALVTVSS (SEQ ID NO: 116) or a sequence that has at least 95% sequence identity thereto, and an LCVR region including the amino acid sequence DIQLTQSPSTLSASVGDRVTITCRASQTIDTWLAWYQQKPGKAPKLLIYGASSLQSGV PSRFSASGSGTEFTLTISSLQPDDFAIYYCQQYKDYSTFGQGSKVEFM (SEQ ID NO: 129) or a sequence that has at least 95% sequence identity thereto.

In some embodiments the antibody or antigen-binding fragment or variant thereof can comprise a heavy chain variable region HCVR including the amino acid sequence QVQLQQWGAGLLKPSETLSLTCSFYGGSFSGYCWSWIRQSPGKGLEWIGEINHSGST NYNPSLKTRVTILIDTSNNQFSLRLSSVTAADTAVYYCARERAIRRCTSTSCYRVGGV AGLDVWGQGTTVSVSS (SEQ ID NO: 117) or a sequence that has at least 95% sequence identity thereto, and an LCVR region including the amino acid sequence DIVMTQSPSSLSASVGDRVTITCQASRDIYKYLNWYQQKPGQAPKLLISDASNLETG VPSRFSGSGSGTDFTFTISSLQPEDIAAYYCQQYDNPLITFGQGTRLEIK (SEQ ID NO: 130) or a sequence that has at least 95% sequence identity thereto.

In some embodiments the antibody or antigen-binding fragment or variant thereof can comprise a heavy chain variable region HCVR including the amino acid sequence QVQLVQSGGGLAQPGESLRLSCAASGFTFSSYAMTWVRQAPGKGLEWVSSISGKGE NIEYAESVKGRFTISRDNAKNTVDLEMNSLRAEDTATYFCAKHIYGAFIVVPNTLYD ALDVWGQATKVTVSS (SEQ ID NO: 118) or a sequence that has at least 95% sequence identity thereto, and an LCVR region including the amino acid sequence EIVLTQSPGTLSLSPGERATLSCRASQSVSRNYLAWYQQKPGQAPRILVYDASNRAIG IPDRFSGSGSGTDFTLTISRLEPEDFAVYYCQQYGSSPGTFGQGTRLEIK (SEQ ID NO: 131) or a sequence that has at least 95% sequence identity thereto.

In some embodiments the antibody or antigen-binding fragment or variant thereof can comprise a heavy chain variable region HCVR including the amino acid sequence QVQLQESGPGLVKPSETLSLTCAVSGGSVSSDTYYWSWIRQPPGKGLEWIGYIYNSG STNYNPSLKSRVTISVDTSKSQFSLNLGSVTAADTAVYYCAREGAGTYPPKNDAFDI WGQGTLVTVSS (SEQ ID NO: 119) or a sequence that has at least 95% sequence identity thereto, and an LCVR region including the amino acid sequence EIVLTQSPDSLAVSLGERATINCKSSQSVLFSSDNKNYLAWYQQKPGQPPKLLIYWAS TRESGVPDRFSGSGSGTDFTLTISSLQAEDVAVYYCQQYYNSPRTFGQGTKVEIK (SEQ ID NO: 132) or a sequence that has at least 95% sequence identity thereto.

In some embodiments the antibody or antigen-binding fragment or variant thereof can comprise a heavy chain variable region HCVR including the amino acid sequence QVQLQESGGGLVKPGGSLRLSCAASGFTFSSYSMNWVRQAPGKGLEWVSSISSSSSY IYYADSVKGRFTISRDNAKNSLYLQMNSLRAEDTAVYYCARGIIDYGDYFDYWGQG TLVTVSS (SEQ ID NO: 120) or a sequence that has at least 95% sequence identity thereto, and an LCVR region including the amino acid sequence EIVLTQSPDTLSLSPGERATVSCRASQSVGAGYVAWYQQRPGQPPRLLIYGASVRAT GIPDRFSGSGSGTDFSLTINRVEPEDFAVYYCQNYASSPPRYTFGQGTKLEIK (SEQ ID NO: 133) or a sequence that has at least 95% sequence identity thereof.

In some embodiments the antibody or antigen-binding fragment or variant thereof can comprise a heavy chain variable region HCVR including the amino acid sequence EVQLVESGAEVKKPGASVRVSCKASGFTFTDHYMHWVRQAPGQGLEWMGLINPTG VNTFYAQNFRGRVTMTRDTSTKTDYLEVRSLTSQDTAMYYCARMSRYGAFDIWGQ GTMVTVSL (SEQ ID NO: 121) or a sequence that has at least 95% sequence identity thereto, and an LCVR region including the amino acid sequence EIVLTQSPGTLSLSPGERATLSCRASQSVGSGYLAWYQQKPGQAPRLLIYGASVRAT GIPDRFSGSGSGTDFSLTINRVEPEDFAMYYCQNYGSSPPRYTFGQGTKLEIK (SEQ ID NO: 134) or a sequence that has at least 95% sequence identity thereto.

In some embodiments the antibody or antigen-binding fragment or variant thereof can comprise a heavy chain variable region HCVR including the amino acid sequence EVQLVESGAEVKKPGASVRLSCKASGDTFNNKYVHWVRQAPGQGLEWMGLINPSG SGTAFAQKFQGRVSMTRDTSTRTVYLGLTSLTYEDTAVYYCARMSRAGTFDIWDQG TMVTVSS (SEQ ID NO: 122) or a sequence that has at least 95% sequence identity thereto, and an LCVR region including the amino acid sequence EIVLTQSPGTLSLSPGDRATLSCRASENVGTSYLAWYQQKPGQAPRLLISGTSSRATG VPDRFSGSGSGRDFTLTISRLEPEDFAVYYCQQYGSSPPRYTFGQGTKLDMK (SEQ ID NO: 135) or a sequence that has at least 95% sequence identity thereto.

In some embodiments the antibody or antigen-binding fragment or variant thereof can comprise a heavy chain variable region HCVR including the amino acid sequence QVQLVQSGAEVKKPGASVKLSCRASGYPITSHYMHWVRQAPGQGLEWMGIINPSGT GTSFARNFQGRVTMTRDTATRTVYMELSSLKSEDSAVYYCAGGTMGPLFDYWGQG TLVTVSS (SEQ ID NO: 123) or a sequence that has at least 95% sequence identity thereto, and an LCVR region including the amino acid sequence EIVLTQSPGTLSLSPGERATLSCRASQSVRRNFLAWYQQKPGQAPRLLIYEASTRATG IPDRFSGSGSGTDFTLTISRLEPEDFAVYYCQQYDSSPPGVTFGPGTKVDIR (SEQ ID NO: 136) or a sequence that has at least 95% sequence identity thereto.

In some embodiments the antibody or antigen-binding fragment or variant thereof can comprise a heavy chain variable region HCVR including the amino acid sequence QVQLVESGGGVVQPGKSLKLSCAASGFTFSTYSMHWVRQAPGKGLEWVAIISYDGR NKYYANSVKGRFTISRDNSKNTLYLEINNLRPQDTAVYYCATDASNVEVPGKFAPLD KWGQGTLVTVSS (SEQ ID NO: 124) or a sequence that has at least 95% sequence identity thereto, and an LCVR region including the amino acid sequence EIVMTQSPATLSVSPGERATLSCRASQSVSSDLAWYQQKPGQAPRLLIYGATTRATG VPARFSGSGSGAEFTLTISSLQSGDFAVYYCQQYNDWPLITFGQGTRLEIK (SEQ ID NO: 137) a sequence that has at least 95% sequence identity thereto.

In some embodiments, vaccination of a subject with any of the epitopes and/or epitope scaffolds described herein can elicit an immune response. In some embodiments, the immune response can have broad reactivity against betacoronavirus (e.g., diverse CoVs). In some embodiments, the immune response can be elicited by vaccination of a subject with any of the stem helix scaffolds described herein. In some embodiments, the immune response can be elicited by vaccination of a subject with any of the fusion peptide scaffolds described herein. In some embodiments, the immune response can be elicited by vaccination of a subject with a plurality of any of the epitopes and/or epitope scaffolds described herein. In some embodiments, the immune response can be elicited by stem helix epitopes and/or epitope scaffolds belonging to the S2hlx-EX2 family. In some embodiments, the immune response can be elicited by a plurality of S2hlx-Ex15, S2hlx-Ex17, S2hlx-Ex19, S2hlx-Ex20 epitope scaffolds, or a combination thereof. In some embodiments, the immune response of any of the subjects vaccinated with one or more of the epitopes and/or epitope scaffolds described herein includes antibodies (elicited antibodies).

In some embodiments, the epitope scaffolds described herein can be multimerized on nanoparticles (NPs), on mi03 NPs, for example. In some embodiments, the multimerization can be done using the SpyCatcher/Spytag conjugation system. In some embodiments, used of the SpyCatcher/Spytag conjugation system can improve immune presentation of the epitope scaffolds.

In some embodiments, the NP can be a homotypic NP and can display one type of epitope scaffold. In some embodiments, the homotypic NP can display a plurality of any of S2hlx-15Ex, S2hlx-Ex19, or S2hlx-Ex20 epitope scaffolds. In some embodiments, a homotypic NP can display between 20 and 100 copies of an epitope scaffold. In some embodiments, the NP can display 20, 30, 40, 50, 60, 70, 80, 90, 100 epitope scaffolds. In some embodiments, the NP can display 60 epitope scaffolds.

In some embodiments, the NP can be a mosaic NP and can display more than one type of epitope scaffold. In some embodiments, the mosaic NP can display a combination of any of the epitope scaffolds described herein. In some embodiments, the mosaic NP can display a combination of S2hlx-Ex15, S2hlx-Ex17, S2hlx-Ex19, S2hlx-Ex20.

In some embodiments, one or more NPs displaying any of the epitopes and/or epitope scaffolds, or a combination thereof, described herein, can be used to elicit an immune response (e.g., antibodies) in a subject. In embodiments, the subject can be a mouse. In embodiments, the subject can be a human. In some embodiments, the subject (e.g., mice) can be inoculated with S2hlx-Ex15 epitope scaffold only (Ex15-NP). In some embodiments, subjects can be inoculated with NPs including a mosaic of epitope scaffolds (Ex_mosaic-NP). In some embodiments, subjects can be inoculated with NPs including a combination of S2hlx-Ex15-NP, S2hlx-Ex19-NP and S2hlx-Ex20-NP.

In some embodiments, inoculation of the NPs into a subject can include a sequential regimen including an S2hlx-Ex15-NP prime, followed by S2hlx-Ex19. In some embodiments, subjects can be inoculated with a sequential regimen of S2hlx-Ex15-NP prime, followed by S2hlx-Ex19 NP and S2hlx-Ex20 NP boosts. In some embodiments, the inoculation can include an adjuvant. In some embodiments, the adjuvant can include toll-like receptor 4 agonist glucopyranosyl lipid adjuvant-stable emulsion (GLA-SE). In some embodiments, subjects can be inoculated twice.

In some embodiments, sera from subjects (e.g., mice) vaccinated with any of the vaccination protocols described herein can react (e.g., produce antibodies that can bind) with WA-2 and XBB SARS-COV-2 spike. In some embodiments, sera from subjects vaccinated with any of the vaccination protocols described herein can react with all human beta coronaviruses. In some embodiments, sera from subjects vaccinated with any of the vaccination protocols described herein can react with pre-emergent animal viruses from bats and pangolins (e.g., RsSHC014 and GXP4L). In some embodiments, sera from subjects vaccinated with any of the vaccination protocols described herein can react with heterologous human Betacoronaviruses. In some embodiments, sera from subjects vaccinated with any of the vaccination protocols described herein can cross react with OC43, MERS, and HKU-1. In some embodiments, sera from subjects vaccinated with any of the vaccination protocols described herein can induce a broad immune against stem helix epitopes.

In some embodiments, the Ex_mosaic-NP can boost stem helix specific immune responses in subjects previously vaccinated with mRNA encoding the SARS-CoV-2 WA-2 spike. In some embodiments, the Ex_mosaic-NP can protect against a viral challenge with a heterologous virus. In some embodiments, subjects vaccinated with Lx_mosaic-NP can elicit higher average titers of antibodies targeting the stem helix epitope compared to subjects vaccinated with only spike mRNA. In some embodiments, viral RNA cannot be detected in the lungs of subjects immunized with Lx_mosaic-NPs and their congestion scores can be significantly lower when compared to those of mRNA spike vaccinated subjects.

TABLE 2
Heavy chain variable region sequence of
antibodies that bind the epitope
scaffold described herein.

| | | SEQ |
|---|---|---|
| Antibody | Heavy chain variable region | ID |
| name | sequence | NO |
| DH1490 | QVQLQQSGPGLVKPSETLSLTCTVS | 113 |
| | GGSISSSSYYWGWIRQPPGKGLEWI | |
| | GSIYYSGSTYYNPSLKSRVTISVDT | |
| | SKNQFSLKLSSVTAADTAVYYCARH | |
| | GPLRFRLGYYDSSGYLFQHWGQGTL | |
| | VTVSS | |
| DH1491 | QVQLVQSGGGLVQPGGSLRLSCATS | 114 |
| | GFNFRAFAMSWVRQAPGKGLEWAAV | |
| | MSGTDDGTYYVESVKGRFTIFRDNS | |
| | ENTVYLQMNSLRADDSAIYYCAKGT | |
| | LGHCSGVDCYYLDYWGRGTLVTVSS | |
| DH1501.3 | QVQLVQSGAEVKKPGASVRLSCKAS | 115 |
| | GDTFTNEYVQWVRQAPGQGLEWMGL | |
| | INPSGSGTAFARNFQGRVSMTRDTS | |
| | TRTVYMDLTSLRYEDTAVYYCARMS | |
| | RAGTFDLWGQGTMVTVSS | |
| DH1495 | EVQLVESGGGLVQPGGSLRLSCAAS | 116 |
| | GFTFDNYAMGWVRQPPGKGLEWVSS | |
| | FSGRGVSTYYADSVKGRFTVSRDSS | |
| | KNTLFLQMNYLRVEDTAVYYCATYP | |
| | YDILTGYYGAFDYWGQGALVTVSS | |
| DH1496 | QVQLQQWGAGLLKPSETLSLTCSFY | 117 |
| | GGSFSGYCWSWIRQSPGKGLEWIGE | |
| | INHSGSTNYNPSLKTRVTILIDTSN | |
| | NQFSLRLSSVTAADTAVYYCARER | |
| | AIRRCTSTSCYRVGGVAGLDVWGQG | |
| | TTVSVSS | |
| DH1497 | QVQLVQSGGGLAQPGESLRLSCAAS | 118 |
| | GFTFSSYAMTWVRQAPGKGLEWVSS | |
| | ISGKGENIEYAESVKGRFTISRDNA | |
| | KNTVDLEMNSLRAEDTATYFCAKHI | |
| | YGAFIVVPNTLYDALDVWGQATKVT | |
| | VSS | |
| DH1498 | QVQLQESGPGLVKPSETLSLTCAVS | 119 |
| | GGSVSSDTYYWSWIRQPPGKGLEWI | |
| | GYIYNSGSTNYNPSLKSRVTISVDT | |
| | SKSQFSLNLGSVTAADTAVYYCARE | |
| | GAGTYPPKNDAFDIWGQGTLVTVSS | |
| DH1499 | QVQLQESGGGLVKPGGSLRLSCAAS | 120 |
| | GFTFSSYSMNWVRQAPGKGLEWVSS | |
| | ISSSSSYIYYADSVKGRFTISRDNA | |
| | KNSLYLQMNSLRAEDTAVYYCARGI | |
| | IDYGDYFDYWGQGTLVTVSS | |
| DH1501.1 | EVQLVESGAEVKKPGASVRVSCKAS | 121 |
| | GFTFTDHYMHWVRQAPGQGLEWMGL | |
| | INPTGVNTFYAQNFRGRVTMTRDTS | |
| | TKTDYLEVRSLTSQDTAMYYCARMS | |
| | RYGAFDIWGQGTMVTVSL | |
| DH1501.2 | EVQLVESGAEVKKPGASVRLSCKAS | 122 |
| | GDTFNNKYVHWVRQAPGQGLEWMGL | |
| | INPSGSGTAFAQKFQGRVSMTRDTS | |
| | TRTVYLGLTSLTYEDTAVYYCARMS | |
| | RAGTFDIWDQGTMVTVSS | |
| DH1506 | QVQLVQSGAEVKKPGASVKLSCRAS | 123 |
| | GYPITSHYMHWVRQAPGQGLEWMGI | |
| | INPSGTGTSFARNFQGRVTMTRDTA | |
| | TRTVYMELSSLKSEDSAVYYCAGGT | |
| | MGPLFDYWGQGTLVTVSS | |
| DH1508 | QVQLVESGGGVVQPGKSLKLSCAAS | 124 |
| | GFTFSTYSMHWVRQAPGKGLEWVAI | |
| | ISYDGRNKYYANSVKGRFTISRDNS | |
| | KNTLYLEINNLRPQDTAVYYCATDA | |
| | SNVEVPGKFAPLDKWGQGTLVTVSS | |
| DH1509 | QVQLVESGGGVVQPGRSLRLSCAAS | 125 |
| | GFTLSSHGIHWVRQAPGKGLEWVAV | |
| | TSFDGRNKKFGDSVKGRFTISRDNS | |
| | KNTVYLQMNSLRTEDTAVYYCAKDW | |
| | GDDYSRWYFDLWGRGTLVTVSS | |

TABLE 3
Light chain variable region sequence of
antibodies that bind the epitope
scaffold described herein.

| | | SEQ |
|---|---|---|
| Antibody | Light chain variable | ID |
| name | region sequence | NO |
| DH1490 | DIQLTQSPSTLSASVGDRVTITCRA | 126 |
| | SQSISSWLAWYQQKPGKAPKLLIYD | |
| | ASSLESGVPSRFSGSGSGTEFTLTI | |
| | SSLQPDDFATYYCQQYNSYSRWTFG | |
| | QGTKVEIK | |
| DH1491 | DIVLTQSPATLSLSPGERATLSCRA | 127 |
| | SQSVGTYVAWYQHKVGQAPRLLIYD | |
| | ASTRATDIPARFSGSGSGTDFTLTI | |
| | TSLEPEDVAIYYCQQRVNLVTFGGG | |
| | TKLEIK | |
| DH1501.3 | EIVLTQSPDTLSLSLGERATLFCRA | 128 |
| | SETIDSRYLAWYQQKPGRAPRLLMS | |
| | GTSVRATGIPDRFSGSGSGTDFTLT | |
| | ITRLEPEDFAVYYCQQYGSSPPRYT | |
| | FGQGTKLAMK | |
| DH1495 | DIQLTQSPSTLSASVGDRVTITCRA | 129 |
| | SQTIDTWLAWYQQKPGKAPKLLIYG | |
| | ASSLQSGVPSRESASGSGTEFTLTI | |
| | SSLQPDDFAIYYCQQYKDYSTFGQG | |
| | SKVEFM | |
| DH1496 | DIVMTQSPSSLSASVGDRVTITCQA | 130 |
| | SRDIYKYLNWYQQKPGQAPKLLISD | |
| | ASNLETGVPSRFSGSGSGTDFTFTI | |
| | SSLQPEDIAAYYCQQYDNPLITFGQ | |
| | GTRLEIK | |
| DH1497 | EIVLTQSPGTLSLSPGERATLSCRA | 131 |
| | SQSVSRNYLAWYQQKPGQAPRILVY | |
| | DASNRAIGIPDRFSGSGSGTDFTLT | |
| | ISRLEPEDFAVYYCQQYGSSPGTFG | |
| | QGTRLEIK | |
| DH1498 | EIVLTQSPDSLAVSLGERATINCKS | 132 |
| | SQSVLFSSDNKNYLAWYQQKPGQPP | |
| | KLLIYWASTRESGVPDRFSGSGSGT | |
| | DFTLTISSLQAEDVAVYYCQQYYNS | |
| | PRTFGQGTKVEIK | |
| DH1499 | EIVLTQSPDTLSLSPGERATVSCRA | 133 |
| | SQSVGAGYVAWYQQRPGQPPRLLIY | |
| | GASVRATGIPDRESGSGSGTDFSLT | |
| | INRVEPEDFAVYYCQNYASSPPRYT | |
| | FGQGTKLEIK | |
| DH1501.1 | EIVLTQSPGTLSLSPGERATLSCRA | 134 |
| | SQSVGSGYLAWYQQKPGQAPRLLIY | |
| | GASVRATGIPDRFSGSGSGTDFSLT | |
| | INRVEPEDFAMYYCQNYGSSPPRYT | |
| | FGQGTKLEIK | |
| DH1501.2 | EIVLTQSPGTLSLSPGDRATLSCRA | 135 |
| | SENVGTSYLAWYQQKPGQAPRLLIS | |
| | GTSSRATGVPDRFSGSGSGRDFTLT | |
| | ISRLEPEDFAVYYCQQYGSSPPRYT | |
| | FGQGTKLDMK | |
| DH1506 | EIVLTQSPGTLSLSPGERATLSCRA | 136 |
| | SQSVRRNFLAWYQQKPGQAPRLLIY | |
| | EASTRATGIPDRESGSGSGTDFTLT | |
| | ISRLEPEDFAVYYCQQYDSSPPGVT | |
| | FGPGTKVDIR | |
| DH1508 | EIVMTQSPATLSVSPGERATLSCRA | 137 |
| | SQSVSSDLAWYQQKPGQAPRLLIYG | |
| | ATTRATGVPARFSGSGSGAEFTLTI | |
| | SSLQSGDFAVYYCQQYNDWPLITFG | |
| | QGTRLEIK | |
| DH1509 | DIQMTQSPSSLSASVGDRVTITCQA | 138 |
| | SHDITKYLNWYQQKPGRAPKLLIYD | |
| | ASNLKTGVPSRFSGSGSGTDFTFTI | |
| | TSLQPEDLATYYCQQSDVLPITFGQ | |
| | GTRLEIK | |

### Methods

The immune response against the compositions of the invention can be generated by one or more inoculations of a subject with an immunogenic composition of the invention. A first inoculation can be termed a “primary inoculation” and subsequent immunizations can be termed “booster inoculations”. Booster inoculations can enhance the immune response, and immunization regimens including at least one booster inoculation can be used. Any composition of the invention may be used for a primary or booster immunization. The adequacy of the vaccination parameters chosen, e.g., formulation, dose, regimen and the like, can be determined by taking aliquots of serum from the subject and assaying antibody titers during the course of the immunization program. Alternatively, T cell populations can by monitored by conventional methods. The clinical condition of a subject can be monitored for the desired effect, e.g., limiting SARS-CoV-2 infection, improvement in disease state (e.g., reduction in viral load), etc. If such monitoring indicates that vaccination is sub-optimal, the subject can be boosted with an additional dose of composition, and the vaccination parameters can be modified in a fashion expected to potentiate the immune response. Thus, for example, the dose of the polypeptide, VLP, or composition, and/or adjuvant, can be increased or the route of administration can be changed.

In a further aspect, disclosed herein are methods for monitoring a SARS-CoV-2-induced disease in a subject and/or monitoring response of the subject to immunization by a SARS-CoV-2 vaccine, comprising contacting the epitope scaffolds of the invention with a bodily fluid from the subject and detecting SARS-CoV-2-binding antibodies in the bodily fluid of the subject. By “SARS-CoV-2-induced disease” is intended any disease caused, directly or indirectly, by SARS-CoV-2. The method comprises contacting a epitope scaffold of the invention with an amount of bodily fluid (such as serum, whole blood, etc.) from the subject; and detecting SARS-CoV-2-binding antibodies in the bodily fluid of the subject. The detection of the SARS-CoV-2 binding antibodies allows the SARS-CoV-2 disease in the subject to be monitored. In addition, the detection of SARS-CoV-2 binding antibody also allows the response of the subject to immunization by a SARS-CoV-2 vaccine to be monitored. In still other methods, the titer of the SARS-CoV-2-binding antibodies is determined. Any suitable detection assay can be used, including but not limited to homogeneous and heterogeneous binding immunoassays, such as radioimmunoassays (RIA), ELISA, immunofluorescence, immunohistochemistry, FACS, BIACORE and Western blot analyses. The methods may be carried in solution, or the polypeptide(s) of the invention may be bound or attached to a carrier or substrate, e.g., microtiter plates (ex: for ELISA), membranes and beads, etc. Carriers or substrates may be made of glass, plastic (e.g., polystyrene), polysaccharides, nylon, nitrocellulose, or teflon, etc. The surface of such supports may be solid or porous and of any convenient shape. The polypeptides of the invention for use in this aspect may comprise a conjugate as disclosed above, to provide a tag useful for any detection technique suitable for a given assay.

As described herein, aspects of the invention are drawn to compositions and methods of preventing a viral infection in a subject.

Aspects of the invention are drawn to compositions and methods of treating a viral infection in a subject.

For example, one or more of the following effects can result from the administration of a therapy or a combination of therapies as described herein: (i) the reduction or amelioration of the severity of a viral infection and/or a symptom associated therewith; (ii) the reduction in the duration of a viral infection and/or a symptom associated therewith; (iii) the regression of a viral infection and/or a symptom associated therewith; (iv) the reduction of the titer of a virus; (v) the reduction in organ reduced function or failure associated with a viral infection; (vi) the reduction in hospitalization of a subject; (vii) the reduction in hospitalization length; (viii) the increase in the survival of a subject; (ix) the elimination of a virus infection; (x) the inhibition of the progression of a viral infection and/or a symptom associated therewith; (xi) the prevention of the spread of a virus from a cell, tissue or subject to another cell, tissue or subject; and/or (xii) the enhancement or improvement the therapeutic effect of another therapy.

In embodiments, a therapeutically effective amount can comprise less than about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject.

In embodiments, the therapeutically effective amount comprises less than about 0.1 mg, about 0.1 mg, about 0.5 mg, about 1.0 mg, about 2.5 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 70 mg, about 80 mg, about 90 mg, about 100 mg, about 120 mg, about 135 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 1.0 g, about 1.5 g, about 2.0 g, about 2.5 g, about 5 g, about 10 g, about 25 g, about 50 g, or more than 50 g.

Aspects of the invention are also drawn to managing a subject afflicted with or at risk of a viral infection.

Aspects of the invention can comprise administering to a subject an immunogenic composition comprising an epitope from an S2 region of a human coronavirus spike protein.

In embodiments, administering can refer to providing a therapeutically effective amount of the composition to a subject. The formulation or pharmaceutical compound can be administered alone, but can be administered with other compounds, excipients, fillers, binders, carriers or other vehicles selected based upon the chosen route of administration and standard pharmaceutical practice. In some embodiments, an immune response can be stimulated in an individual by administering to the individual the immunogenic composition described herein or the amino acid epitope grafted into the scaffold protein described herein. In embodiments, the method is for eliciting an immune response to a fusion peptide epitope and/or an S2 stem helix of human coronavirus spike protein.

Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

In embodiments, the compound can be administered alone, or can be administered as a pharmaceutical composition together with other compounds, excipients, carriers, diluents, fillers, binders, or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

Embodiments can be administered to a subject in one or more doses. The dose level can vary as a function of the specific composition or pharmaceutical composition administered, the severity of the symptoms and the susceptibility of the subject to side effects. Dosages for a given compound are readily determinable by a variety of means. For example, dosages can be determined by standard clinical techniques. In addition, in vitro or in vivo assays can be employed to help identify optimal dosage ranges. The precise dose to be employed can also depend on the route of administration and can be decided according to the judgment of the practitioner and each patient's circumstances.

In some embodiments, multiple doses of the composition can be administered. The frequency of administration and the duration of administration of the composition can vary depending on any of a variety of factors, e.g., patient response, severity of the symptoms, and the like. For example, in an embodiment, the pharmaceutical composition can be administered once per month, twice per month, three times per month, every other week (qow), once per week (qw), twice per week (biw), three times per week (tiw), four times per week, five times per week, six times per week, every other day (qod), daily (ad), twice a day (qid), three times a day (tid), or four times a day. In an embodiment, the pharmaceutical composition can be administered 1 to 4 times a day over a period of time, such as 1 to 10-day time period, or longer than a 10-day period of time.

In embodiments, the composition can be administered in combination with one or more additional active agents. For example, a first agent (e.g., a prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second agent (e.g., a prophylactic or therapeutic agent) to a subject with a disease or disorder or a symptom thereof.

Embodiments as described herein further comprises administering one or more additional active agents to a subject together with the epitope from an S2 region of a human coronavirus spike protein. Non-limiting examples of such additional active agents can comprise an anti-viral agent (e.g., remdesivir, molunpiravir, paxlovid, or any combination thereof), a vaccine, an anti-inflammatory agent, a pain reliever, a steroid, or any combination thereof.

In some embodiments, disclosed herein are methods for stimulating an immune response in an individual, comprising administering to the individual the immunogenic composition as disclosed herein or the amino acid epitope grafted into the scaffold protein (i.e., epitope scaffold) as disclosed herein.

In some embodiments, disclosed herein are methods for making an immunogenic composition or the amino acid epitope grafted into the scaffold protein as disclosed herein. In some embodiments, the method can be selecting an epitope from an S2 region of a human coronavirus spike protein, including a fusion peptide epitope or a S2 stem helix epitope; identifying a scaffold protein with an exposed backbone region that matches the selected epitope (RMSD ≤0.5 Å); and incorporating the epitope into the scaffold protein to obtain an ES having an RMSD of less than about 1.0 Å as compared to the scaffold protein that has not incorporated the epitope.

### Kits

Aspects of the invention are also directed towards kits, such as kits comprising compositions as described herein. For example, the kit can comprise therapeutic combination compositions described herein.

In one embodiment, the kit includes (a) a container that contains a composition, such as that described herein, and optionally (b) informational material. The informational material can be descriptive, instructional, marketing or other material that relates to the methods described herein and/or the use of the agents for therapeutic benefit.

In an embodiment, the kit includes two or more agents. For example, the kit can include a container comprising an immunogenic composition comprising an epitope from an S2 region of a human coronavirus spike protein, and a second container comprising a second active agent.

In embodiments, the kit further comprises a third container comprising a third active agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the therapeutic combination composition, e.g., in a suitable dose, dosage form, or mode of administration (e.g., a dose, dosage form, or mode of administration described herein), to treat a subject who has a nerve disconnectivity disorder). The information can be provided in a variety of formats, include printed text, computer readable material, video recording, or audio recording, or information that provides a link or address to substantive material.

The composition in the kit can include other ingredients, such as a solvent or buffer, a stabilizer, or a preservative. The immunogenic composition can be provided in any form, e.g., liquid, dried or lyophilized form, or for example, substantially pure and/or sterile. When the agents are provided in a liquid solution, the liquid solution is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., in a desired ratio. For example, the kit includes a plurality of syringes, ampules, foil packets, blister packs, or medical devices, e.g., each containing a single combination unit dose. The containers of the kits can be airtight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Engineered Immunogens to Expose Conserved Epitopes Targeted by Broad Coronavirus Antibodies

Antibody responses to SARS-CoV-2 primarily target the receptor binding domain, which mutates to escape acquired immunity. Other SARS-CoV-2 spike regions in the S2 subunit, such as the fusion peptide and the stem helix, are highly conserved across sarbecoviruses and engaged by broadly reactive antibodies, indicating that targeting these epitopes by vaccination could offer protection against both current and emergent viruses. Here computational modeling was employed to design epitope scaffolds that display the fusion peptide and the stem helix epitopes. These molecules bound both mature and germline versions of multiple known human antibodies with high affinity and specificity, and showed potent engagement of pre-existing SARS-CoV-2 immunity, thus illustrating their potential to preferentially elicit responses against the fusion peptide and the stem helix by vaccination.

Computational design of SARS-CoV-2 stem helix and fusion peptide immunogens that engage broad coronavirus-reactive antibodies.

The majority of immune responses elicited against SARS-CoV-2, by either natural infection, vaccination, or a combination of both, are focused on the receptor binding domain (RBD) (1-4). However, deep scanning mutagenesis studies (5) and the emergence of novel variants that evade pre-existing immunity have revealed the plastic nature of RBD (2, 6), indicating that next-generation coronavirus vaccines that aim to protect against both current and emergent CoV will likely need to induce antibodies against CoV spike regions beyond the RBD.

Two regions in the S2 subunit of the SARS-CoV-2 spike, the fusion peptide (FP) and the stem helix, show high conservation across diverse coronaviruses. Antibodies isolated against these sites cross-react with alpha and beta human coronaviruses and with diverse animal viruses (7-10). Fusion peptide antibodies, such as DH1058, DH1294, VN01H1, Cov44-79, and Cov44-62, bind to SARS-CoV-2 spike residues 813-824, cross-react with all seven human CoVs, and protect by either neutralization, Fc mediated mechanisms or both. Two major classes of antibodies with overlapping epitopes have been identified against the stem helix region (9-12). S2P6 and CC40.8, which are representative of these two classes, bound spike glycoproteins representative of all sarbecovirus clades, and protected against challenge in animals by inhibiting S-mediated membrane fusion. However, humoral responses against the stem helix or the FP are not robustly induced by existing vaccines or by natural infection. This is likely due to a combination of factors, including the occlusion of the stem helix and fusion peptide on the pre-fusion spike, requiring ACE2 binding for exposure (8), as well as the presence of the prominently displayed, immunodominant RBD domain. Given their ability to recognize diverse coronaviruses, immunogens that induce strong responses against the FP and stem helix regions can be a key component of a future “pan-coronavirus” vaccine offering broad protection against both currently circulating and emergent coronaviruses.

For the design of immunogens that expose occluded epitopes in novel molecular contexts, the development of “epitope scaffold” protein have been described (13-15). These molecules are engineered by transplanting the structure of the antibody-bound epitopes from viral molecules onto unrelated protein scaffolds. Unlike peptide-based immunogens that can sample diverse epitope conformations, epitope scaffolds present only the antibody-bound conformation of the target epitope on their surface, which typically leads to higher antibody affinity and the elicitation of antibodies specific for the structure of the target epitope (16, 17). Here, epitope grafting was applied to design stem helix or FP protein scaffolds that strongly interact with broadly cross-reactive antibodies against the fusion peptide or the stem helix regions of spike, for inducing such antibodies by vaccination with these new immunogens.

Design of Epitope Scaffolds that Bind Broadly Cross-Reactive Antibodies Against the Fusion Peptide.

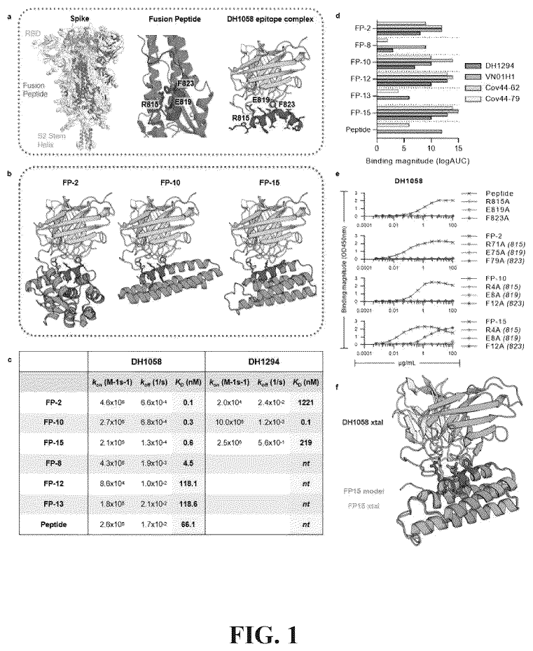

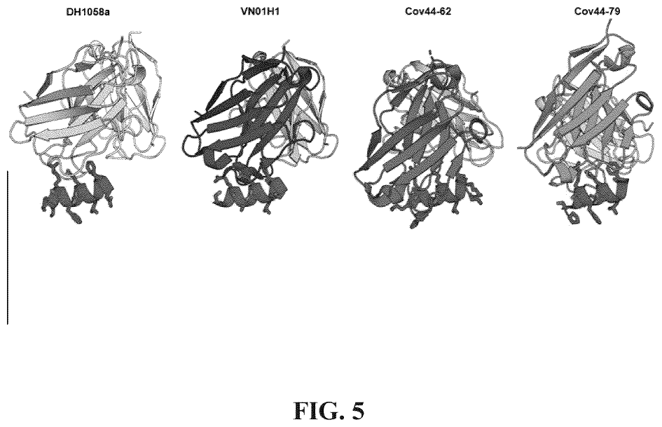

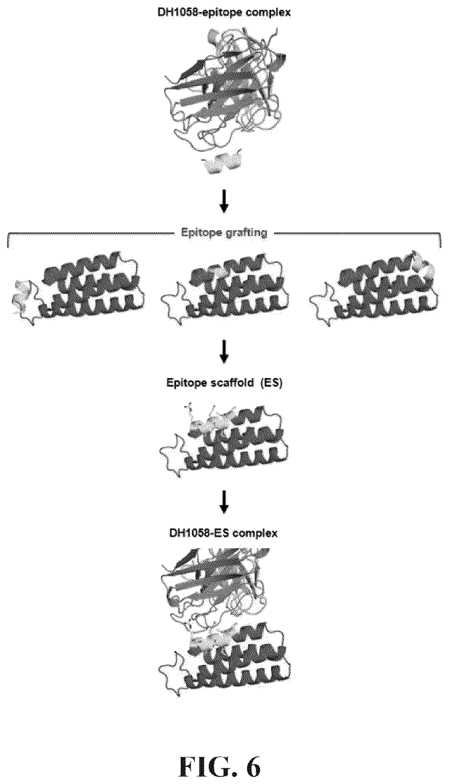

The FP domain of the SARS-CoV-2 encompasses residues 808-833 (FIG. 1a). Multiple FP antibodies, such as DH1058, DH1294, VN01H1, Cov44-79, and Cov44-62 target epitopes centered on residues 813-824, with key contacts made with virus residues R815, E819 and F823 (SARS-CoV-2 numbering, FIG. 1a, FIG. 5) (7, 8, 18, 19). These residues are occluded in the structure of the prefusion SARS-CoV-2 spike, likely limiting their immune recognition (FIG. 1a). To improve the accessibility and immune recognition of this epitope, fusion peptide ES were developed using “side chain” grafting computational methods (FIG. 6) (13-15). A large library (˜10,000) of small monomeric scaffolding proteins was queried computationally to identify proteins with exposed backbone regions that closely matched (<0.5 Å RMS) the structure of the FP epitope residues 815-823 (FIG. 6) In regions with high structural mimicry to the DH1058-bound epitope, the epitope sequence replaced that of the parent scaffold and additional mutations were introduced to accommodate the grafted sequence (FIG. 6).

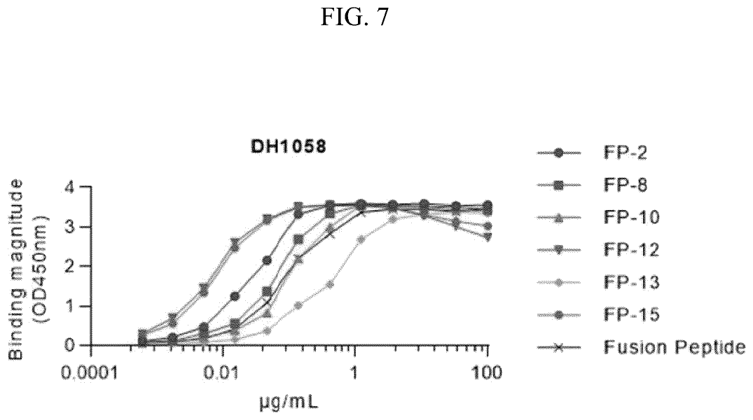

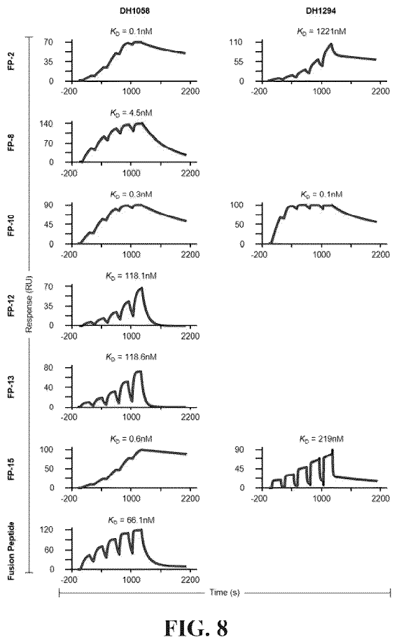

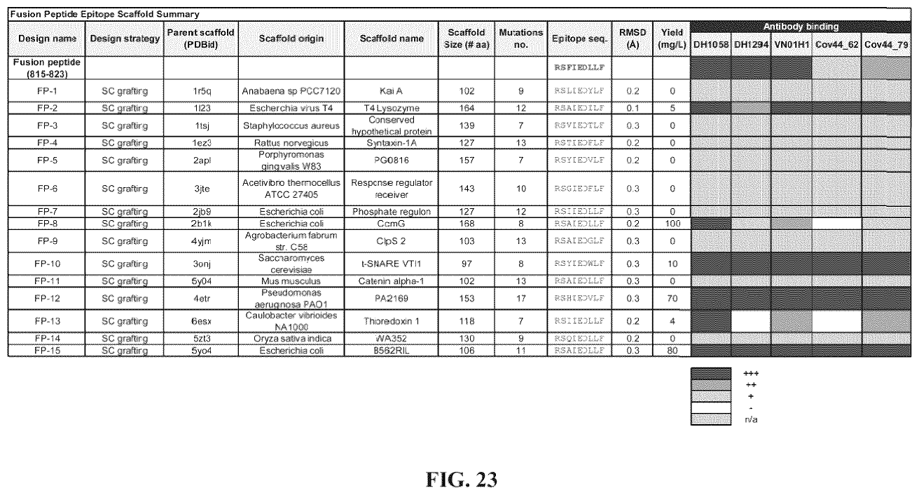

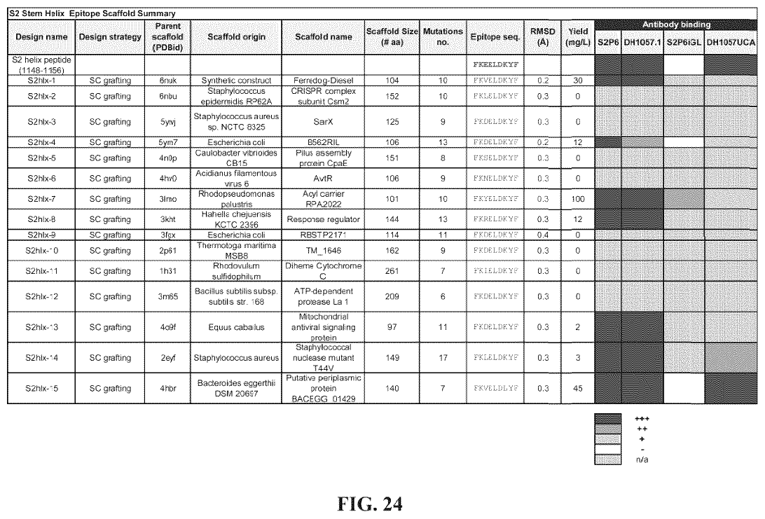

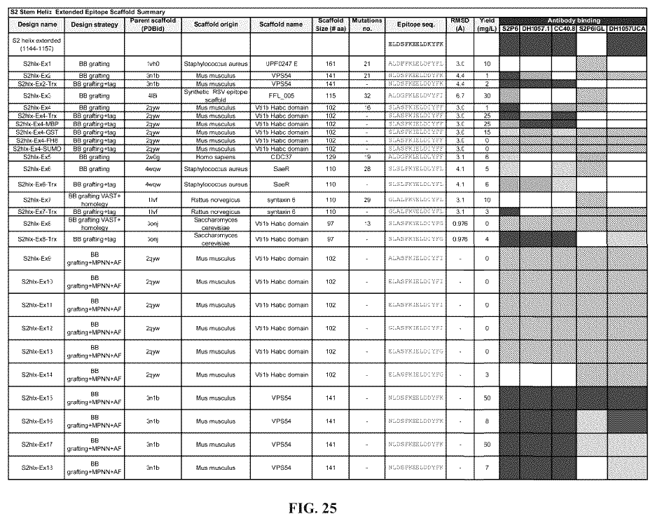

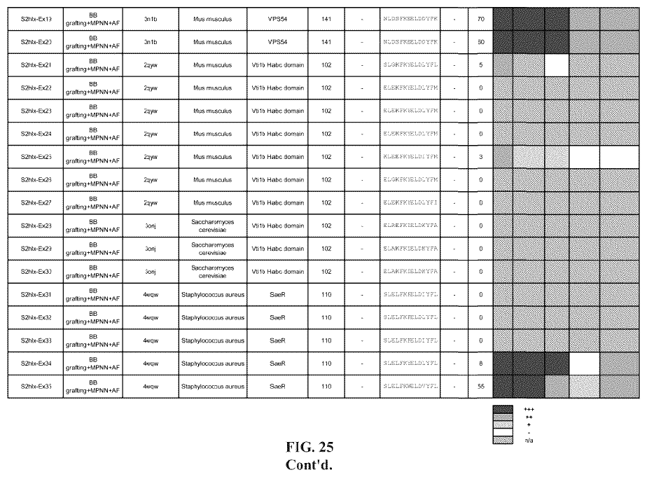

Fifteen engineered FP ESs were expressed recombinantly (FIG. 1b for representative designs) with six out of 15 FP ESs producing soluble and stable proteins that bound DH1058 by ELISA (FIG. 7, FIGS. 23-25 for representative tables). DH1058 mAb dissociation constants were determined by surface plasmon resonance (SPR) for all six ESs, and to DH1294 mAb for the three highest DH1058 mAb binders (FIG. 1c, FIG. 8). Designs FP-2, FP-10, and FP-15 bound to DH1058 mAb with picomolar affinity and to DH1294 mAb with K_{D}s of 1221 nM, 0.1 nM and 219 nM respectively.

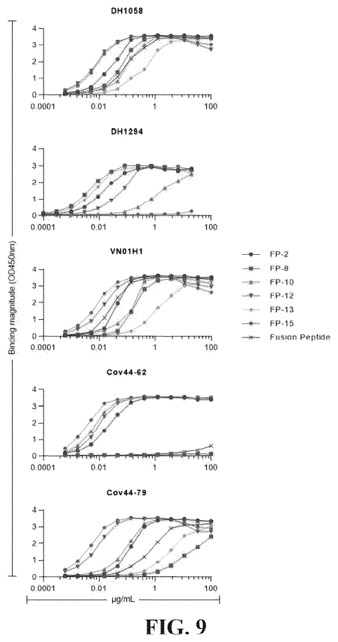

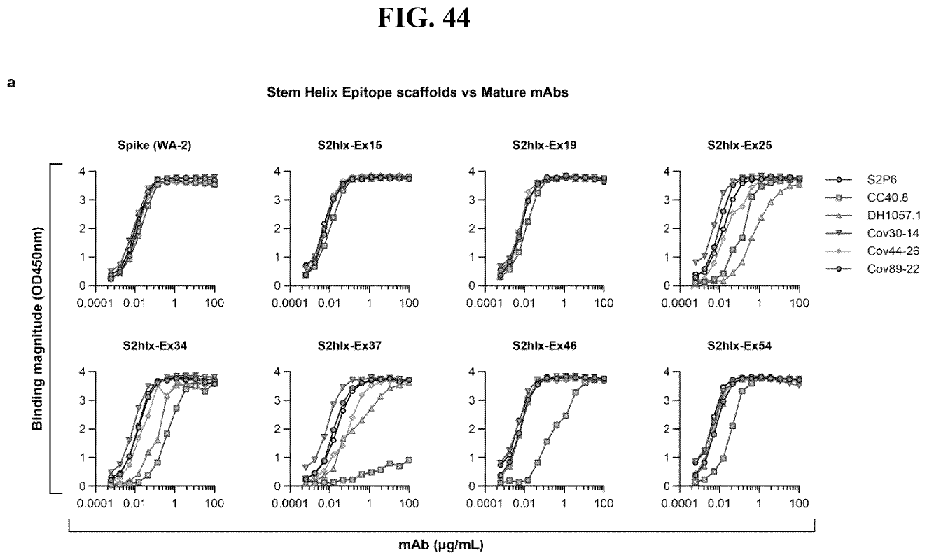

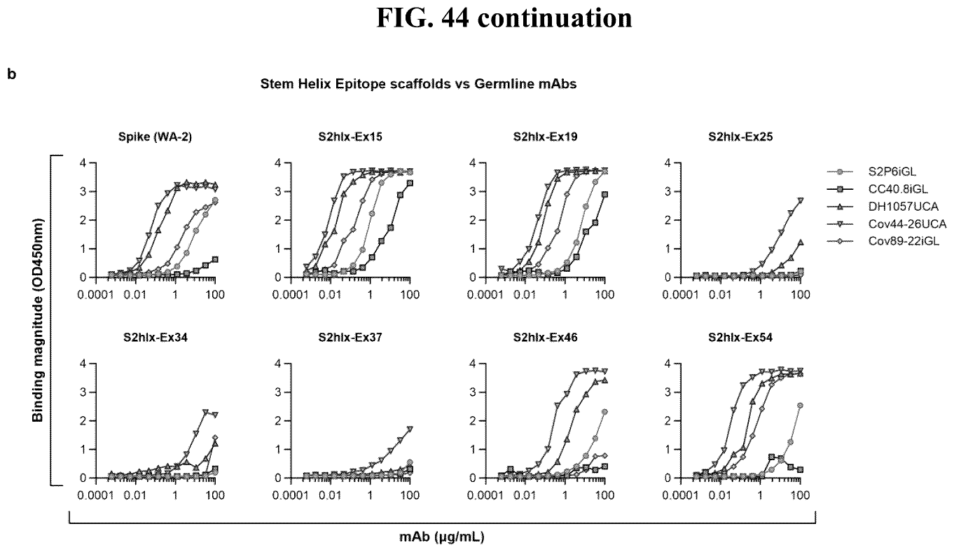

To probe the breadth of their recognition, the binding of spike epitope scaffolds was measured against antibodies VN01H1, C77G12, VP12E7, COV44-79, and COV44-62, which were not explicitly considered at the design stage (FIG. 1d; FIG. 9 and FIG. 44). Five designs bound at least 6 out of the 7 antibodies tested, with FP-12 and FP-15 binding all of them tighter than spike. In addition to the mature antibodies, binding was also measured to their inferred unmutated common ancestors (UCAs) or germline-reverted forms (iGLs). Spike showed measurable binding to the DH1058 UCA, while FP-12 and FP-2 were bound by DH1058 UCA, DH1294 UCA and VP12E7 iGL (FIG. 1d, FIG. 9 and FIG. 44). Taken together, these data show that the engineered FP epitope scaffolds can have broad recognition of genetically diverse FP antibodies and their precursors.

DH1058 mAb bound four out of the six FP ESs with affinities 100-fold higher than that measured here for the synthesized fusion peptide (K_{D}=66.1 nM), and those previously reported for different SARS-CoV-2 spike proteins (K_{D}s of 99 nM for WA-2 and 106 nM for Omicron) (18). To ensure that the ES affinity gains are not due to contacts between the scaffold and the mAb that are not present in the native DH1058-epitope complex, interface hotspot residues corresponding to fusion peptide amino acids R815, E819, and F823 were mutated to alanine in designs FP-2, FP-10, and FP-15. The alanine substitutions eliminated DH1058 binding to the synthetic fusion peptide and to designs FP-2 and FP-10. Two of the three substitutions similarly eliminated DH1058 binding to FP-15, with the F823A equivalent retaining some low-level antibody binding (FIG. 1e).

A high resolution (2.2 Å) crystal structure of FP-15 in complex with DH1058 revealed the binding mode and validated the computational modeling (Figure if, Table 4).

TABLE 4
Crystallographic table.

| | 52hlx-Ex19 | | 52hlx-7-DH1057.1 | | FP15-DH1058 | |
|---|---|---|---|---|---|---|
| PDB ID | 8F5H | | 8F5I | | 8FDO | |
| Data collection | | | | | | |
| Space group | C2 | | P1 | | P2₁ | |
| Cell dimensions | | | | | | |
| a, b, c (Å) | 175.6, 28.7, 65.5 | | 56.7, 78.4, 79.0 | | 48.4, 50.6, 146.2 | |
| α, β, γ (°) | 90.0, 106.7, 90.0 | | 71.6, 70.0, 88.3 | | 90.0, 96.9, 90.0 | |
| Resolution (Å) | 44.55-2.30 | (2.38-2.30) | 45.68-1.90 | (1.97-1.90) | 47.33-2.20 | (2.28-2.20) |
| R_{merge} | 0.705 | (1.651) | 0.664 | (0.933) | 0.745 | (1.051) |
| I/σI | 16.1 | (2.4) | 29.1 | (3.2) | 23.6 | (2.3) |
| CC1/2 | 0.765 | (0.613) | 0.474 | (0.207) | 0.501 | (0.529) |
| Completeness (%) | 95.1 | (92.6) | 93.9 | (87.6) | 98.2 | (98.6) |
| Redundancy | 13.5 | (14.0) | 4.7 | (3.7) | 7.5 | (7.5) |

| | | | |
|---|---|---|---|
| Refinement | | | |
| R_{work}/R_{free} (%) | 25.4/27.5 | 21.1/24.5 | 18.9/22.9 |
| No. atoms | | | |
| Protein | 2,212 | 8,003 | 4,264 |
| Glycan | 0 | 111 | 0 |
| Water | 69 | 1,353 | 189 |
| Average B-factors | | | |
| Proteins | 45.6 | 24.7 | 61.6 |
| Ligands | 46.4 | 44 | 49.5 |
| R.m.s deviations | | | |
| Bond lengths (Å) | 0.007 | 0.008 | 0.006 |
| Bond angles (Å) | 1.00 | 0.86 | 0.85 |
| Ramachandran | | | |
| Favored (%) | 99.2 | 98.5 | 97.1 |
| Alowed (%) | 0.8 | 1.5 | 2.7 |
| Outliers (%) | 0 | 0 | 0.2 |

The structure was in close agreement with that of the computationally generated antibody-ES complex, both overall and over the epitope scaffold only (RMSD<1 Å). The epitope conformation engaged by DH1058 on FP-15 was essentially identical to that induced by the antibody on spike (RMSD=0.3 Å) (18), confirming that the antibody interacts with the epitope presented by the ES in the same manner it binds to its natural target.

### Design of Epitope Scaffolds to Engage S2P6-Like Antibodies Against the Stem Helix Region of the CoV Spike.

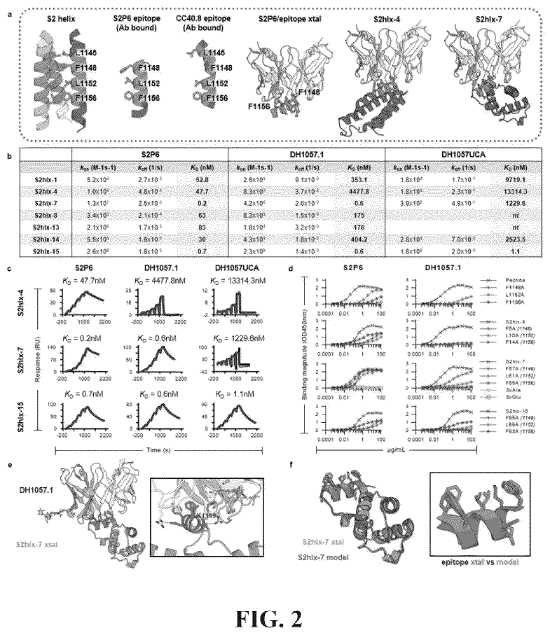

Next, epitope scaffolds were engineered to engage antibodies against the stem helix region of the CoV spike. Antibodies such as S2P6, DH1057.1, and CC40.8 bind to an epitope located between residues 1144 and 1158, which is almost completely occluded by the trimerization interface in the pre-fusion spike structure (FIG. 2a) (9, 10, 20). While both CC40.8 and S2P6 target the same spike region and make critical contacts with residues Phe1148 and Phe1156, CC40.8 makes additional interactions with Leu1145 and induces a more extended epitope conformation upon binding (FIG. 2a) (10). Because the CC40.8 epitope is longer and includes the S2P6 epitope, we first attempted to engineer epitope scaffolds that display the antibody-bound conformation of the CC40.8 epitope, with the expectation that these molecules would also be capable of binding to S2P6-class antibodies. However, the “side chain” grafting protocol used for FP epitope scaffolds above, did not identify any candidate scaffolds with exposed backbones similar to that of the CC40.8 epitope, likely due to the unusual conformation of this epitope that adopts a distorted alpha-helical shape near the N-terminus (FIG. 2a). In contrast, multiple candidate epitope scaffolds that displayed the S2P6-bound conformation of the epitope segment 1148-1156 were successfully designed computationally using side chain grafting. This was possible because the S2P6 epitope adopts a canonical alpha-helical structure, with most of the antibody contacting residues contained within three helical turns (FIG. 2a).

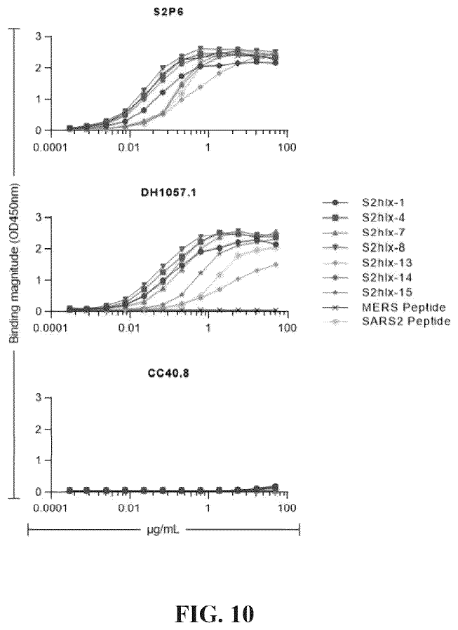

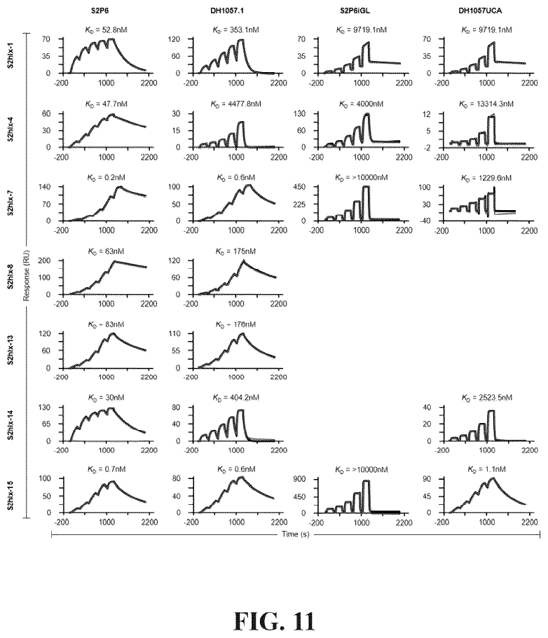

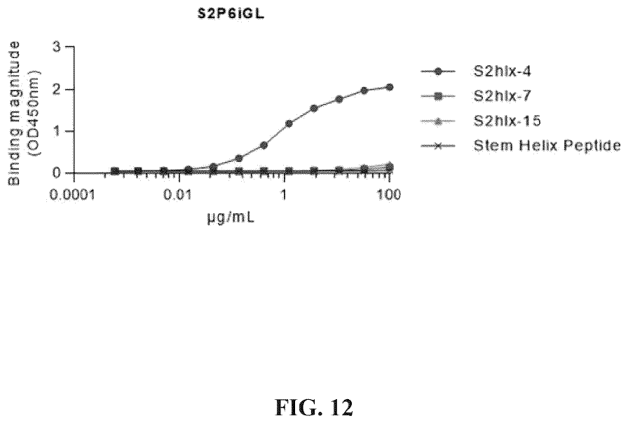

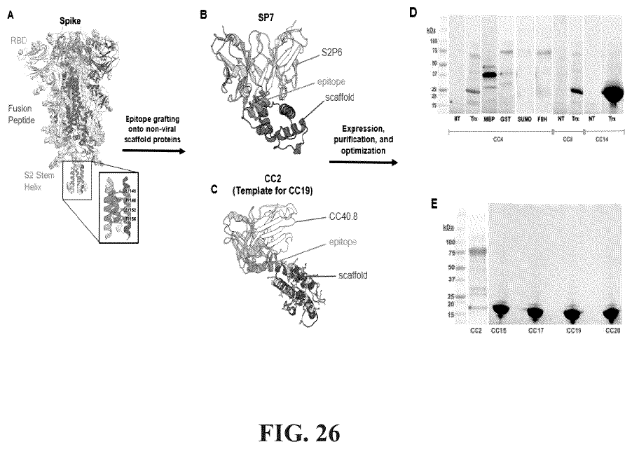

Fifteen S2P6-targeted designs, named S2hlx-1 to 15, were chosen for recombinant production in E. coli (FIG. 2a for representative designs; FIGS. 26-28) and seven yielded soluble protein. In an initial ELISA screen, all seven designs bound both S2P6 and DH1057.1 mAbs comparable to or better than a synthesized SARS-CoV-2 stem helix peptide (residues 1147-1161). Neither this peptide nor the ESs bound CC40.8 mAb due to the absence of epitope residue L1145 (FIG. 10). Upon SPR analysis, S2hlx ESs were measured to bind S2P6 mAb with dissociation constants between 0.2 nM and 83 nM and DH1057.1 mAb with K_{D}s between 0.6 nM and 4.5 uM (FIG. 2b-c, FIG. 11). S2P6 mAb was previously reported to bind spike proteins with dissociation constants of 7 nM, for those of SARS-CoV-1 and SARS-CoV-2, and 12 nM for the MERS-CoV spike (9). S2hlx-7 and S2hlx-15 bound S2P6 mAb with substantially higher affinities (K_{D}s of 0.2 nM and 0.7 nM), while the other five designs had lower affinities than native spikes (K_{D}s between 47 nM and 83 nM). S2hlx-7 and S2hlx-15 also showed sub-nanomolar binding to DH1057.1 mAb, while other designs had weaker binding, in the micromolar range, for this antibody (FIG. 2b). In addition to the mature mAbs, binding was also tested to their inferred unmutated common ancestors (UCAs) or germline-reverted forms (iGLs). While all the S2hlx ESs interacted with DH1057UCA, S2hlx-15 displayed the highest affinity (K_{D}s=1.1 nM) (FIG. 2b). For S2P6-iGL, S2hlx-4 is the only design that had measurable binding by ELISA, although its interactions were reduced >10-fold compared to those of the affinity-matured antibody, likely due to a significantly faster off-rate as measured by SPR (FIG. 11, FIG. 12).

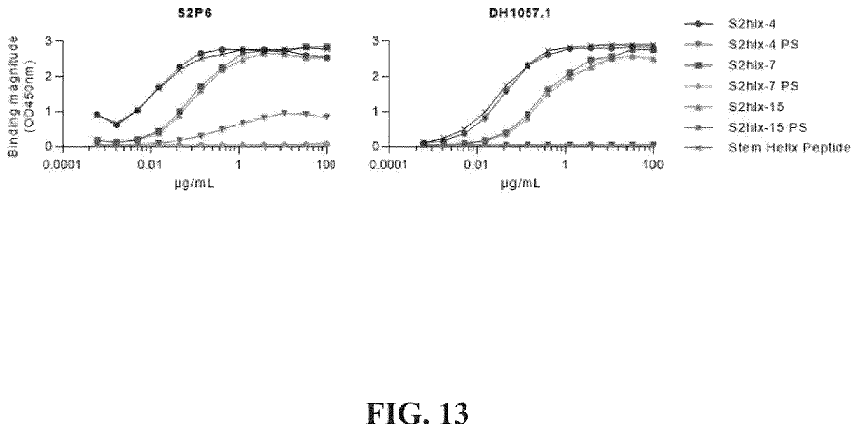

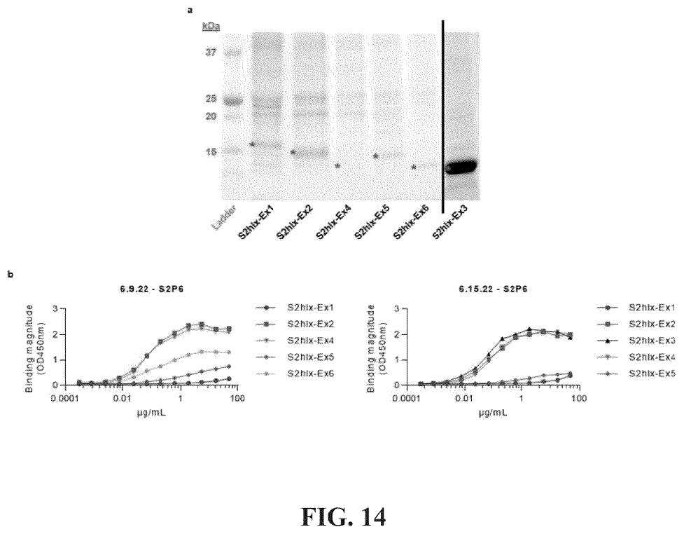

The binding specificity of S2hlx-4, S2hlx-7 and S2hlx-15 was confirmed by mutating epitope residues equivalent to spike Phe1148, Leu1152 and Phe1156. Alanine mutations limited the binding to both S2P6 and DH1057.1 mAbs in S2hlx-4 and -15 to a similar extent to what as observed for synthesized stem helix peptides (FIG. 2d) (9). For S2hlx-7, the individual epitope mutations greatly decreased DH1057.1 mAb interactions but had almost no effect on S2P6 mAb recognition. However, an S2hlx-7 variant that contained alanine substitutions at all three epitope positions completely abrogated binding, thus confirming that antibody interactions were mediated by the grafted epitope residues. In addition, none of the parent scaffolds these designs were based on showed binding to S2P6 or DH1057, further demonstrating epitope specificity (FIG. 13).

Crystallographic analysis of the high resolution (1.9 Å) S2hlx 7/DH1057.1 complex revealed for the first time the structure and interaction mode of the DH1057.1 antibody with the stem helix. DH1057.1 approached the epitope at the same angle as S2P6 mAb and similarly interacted with residues Phe1148, Leu1152, and Phe1156 (SARS-CoV-2 spike numbering) (FIG. 2e), as predicted by the alanine mutations (FIG. 2d). Unlike S2P6, DH1057.1 interacts with residue K1149 through salt bridges mediated by heavy chain residues Asp95 and Asp100C (FIG. 2e). The structure of S2hlx-7 in the complex was closely aligned with that of the computational model both overall (RMSD=0.5 Å) and, most importantly, over the grafted epitope region (RMSD=0.25 Å) (FIG. 2e). The epitope conformation displayed on S2hlx-7 closely matched that of the antibody-bound S2P6 epitope and that of the corresponding region on the prefusion spike (RMSD<0.2 Å, FIG. 2e). These data confirmed the design of multiple protein antigens that have high affinity and specificity for S2P6-like mAbs by mimicking the conformation of their epitope on native spikes.

### Design of Epitope Scaffolds to Engage the Two Major Classes of Antibodies Against the Stem Helix.

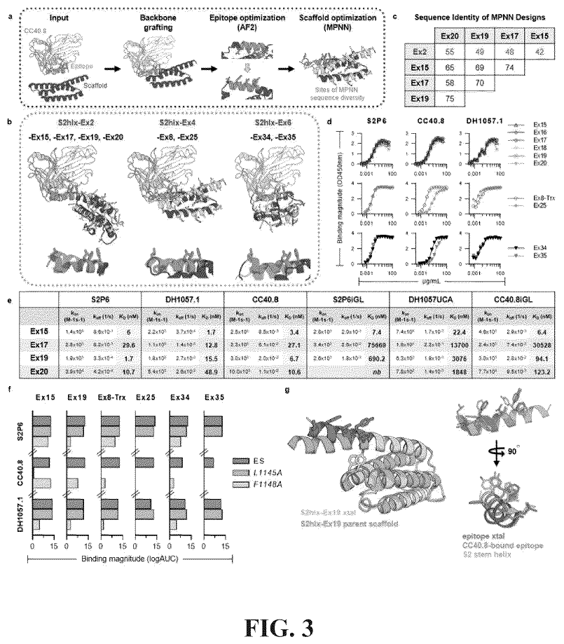

An alternative design technique, termed backbone grafting, was employed in these embodiments. Here, rather than transplanting epitope side chains onto a preexisting scaffold backbone, the parent scaffold backbone is replaced with the backbone of the desired epitope. This allows the grafting of more structurally complex epitopes that have no existing structural match, such as the CC40.8 S2-helix epitope. Candidate scaffolds were identified by aligning the N- and C-termini of the stem helix epitope in its CC40.8-bound conformation with the N- and C-termini of possible insertion sites on a candidate scaffold (FIG. 3a, b). For scaffolds where the RMSD of this alignment was below 0.75 Å, the epitope replaced the overlapping backbone of the parent scaffold, and additional mutations were introduced to integrate the epitope into the scaffold and to ensure productive interactions with CC40.8 mAb (FIG. 3a). The structures of candidate designs were predicted using Alphafold2 (21), and additional mutations were introduced to ensure proper epitope conformation. Final designs contained between 16 to 32 mutations relative to the parent scaffolds, and the grafted epitope was 3.1 to 6.7 Å away by RMSD from the backbone it replaced.

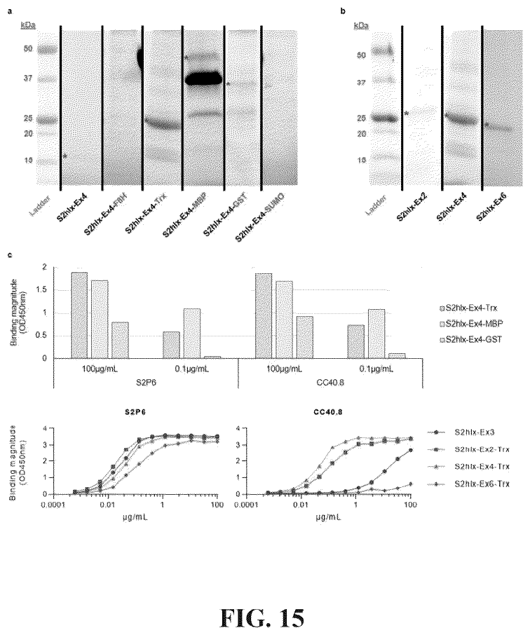

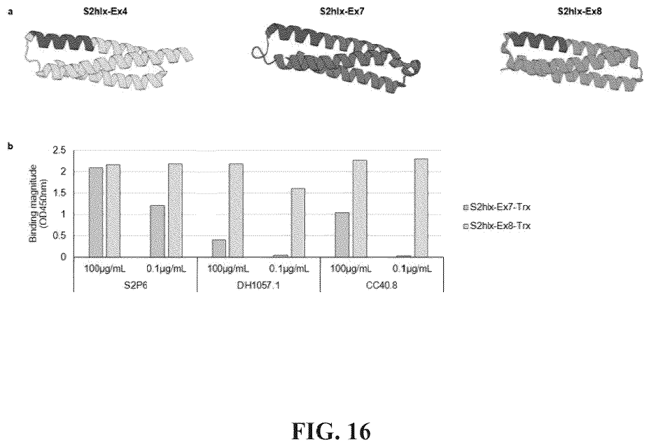

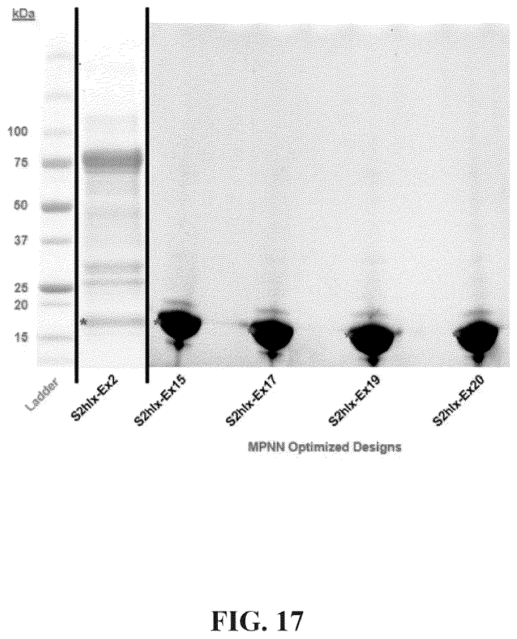

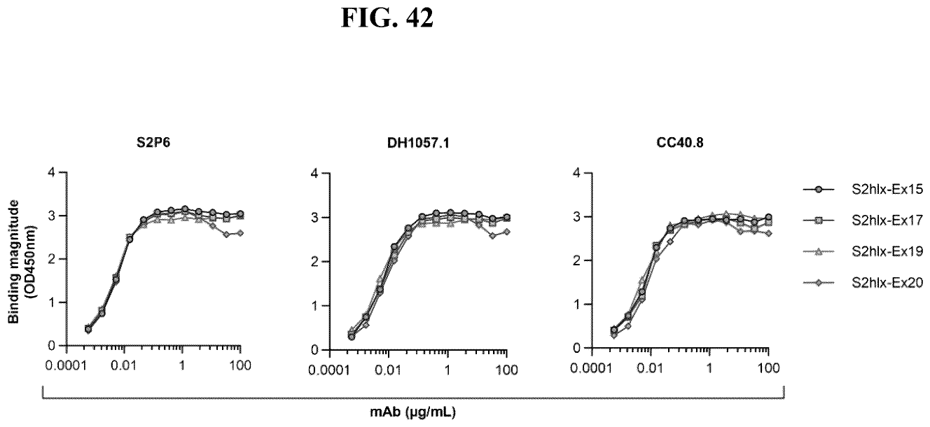

Six ESs, named S2hlx-Ex1 to 6 (FIG. 3b for representative models, FIGS. 23-25) were tested for bacterial expression and analyzed for binding to S2P6 mAb in an initial screen. While four of the six ESs, S2hlx-Ex2, -Ex3, -Ex4 and -Ex6, bound to S2P6, three of them had poor expression (S2hlx-Ex2, -Ex4 and -Ex6) (FIG. 14). To improve expression, we tested different protein expression tags and evaluated structural homologs of the parent scaffolds as alternative design templates with limited success (FIGS. 15, 16). Next, we optimized the sequence of the S2hlx-Ex designs using ProteinMPNN (22), a recently described deep learning algorithm that takes a protein structure as input and generates amino acid sequences that are predicted to generate the same fold. Three backbone grafting designs, S2hlx-Ex2, -Ex4, and -Ex6 were optimized with this approach, and sequences that expressed with high yield (40 mg/L of culture on average, FIG. 17) were identified for all of them, albeit with variable success rates. For a given structure, ProteinMPNN generated designs that had highly different sequences outside the epitope. For example, S2hlx-Ex2 based designs -Ex15, -Ex17, -Ex19, -Ex20 had between 42% and 55% sequence identity with the S2hlx-Ex2 template (FIG. 3c), and only 58-75% identity with each other, yet all bound to the stem helix mAbs by ELISA (FIG. 42).

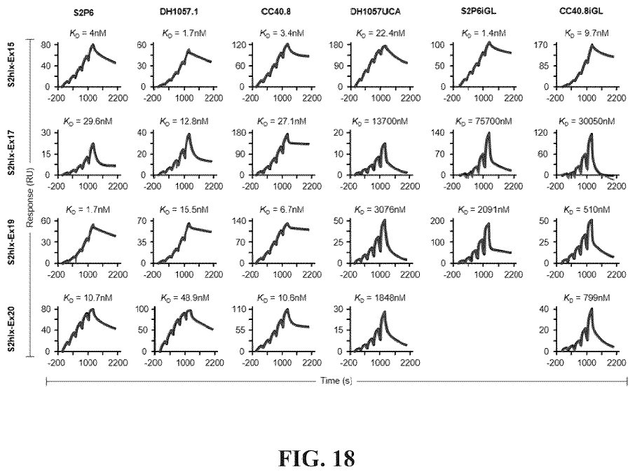

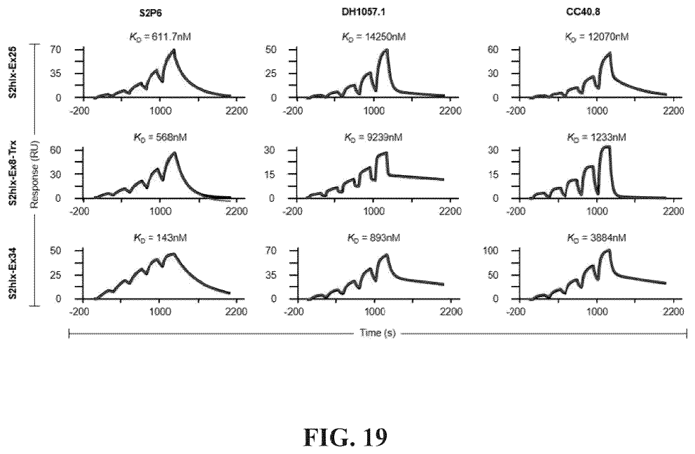

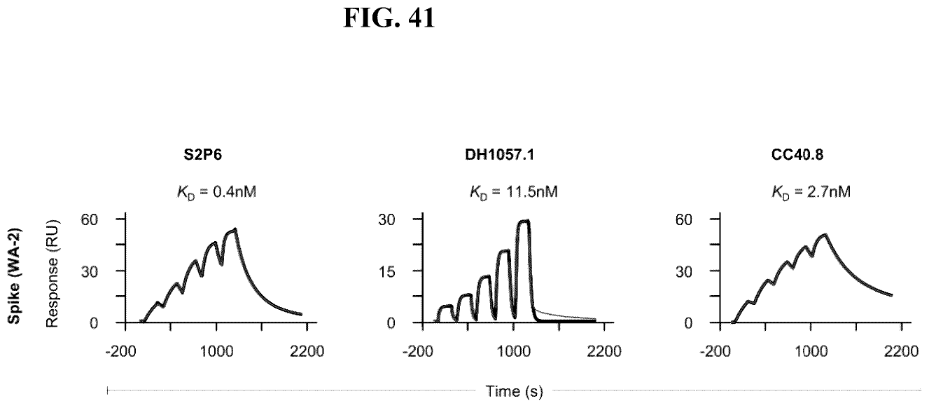

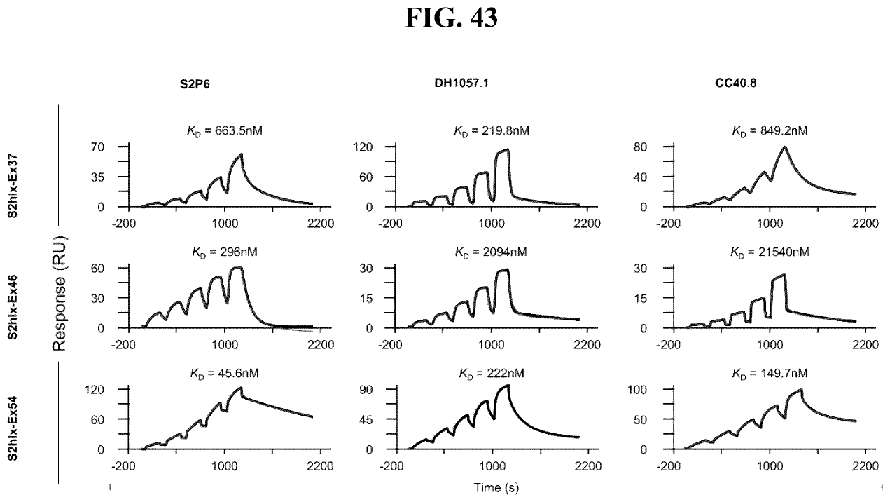

The binding affinities for the designs based on the backbone structures of S2hlx-Ex2 (S2hlx-Ex15, -Ex17, -Ex19, and -Ex20), S2hlx-Ex4 (S2hlx-Ex8-Trx and S2hlx-Ex25), and S2hlx-Ex6 (S2hlx-Ex34 and -Ex35) to CC40.8, S2P6, and DH1057.1 mAbs were determined by SPR (FIG. 18, FIG. 19 and FIG. 43). S2hlx-Ex2 derived ESs bound S2P6 mAb with dissociation constants between 2 nM and 30 nM, DH1057.1 mAb with K_{D}s between 2 nM and 49 nM, and CC40.8 mAb with K_{D}s between 3 nM and 27 nM (FIG. 3e; FIG. 18). S2hlx-Ex15 and -Ex19 had the highest binding affinities, in a similar range to those measured for spike (FIG. 41) and those reported for the stem helix peptide (9, 10). Although S2hlx-Ex25 and Ex8-Trx showed similar binding to the mature antibody panel by ELISA, the calculated binding affinities were substantially lower, with only S2P6 binding in the sub-micromolar range (611.7 nM and 330 nM respectively, FIG. 19). Finally, S2hlx-Ex34, derived from S2hlx-Ex6, bound to S2P6, DH1057.1, and CC40.8 at 82 nM, 180 nM and 486 nM respectively (FIG. 19). S2hlx-Ex54, derived from the backbone of S2hlx-Ex3, bound to S2P6, DH1057.1, and CC40.8 with equilibrium dissociation constants of 45 nM, 222 nM and 150 nM, respectively (FIG. 43), while S2hlx-Ex37, based on the fold of S2hlx-Ex6, had affinities of 664 nM, 220 nM and 849 nM to the three target mAbs (FIG. 43).

To test the ability of epitope scaffolds to engage diverse antibodies that may be elicited as part of a polyclonal response against the stem helix by vaccination, the designed antigens were tested against additional broadly reactive stem helix antibodies that were recently described and were not explicitly targeted at the design stage. The epitope scaffolds bound all tested antibodies by ELISA (FIG. 3e, FIG. 44a), indicative of their ability to recognize diverse antibodies that target the displayed epitope. In addition to the mature antibodies, binding was also tested to five of their UCA or inferred germline precursors (FIG. 3a, FIG. 44b). Kinetic analysis revealed that S2hlx-Ex15 had low nanomolar affinities for DH1057 UCA and the iGLs for S2P6 and CC40.8 (K_{D}s of 22.4 nM 1.4 nM and 9.7 nM), and also bound strongly to Cov44-26 UCA and Cov89-22iGL by ELISA. Other S2hlx-Ex2 and S2hlx-Ex3 derived designs interacted similarly with three to five of the antibody precursors tested (FIG. 3d, FIG. 18). Four out of the five antibody precursors recognized spike, but with significantly weaker affinity than S2hlx-Ex15 and S2hlx-Ex19 (FIG. 44).

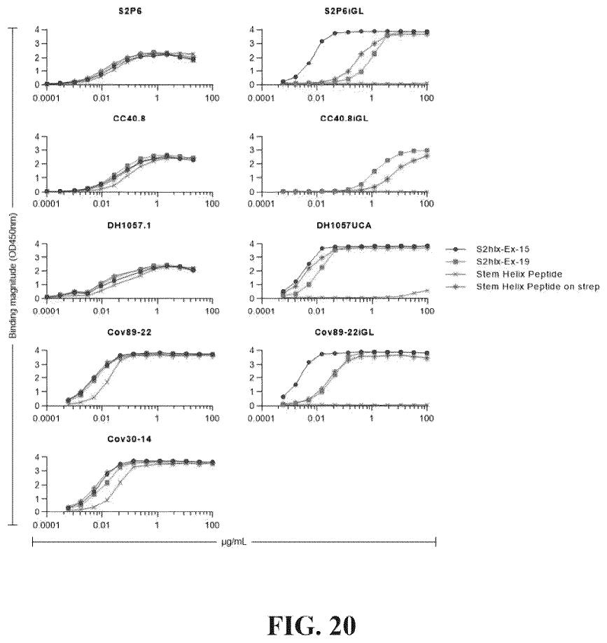

S2hlx-Ex15 and -Ex19 binding was also tested against broadly reactive stem helix antibodies Cov89-22 and Cov30-14, which were recently discovered and were not explicitly targeted by the design (23). Both ESs bound tightly by ELISA (FIG. 20), indicative of their ability to recognize diverse antibodies that target the stem helix epitope. In addition to the mature mAbs, S2hlx-Ex15 and -Ex19 binding was also tested to the inferred precursors of S2P6, DH1057.1, CC40.8 and Cov89-22, and compared to the interactions of the stem helix peptide with the same antibodies by ELISA. Interestingly, when the peptide was absorbed directly onto the plate, strong binding of the mature antibodies was detected as expected, but no interactions were measured with the precursors (FIG. 20). Binding between the peptide and the precursor antibodies was detected when the biotinylated peptide was captured onto streptavidin-coated wells, suggesting that epitope presentation plays a critical role in precursor recognition. S2hlx-Ex15 bound at least an order of magnitude tighter to the germline antibodies than the strep-captured peptide by ELISA. Kinetic analysis revealed that S2hlx-Ex15 bound to the UCA for DH1057, and the iGLs for S2P6 and CC40.8. with low nanomolar affinities (K_{D}s of 22.4 nM 1.4 nM and 9.7 nM, respectively), and all the related S2hlx-Ex2 derived designs showed measurable interactions (FIG. 18).

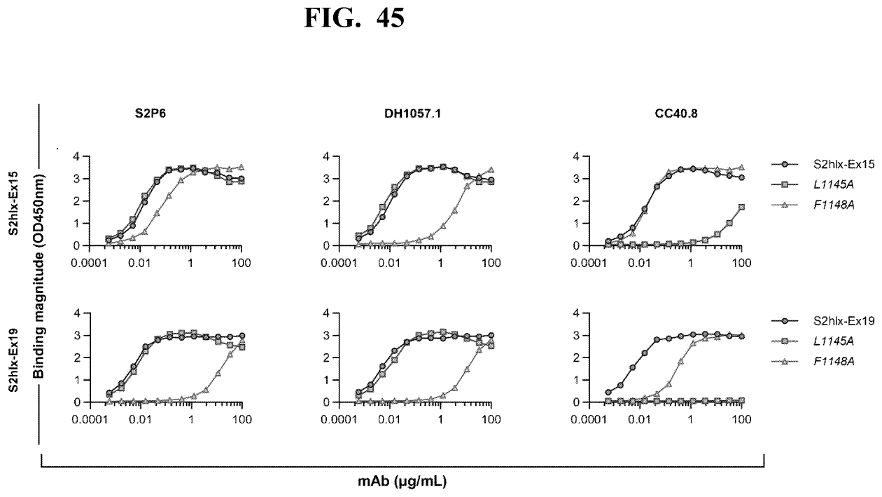

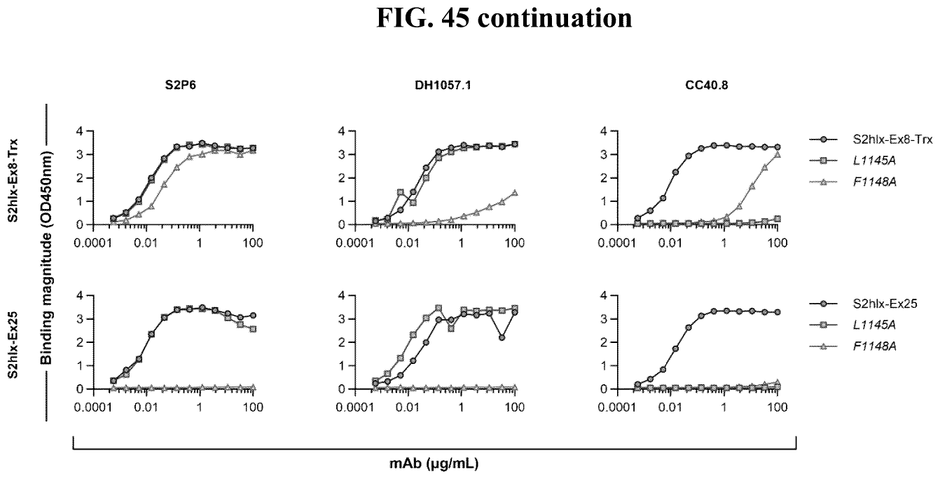

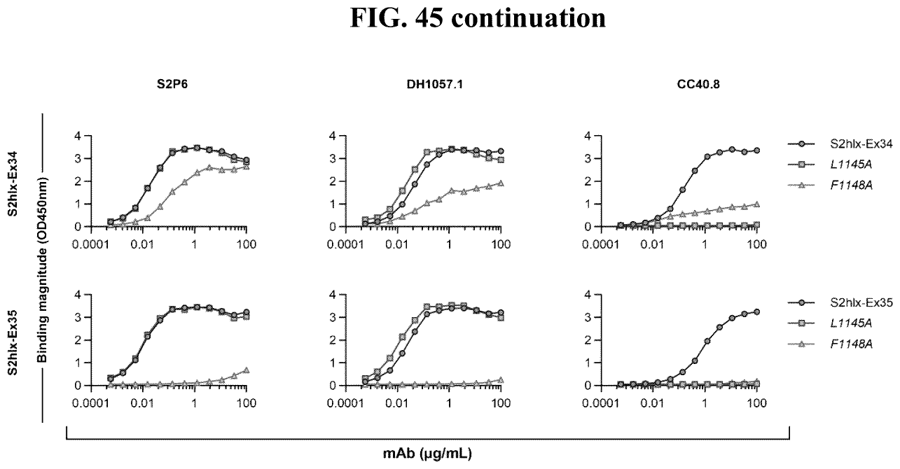

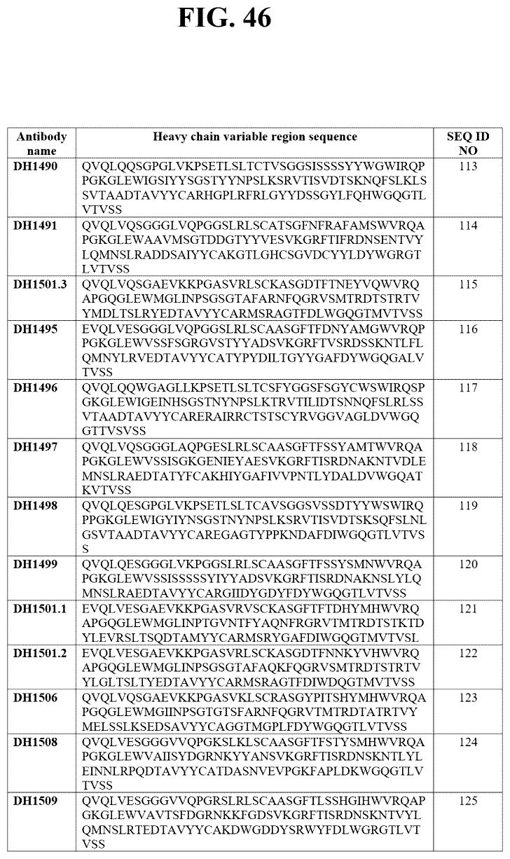

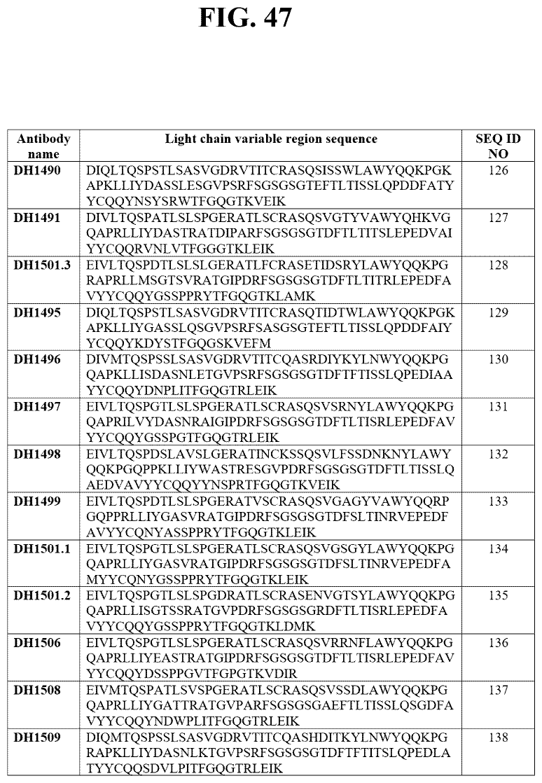

To ensure that the S2hlx ESs interacted with the target mAbs in a similar fashion to the epitope on CoV spike, we analyzed the binding of S2hlx-Ex15 and -Ex19; -Ex8 and -Ex25; and -Ex34 and -Ex35 variants containing alanine mutations at key epitope residues corresponding to spike sites Leu1145 and Phe1148 in the stem helix (FIG. 3f and FIG. 45). As predicted based on existing structural and mutagenesis data, all the ESs lost binding to CC40.8 when the Leu1145 equivalent residue was mutated, while binding to S2P6 and DH1057.1 was not affected. When the epitope residue equivalent to spike Phe1148 was mutated, binding was lost to DH1057.1 and S2P6 in all designs. When the epitope residue equivalent to spike Phe1148 was mutated, binding was lost to DH1057.1 and S2P6 in all designs, confirming that antibody interactions are mediated by the same residues on spike and the designed antigens.

Analysis of a high resolution (2.3 Å) crystal structure of unbound S2hlx-Ex19 confirmed the successful transplantation of the CC40.8 mAb epitope (Table 4). The grafted epitope region in S2hlx-Ex19 diverged considerably from the backbone region it replaced in the parent scaffold (RMSD=5.8 Å), with the epitope adopting a more exposed conformation that allows for antibody engagement (FIG. 3g). This was likely facilitated by the design of two aromatic residues (Tyr62 and Phe66) under the epitope helix, which effectively pushed the epitope segment away from the rest of the protein core. Interestingly, the epitope conformation on S2hlx-Ex19 was almost identical to that present on the prefusion stem helix, rather than the more extended one induced by CC40.8 (FIG. 3g). Nevertheless, given the high affinity of S2hlx-Ex19 for CC40.8 (K_{D}=6.7 nM), it is likely that the antibody can readily induce the necessary conformation upon binding. Thus, by combining epitope backbone grafting and ProteinMPNN, we engineered antigens that bind with high affinity and specificity to the major classes of stem helix broadly reactive antibodies as well as to their inferred precursors.

Cross-Reactivity of Engineered Epitope Scaffolds with Pre-Existing Immune Responses Induced by SARS-CoV-2 Spike.

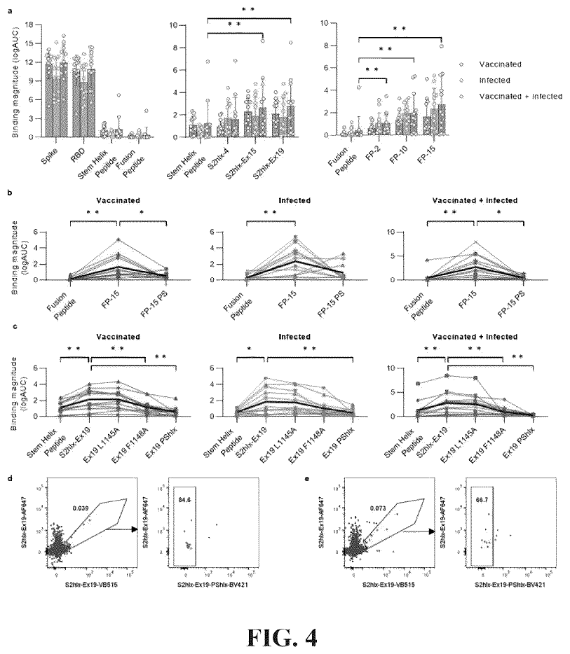

Next, we tested the ability of the engineered epitope scaffolds to cross-react with pre-existing immune responses elicited by SARS-CoV-2 spike in order to evaluate their potential to preferentially amplify fusion peptide or stem helix directed responses by vaccination. Sera or plasma samples isolated from three groups of 12 study participants who acquired SARS-CoV-2 immunity either through vaccination, infection, or vaccination followed by infection respectively, were first tested for binding to WA-2 spike, RBD and synthesized peptides encoding the fusion peptide and stem helix domains (see Table 5 for full patient information). This analysis revealed that only a small fraction of spike-directed humoral responses, regardless of their acquisition route, targeted the fusion peptide or stem helix regions (FIG. 4a). On average, fusion and stem helix peptide binding levels were 23.3 and 8.4-fold lower respectively than RBD-focused responses across all groups, underscoring that the fusion peptide and the stem helix domains are sub-dominant targets of antibodies elicited by spike.

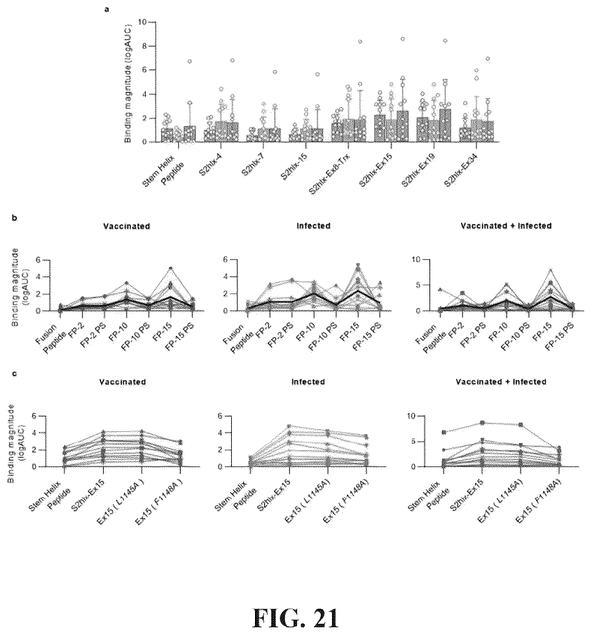

Both fusion peptide and S2hlx-Ex epitope scaffolds showed significantly higher recognition of human sera than the corresponding spike peptide domains (FIG. 4a, FIG. 21). From the FP ES designs, FP-15 had the best sera recognition, showing 11.3, 7.7, and 5.8-fold higher IgG binding than the synthesized fusion peptide in vaccinated, infected and hybrid immunity samples respectively (FIG. 4b). These interactions were mediated by the engineered epitope contacts, as binding to the parent scaffold of FP-15 that does not contain the grafted epitope, was significantly reduced (3.5-fold in vaccinated, 2.5-fold in infected and 5.5-fold in hybrid, FIG. 4b). A similar trend was observed for FP-10 and FP-2, but their overall binding levels were lower than FP-15, although still above those of the synthesized fusion peptide.

From the stem helix designs, S2hlx-Ex19 and S2hlx-Ex15 had the best recognition of spike-induced immune responses (FIG. 21a). Compared to the synthesized stem helix peptide, S2hlx-Ex19 bound on average 2.4 times better across all three groups (FIG. 4c). The binding was epitope specific, as evidenced by the decrease in interactions with S2hlx-Ex19 variants that contained alanine mutations at hotspot epitope residues Leu1145 (LA) and Phe1148 (FA), or that had the whole epitope replaced by the sequence of the parent scaffold (S2hlx-Ex19 PShlx, FIG. 4c). As shown by the larger decrease in binding to the FA than LA mutants, cross-reactive spike responses were more dependent on stem helix residue Phe1148 which is recognized by both S2P6 and CC40.8 classes of antibodies (FIG. 4c, FIG. 21). Compared to S2hlx designs, the ability of the S2hlx-Ex epitope scaffolds to recognize CC40.8 mAb translated into a broader engagement of spike elicited humoral responses for these types of molecules (FIG. 4a, FIG. 21a-c).

TABLE 5
SARS-CoV-2 strain and vaccine information of patient samples analyzed for binding
to native protein domains and the engineered ESs. Study participants in the vaccinated
group received two doses (prime and boost) of the indicated vaccine.

| | | | Days post | COVID | Variant of | Days post |
|---|---|---|---|---|---|---|
| | Patient # | Vaccine | vaccination | strain | concern | symptom onset |
| Vaccinated | 1 | Ad26.Cov2.S plus BNT162b2 | 42 | | | |
| | 2 | mRNA-1273 | 28 | | | |
| | 3 | mRNA-1273 | 29 | | | |
| | 4 | mRNA-1273 | 20 | | | |
| | 5 | mRNA-1273 | 14 | | | |
| | 6 | mRNA-1273 | 44 | | | |
| | 7 | mRNA-1273 | 13 | | | |
| | 8 | BNT162b2 | 52 | | | |
| | 9 | BNT162b2 | 33 | | | |
| | 10 | BNT162b2 | 26 | | | |
| | 11 | BNT162b2 | 26 | | | |
| | 12 | BNT162b2 | 33 | | | |
| Infected | 13 | | | B.1.2 | | 21 |
| | 14 | | | B.1.2 | | 28 |
| | 15 | | | AY.103 | DELTA | 14 |
| | 16 | | | AY.118 | DELTA | 21 |
| | 17 | | | AY.44 | DELTA | 21 |
| | 18 | | | AY.118 | DELTA | 28 |
| | 19 | | | AY.44 | DELTA | 28 |
| | 20 | | | BA.1.1 | OMICRON | 21 |
| | 21 | | | BA.1.1 | OMICRON | 21 |
| | 22 | | | BA.1.1 | OMICRON | 28 |
| | 23 | | | BA.1.1 | OMICRON | 28 |
| | 24 | | | BA.1.1 | OMICRON | 7 |
| Vaccinated + | 25 | Ad26.Cov2.S | 51 | AY.103 | DELTA | 14 |
| Infected | 26 | Ad26.Cov2.S | 192 | AY.44 | DELTA | 21 |
| | 27 | Ad26.Cov2.S | 197 | AY.103 | DELTA | 21 |
| | 28 | Ad26.Cov2.S | 98 | AY.44 | DELTA | 28 |
| | 29 | Ad26.Cov2.S | 193 | BA.1.1 | OMICRON | 28 |
| | 30 | mRNA-1273 | 376 | BA.1.1 | OMICRON | 21 |
| | 31 | BNT162b2 | 65 | B.1.1.207 | | 28 |
| | 32 | BNT162b2 | 38 | AY.44 | DELTA | 21 |
| | 33 | BNT162b2 | 131 | AY.44 | DELTA | 28 |
| | 34 | BNT162b2 | 41 | AY.44 | DELTA | 28 |
| | 35 | BNT162b2 | 516 | BA.2.12.1 | OMICRON | 28 |
| | 36 | BNT162b2 | 437 | BA.2.12.1 | OMICRON | 28 |

### REFERENCES

1. L. Piccoli et al., Mapping Neutralizing and Immunodominant Sites on the SARS-CoV-2 Spike Receptor-Binding Domain by Structure-Guided High-Resolution Serology. Cell 183, 1024-1042 e1021 (2020).
2. Z. Wang et al., mRNA vaccine-elicited antibodies to SARS CoV-2 and circulating variants. Nature 592, 616-622 (2021).
3. R. R. Goel et al., Distinct antibody and memory B cell responses in SARS-CoV-2 naïve and recovered individuals after mRNA vaccination. Science Immunology 6, eabi6950 (2021).
4. D. F. Robbiani et al., Convergent antibody responses to SARS CoV-2 in convalescent individuals. Nature 584, 437-442 (2020).
5. T. N. Starr et al., Deep mutational scans for ACE2 binding, RBD expression, and antibody escape in the SARS-CoV-2 Omicron BA.1 and BA.2 receptor-binding domains. PLoS Pathog 18, e1010951 (2022).
6. P. Qu et al., Enhanced neutralization resistance of SARS-CoV 2 Omicron subvariants BQ.1, BQ.1.1, BA.4.6, BF.7, and BA.2.75.2. Cell Host Microbe, (2022).
7. C. Dacon et al., Broadly neutralizing antibodies target the coronavirus fusion peptide. Science 377, 728-735 (2022).
8. J. S. Low et al., ACE2-binding exposes the SARS-CoV-2 fusion peptide to broadly neutralizing coronavirus antibodies. Science 377, 735-742 (2022).
9. D. Pinto et al., Broad betacoronavirus neutralization by a stem helix-specific human antibody. Science 373, 1109-1116 (2021).
10. P. Zhou et al., A human antibody reveals a conserved site on beta-coronavirus spike proteins and confers protection against SARS-CoV-2 infection. Science Translational Medicine 14, eabi9215 (2022).
11. C. Dacon et al., Rare, convergent antibodies targeting the stem helix broadly neutralize diverse betacoronaviruses. Cell Host & Microbe 0, (2022).
12. P. Zhou et al., “Broadly neutralizing anti-S2 antibodies protect against all three human betacoronaviruses that cause severe disease,” (Immunology, 2022).
13. M. L. Azoitei et al., Computation-guided backbone grafting of a discontinuous motif onto a protein scaffold. Science 334, 373-376 (2011).
14. B. E. Correia et al., Computational Design of Epitope-Scaffolds Allows Induction of Antibodies Specific for a Poorly Immunogenic HIV Vaccine Epitope. Structure 18, 1116-1126 (2010).
15. G. Ofek et al., Elicitation of structure-specific antibodies by epitope scaffolds. Proceedings of the National Academy of Sciences 107, 17880-17887 (2010).
16. G. Ofek et al., Elicitation of structure-specific antibodies by epitope scaffolds. Proc Natl Acad Sci USA 107, 17880-17887 (2010).
17. F. Sesterhenn et al., Trivalent cocktail of <em>de novo</em> designed immunogens enables the robust induction and focusing of functional antibodies <em>in vivo</em>. bioRxiv, 685867 (2019).
18. S. M.-C. Gobeil et al., Structural diversity of the SARS-CoV-2 Omicron spike. Molecular Cell 82, 2050-2068.e2056 (2022).
19. V. Stalls et al., Cryo-EM structures of SARS-CoV-2 Omicron BA.2 spike. Cell Reports 39, 111009 (2022).
20. D. Li et al., In vitro and in vivo functions of SARS-CoV-2 infection-enhancing and neutralizing antibodies. Cell 184, 4203-4219.e4232 (2021).
21. J. Jumper et al., Highly accurate protein structure prediction with AlphaFold. Nature 596, 583-589 (2021).
22. J. Dauparas et al., Robust deep learning-based protein sequence design using ProteinMPNN. Science 378, 49-56 (2022).
23. C. Dacon et al., Rare, convergent antibodies targeting the stem helix broadly neutralize diverse betacoronaviruses. Cell Host & Microbe.
24. K. O. Saunders et al., Neutralizing antibody vaccine for pandemic and pre-emergent coronaviruses. Nature 594, 553-559 (2021).
25. P. Shah, G. A. Canziani, E. P. Carter, I. Chaiken, The Case for S2: The Potential Benefits of the S2 Subunit of the SARS-CoV-2 Spike Protein as an Immunogen in Fighting the COVID-19 Pandemic. Frontiers in Immunology 12, (2021).
26. M. L. Azoitei et al., Computational design of high-affinity epitope scaffolds by backbone grafting of a linear epitope. J Mol Biol 415, 175-192 (2012).
27. J. Dauparas et al., Robust deep learning& #x2013; based protein sequence design using ProteinMPNN. Science 378, 49-56 (2022).
28. H. Duan et al., Glycan Masking Focuses Immune Responses to the HIV-1 CD4-Binding Site and Enhances Elicitation of VRC01-Class Precursor Antibodies. Immunity 49, 301-311.e305 (2018).
29. T. G. Battye, L. Kontogiannis, O. Johnson, H. R. Powell, A. G. Leslie, iMOSFLM: a new graphical interface for diffraction-image processing with MOSFLM. Acta Crystallogr D Biol Crystallogr 67, 271-281 (2011).
30. P. R. Evans, G. N. Murshudov, How good are my data and what is the resolution? Acta Crystallogr D Biol Crystallogr 69, 1204-1214 (2013).
31. A. J. McCoy, Solving structures of protein complexes by molecular replacement with Phaser. Acta Crystallogr D Biol Crystallogr 63, 32-41 (2007).
32. P. Emsley, K. Cowtan, Coot: model-building tools for molecular graphics. Acta Crystallogr D Biol Crystallogr 60, 2126-2132 (2004).
33. T. I. Croll, ISOLDE: a physically realistic environment for model building into low-resolution electron-density maps. Acta Crystallogr D Struct Biol 74, 519-530 (2018).
34. P. D. Adams et al., PHENIX: a comprehensive Python-based system for macromolecular structure solution. Acta Crystallogr D Biol Crystallogr 66, 213-221 (2010).

### Methods

### Computational Design of Epitope Scaffolds.

Design of epitope scaffolds by side chain grafting. A database of 9884 candidate scaffolds was created by selecting from the Protein Data Bank (PDB) structures that were: 1) determined by x-ray crystallography; 2) high resolution (<2.8 Å); 3) monomeric; 4) expressed in E. Coli; 5) not of human origin and 6) that did not contain ligands. Epitope scaffolds were designed with Rosetta as previously reported, using the RosettaScripts for side chain grafting described in Silva et. al., with the following parameters in the MotifGraft mover: RMSD_tolerance=“0.3”; NC_points_RMSD_tolerance=“0.5”; clash_test_residue=“ALA”; clash_score_cutoff=“5”. For the design of fusion peptide (FP) epitope scaffolds, the structure of fusion peptide fragment ⁸¹⁵RSFIEDLLF⁸²³ (SEQ ID NO: 66) as described in the crystal structure of the DH1058-FP peptide complex (PDBid: 7tow) was used as the target epitope to graft. With the exception of residues F817 and L821 which were allowed to change in the designed epitope scaffold because they did not contribute to antibody binding, the identity of all the other epitope residues was maintained. For the design of epitope scaffolds that bind S2P6 and DH1057.1 mAbs, the structure of the stem helix fragment ¹¹⁴⁸FKEELDKYF¹¹⁵⁶ (SEQ ID NO: 79) from PDBid:7rnj was grafted. The identity of residues E1150 and K1154 was allowed to change during the design process while the other epitope residues were kept fixed. From the epitope scaffold generated by the automated protocol, the top 100 models by Rosetta ddG that were also smaller than 150 amino acids were visually examined to ensure appropriate epitope transplantation and antibody-scaffold interaction. At this stage, the suitability of a given parent scaffold in terms of function, conformational flexibility, and expression protocol was also investigated by referencing the publication it originated from. If necessary, additional changes were introduced in a candidate epitope scaffold using Rosetta fixed backbone design to remove antibody-epitope scaffold contacts that were not due to the target epitope and to ensure proper interactions between the epitope and the rest of the scaffold. The best 15 designs for each of the target epitopes were chosen for experimental characterization.

### Design of Stem Helix Epitope Scaffolds by Backbone Grafting and MPNN Optimization.

Epitope scaffolds that display the stem helix epitope recognized by the CC40.8 antibody (PDBid: 7sjs) were designed by backbone grafting as previously reported and using the RosettaScripts described by Silva et. al. The same set of curated parent scaffolds as the one used for side chain grafting above was searched here. The structure of the stem helix fragment ¹¹⁴⁴ELDSFKEELDKYFK¹¹⁵⁷ (SEQ ID NO: 89) from PDBid:7sjs was grafted. The identity of residues E1144, D1146, S1147, E1150, K1154, and K1157 were allowed to change during the design process while the other epitope residues were kept fixed. The following parameters were used:
RMSD_tolerance=“5.0” NC_points_RMSD_tolerance=“0.75”
clash_score_cutoff=“5” clash_test_residue=“ALA”
hotspots=“2:5:6:8:9:10:12:13”
combinatory_fragment_size_delta=“1:1”
max_fragment_replacement_size_delta=“−4:4”
full_motif_bb_alignment=“0”
allow_independent_alignment_per_fragment=“0”
graft_only_hotspots_by_replacement=“0”
only_allow_if_N_point_match_aa_identity=“0”
only_allow_if_C_point_match_aa_identity=“0”
revert_graft_to_native_sequence=“0”
allow_repeat_same_graft_output=“0”/>

The initial hits were filtered to remove scaffolds with (1) an average clash score greater than 5 across the epitope, and (2) a Rosetta calculated ddg greater than 0. The top 100 models by Rosetta ddG that were also smaller than 150 amino acids were then visually examined as above and then Alphafold2 was used to predict the structure of the 10 best designs using the following parameters—
model_preset=monomer
db_preset=full_dbs
max_template_date=2021-11-01
uniref90_database_path=/data/uniref90/uniref90.fasta
mgnify_database_path=/data/mgnify/mgy_clusters.fa
uniclust30_database_path=/data/uniclust30/uniclust30_2018_08/uniclust30_2018_08
bfd_database_path=/data/bfd/bfd_metaclust_clu_complete_id30_c90_final_seq.sorted_opt
template_mmcif_dir-/data/pdb_mmcif/mmcif_files
obsolete_pdbs_path=/data/pdb_mmcif/obsolete.dat
pdb70_database_path=/data/pdb70/pdb70

The scaffolds were then aligned via the grafted epitope to the stem helix fragment in the CC40.8 structure PDBid:7sjs and inspected for clashes between the scaffold and antibody. Scaffolds with clashes, such as where the antibody binding epitope was occluded or buried, were modified such that key epitope residues above remained fixed while other residues in the epitope and in contacting parts of the scaffold were allowed to vary through iterative rounds of fixed-backbone rotamer-based sequence design in Rosetta. After each round, we checked the Alphafold2 prediction and repeated until the epitope was predicted to be presented in a suitable orientation for antibody binding (FIG. 3a). Expression tags were screened by N-terminally tagging S2hlx-Ex4 with GST, SUMO, Thioredoxin (Trx), Maltose binding protein (MBP), or F8H tags. As Trx gave the highest fraction of full-length protein it was then used to tag S2hlx-Ex2 and -Ex6 (FIG. 15).

The parent structure of S2hlx-Ex4 (PDBid:2QYW) was used to screen the VAST+ database (structure.ncbi.nlm.nih.gov/Structure/VAST/vast.shtml) for homologous structures, which were identified by RMSD (<2 Å) and high fraction of alignment (>80%). Two homologs identified were expressed with Trx tags (FIG. 16).

For ESs where protein expression was low the protein structures, either the Rosetta model or Alphafold prediction, were entered into ProteinMPNN at the website, huggingface.co/spaces/simonduerr/ProteinMPNN. Initial designs for S2hlx-Ex2 and -4 used sampling temperature of 0.1 and backbone noise of 0.02 and the whole epitope sequence was fixed, For S2hlx-Ex2, this approach produced six related soluble ESs but failed for S2hlx-Ex4. The second set of S2hlx-Ex4 and S2hlx-Ex6 designs used the higher sampling temperature (0.25) and higher backbone noise (0.2) to increase sequence diversity in designs, and only L¹¹⁴⁵xxFKxELDxYF¹¹⁵⁶ (SEQ ID NO: 185) residues were fixed. ES structures were modelled using the integrated Colabfold prediction and scored by RMSD and pLDDT, and the highest scoring were re-predicted using Alphafold2. For the S2hlx-Ex6 designs, the epitope was occluded or buried in several designs. In these cases, residues in the epitope or surrounding parts of the ES were manually modified and the ES structure prediction repeated using Alphafold2 until the epitope was predicted to be suitable to binding to S2P6. Figures showing structures were prepared in Pymol v2.5.5 (Schroedinger).

### Plasmids and DNA Synthesis.

Genes encoding designed epitope scaffolds were commercially synthesized and cloned into pET29b (Genscript). Genes encoding the antibody heavy and light chains were similarly synthesized and cloned into the pcDNA3.1 vector (GenScript). Oligonucleotides were synthesized by IDT. Mutations were introduced in the synthesized plasmids by Q5 mutagenesis (New England Biolabs, Ipswich, MA).

### Recombinant Protein Expression and Purification.

### Epitope-Scaffolds.

Plasmids encoding epitope-scaffolds were transformed into E. coli BL-21 (DE3) (New England Biolabs) cells and 5 mL starter cultures were grown overnight at 37° C. in Lysogeny Broth (LB) supplemented with 50 g/mL kanamycin. The cultures were diluted 1:100 in Terrific BrothTB media (RPI) supplemented with kanamycin and grown at 37° C. to an OD600 of ˜0.6. The temperature was subsequently lowered to 16° C. and the cultures were grown to OD600 of ˜0.8 and induced with isopropyl β-D-1-thiogalactopyranoside (IPTG) to a final concentration of 0.5 mM. The cultures were shaken for 16-18 hours at 200 rpm. Cell pellets were collected by centrifugation at 14,500×g and lysed in B-PER Reagent (ThermoFisher Scientific) according to the manufacturer's protocol. The lysates were centrifuged at 14,000×g for 30 minutes at 4° C. The supernatant was incubated with Ni-NTA beads (Qiagen) equilibrated with native binding buffer (20 mM NaH₂PO4, 500M NaCl, 2% glycerol, 10 mM imidazole, pH=7.5) for an hour at 4° C. The beads were settled by centrifugation and the supernatant was removed by pipetting. The beads were washed with wash buffer (50 mM NaH₂PO4, 500M NaCl, 2% glycerol, 30 mM imidazole, pH=7.5) after which the protein was eluted with elution buffer (20 mM NaH₂PO4, 500M NaCl, 250 mM imidazole, pH=7.5). Protein expression and purity was confirmed by SDS-PAGE analysis, and quantified spectrophotochemically at 280 nm on a Nanodrop 2000 (ThermoFisher Scientific). The eluted protein was concentrated on 3 kDa MW spinMW spin columns (ThermoFisher Scientific) and further purified by size exclusion chromatography in 20 mM NaH₂PO4, 150 mM NaCl, pH=7.5, on an AKTA-Go FPLC (Cytiva) using a Superdex 200 Increase 10/300GL column. Fractions containing monomeric protein were pooled and concentrated as above.

### Monoclonal Antibodies.

Expi293F cells (ThermoFisher Scientific) were split to a density of 2.5×10⁶ cells/mL in Expi293 Expression Medium with GlutaMAX (Gibco) and were transiently transfected with an equimolar plasmid mixture of heavy and light chain using Expifectamine (Invitrogen). For a typical 100 mL size transfection 100 μg amount of total DNA and 270 μL of lipofectamine were used. After overnight incubation, enhancers were added as per the manufacturer's protocol and the cultures were incubated with shaking for five days at 37° C. and 5% CO₂. The cell culture was centrifuged to remove the cells and the supernatant was filtered with a 0.8-micron filter. The filtered supernatant was incubated with equilibrated Protein A beads (ThermoFisher) for one hour at 4° C. and washed with 20 mM Tris, 350 mM NaCl at pH=7. The antibodies were eluted with a 2.5% Glacial Acetic Acid Elution Buffer and were buffer exchanged into 25 mM Citric Acid, 125 mM NaCl buffer at pH=6. IgG expression was confirmed by reducing SDS-PAGE and quantified by measuring absorbance at 280 nm (Nanodrop 2000).

### Antigenic Characterization of Epitope-Scaffolds.

### Binding by Surface Plasmon Resonance.

To determine the dissociation constants (K_{D}s) between epitope scaffolds and target antibodies, binding was measured by Surface Plasmon Resonance (SPR) on a Biacore T200 instrument using Protein-A coated S series chips. The target antibodies were individually captured for 60 sec at 30 μL/min to a level of 1000-2000RU onto this surface. The epitope scaffolds were subsequently injected as analytes at five concentrations using the single cycle injection method. The association phase was carried out for 180 seconds and the dissociation was done for 900 seconds with HBS-EP+ buffer flowing at 30 μL/min. Regeneration of the binding surface was done in 10 mM glycine-HC, pH=2 for 30 seconds at 30 μL/min with a 30 second baseline stabilization. A 1:1 Langmuir or Heterogenous ligand model was used for data fitting and analysis.

For KDs between Spike proteins and target antibodies, neutravidin (Thermo Scientific) was first captured for 30-60 sec at 30 μL/min to a level of 1000-2000RU using CM5 S-series chips. Then biotinylated Spike proteins (WA-2, 2 P, R&D systems) were subsequently captured to a level of 3-500RU by the neutravidin. FAB fragments of antibodies were then injected as analytes at five concentrations using the single cycle method described above.

### Antibody Binding by ELISA.

Spike815-823 peptide (residues 808-833) and stem helix (1140-1163) peptides and mutated variants were com-mercially synthesized (Genscript). The following Spike proteins were used: WA-2, 2P; SARS-CoV-1, 2P; MERS, 2P; OC43, 2P; HKU-1, 2P; GXP4L, 2 P; SHC014, 2 P; RaTG13, 2 P, (all produced according to 32,33); and Delta; BA1.1, Hexapro; XBB, Hexapro; 229E; NL63; (all from Sino Biological). Peptides and Spikes were diluted to 2 μg/mL in 0.1 M Sodium Bicarbonate and epitope scaffolds were diluted to 100 μg/mL. Antigens were coated onto 96 or 384 well high binding enzyme-linked immunosorbent assay (ELISA) plates (Corning) by overnight incuba-tion at 4° C. Plates were washed with Superwash buffer (1×PBS supplemented with 0.1% Tween-20) and blocked for 1 hour at room temperature with Superblock buffer supplemented with azide (80 g Whey Protein, 300 mL Goat Serum, 20 mL aqueous 5% Sodium Azide, 10 mL Tween20, 80 mL of 25×PBS, diluted to 2 L). Antibodies starting at 100 μg/mL were serially diluted in Superblock with azide using a threefold serial dilution, added to the plates, and incubated at room temperature for 1 hour. Plates were then washed twice with Superwash and goat anti Human IgG-HRP secondary antibody (Jackson Immu-noResearch Laboratories; Code Number: 109-035-098; Lot number: 154823) diluted 1:15,000 in SuperBlock (without Sodium azide) was subsequently added. After incubating at room temperature for 1 hour, the plates were washed four times in Superwash. Room temperature TMB (Tetramethylbenzidine) substrate was added and after 5 minutes elapsed, the reaction was stopped by acid stop solution (0.33 N HCl). Absorption was measured at 405 nm on a Cytation 1 plate reader (BioTek). Data was analyzed and plotted with Prism version 10.0.0 (Graphpad).

### Sera Binding by ELISA

Antigens were diluted to 2 μg/mL in 0.1 M Sodium Bicarbonate and coated onto 384 well high binding enzyme-linked immunosorbent assay (ELISA) plates (Corning) by overnight incubation at 4° C. Plates were washed with Superwash buffer (1×PBS supplemented with 0.1% Tween-20) and blocked for 1 hour at room temperature with Superblock buffer supplemented with azide (80 g Whey Protein, 300 mL Goat Serum, 20 mL aqueous 5% Sodium Azide, 10 mL Tween20, 80 mL of 25×PBS, diluted to 2 L). Mouse sera starting at a dilution ratio of 1:50 and control antibodies starting at 100 μg/mL were serially diluted in Superblock with azide using a fivefold serial dilution scheme. Human sera starting at a dilution ratio of 1:30 and control antibodies starting at 100 μg/mL were serially diluted in Superblock with azide using a threefold serial dilution scheme. Sera and controls were added to the plates and incubated at room temperature for 1.5 hours. Plates were then washed twice with Superwash and the corresponding IgG-HRP secondary antibody (goat anti mouse IgG-HRP, 1:10,000 dilution, Jackson ImmunoResearch Laboratories; Code Number: 115-035-071; Lot number: 158206; or goat anti human IgG-HRP, 1:15,000 dilution, Jackson ImmunoResearch Laboratories, Code Number: 109-035-098; Lot number: 154823) in SuperBlock (without Sodium azide) was subsequently added. After incubating at room temperature for 1 hour, the plates were washed four times in Superwash. Room temperature TMB (Tetramethylbenzidine) substrate was added and after 15 minutes elapsed, the reaction was stop-ped by acid stop solution (0.33 N HCl). Absorption was measured at 405 nm on a SpectraMax Plus plate reader (Molecular Devices). Data was analyzed and plotted with Prism version10.0.0 (Graphpad)

Structural Analysis by x-Ray Crystallography.

Crystallography experiments were performed using the sitting drop vapor diffusion technique. 96-well crystallization plates were set, in which the protein of interest was mixed in a 1:1 ratio with reservoir solution. For S2hlx-Ex19, crystals were obtained when a 9.5 mg/mL sample was mixed with 0.1 M citric acid, 3.0 M sodium chloride, pH 3.5 (Index Screen) at 4° C. S2hlx-7 and antibody DH1057.1 were mixed in 1:1 molar ratio (300 uM) and incubated for one hour at 4° C. Crystals for the complex were obtained in 25% PEG 3350 at room temperature. FP-15 and antibody DH1058 were mixed in 1:1 molar ratio (60 μM) and incubated for one hour at 4° C. Crystals for the complex were obtained in 0.2 M sodium malonate pH 7 and 25% PEG 3350 at room temperature. Prior to data collection, crystals were cryoprotected in mother liquor supplemented with 20% glycerol before being plunge-frozen into liquid nitrogen. Diffraction data for S2hlx-Ex19 and FP15-DH1058 were collected at APS SER-CAT 22-ID-D and diffraction data for S2hlx-7-DH1057.1 were collected at APS SER-CAT 22-BM-D. All datasets were collected at cryogenic conditions with a wavelength of 1.00 Å. A full description of the crystallographic data collection and refinement statistics can be found in Table 1. Diffraction data were indexed in iMOSFLM (29) and scaled in AIMLESS (30). Molecular replacement solutions for both S2hlx-Ex19 and the S2hlx-7-DH1057.1 complex were found in PhaserMR (31), using PDB ID: 3N1B as a search ensemble for S2hlx-Ex19, PDB ID: 3LMO as a search ensemble for S2hlx-7 and PDB ID: 6UOE as a search ensemble for DH1057.1. Coordinates for S2hlx-Ex19 and S2hlx-7-DH1057.1 were iteratively built and refined using Coot (32), ISOLDE (33) and Phenix (34) to Rwork/Rfree values of 25.4/27.5 and 21.1/24.5, respectively. The presence of translational non-crystallographic symmetry in the S2hlx-7-DH1057.1 crystal likely accounts for its relatively high Rfree value and the L-test did not indicate the presence of any twinning. Figures showing structures were prepared in Pymol v2.5.5 (Schroedinger).

Reactivity of Designed Epitope Scaffolds with Human Samples

Human subject studies were approved by the Duke University Health System Institutional Review Board (IRB) and conducted in agreement with the policies and protocols approved by the Duke IRB, consistent with the Declaration of Helsinki. Written informed consent was obtained from all research subjects or their legally authorized representatives. Study participants with SARS-CoV-2 acute infection were enrolled in the Molecular and Epidemiological Study of Suspected Infection protocol (MESSI, IRB Pro00100241) at Duke University, and were followed longitudinally. Samples were selected for this study from participants who had seroconverted and had symptom onset more than 10 days prior. From patient 26 and patient 29, PBMCs were collected and analyzed by FACS at the same time point used for sera analysis for patient 26 and 7 days later for patient 29. Samples from vaccinated only participants were obtained from subjects enrolled in the Study of Immune Response to COVID-19 Vaccines (IRB Pro00107929) at Duke University. Human sera were analyzed as described above in sera binding by ELISA method. Statistical differences were tested using the Wilcoxon signed rank test.

### Isolation and Analysis of Epitope Scaffold Reactive B Cells

We prepared B cell tetramers of biotinylated S2hlx-Ex19 or FP-10 by mixing with Streptavidin-VB515 (Miltenyi Biotec) and Streptavidin-AlexaFluor 647 (ThermoFisher Scientific) at 4:1 molar ratio. A scaffold-only tetramer was also made with biotinylated S2hlx-Ex19-PShlx or FP-10-wt mixed with Streptavidin-BV421 (Biolegend). These probes did not contain the grafted epitope.

Cryopreserved PBMCs were thawed in warm RPMI-1640 contain-ing 10% FBS, then counted. Cells were stained with pre-optimized concentrations of the following antibodies: PE anti-human IgD (clone IA6-2, BD Biosciences, 1:300 final concentration), PE-TXRD anti-human CD10 (clone HI10A, BD Biosciences, 1:300), PE-Cy5 anti-human CD3 (clone HIT3a, BD Biosciences, 1:40), PE-Cy7 anti-human C27 (clone 0323, ThermoFisher Scientific, 1:150), AlexaFluor 700 anti-human CD38 (clone LS198-4-3, Beckman Coulter, 1:40), APC-Cy7 anti-human CD19 (clone SJ25C1, BD Biosciences, 1:80), BV570 anti-human CD16 (clone 3G8, Biolegend, 1:40), BV605 anti-human CD14 (clone M5E2, Biolegend, 1:40), and BV711 anti-human IgM (clone G20-127, BD Bios-ciences, 1:160). S2hlx-Ex19-VB515- and -AF647, and S2hlx-Ex19-PShlx-BV421 were added to identify stem helix epitope-specific B cells. Cells were incubated with Aqua Live/Dead (ThermoFisher Scientific, 1:1000) to exclude dead cells. Cells were acquired on a BD S6 cell sorter (BD Biosciences). Data were analyzed using FlowJo v10.8 (BD Biosciences).

Immunoglobulin heavy and light chain variable regions (VH and VK/L) from singly sorted antigen-specific memory B cells were RT-PCR amplified using SuperScript III and AmpliTaq Gold 360 Master Mix (Thermo Fisher Scientific, Waltham, MA) under conditions previously described22. PCR products were purified in Biomek FX Laboratory Automation Workstation (Beckman Coulter, Indianapolis, IN) and sequenced by Sanger sequencing. The V(D)J rearrangement, somatic hypermutation frequency, CDR3 length of VH and VK/L chains, and antibody clonal lineages were analyzed using the software Cloanalyst40. The heavy chains of mAbs DH1493, DH1501, and DH1502 were assigned to the same clone by the software package Cloanalyst. Unmutated common ancestor (UCA) inference was performed using the heavy and kappa chain pairs of the three mAbs using the paired-chain inference implementation of the UCA inference part of the software package Cloanalyst.

RT-PCR amplified sequences were transiently expressed as pre-viously described22. Briefly, the linear expression cassettes were con-structed by overlapping PCR to place the PCR-amplified VH and VK/L chain genes under the control of a CMV promoter along with heavy chain IgG1 constant region or light chain constant region and a BGH ploy A signal sequence. The linear expression cassettes of heavy and light chains were then co-transfected into 293 T cells in 6-well plates. After 3 days, the cell culture supernatants were harvested and concentrated for binding assays. For antibodies of interest, recombinant IgG1 monoclonals were expressed and purified as described above.

### Flow Cytometry

We prepared B cell tetramers of biotinylated S2hlx-Ex19 by mixing with Streptavidin-VB515 (Miltenyi Biotec) and Streptavidin-AlexaFluor 647 (ThermoFisher Scientific) at 4:1 molar ratio. A scaffold-only tetramer was made with biotinylated S2hlx-Ex19-PShlx mixed with Streptavidin-BV421 (Biolegend).

Cryopreserved PBMCs were thawed in warm RPMI-1640 containing 10% FBS, then counted. Cells were stained with pre-optimized concentrations of the following antibodies: PE anti-human IgD (clone IA6-2, BD Biosciences), PE-TXRD anti-human CD10 (clone HI10A, BD Biosciences), PE-Cy5 anti-human CD3 (clone HIT3a, BD Biosciences), PE-Cy7 anti-human C27 (clone 0323, ThermoFisher Scientific), AlexaFluor 700 anti-human CD38 (clone LS198-4-3, Beckman Coulter), APC-Cy7 anti-human CD19 (clone SJ25C1, BD Biosciences), BV570 anti-human CD16 (clone 3G8, Biolegend), BV605 anti-human CD14 (clone M5E2, Biolegend), and BV711 anti-human IgM (clone G20-127, BD Biosciences). S2hlx-Ex19-VB515- and -AF647, and S2hlx-Ex19-PShlx-BV421 were added to identify stem helix epitope-specific B cells. Cells were incubated with Aqua Live/Dead (ThermoFisher Scientific) to exclude dead cells. Cells were acquired on a BD S6 cell sorter (BD Biosciences). Data were analyzed using FlowJo v10.8 (BD Biosciences).

### Pseudovirus Neutralization Assay

The pseudovirus neutralization assay performed at Duke has been described in detail (41) and is a formally validated adaptation of the assay utilized by the Vaccine Research Center; the Duke assay is FDA approved for D614G and other SARS-CoV-2 variants. For measurements of neutralization, pseudovirus was incubated with 8 serial 5-fold dilutions of antibody samples in duplicate in a total volume of 150 μl for 1 hr at 37° C. in 96-well flat-bottom culture plates. 293 T/ACE2-MF cells (obtained from Drs. Mike Farzan and Huihui Mu at The Scripps Research Institute) were detached from T75 culture flasks using TrypLE Select Enzyme solution, suspended in growth medium (100,000 cells/ml) and immediately added to all wells (10,000 cells in 100 μL of growth medium per well). One set of 8 wells received cells+virus (virus control) and another set of 8 wells received cells only (background control). After 71-73 hrs of incubation, medium was removed by gentle aspiration and 30 μl of Promega 1× lysis buffer was added to all wells. After a 10-minute incubation at room temperature, 100 μl of Bright-Glo luciferase reagent was added to all wells. After 1-2 minutes, 110 μl of the cell lysate was transferred to a black/white plate. Luminescence was measured using a GloMax Navigator luminometer (Promega). Neutralization titers are the inhibitory dilution (ID) of serum samples at which RLUs were reduced by 50% (ID₅₀) compared to virus control wells after subtraction of background RLUs. Serum samples were heat-inactivated for 30 minutes at 56° C. prior to assay.

### Infected-Cell Antibody Binding Assay

The binding of isolated anti-SARS-CoV-2 monoclonal antibodies to infected cells was measured as previously reported42. Briefly, Vero E6 cells (ATCC CRL-1587) expressing TMPRSS2 and ACE2 and infected with either D614G (Germany/BavPat1/2020) or BA.1 (hCoV-19/USA/MD-HP20874/2021) variants were incubated with TrypLE Select (Gibco) for 15 minutes at 37° C. to detach cells and washed with PBS. Monoclonal antibodies were then added to infected cells at 2, 10 or 50 μg/mL. Approximately 2×10⁵ infected cells were incubated with the mAbs for 30 minutes at room temperature, washed and then incu-bated with vital dye (Live/Dead Far Red Dead Cell Stain, Invitrogen) for 15 minutes at room temperature to exclude nonviable cells from sub-sequent analysis. Cells were then washed with Wash Buffer (1% FBS-PBS; WB), pelleted by centrifugation and incubated with 1 mL of 4% Methanol-free Formaldehyde (Duke GHRB SOP 38; Attachment 17) for 30 minutes at room temperature. Cells were then washed twice with Wash Buffer, permeabilized with CytoFix/CytoPerm (BD Biosciences) and stained with A568-conjugated anti-SARS-CoV-2 nucleocapsid antibody (1 μg/mL; 40143-MM08, Sino Biological) and PE/Cy7-con-jugated secondary anti-Human IgG Fc antibody (10 μL/mL, Clone: HP6017, Biolegend) for 30 minutes at room temperature. Cells were washed and resuspended in 250 LPBS-1% paraformaldehyde. Samples were acquired within 24 h using a BD Fortessa cytometer and a High Throughput Sampler (HTS, BD Biosciences). Data analysis was performed using FlowJo 10 software (BD Biosciences). Gates were set to include singlet, live, nucleocapsid+ (NC+) and IgG+ events. Binding to mock infected cells was measured using the live cell gate as there were no NC+ events. All final data represent specific binding, determined by subtraction of non-specific binding observed in assays performed with mock-infected cells.

### Antibody-Dependent NK Cell Degranulation Assays (Infected and Spike-Transfected)

Cell-surface expression of CD107a was used as a marker for NK cell degranulation and performed as previously described42. Briefly, target cells were 293 T (ATCC, CRL-3216) cells 2-days post transfection with a SARS-CoV-2 S protein (D614G) expression plasmid. Natural killer cells purified by negative selection (Miltenyi Biotech) from peripheral blood mononuclear cells obtained by leukapheresis from a healthy, SARS-CoV-2-seronegative individual (Fc-gamma-receptor IIIA (Fcy-RIIIA) 158 V/F heterozygous) previously assessed for Fcy-RIIIA genotype and frequency of NK cells were used as a source of effector cells. NK cells were incubated with target cells at a 1:1 ratio in the presence of monoclonal antibodies, Brefeldin A (GolgiPlug, 1 μl/ml, BD Bios-ciences), monensin (GolgiStop, 4 μl/6 mL, BD Biosciences), and anti-CD107a-FITC (25 μL/mL BD Biosciences, clone H4A3) in 96-well flat bottom plates for 6 hours at 37° C. and 5% CO₂. NK cells were removed from the wells and stained for viability prior to staining with CD56-PECy7 (3.125 μL/mL, BD Biosciences, clone NCAM16.2), CD16-PacBlue (12.5 μL/mL, BD Biosciences, clone3G8), and CD69-BV785 (6 μL/mL, Biolegend, Clone FN50). After three washes, cells were resuspended in 115 LPBS-1% paraformaldehyde. Flow cytometry data analysis was performed using FlowJo software (v10). Data is reported as the area under the curve (AUC) of % CD107a+ live NK cells (gates included singlets, lymphocytes, aqua blue-, CD56+ and/or CD16+, CD107a+), calculated as previously described42 at three concentrations of monoclonal antibody: 2, 10 and 50 μg/mL. All final data represent specific activity, determined by subtraction of non-specific activity observed in assays performed with mock-infected cells and in the absence of antibodies, and in the presence of a non-specific mono-clonal antibody, the anti-HIV-1 antibody VRC01.

Development of Mi03 Nanoparticles Conjugated with Epitope Scaffolds for Immunizations.

SpyCatcher003-Mi3 Nanoparticles. Plasmid encoding for the spycatcher003-mi3 particles was acquired from Addgene (Plasmid #159995) and transformed into E. coli BL-21 (RIPL) (Agilent Technolo-gies) cells. Five milliliters starter cultures were grown overnight at 37 C in Lysogeny Broth (LB) supplemented with 1% glucose and 50 μg/mL kanamycin. Cultures were diluted 1:100 in Terrific Broth (TB) media (RPI) supplemented with kanamycin and grown at 37° C. to an OD600 of ˜0.6. The temperature was subsequently lowered to 20 C, and the cultures were grown to OD600 of ˜0.8 and induced with isopropyl β-D-1-thiogalactopyranoside (IPTG) to a final concentration of 0.5 mM. The cultures were shaken for 16-18 hours at 250 rpm. Cell pellets were collected by centrifugation at 6,000×g and the pellets were stored overnight at −20° C. Pellets were resuspended in 40 mL of 25 mM Tris-HCl, 300 mM NaCl, pH=8.5 supplemented with a working concentration of 2 mM PMSF dissolved in isopropanol, 40 mg lysozyme (0.1 mg/mL) and 1 tablet of protease inhibitor. The lysates were incubated at room temperature for 30 minutes on a platform shaker and later sonicated for 13 minutes with 10 s on and 30 s off 50% duty-cycle. The sonicated lysates were centrifuged for 45 minutes at 18,000×g at 4 C. To precipitate the spycatcher003-mi3 nanoparticles, 170 mg/mL of ammonium sulfate was added to the supernatant solution and subsequently shaken at 4° C. for 1 hour. Precipitated particles were collected by centrifuging at 16,000×g for 30 minutes at 4° C. The supernatant was discarded, and the pellet was washed with endotoxin-free water to remove residual salty buffer. The nanoparticles were resolubilized in 8 mL of 25 mM Tris-HCl, 150 mM NaCl, pH=8.5. To remove insoluble aggregates, the solubilized nanoparticles were cen-trifuged at 18,000×g for 30 minutes. Nanoparticle expression and purity was confirmed by SDS-PAGE analysis, and quantified spectrophotochemically at 280 nm on a Nanodrop 2000 (ThermoFisher Scientific). The spycatcher003-mi3 nanoparticles were concentrated in a 10 kDa MW spin columns (ThermoFisher Scientific) and further purified by size exclusion chromatography in 25 mM Tris, 150 mM NaCl, pH=8.0, on an AKTA-Go FPLC (Cytiva) using a Superose 6 Increase 10/300GL column. Fractions containing the spycatcher003-mi3 nano-particles were pooled and concentrated as above. The nanoparticles were stored at −80° C.

Conjugation of epitope scaffolds to Spycatcher003-mi3 nano-particles. Epitope scaffolds were mixed with spycatcher003-mi3 nanoparticles in a 1.2:1 molar ratio. The complex was mixed thoroughly and incubated overnight on ice at 4 C. Conjugation was confirmed by SDS-PAGE. The conjugated nanoparticles were separated from excess monomeric epitope scaffolds by size-exclusion chroma-tography in 25 mM Tris, 150 mM NaCl, pH 8.0, on an AKTA-Go FPLC (Cytiva) using a Superose 6 Increase 10/300GL column. Fractions containing the epitope-scaffold-mi3 nanoparticles were pooled and concentrated as above. Endotoxins were removed by treating the nanoparticles with 2% triton X-100 as previously described43.Endo-toxin levels were tested by a chromogenic endotoxin quantification kit (ThermoFisher Scientific). The endotoxin-free nanoparticles were stored at −80 C.

Nanoparticles were analyzed by Negative Stain Electron Micro-scopy to confirm that they were well formed. A frozen aliquot was thawed in RT water bath, then placed on ice. The sample was then diluted to 400 μg/ml with 5 g/dl Glycerol in HBS (20 mM HEPES, 150 mM NaCl pH 7.4) buffer containing 8 mM glutaraldehyde. After 5 min incubation, glutaraldehyde was quenched by adding sufficient 1 M Tris stock, pH 7.4, to give 80 mM final Tris concentration and incubated for 5 min. Quenched sample was applied to a glow-discharged carbon-coated EM grid for 10-12 second, then blotted, and stained with 2 g/dL uranyl formate for 1 min, blotted and air-dried. Grids were examined on a Philips EM420 electron microscope oper-ating at 120 kV and nominal magnification of 49,000×, and 30 images were collected on a 76 Mpix CCD camera at 2.4 Å/pixel. Images were analyzed by 2D and 3D class averages using standard protocols with Relion 3.044.

### Mouse Immunization and Protection Studies

Immunization studies in BALB/c mice. BALB/c (#028) female mice were purchased from Charles River. All mouse studies were per-formed under an approved Duke University IACUC protocol. All animal rooms were kept on a 12/12 light cycle unless otherwise requested. Heat and humidity were maintained within the para-meters outlined in The Guide for the Care and Use of Laboratory Animals and animals were fed a standard rodent diet. Mice were housed in individually ventilated micro-isolator caging on corn cob bedding. The Toll-like receptor 4 agonist glucopyranosyl lipid adjuvant-stable emulsion (GLA-SE) was used as the adjuvant for the vaccine immunogens. Groups of mice (n=8) were vaccinated intra-muscularly with GLA-SE-adjuvanted Ex15-NP; GLA-SE-adjuvanted Ex_mosaic-NP; GLA-SE-adjuvanted mixture of equal parts Ex15-NP, Ex17_NP, Ex19_NP and Ex20_NP; or GLA-SE-adjuvanted Ex15-NP (prime), followed by Ex19_NP (boost 1) and Ex20_NP (boost 2). An adjuvant-only group (n=4) was included for comparison. Vaccine immunogens were administered at 5 μg and formulated with 5 gof adjuvant. Mice were immunized at week 0, week 4, and week 8. Blood samples were collected either 7 days prior to immunization (pre-bleed), or 7 days after each immunization. Seven days after the final dose mice were sacrificed for terminal analysis.

Live virus protection studies. Nineteen to twenty-one female k18-hACE2 mice purchased from Jackson Laboratory (B6.Cg-Tg(K18-ACE2) 2 Prlmn/J; JAX strain number #034860) were used for the WIV-1 viral challenge studies. The Toll-like receptor 4 agonist glucopyranosyl lipid adjuvant-stable emulsion (GLA-SE) was used as the adjuvant for the vaccine immunogens. Mouse vaccination studies were performed intramuscularly twice with mRNA ACLNP 307 2019NCOV WUHAN S-2P spike followed by two additional shots of either mRNA spike or GLA-SE-adjuvanted Ex_mosaic-NP. Protein immunogens were administered at 10 μg formulated with 5 μg of adjuvant. mRNA immunogens were administered at 20 μg. Mice were immunized at week 0, week 4, week 8, and week 12. Blood samples were collected either 7 days prior to immunization (pre-bleed), or 7 days after each immunization. Mice were then moved into the BSL3 and acclimated. For infection, vaccinated mice were anaesthetized with ketamine/xylazine and infected with 1×10⁴ PFU WIV1-CoV intranasally in a total volume of 50 ul. Infected mice were weighed daily.

Mouse studies were performed according to the recommendations for the care and use of animals by the Office of Laboratory Animal Welfare (OLAW), National Institutes of Health, and the Institutional Animal Care and Use Committee (IACUC) at the University of North Carolina (UNC permit no. A-3410-01). All infectious work was performed in Biosafety Level 3 laboratories (BSL-3) with approved standard operating procedures and safety conditions for SARS-CoV-2. All animal rooms were kept on a 12/12 light cycle unless otherwise requested. Temperature was maintained between 20-23.3° C. and humidity was kept at 30-70%. Animals were fed a standard rodent diet (PicoLab Select Rodent 50 IF/6 F-5V5R). Mice were housed in individually ventilated micro-isolator caging on corn cob bedding.

For virus titration by plaque assay, the caudal lobe of the right lung was homogenized in phosphate-buffered saline (PBS), and the homogenate was serial diluted and inoculated onto Vero E6 cells (American Type Culture Collection (ATCC), CRL1586), followed by agarose overlay. Plaques were visualized with an overlay of neutral red dye on day 2 after infection. At indicated time points, mice were euthanized and gross pathology (congestion score) of the lung tissue was assessed and scored on a scale from 0 (no lung congestion) to 4 (severe congestion affecting all lung lobes).

### Statistics and Reproducibility

The statistical analyses performed are described in Methods and figure legends. The Investigators were not blinded to the selection of the human specimens used for sera analysis. Samples were preferentially selected from participants who had seroconverted and had symptom onset more than 10 days prior to collection. No statistical method was used to predetermine the number of samples analyzed in the vaccinated, infected and vaccinated and infected groups. No statistical method was used to predetermine the number of animals in a group for the animal studies. No collected data were excluded from the analyses.

### Example 2

The data shown herein demonstrate production of stably-expressed epitope-scaffold immunogens designed to engage major classes of broadly neutralizing anti-S2 stem helix antibodies. These epitope scaffold immunogens can be used for a pancoronavirus vaccine.

FIG. 26 shows that scaffolds that display epitopes from the S2 stem helix can be computationally designed and physically produced at high yields.

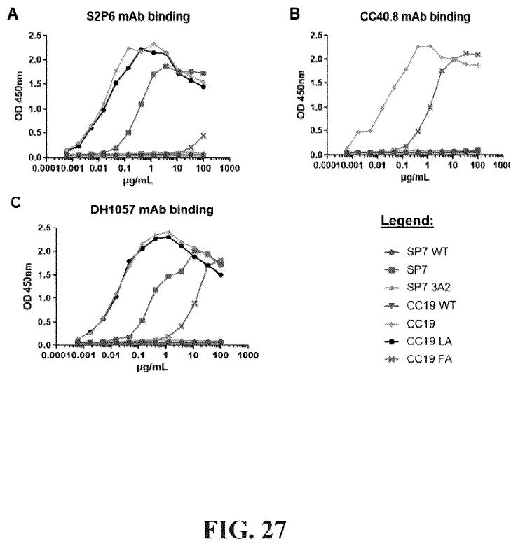

FIG. 27 shows that the scaffolds that display epitopes from the S2 stem helix can be bound by antibodies specific for the epitopes.

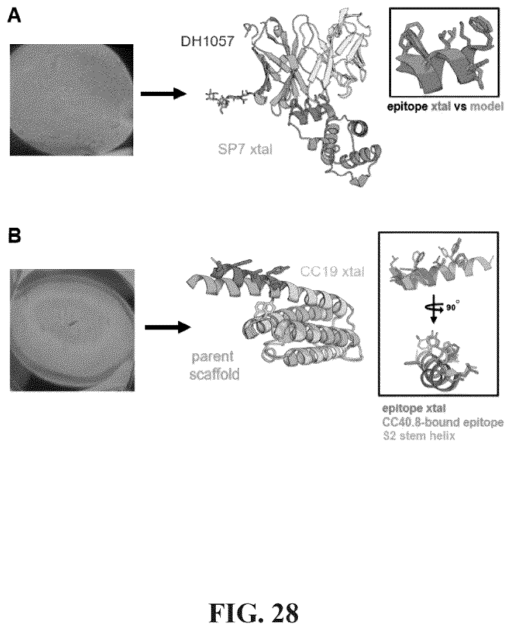

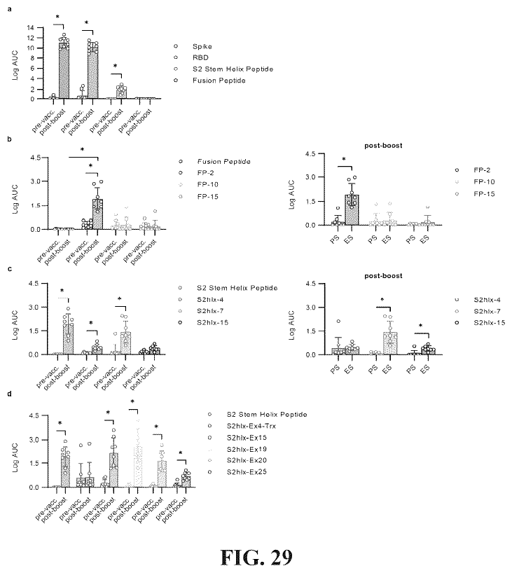

FIG. 28 shows in (A) a crystal structure of a scaffold that displays a grafted S2 stem helix epitope in complex with an antibody specific for the epitope. The grafted epitope from the crystal structure is compared with computational model. In (B) is shown a comparison of an S2 stem helix epitope presented to an antibody specific for the epitope by a scaffold and comparison of this to the conformation of the epitope in the native spike protein.

The data shown herein demonstrate that these immunogens (grated into the scaffolds) bind their target antibodies with high affinity and specificity, and structurally validated successful epitope presentation on the scaffold proteins in accordance with computational models

Additional crystallography studies with additional designs and antibodies are being done. Immunization studies are being done.

### Example 3

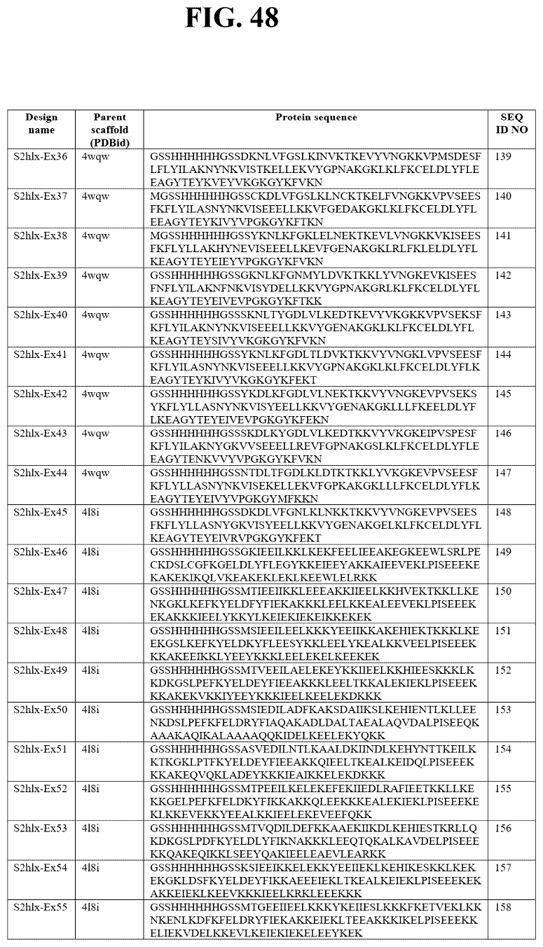

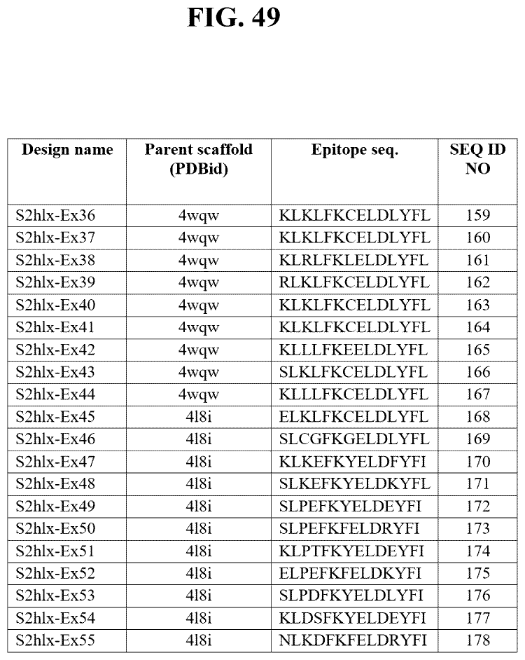

Amino acid sequences of example epitope scaffold molecules are included below, in Table 6 and in FIG. 48 (SEQ ID NOs: 139-158).

FP-1, (SEQ ID NO: 1):
MHHHHHHEVDQQILLQQLKSDYRQILLSYFTTDKALKEKIDKFAN
AVFCANIPVPEIIEIHMELIDEFSKQLRLEGRGDEDLMDYRLTL
EDILRSLIEDYLFAIFK
FP-2, (SEQ ID NO: 2):
MNIFEALRIDQGLRLKIYKNTEGYYTIGIGHLLTKSPSLNAAKSE
LDKAIGRNCNGVITKDEAEKLFNQDVRSAVEDILFNAKLSPVFD
SLDAVRRAALIIMVFQMGETGVAGFTNSLRMLQQKRWDEAAVNLA
KSRWYNQTPNRAKRVITTFRTGTWDAYKLEHHHHHH
FP-3, (SEQ ID NO: 3):
MDIPKITTFLMFNNQAERSVIEDTLFFEDSEIITMAKYGENGPGD
PGTVQHSIFTLNGQVFMAIDANSGTELPISLFVTVKDTIEMERL
ENGLKDEGAILMPKTNMPPYREFAWVQDKFGVSFQLALPELEHHH
HHH
FP-4, (SEQ ID NO: 4):
HMDRDRFMDEFFEQVEEIRGFIDKIAENVEEVKRKHSAILASPNP
DEKTKEELEELMSDIKKTANKVSSKLKSIEQSIEQEEGLNRSSAD
LRIRKTQHSTLSRKFRSVIEDFLFTQALYRERASGRILEHHHHHH
FP-5, (SEQ ID NO: 5):
MKSTEKKELSHFRLKLETYLNEHFPEMSGNNPFITARSDEALRSY
IEDVLFGFSHPEAESMASEVLYQGLHFSRYDTLVSVLEREFEQE
LPSPLPERLAPILLKNKAIQSVFAKYDLTDDFEASPEYEHLYTEL
TGTIVLLIESNHLPTILEHHHHHH
FP-6, (SEQ ID NO: 6):
MLAKILVIDDESTILQNIKFLLEIDGNEVLTASSSRSGIEDFLFN
ANSIDVVITDMKMPKSSGMDILRAIKKITPHMAVIILTGHGDLD
NAILAMKEGAFEYLRKPVTAQDLSIAINNAINRKKLLMENERLEH
HHHHH
FP-7, (SEQ ID NO: 7):
MARRILVVAAEAPIARSVIEDLLFNGFQPVAAEDYDSAVNSLNEP
WPDLILLNWMLPGGSGIQFIKHLKRESMTRDIPVVMLTARGEEE
DRVRGLETGADDYITKPFSPKELVARIKAVMRRILEHHHHHH
FP-8, (SEQ ID NO: 8):
EGDDPTNLESALIGKPVPKFRLESLDNPGQFYQADVLTQGKPVLL
NVWATWCPTCRAEHQYLNQLSAQGIRVVGMNYKDDRRSAIEWLL
FLGNPYALSLFDGDGMLGLDLGVEGAAATFLIDGNGIIRYRHAGD
LNPRVWEEEIKPLWEKYSKEAAQLEHHHHHH
FP-9, (SEQ ID NO: 9):
MRPKLYKVMLLNDDYTPREFVTVVLKAVFRMSEDTGRRVMMTAHR
FGSAVVGVFTRDVADTKARSAIEDGLFAGFPLTFTTEPEELEHH
HHHH
FP-10, (SEQ ID NO: 10):
MSLLRSYAEDFLFTLAQAKASLAEAPSQPLSQRNTTLKHVEQQQD
ELFDLLDQMDVEVNNSIGDASERATFKASLREWKKTIQSDIKRP
LQSLVDSGDLEHHHHHH
FP-11, (SEQ ID NO: 11):
MADLRSNIEDLLFNLAQIEKADNAAQVKDALTKMRAAALDAQKAT
PPKLEDKSPDSPEMKDFRHGFDILVGQIDDALKLANEGKVKEAQ
AAAEQLKTTLKAYIAKYLLEHHHHHH
FP-12, (SEQ ID NO: 12):
MNLDDTLDVLNDLLQTSKDGEAGFHACAEDLRDPQLKAAMLEASR
DAAAAADELERIVLELGGKPKDSTSFAGDLHRRWVDLKSLVTGK
DEEAVLNECERGLAVAAARYAAALEKSLPAEIHQVIERQAQGVRS
HIEDVLFLRASRALEHHHHHH
FP-13, (SEQ ID NO: 13):
MSTVAVTDATFEADVLKSSKPVLVDFWAEWSGPSKQIAPALEQLS
EELADVVTIAKVNIEDSPTTPSRYGVRGIPTMMLFRDGQMTSMK
VGAMPKRSILEWLLFAGAQAALLEHHHHHH
FP-14, (SEQ ID NO: 14):
MQEAANRSPPYAPNPYPVDEIIGGDSVQSIQRRLLGTNWNPSAHD
MQMSRIQAEDLFLAKVSIIRKMAGLHPSGDWMGWGARALDNPRT
ATGEEDLARLRSMLEDLLFRNEQSATFWRLVERVRLRADLEHHHH
HH
FP-15, (SEQ ID NO: 15):
MADLRSNWEDLLFNLAQIEKADNAAQVKDALTKMRAAALDAQKAT
PPKLEDKSPDSPEMKDFRHGFDILVGQIDDALKLANEGKVKEAQ
AAAEQLKTTRKAYIAKYLLEHHHHHH
S2hlx-1, (SEQ ID NO: 16):
MHHHHHHGWSENLYFQGSTVRIEIRFTNMARFKVELDLYFFKHSL
KELEKRTGSEIRIEIEERDGEVRVEVEIRNSHEEEVRQIIEEIE
RWVRKMGGELRVEK
S2hlx-2, (SEQ ID NO: 17):
HMQSNAMTFAHEVVKSNVKNVKDRKGKEKQVLFNGLTTSKLRNLM
EQVNRLYTIAFNSNEDQLAFKFELDLYFLKHSFYYEAGREKSVD
EFLKKTLMFPIIDRVIKKESKKFFLDYCKYFEALVAYAKYYQKED
LEHHHHHH
S2hlx-3, (SEQ ID NO: 18):
MHHHHHHLLGFYKQYKALSEYIDKKYKLSLNDLAVLDLTMKHATD
EKVLMQSFLKTAMDELDLSRTKLLVSIRRLIEKERLSKVRSSKD
ERKIYIYLNFKDELDFYFLFEDVE
S2hlx-4, (SEQ ID NO: 19):
MHHHHHHADLEDNWETLNDNLKVIANADNAAQVKDALTKMRAAAL
DA
QKATPPKLEDKSPDSPEMKDFRHGFDILVGQIDDALKAANEG
GVFKAELDAYFLHYTRNAYIQKYL
S2hlx-5, (SEQ ID NO: 20):
MHHHHHHENLYFQGIPRITIHAFCARPETAALIEKAAADRRMSRA
ATIVRDGGLEAAVDYYQNQPTPSLVMVETLDGAQRLLHLLDSLA
QVCDPGTKVVVVGQTNDFKLELDLYFRGVSAYLTQPLGPLQVIRA
VGALYADPA
S2hlx-6, (SEQ ID NO: 21):
MVTVEEEVYEFLKKKAKEEGTSVPAVIRKILKEYFGIEDRTRDYK
RQDLEGSYIIVNGKKYYRINASLEFKNELDVYFELKSRGTTLNR
FLKEMIMITVLEHHHHHH
S2hlx-7, (SEQ ID NO: 22):
MTSTFDRVATIIAETADIPRETITPESHAIDDLGIDELDFLDIAF
AIDKAFGISLPLFKWELDVYFGSATTEQYFVLKNLAARIDELVA
AKGALEHHHHHH
S2hlx-8, (SEQ ID NO: 23):
MRSKRVLVVEDNPDDIALIRRVLDRKDIHAQLEFVDNGAKALYQV
QQAKYDLIILDIGLPIANGFEVMSAVRKPGANQHTPIVILTDNA
SFKRELDAYFAGASEAVDKSSNNVTDFYGRIYAIFSYWLTVNHAQ
LEHHHHHH
S2hlx-9, (SEQ ID NO: 24):
MHHHHHHNTDELKQKYGRVYEIRIEGAEYENGEEAEFVFYFTRPS
FKDELDFYFELHSKPDMAMKNLTFSAIVPEQEEELRQAAEEFPG
LTFNTASRLMEIV
S2hlx-10, (SEQ ID NO: 25):
MEFFDILEDVKEDHFEKLLEEAVEEVIDSGNELVRSPTPSNLKRY
KNAIKEFLKLIEKKIYKLAGSFDMFKGELDLYFVVHAVNEKLMDL
TEKIMKNEWQTINLAARIEEINGLILNLYRLEHHHHHH
S2hlx-11, (SEQ ID NO: 26):
MHHHHHHGAESTAERSARFERDALAFAFKMELDAYFMTRNPADAE
DLVQETYAKAYASFHQFREGTNLKAWLSEILTNTFSNSYRKKQR
S2hlx-12, (SEQ ID NO: 27):
KDTEDEALMRTLLDHFDQYIKISKKISAETYAAVTDIEEPGRMAD
IVASHLPLDFKDELDIYFTADVKDRLNKVIDFINNEKEVLEIEK
LEHHHHHH
S2hlx-13, (SEQ ID NO: 28):
HMMTVAEDKTFQYIRQHHSNFSRIHVLEILPYLSALTFKDELDLY
FTYHRWGNQDTLLELFTSLRSRNGWVHSLIGALRAAELSGLADE
VARIYHSLEHHHHHH
S2hlx-14, (SEQ ID NO: 29):
MSHKEPATLIKAIDGDTVKLMYKGQPMTFRLLLVDTPEVKHPKKG
VEKYGPEASAFTKKMVENAKKIEVEFDKGQRTDQAGEGLAYIYA
DGKMVNAALVRQGLAKVAYVYKPNNAASFKLELDKYFAKHDKLNI
WSLEHHHHHH
S2hlx-15, (SEQ ID NO: 30):
MGGYAPFKVELDLYFMYPTAAGIAWSQDKAYYVADFVMNGFDTRV
WFTPDAEWVMKQTDWETLDEVPAAVFNAFAASEFSDGVVQNVTW
VQFPEWQPIVAIQVGKPNMQMKYQILFTPKGEVLRQQNITNAYNT
LGASTFLLEHHHHHH
S2hlx-Ex1, (SEQ ID NO: 31):
MHHHHHHGGSLKITILCVGKGKALDFFKLELDFYFLGPYTKFDLI
EVPDEKAPENMSDKEIEQVKEKEGQRILAKIKPOSTVITLEIQG
KMLSSEGLAQELNQRMTQGQSDFVFVIGGSNGLHKDVLQRSNYAL
SFSKMTFPHIMMRVVLAEQVYRAFKIMRGSAS
S2hlx-Ex2, (SEQ ID NO: 32):
MNLDSFKEELDDYFNESYVAIMDSLFDKLLSKYEVKAPVPSPCFR
NICKQMTKLHEKLFDVLPEEQTQMLFLRINASYKLHLKKQLSHL
NVINDGGPQNGLVTADVAFYTGNLQALKGLKDLDLNMAEIWELEH
HHHHH
S2hlx-Ex3, (SEQ ID NO: 33):
MHHHHHHGGSGSMSDIRKDLEERFDKIVEALKNSVDAAKASFRDA
QFALDGFKLELDVYFIGSSFKEVRNYASEALSKINDLPITNDDK
KLASNDVLKLVAEVWKKLEAIMADVYAWNTH
S2hlx-Ex4, (SEQ ID NO: 34):
MHHHHHHGGSAASAASSEHFEKLHEIFRGLLENLOGGPERLLGSA
GSLASFKIELDIYFFQEANETLAEMEEELRYAPLTFRNPMMSKL
RNYRKDLAKLHREVR
S2hlx-Ex5, (SEQ ID NO: 35):
TFVEKYEKQIKHFGMLTRWDDSQKYLSDNVHLVCEETANYLVIWC
IDLEVEEKCALMSIVAHQTIVMQFILELAKSLKVDPRACFRQFF
TKIKSDALDGFKLELDLYFFIEEVRGRAKLRIELEHHHHHH
S2hlx-Ex6, (SEQ ID NO: 36):
EQLSFDELTLINLSKVVTVNGHEVPMSIKAFEFLWYLASRENEVI
SESELAEKVIGDSSLSLFKYELDLYFLEKESFTTYSVTTVEGLG
YKFERSLEHHHHHH
S2hlx-Ex7, (SEQ ID NO: 37):
MHHHHHHGGSEDPFFVIKGEIQKAVNSAQGALQRWTEALQGPSSA
GLALFKVELDIYFLSEIEALLEDLDETISIVEANPRKFNLDATE
LSIRKAFITSTAQIVRDITDQAAAS
S2hlx-Ex8, (SEQ ID NO: 38):
MHHHHHHGGSSLLISYESDFKTTLEQAKASLAEAPSQPLSLASFK
YELDIYFGDELFDLLDQMDVEVNNSIGDASERATYKAKLREWKK
TIQSDIKRPLQSLVDSGD
S2hlx-Ex9, (SEQ ID NO: 39):
MHHHHHHGGSGAAAAALLARAAAARAELAALLAELAAAPARLAAA
TGALASFKIELDIYFIAAARATLAELEALLAELPPELAAPLRAE
LAARRAELAALAAEVE
S2hlx-Ex10, (SEQ ID NO: 40):
MHHHHHHGGSMEKEKELLEKLEKLKEELKKLMEELKKAPAELAAA
SGELASFKIELDIYFIKEAEETLKKIEELLKSLPEEIKEPLLKE
LEKKKKELEELKKQIE
S2hlx-Ex11, (SEQ ID NO: 41):
MHHHHHHGGSGEEEEELLEELEELKEELERKMEELKKAPKRLEEA
EGELASFKIELDIYFIEEARRLLEEIEELLEELPEEVREPLLEE
LEERKKELEELEKKIE
S2hlx-Ex12, (SEQ ID NO: 42):
MHHHHHHGGSSAAAAARAARLAALLARLDALLAEIRAAPAALAEL
ASGLASFKYELDIYFAEEAEATLAEIRALLAGAPPAVAAPLRAE
LAAREAELAALRARLE
S2hlx-Ex13, (SEQ ID NO: 43):
MHHHHHHGGSAKAAEEFLKELKKLKEELKKLLKEIKKAPEEKKKL
KSELASFKYELDIYFGEEAEAKLKEIKKMLETAPEEIREPELEE
LKEIEKELKKLREKLE
S2hlx-Ex14, (SEQ ID NO: 44):
MHHHHHHGGSSMAMEEFLKELEKLKEEMKKLLEEMKKAPKKKEEI
KSELASFKYELDIYFGKELKKTLEKIKELLKTAPKELAEPLLKE
LEKIEKEIEELKKKLE
S2hlx-Ex15, (SEQ ID NO: 45):
NLDSFKEELDDYFKEKIVKEFKKFCEETLSKYKVERPTPSKEIKE
ICAKIREIHEEKVNEWPEEEVRELMLKMWEEFKKELNKRLKELG
VTNDGGEKYKIVKEDLDYLESTIRSLPGLENLDLNTESIWDLEHH
HHHH
S2hlx-Ex16, (SEQ ID NO: 46):
NLDSFKEELDDYFKKKIVEEFDKFCEEKLSKYKVETPTPSPVFKE
IAAKFKALHDELVGKWPEEDLRELVLELNKEFKKHLNKRLKELG
VTNDGGEKYKIVKADLAYLESTIRSLPGLSDLDLNWEEVFKLEHH
HHHH
S2hlx-Ex17, (SEQ ID NO: 47):
NLDSFKEELDDYFKEEIVKEFDEFCKKVLSKYKVEKPTPSKEFKE
ICSKIKEIHEKLKNVLPEEELKELMKKMNEVLKKHLNERLKELG
VTDDGGEKYKIVKEDIDYIVDTIRSLEGLSDLDLNWEEIWKLEHH
HHHH
S2hlx-Ex18, (SEQ ID NO: 48):
NLDSFKEELDDYFKEKIVEEFKKKCEELISKYVVKEPVPSPEIKE
LCKYIKELHEKIYNKYPEEFVQEIFKKIWDIFIEELSKRLKELG
VTNDGGKKYKLVKKDIDYLVSVIKSLPGLSNLDLNWERIWNLEHH
HHHH
S2hlx-Ex19, (SEQ ID NO: 49):
NLDSFKEELDDYFKEKIVKEFEKLCKELISKYEVKKPTPSPEIKK
ICEYLKKKHEELKDKYPEEFVKEIFKKMWEVFKKELSKQLKKLG
VTNDGGEKYKIVKEDLNYLVDVIKSLEGLSDLDLNWEEIWNLEHH
HHHH
S2hlx-Ex20, (SEQ ID NO: 50):
NLDSFKEELDDYFKEKIVAEFKKLCEELLSKYEVKSPVPSPEFKK
IAEYLKKLHEELVDKWPLEYVRKLFLKIMKIFKEELKKQLDKLG
VTNDGGEKYKQVKADVDYIISVVKSLPGLSDLDLNWEEIWNLEHH
HHHH
S2hlx-Ex21, (SEQ ID NO: 51):
MHHHHHHGGSMKALEEFLKKLEELKKKLKALLEKIEKAPEEKEKE
KSSLGKFKYELDLYFLKEAEKVLKEIKEMLKTAPEEIREPELEE
LKKIEEKIKELKKKLE
S2hlx-Ex22, (SEQ ID NO: 52):
MHHHHHHGGSAAAAEEFLAKLEELKKELKELLERIKKAPKELEED
KSELEKFKYELDLYFMEKAEETLEKIRKMLETAPEELREPLLEE
LKKIEKELKELKKKLN
S2hlx-Ex23, (SEQ ID NO: 53):
MHHHHHHGGSAEEEEAFLRELAALREERDALLERIKKAPEEKKED
KSELEKFKYELDLYFMEELEATLKRIKAMLATAPEELAKPLREE
LEKVEKEIKELKKELE
S2hlx-Ex24, (SEQ ID NO: 54):
MHHHHHHGGSSSHMKEFLEELEKLLKELEKLMKRIEKAPKEKEEE
KSELEKFKYELDLYFMEEAKKTLKKIKELLATAPKEISEPLLKE
LKKIEEKLKKLEEELK
S2hlx-Ex25, (SEQ ID NO: 55):
MHHHHHHGGSSALTEEYIKRLEELKRELKEKLERIKKAPEEKEKE
KSKLEEFKYELDIYFMKEAEKLLEEIKELLKTAPPELAEPLLKE
LEKIEKELEELKKKLY
S2hlx-Ex26, (SEQ ID NO: 56):
MHHHHHHGGSAAAAAARAAELAALRARLDALLERIEKAPEEKKKD
DSELGKFKYELDLYFMEEAEATLAQIRALLATAPAELAAPALAE
LDAIEKKLAELRKKLK
S2hlx-Ex27, (SEQ ID NO: 57):
MHHHHHHGGSMADLEEFLKKLEELKEELNKLLERIKKAPEEKKKT
KDELEKFKYELDLYFIEEANKVLKEIKKLLEKAPQEISEPMLEE
LKKIEKEIEELKKKLK
S2hlx-Ex28, (SEQ ID NO: 58):
MHHHHHHGGSSKLEELKKKFEETFKKAVETLKEAPKLPKELREFK
IELDKYFAEKLKELLEKMKEEVEKEVKDKKKKEELLKEIEEKEK
EVEEKIEKPLKKLIEESK
S2hlx-Ex29, (SEQ ID NO: 59):
MHHHHHHGGSSRLEELKKEFEETFEKAVKILKETPTLPPELAKFK
IELDKYFAKKLEDLLKEMEEEVEEEVEDEEERERLLEEIKERRK
EVEEKIVKPLKELIKKSK
S2hlx-Ex30, (SEQ ID NO: 60):
MHHHHHHGGSSRLEQLRKEYDETFQKAKETLAEAPNLPPELAKFK
IELDKYFAQKLQELLKKMEEAVAEEVADEKERAALLEELAKRRQ
EVEEKIVKPLKELIEKSK
S2hlx-Ex31(SEQ ID NO: 61):
MGSSHHHHHHGSSKDLQFGDLEIYEDKNSIKVKGKEIPMSEESFK
KIYLLAKNPNVVLSEEELLKEVTGDKSLELFKFELDIYFLKKAG
ITEYEIEKVEGKGYKFKKK
S2hlx-Ex32, (SEQ ID NO: 62):
MGSSHHHHHHGSSPDLVYGGLEINKETKELYVNGKKIPMSEKSFK
VLYVLAKRPNEVVSYEELREEVWGCCSLELFKFELDLYFLKEVG
FDEIAVEYVPGKGYVFTKR
S2hlx-Ex33, (SEQ ID NO: 63):
MGSSHHHHHHGSSIDLKFGDLELEIERKKLFVNGKEIPMSEKSFL
RIYLLAKRPNEVISLEELLKEVCGCCSLELFKFELDIYFLKEAG
YTEYAIEYVPGKGFMFRKT
S2hlx-Ex34, (SEQ ID NO: 64):
MGSSHHHHHHGSSVDLKYGDLELKEKTKTLLVKGKEIPCSDESFK
FLWLLAKNYNKVISLEELLEKVLGSRSLELFKYELDLYFLKEAG
YTEYAIVKVPGKGYKFEKV
S2hlx-Ex35, (SEQ ID NO: 65):
MGSSHHHHHHGSSVDLQYGSLTLVEETKKIYVNGKEIPCSDKSFQ
LIYLLAKRPNEVISKEELAEEVTGDKSLELFKWELDVYFLKKAGY
TEYAIIEVPGKGYKFAKV

TABLE 6
Design strategy: BB grafting + MPNN + AF.

| | Parent | | SEQ |
|---|---|---|---|
| Design | scaffold | | ID |
| name | (PDBid) | Protein sequence | NO |
| S2hlx- | 4wqw | GSSHHHHHHGSSDKNLVFGSLKINV | 139 |
| Ex36 | | KTKEVYVNGKKVPMSDESFLFLYIL | |
| | | AKNYNKVISTKELLEKVYGPNAKGK | |
| | | LKLFKCELDLYFLEEAGYTEYKVEY | |
| | | VKGKGYKFVKN | |
| S2hlx- | 4wqw | MGSSHHHHHHGSSCKDLVFGSLKLN | 140 |
| Ex37 | | CKTKELFVNGKKVPVSEESFKFLYI | |
| | | LASNYNKVISEEELLKKVFGEDAKG | |
| | | KLKLFKCELDLYFLEEAGYTEYKIV | |
| | | YVPGKGYKFTKN | |
| S2hlx- | 4wqw | MGSSHHHHHHGSSYKNLKFGKLELN | 141 |
| Ex38 | | EKTKEVLVNGKKVKISEESFKFLYL | |
| | | LAKHYNEVISEEELLKEVFGENAKG | |
| | | KLRLFKLELDLYFLKEAGYTEYEIE | |
| | | YVPGKGYKFVKN | |
| S2hlx- | 4wqw | GSSHHHHHHGSSGKNLKFGNMYLDV | 142 |
| Ex39 | | KTKKLYVNGKEVKISEESFNFLYIL | |
| | | AKNFNKVISYDELLKKVYGPNAKGR | |
| | | LKLFKCELDLYFLKEAGYTEYEIVE | |
| | | VPGKGYKFTKK | |
| S2hlx- | 4wqw | GSSHHHHHHGSSSKNLTYGDLVLKE | 143 |
| Ex40 | | DTKEVYVKGKKVPVSEKSFKFLYIL | |
| | | AKNYNKVISEEELLKKVYGENAKGK | |
| | | LKLFKCELDLYFLKEAGYTEYSIVY | |
| | | VKGKGYKFVKN | |
| S2hlx- | 4wqw | GSSHHHHHHGSSYKNLKFGDLTLDV | 144 |
| Ex41 | | KTKKVYVNGKLVPVSEESFKFLYIL | |
| | | ASNYNKVISEEELLKKVYGPNAKGK | |
| | | LKLFKCELDLYFLKEAGYTEYKIVY | |
| | | VKGKGYKFEKT | |
| S2hlx- | 4wqw | GSSHHHHHHGSSYKDLKFGDLVLNE | 145 |
| Ex42 | | KTKKVYVNGKEVPVSEKSYKFLYLL | |
| | | ASNYNKVISYEELLKKVYGENAKGK | |
| | | LLLFKEELDLYFLKEAGYTEYEIVE | |
| | | VPGKGYKFEKN | |
| S2hlx- | 4wqw | GSSHHHHHHGSSSKDLKYGDLVLKE | 146 |
| Ex43 | | DTKKVYVKGKEIPVSPESFKFLYIL | |
| | | AKNYGKVVSEEELLREVFGPNAKGS | |
| | | LKLFKCELDLYFLEEAGYTENKVVY | |
| | | VPGKGYKFVKN | |
| S2hlx- | 4wqw | GSSHHHHHHGSSNTDLTFGDLKLDT | 147 |
| Ex44 | | KTKKLYVKGKEVPVSEESFKFLYLL | |
| | | ASNYNKVISEKELLEKVFGPKAKGK | |
| | | LLLFKCELDLYFLKEAGYTEYEIVY | |
| | | VPGKGYMFKKN | |
| S2hlx- | 418i | GSSHHHHHHGSSDKDLVFGNLKLNK | 148 |
| Ex45 | | KTKKVYVNGKEVPVSEESFKFLYLL | |
| | | ASNYGKVISYEELLKKVYGENAKGE | |
| | | LKLFKCELDLYFLKEAGYTEYEIVR | |
| | | VPGKGYKFEKT | |
| S2hlx- | 418i | GSSHHHHHHGSSGKIEEILKKLKEK | 149 |
| Ex46 | | FEELIEEAKEGKEEWLSRLPECKDS | |
| | | LCGFKGELDLYFLEGYKKEIEEYAK | |
| | | KAIEEVEKLPISEEEKEKAKEKIKQ | |
| | | LVKEAKEKLEKLKEEWLELRKK | |
| S2hlx- | 418i | GSSHHHHHHGSSMTIEEIIKKLEEE | 150 |
| Ex47 | | AKKIIEELKKHVEKTKKLLKENKGK | |
| | | LKEFKYELDFYFIEKAKKKLEELKK | |
| | | EALEEVEKLPISEEEKEKAKKKIEE | |
| | | LYKKYLKEIEKIEKEIKKEKEK | |
| S2hlx- | 418i | GSSHHHHHHGSSMSIEEILEELKKK | 151 |
| Ex48 | | YEEIIKKAKEHIEKTKKKLKEEKGS | |
| | | LKEFKYELDKYFLEESYKKLEELYK | |
| | | EALKKVEELPISEEEKKKAKEEIKK | |
| | | LYEEYKKKLEELEKELKEEKEK | |
| S2hlx- | 418i | GSSHHHHHHGSSMTVEEILAELEKE | 152 |
| Ex49 | | YKKIIEELKKHIEESKKKLKKDKGS | |
| | | LPEFKYELDEYFIEEAKKKLEELTK | |
| | | KALEKIEKLPISEEEKKKAKEKVKK | |
| | | IYEEYKKKIEELKEELEKDKKK | |
| S2hlx- | 418i | GSSHHHHHHGSSMSIEDILADFKAK | 153 |
| Ex50 | | SDAIIKSLKEHIENTLKLLEENKDS | |
| | | LPEFKFELDRYFIAQAKADLDALTA | |
| | | EALAQVDALPISEEQKAAAKAQIKA | |
| | | LAAAAQQKIDELKEELEKYQKK | |
| S2hlx- | 418i | GSSHHHHHHGSSASVEDILNTLKAA | 154 |
| Ex51 | | LDKIINDLKEHYNTTKEILKKTKGK | |
| | | LPTFKYELDEYFIEEAKKQIEELTK | |
| | | EALKEIDQLPISEEEKKKAKEQVQK | |
| | | LADEYKKKIEAIKKELEKDKKK | |
| S2hlx- | 418i | GSSHHHHHHGSSMTPEEILKELEKE | 155 |
| Ex52 | | FEKIIEDLRAFIEETKKLLKEKKGE | |
| | | LPEFKFELDKYFIKKAKKQLEEKKK | |
| | | EALEKIEKLPISEEEKEKLKKEVEK | |
| | | KYEEALKKIEELEKEVEEFQKK | |
| S2hlx- | 418i | GSSHHHHHHGSSMTVQDILDEFKKA | 156 |
| Ex53 | | AEKIIKDLKEHIESTKRLLQKDKGS | |
| | | LPDFKYELDLYFIKNAKKKLEEQT | |
| | | QKALKAVDELPISEEEKKQAKEQIK | |
| | | KLSEEYQAKIEELEAEVLEARKK | |
| S2hlx- | 418i | GSSHHHHHHGSSKSIEEIKKELEKK | 157 |
| Ex54 | | YEEIIEKLKEHIKESKKLKEKEKGK | |
| | | LDSFKYELDEYFIKKAEEEIEKLTK | |
| | | EALKEIEKLPISEEEKEKAKKEIEK | |
| | | LKEEVKKKIEELKRKLEEEKKK | |
| S2hlx- | 418i | GSSHHHHHHGSSMTGEEIIEELKKK | 158 |
| Ex55 | | YKEIIESLKKKFKETVEKLKKNKEN | |
| | | LKDFKFELDRYFIEKAKKEIEKLTE | |
| | | EAKKKIKELPISEEEKKELIEKVDE | |
| | | LKKEVLKEIEKIEKELEEYKEK | |

### Example 4

Isolation of Antibodies with Broad Coronavirus Reactivity from Humans Pre-Exposed to SARS-CoV-2 Using Epitope Scaffolds

The ability of the engineered epitope scaffolds described herein to interact with B cells that give rise to antibodies with broad activity against the spike peptide or the stem helix from people with preexisting SARS-CoV-2 immunity was characterized. The potential of the scaffolds described herein to boost such B cell responses by vaccination was evaluated.

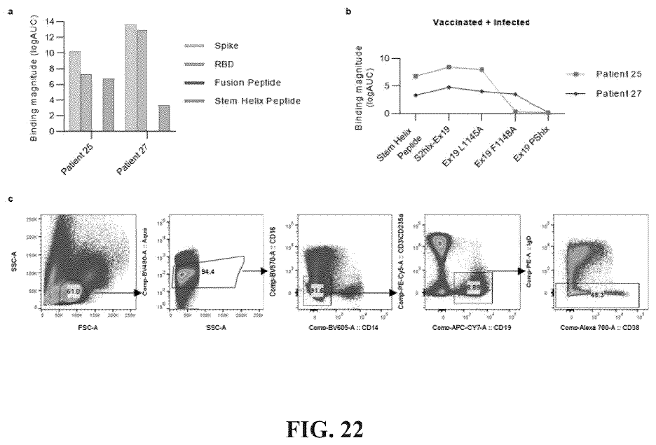

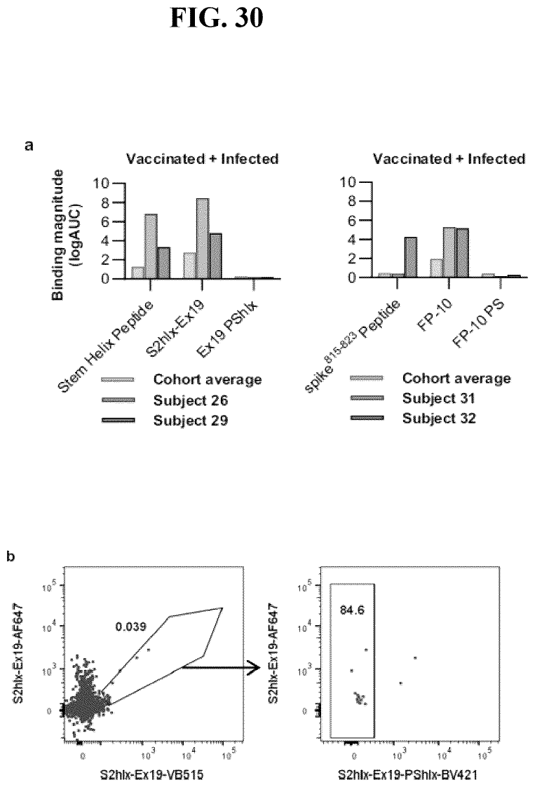

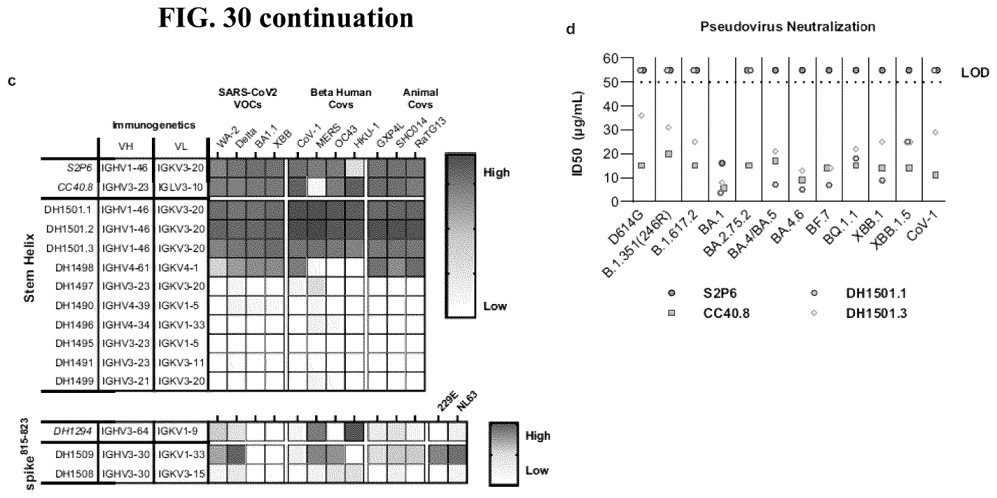

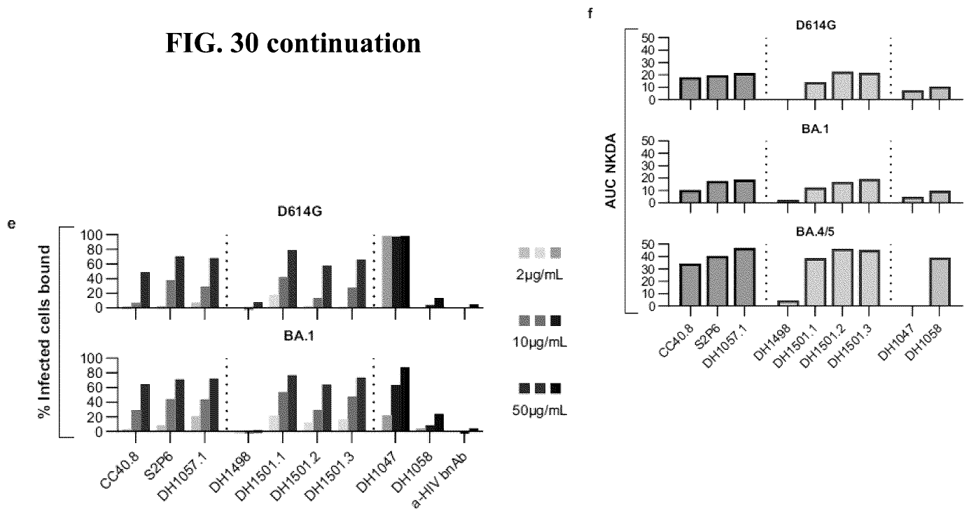

Stem helix reactive BCRs were isolated from IgD-B cells, a pool that included both memory B cells and plasma blasts, from the two subjects with the highest sera titers against S2hlx-Ex19, using positive selection for S2hlx-Ex19 and negative selection for the parent scaffold S2hlx-Ex19-PS that lacks the epitope (FIG. 30a and FIG. 30b). Stem helix epitope-specific memory B cells were isolated at a frequency of ˜1:3000 and ˜1:2100 respectively from the two samples (FIG. 30b and FIG. 22).

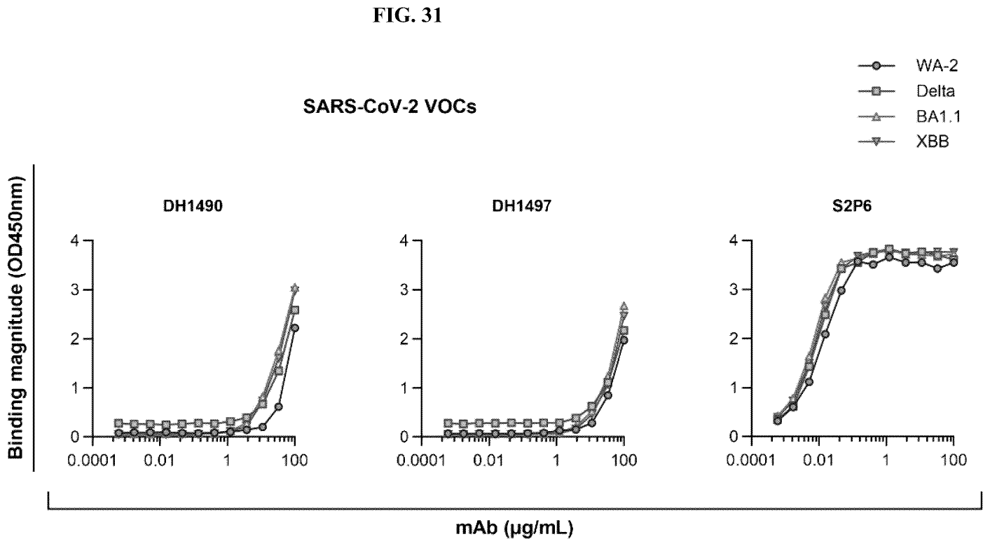

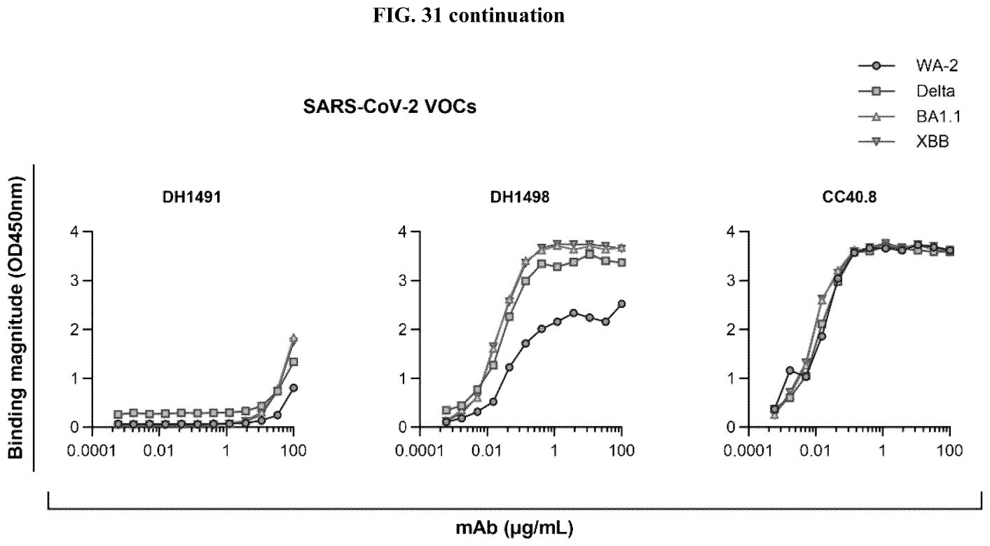

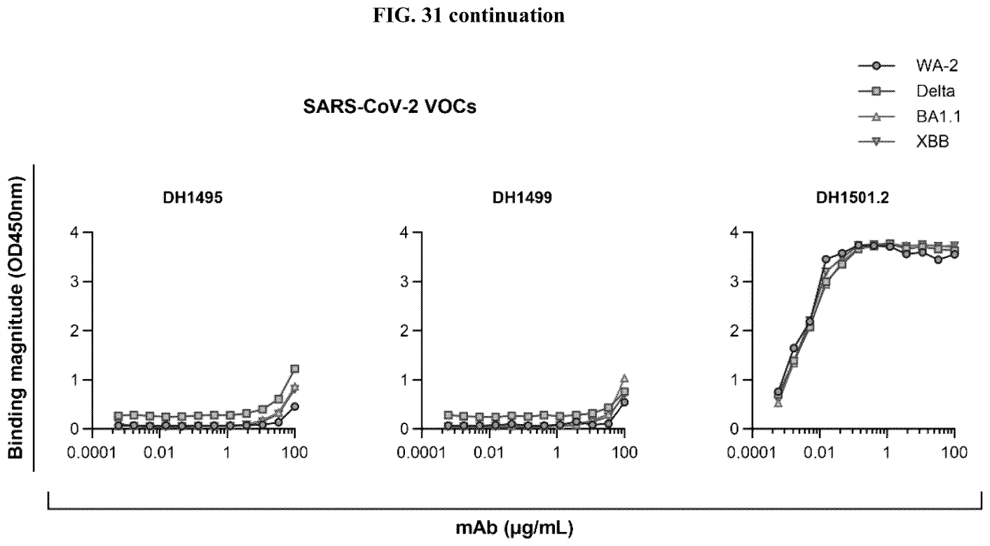

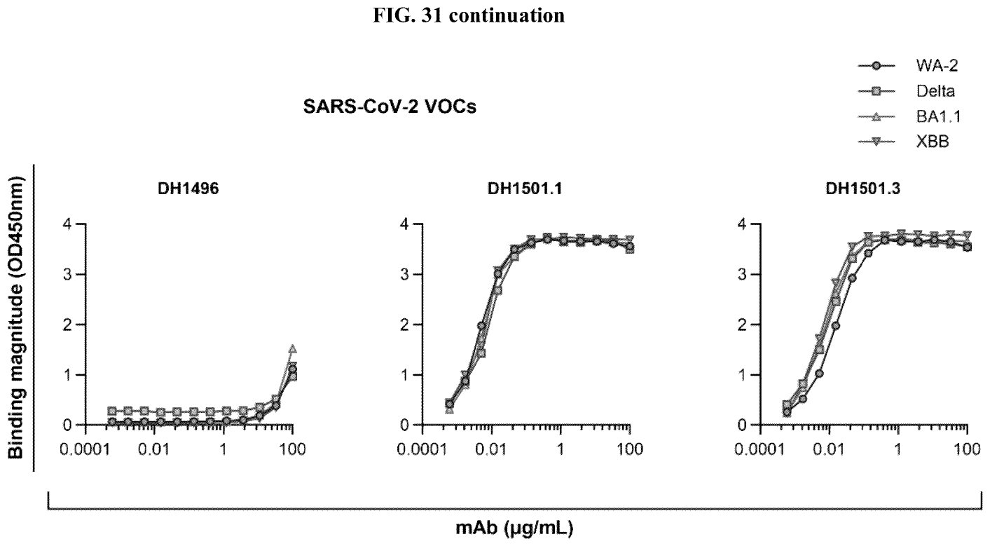

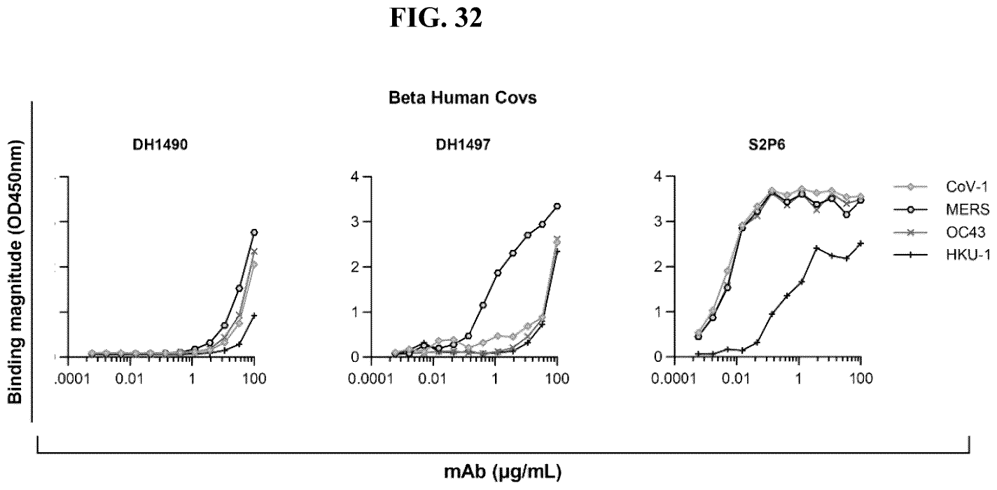

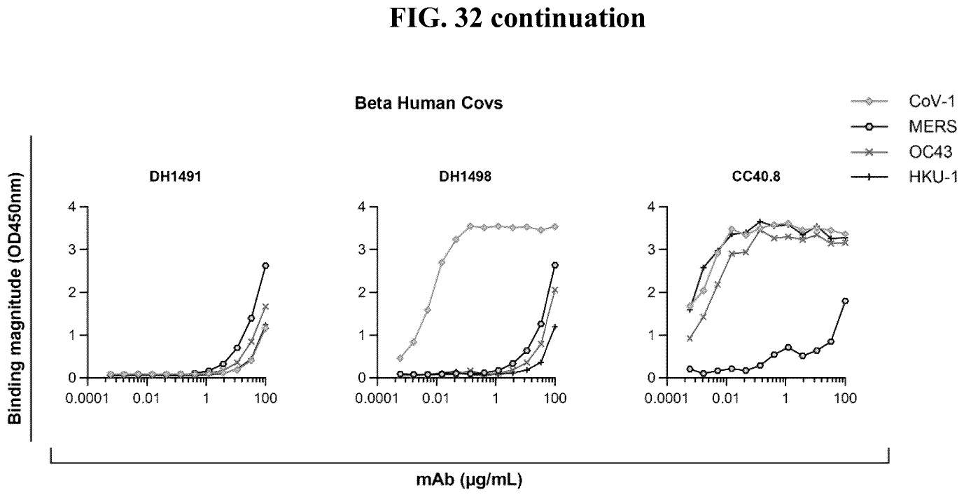

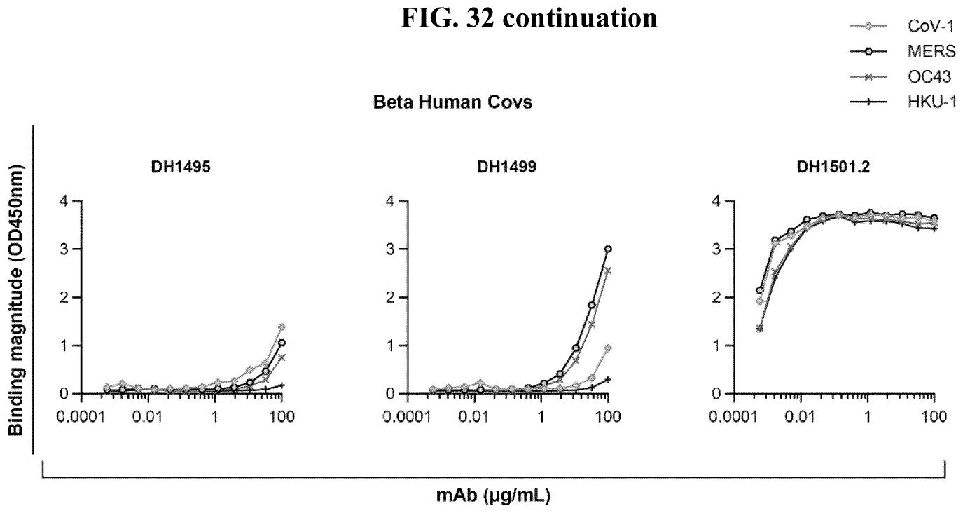

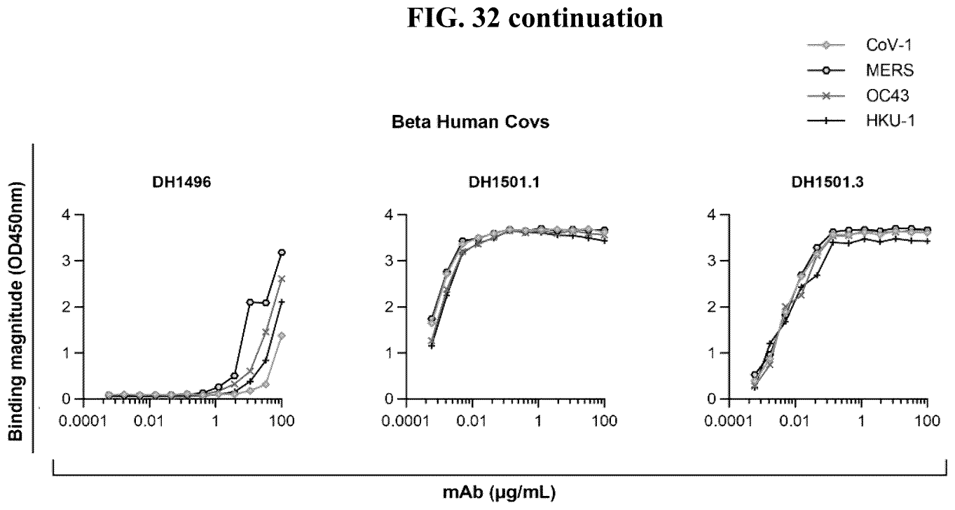

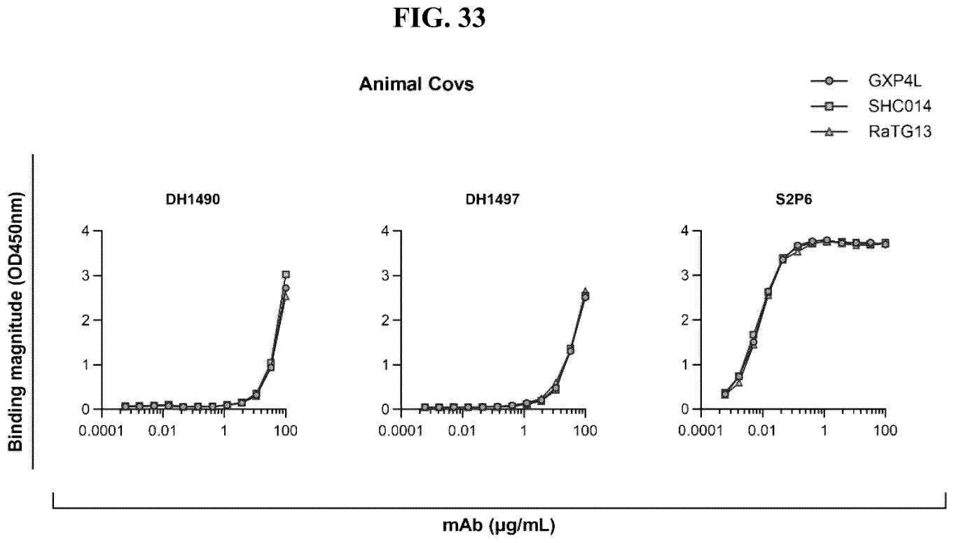

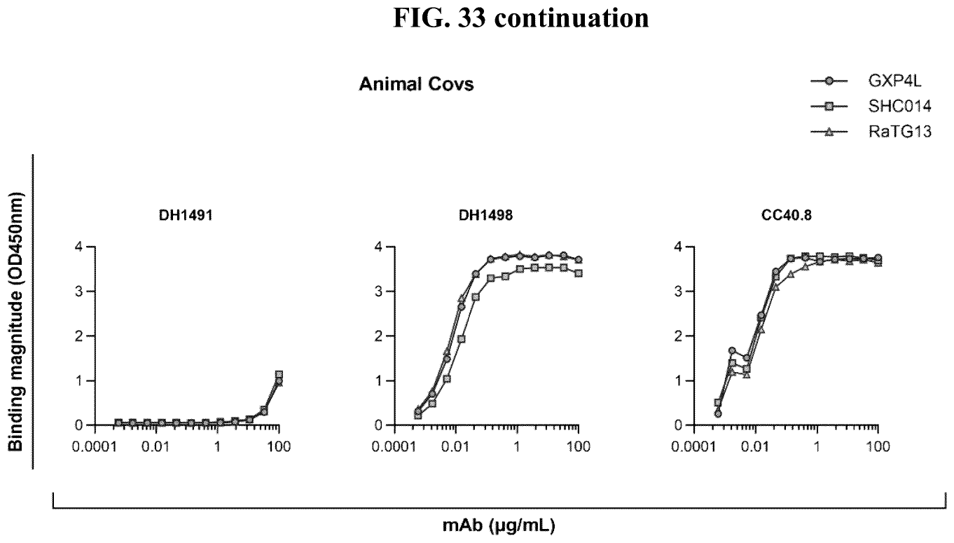

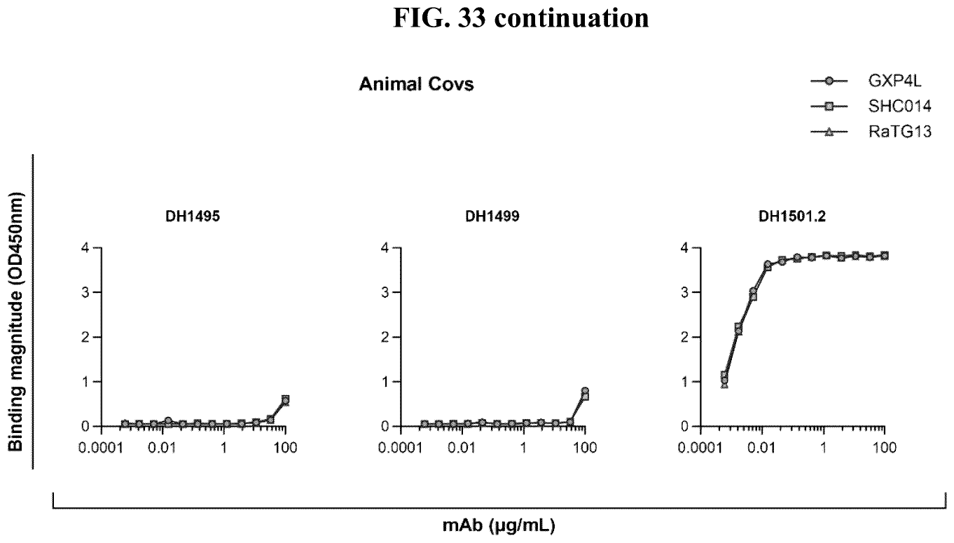

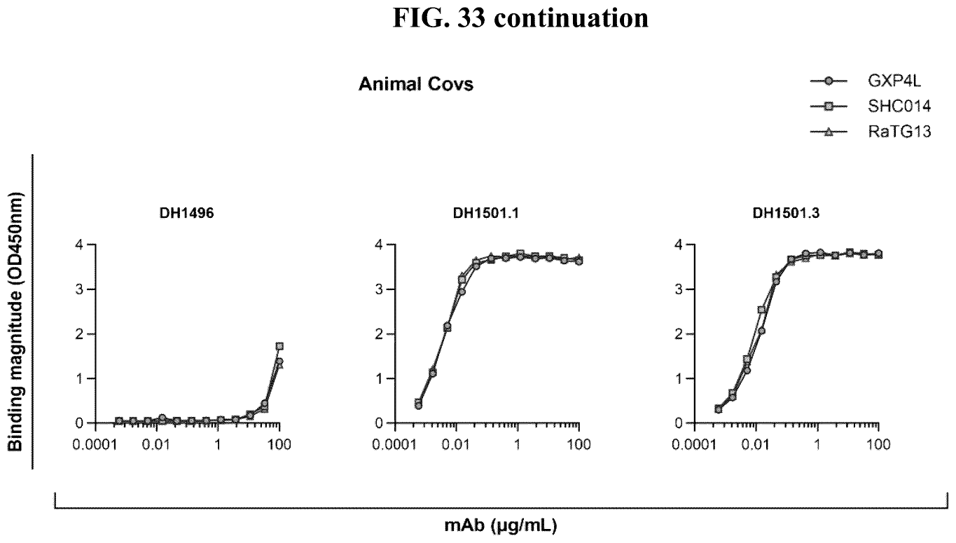

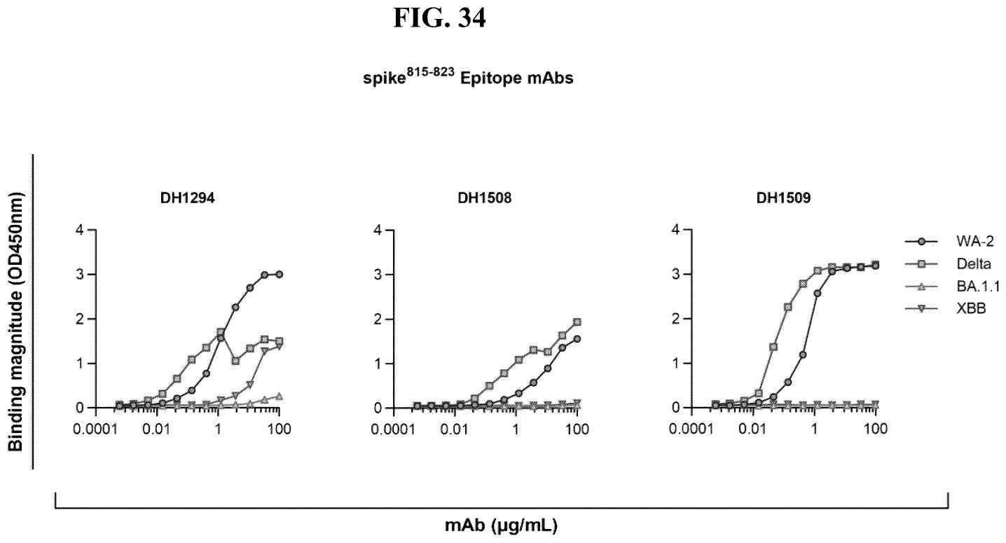

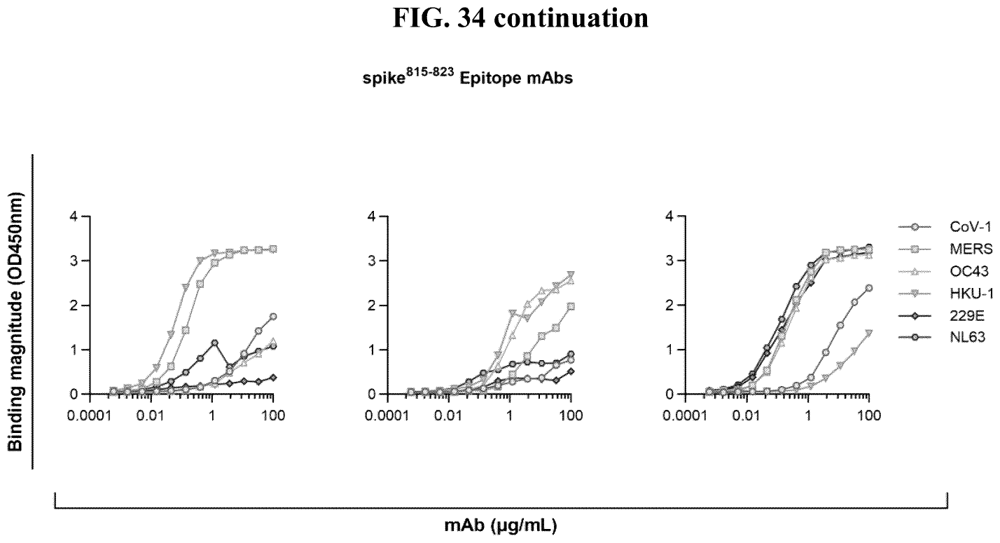

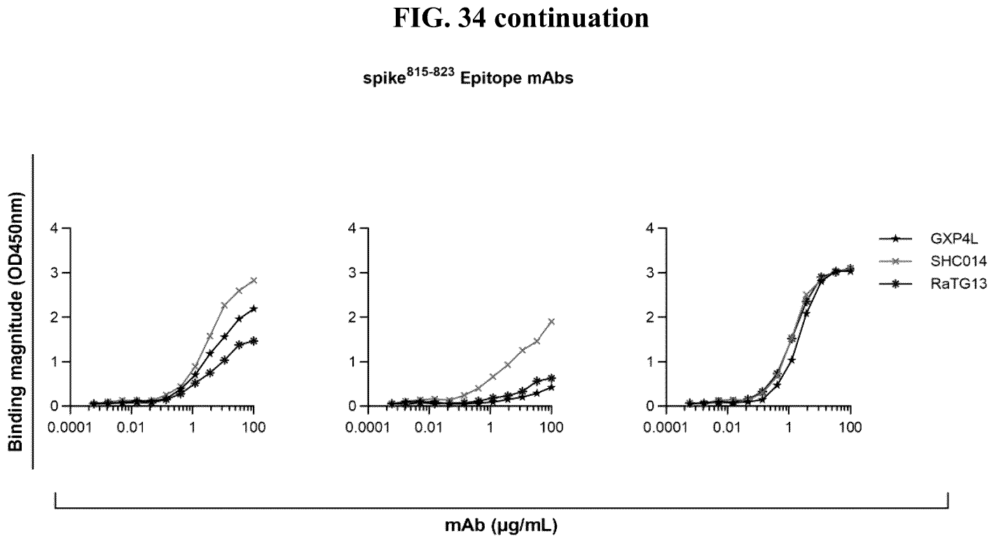

Antibody sequences were amplified from the isolated B cells and preliminarily screened for spike binding in order to select monoclonal antibodies for recombinant production and in-depth functional characterization. Twelve recombinant antibodies selected from B cells that bound S2hlx-Ex19 but not S2hlx-Ex19-PS were tested for binding against a panel of spike proteins from SARS-CoV-2 variants as well as other human and animal betacoronaviruses. Ten antibodies showed measurable affinity to multiple coronavirus spikes, with three of them displaying binding on par to that of S2P6 and CC40.8 mAbs to all the spikes tested, including SARS-CoV-1, MERS, OC43 and HKU-1 (FIG. 30c, FIGS. 31-34). Antibodies DH1501.1, DH1501.2, and DH1501.3 were clonally related and their heavy and light chain V gene segment pairing (IGHV1-46/IGKV3-20) matched those of S2P6 mAb. The inferred UCA of these antibodies displayed tight binding to multiple spikes, similar to what was reported for the iGL of S2P6 mAb (FIG. 35). Beyond the IGHV1-46/IGKV3-20 derived mAbs, multiple other unique VH/VL pairings were observed in the isolated antibodies (FIG. 30c). This suggests the presence of a clonally diverse response against the stem helix epitope that can be engaged by the engineered epitope scaffold. The best six antibodies by affinity and breadth were tested side by side with S2P6 and CC40.8 for their ability to neutralize multiple SARS-CoV-2 variants and SARS-CoV-1 in a pseudovirus neutralization assay. CC40.8 and two isolated mAbs, DH1501.1 and DH1501.3, displayed neutralization activity, albeit with high IC50 values above 1 μg/mL that are typical for anti-bodies against this epitope. DH1501.3 and CC40.8 neutralized all the pseudoviruses in the panel, including XBB1.5 and SARS-CoV-1 (FIG. 30d).

Stem helix antibodies can protect against infection in animal models despite limited neutralization potency. The ability of the herein isolated monoclonal antibodies (mAbs) to control virus infection through antibody-mediated functions was investigated. Binding of the antibodies to virus-infected cells, a prerequisite of antibody-dependent cellular cytotoxicity (ADCC) was assessed to determine the ability of these mAbs to mediate Fc effector functions. Three of the five antibodies tested, DH1501.1, DH1501.2, and DH1501.3, showed binding to cells infected with either the D614G or the BA.1 variant of SARS-CoV-2 in the same range as the previously described stem helix antibodies CC40.8, S2P6 and DH1057.1 (FIG. 30e). Binding of the stem helix antibodies was lower than that of the RBD-directed mAb DH1047, supporting the observation that the stem helix epitope is more occluded on the viral spike.

A Natural Killer (NK) cell degranulation assay was performed to assess ADCC activity. Antibodies DH1489, DH1501.1, DH1501.2, and DH1501.3 induced NK cell degranulation against all three variants tested, with potencies similar to those of known stem helix antibodies (FIG. 30f). All stem helix mAbs induced the most potent degranulation against BA.4/5 spike-transfected cells. Isolated antibodies with IGHV1-46/IGKV3-20 immunogenetics had the highest activity in both the pseudovirus neutralization and ADCC assays.

Antibodies against the spike epitope were isolated, using a process similar to the one described above, by sorting B cells that bound the FP-10 epitope scaffolds and lacked binding to the original parent scaffold that did not contain the grafted epitope. The frequency of epitope specific B cells selected was ˜1:4,000 in two analyzed samples, and two antibody sequences were recovered for recombinant expression and characterizations after preliminary screening. These isolated antibodies had distinct immunogenetics not observed in other previously isolated antibodies against the spike epitope (FIG. 30c). Nevertheless, they bound tightly to diverse coronavirus spikes, including those of human alpha coronaviruses 229E and NL63 as is typical of antibodies with broad activity against this epitope (FIG. 30C). No binding was detected for SARS-CoV-2 variants BA.1 and XBB1.5, however these recombinant spikes contained a “hexapro” mutation that is known to affect the presentation of the spike epitope and to limit the binding of antibodies against this site.

Antibody isolation from subjects with pre-existing SARS-CoV-2 immunity revealed that the engineered epitope scaffolds interact with diverse monoclonals against the target epitopes. Some of the antibodies are derived from the same VH and VL genes observed in anti-bodies previously isolated. While the epitope scaffold reactive mAbs against the stem helix had weak neutralization potency, they exhibited strong ADCC activity in vitro, revealing a potential protection mechanism consistent with previous observations for this antibody class. Taken together, the robust sera binding and memory B cell engagement by epitope scaffolds demonstrate their strong recognition of pre-existing SARS-CoV-2 immune response and support their use for boosting spike and stem helix antibodies by vaccination.

Engineered Stem Helix Epitope Scaffolds Elicit Antibodies with Broad Reactivity Against Diverse CoVs by Vaccination

The ability of the engineered epitope scaffolds to elicit antibodies against the target epitope by vaccination was assessed. The assessment was focused on testing the stem helix immunogens belonging to the S2hlx-Ex2 family of design, Ex15, Ex19, Ex20, and Ex17, because: 1) they have high affinities for all the antibodies tested as well as their precursors; and 2) while they share the same backbone structure, their sequence outside the epitope is highly divergent, which may help focus immune responses on the epitope by vaccinating with combinations of different designs.

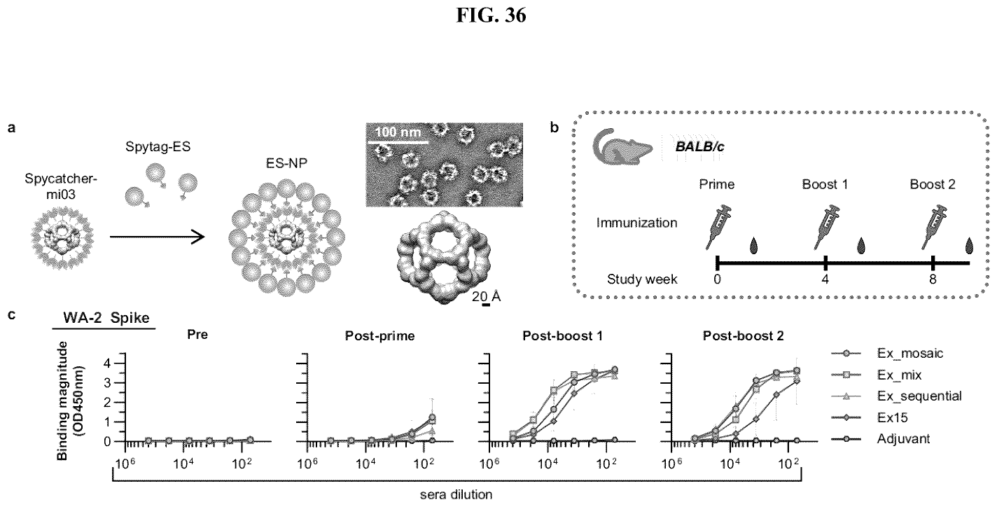

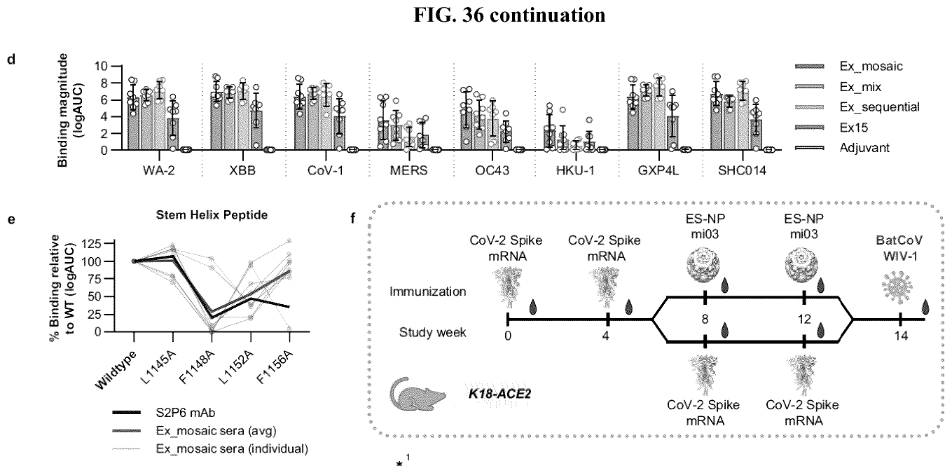

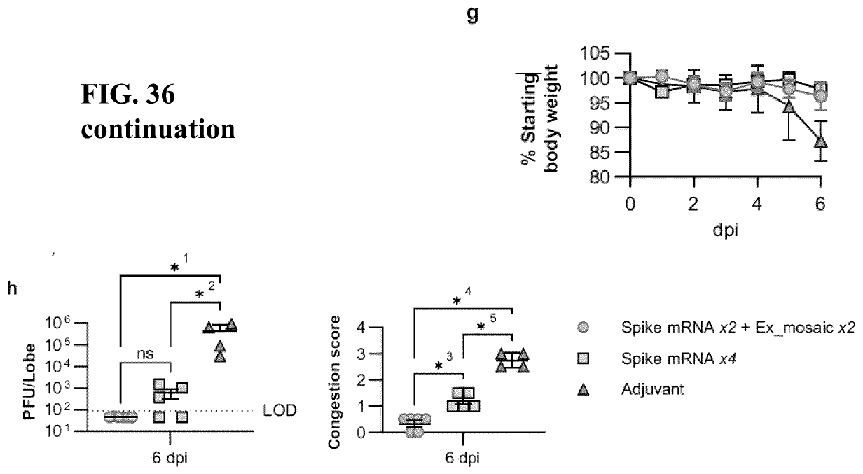

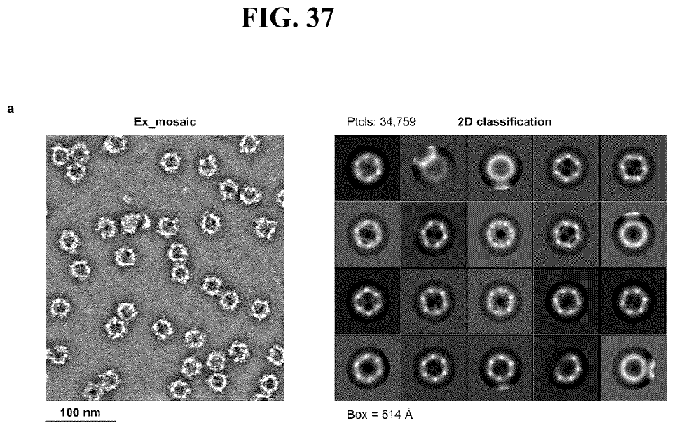

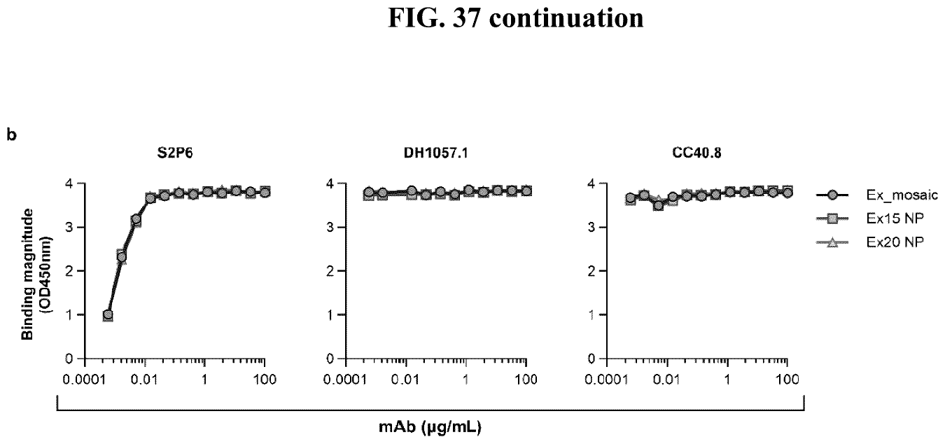

Epitope scaffolds were multimerized on mi03 nanoparticles (NPs) using the SpyCatcher/Spytag23 conjugation system in order to improve immune presentation (FIG. 36A). Homotypic nanoparticles were developed that displayed 60 copies of designs Ex15, Ex19, or Ex20, respectively. A “mosaic” NP was also engineered where Ex15, Ex19, Ex20, and Ex17 were conjugated together. Antigenic and NSEM characterization confirmed that the NPs were well formed and bound S2P6, DH1057.1 and CC40.8 (FIG. 37A and FIG. 37b). Groups of eight BALB/c mice were vaccinated three times, four weeks apart with either: 1) Ex15-NP; 2) Ex_mosaic-NP; 3) a mixture of individual Ex15-NP, Ex19-NP and Ex20-NP; 4) a sequential regimen of Ex15-NP prime, followed by Ex19-NP and Ex20-NP boosts; or 5) GLA-SE only, the adjuvant included in all the immunizations, as control (FIG. 36b). After two immunizations, sera from all animals vaccinated with epitope scaffolds showed strong reactivity to WA-2 and XBB SARS-CoV-2 spikes (FIG. 36C). Sera breadth was tested against spikes from all human beta coronaviruses as well as RsSHC014 and GXP4L, two pre-emergent animal viruses from bats and pangolins. Binding was observed against all the spikes tested, with the highest activity against SARS-CoV-1, RsSHC014, and GXP4L (FIG. 36d).

Sera cross-reacted with OC43, MERS, and HKU-1 spikes, although at lower levels than those measured against SARS-CoV-2, but consistent with the induction of a broad response against the stem helix epitope (FIG. 36D). Of the four immunization regimens tested, the homotypic Ex15 NP elicited the lowest titers and breadth. Combinations of epitope scaffolds whether administered together, in mosaic form, or sequentially, had similar performance, although the Ex_mosaic-NP elicited the highest average titers against heterologous human betacoronaviruses.

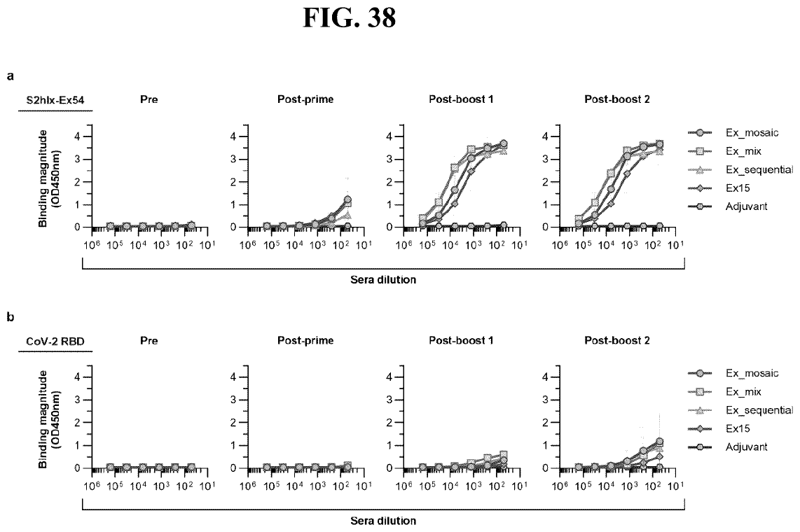

Epitope specificity was demonstrated by strong sera binding to an unrelated epitope scaffold not used in the immunizations (FIG. 38). Epitope specificity was further demonstrated by measuring binding to synthesized peptides encoding the stem helix region as well as mutated variants at sites known to be important for antibody recognition (FIG. 36e). Mutations at residues 1148, 1152, and 1156 decreased sera binding, while alanine substitution at position 1145 had limited effect, consistent with the elicitation of antibodies that resemble the binding mode of S2P6 or DH1057.1, rather than that of CC40.8 (FIG. 36e). No neutralizing activity was observed for the elicited sera, which was unsurprising given the limited activity of S2P6-like recombinant monoclonals in our pseudovirus neutralization assay (FIG. 30d).

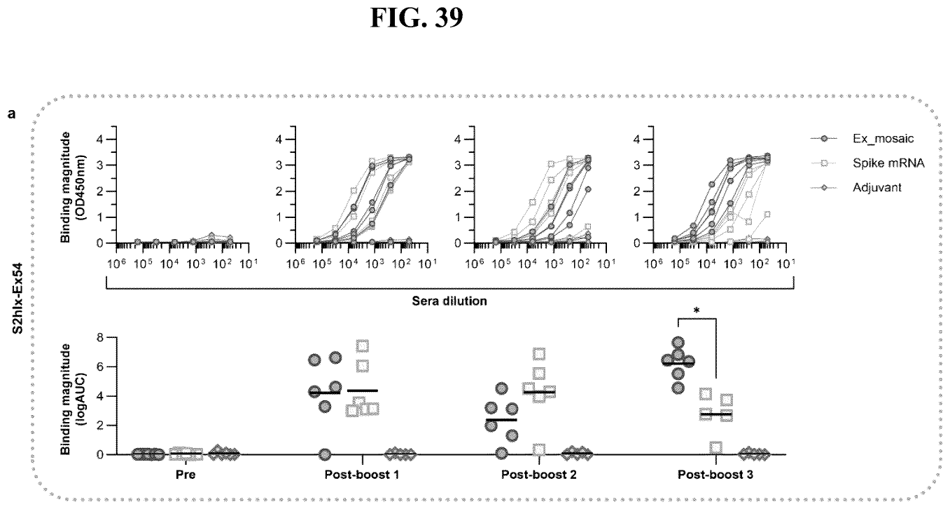

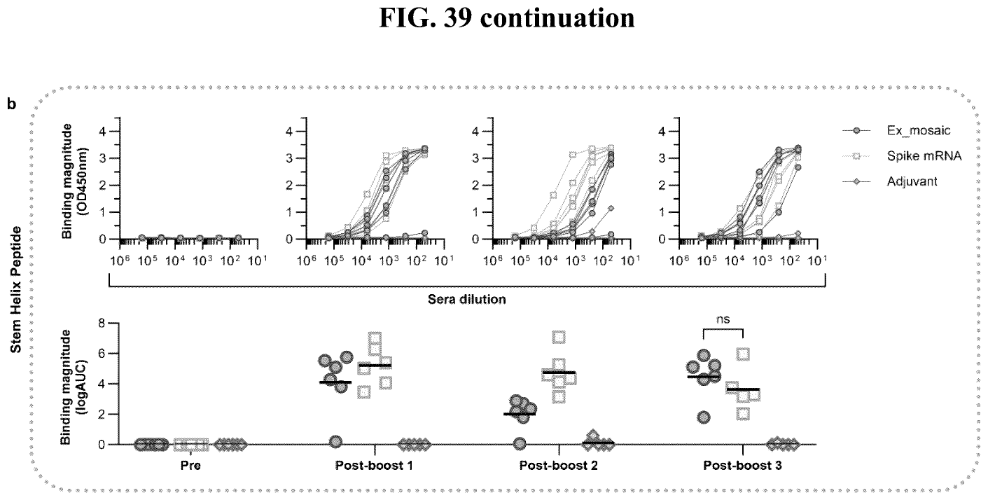

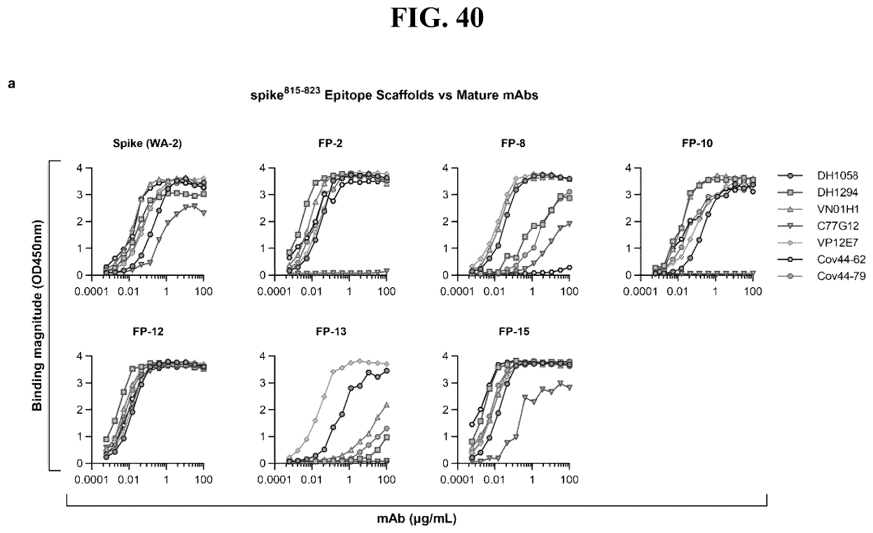

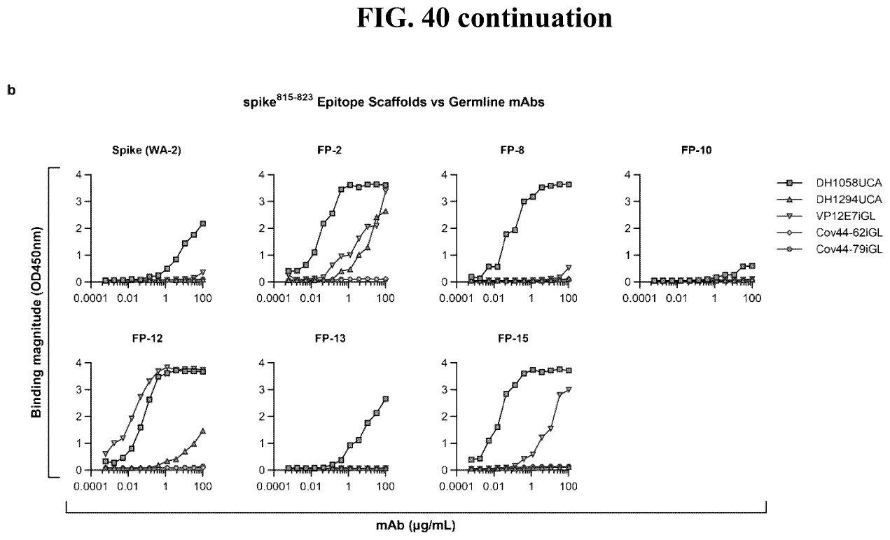

The ability of the Ex_mosaic-NP to boost stem helix specific responses in animals previously vaccinated with mRNA encoding the SARS-CoV-2 WA-2 spike and to protect against a viral challenge with a heterologous virus was assessed. Assessing protection was important because sera elicited by stem helix epitope scaffolds did not neutralize in our assay; however, antibodies that react with the grafted epitope can promote strong ADCC, as shown above (FIG. 30e and FIG. 30f) and can protect through antibody-mediated functions. K18-ACE2 mice were immunized every four weeks with either four shots of mRNA spike or twice with mRNA spike followed by two boosts of the Ex_mosaic-NP. Two weeks after the last immunization, the animals were challenged with live WIV-1 virus (FIG. 36f). WIV-1 was chosen because its spike sequence is significantly different from that of SARS-CoV-2 (78% over the whole spike and 75% over RBD), while the stem helix epitope sequence is conserved, as in most sarbecoviruses. Animals vaccinated with Ex_mosaic-NP had higher average titers of anti-bodies targeting the stem helix epitope compared to those that received only spike mRNA, as measured by binding to an epitope scaffold not used in the immunization; binding to the synthesized stem helix peptide was also higher, but not statistically significant (FIG. 39). Both immunizations regimens provided strong protection from WIV-1 based on the percentage of body weight the animals lost six days after the challenge and when compared to animals that received adjuvant only (FIG. 36g). However, animals boosted with Ex_mosaic-NP scored better on two other clinical measures compared to those that received spike mRNA only. No viral RNA was detected in the lungs of any of the Ex_mosaic-NP immunized animals, and congestion scores of the animals were significantly lower when compared to the congestion scores of mRNA spike vaccinated animals (FIG. 6h). These results demonstrate that stem helix epitopes elicit sera with broad reactivity against beta coronaviruses and can offer protection against divergent viruses when used to boost immune responses initially induced by mRNA spike immunizations.

The ability of engineered immunogens to engage diverse antibodies against a displayed epitope is important because it can elicit a robust polyclonal response in vivo. The epitope scaffolds designed herein can engage multiple spike and stem helix antibodies with high affinity in vitro, which translates to robust reactivity to polyclonal sera elicited by SARS-CoV-2 and the engagement of BCRs with diverse immunogenetics against the target epitopes.

The stem helix epitope scaffolds that also bound CC40.8 mAb in addition to S2P6 and DH1057.1 mAbs, showed the highest level of binding to human sera from subjects exposed to SARS-CoV-2 by vaccination, infection or both. The S2hlx-Ex19 design was used to isolate multiple antibodies with broad reactivity against the stem helix from people with pre-existing immunity. Some of these monoclonal antibodies had the same VH/VL pairings (IGHV1-46/IGKV3-20) as previously isolated antibodies, suggesting that this type of antibody is commonly induced by SARS-CoV-2 immunization and may be targeted for boosting with a next-generation vaccine. However, as found here, other broad antibodies with different immunogenetic characteristics exist against S2 and they can be engaged by the designed epitope scaffolds as well. Isolated stem helix antibodies displayed limited neutralization against SARS-CoV-2 variants, consistent with previous reports describing other antibodies against this epitope. Interestingly, despite their limited neutralization, stem helix antibodies were shown by others to protect well against live virus challenges, likely through their antibody-mediated functions which we demonstrated our antibodies also have.

### Example 5

### Neutralization Assays

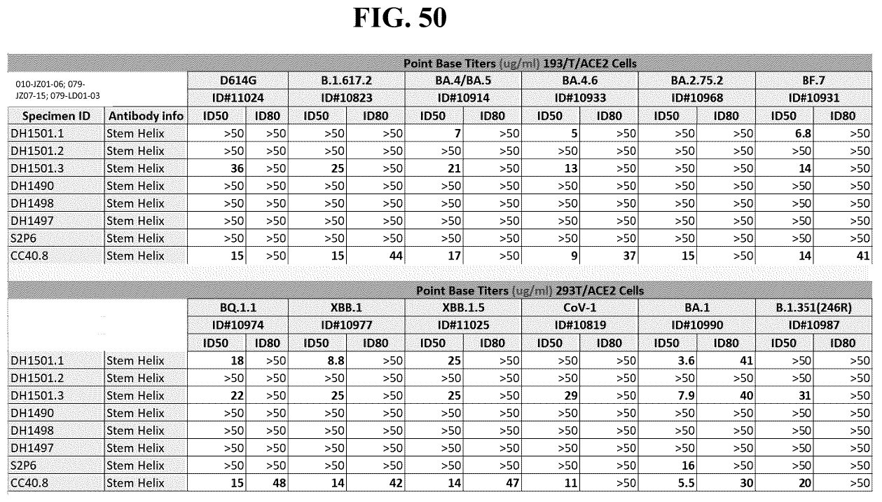

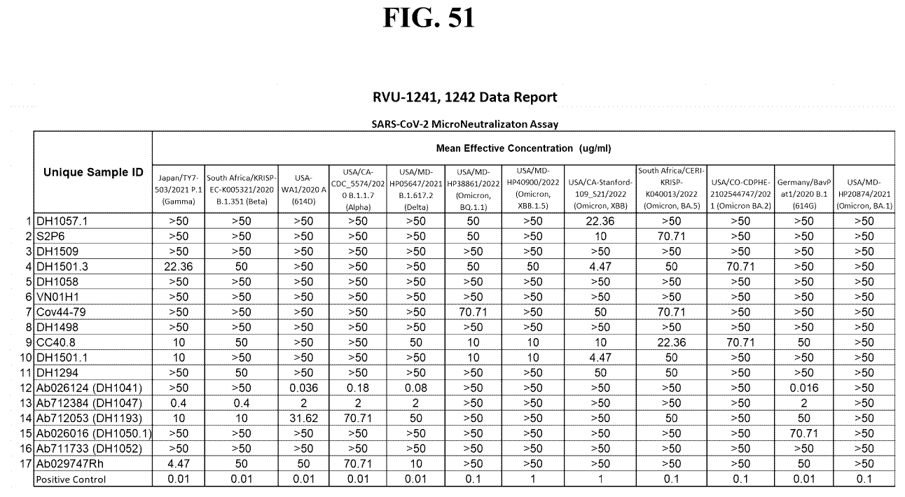

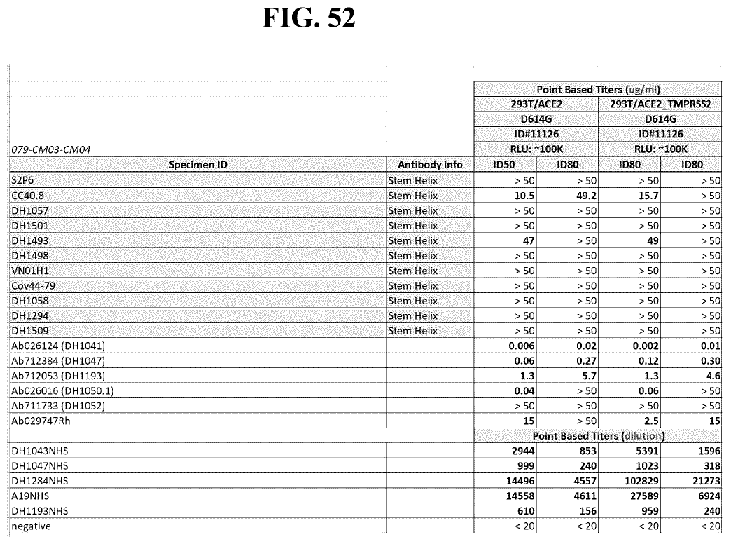

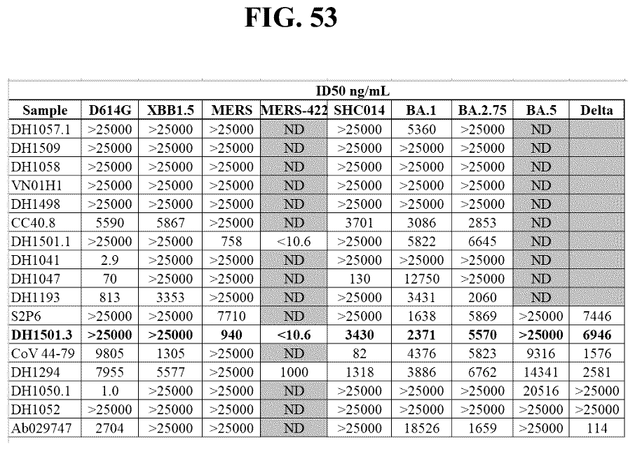

FIGS. 50-53 show results of example neutralization assays.

### EQUIVALENTS

Those skilled in the art will recognize, or be able to ascertain, using no more than routine experimentation, numerous equivalents to the specific substances and procedures described herein. Such equivalents are considered to be within the scope of this invention.

1-55. (canceled)

56. An amino acid epitope grafted into a scaffold protein, to which an antibody stimulated by a SARS-CoV-2 coronavirus can bind, the amino acid epitope comprising:
RSX₁IEDX₂LF (SEQ ID NO: 179), or an amino acid sequence at least 90% identical thereto, wherein X₁ and X₂ comprise any amino acid;
FKX₁ELDX₂YF (SEQ ID NO: 180), or an amino acid sequence at least 90% identical thereto, wherein X₁ and X₂ comprise any amino acid;
(SEQ ID NO: 89)
ELDSFKEELDKYFK,
(SEQ ID NO: 90)
ALASFKIELDIYFI,
(SEQ ID NO: 91)
ALDFFKLELDFYFL,
(SEQ ID NO: 92)
ALDGFKLELDLYFF,
(SEQ ID NO: 93)
ALDGFKLELDVYFI,
(SEQ ID NO: 94)
ELAKFKIELDKYFA,
(SEQ ID NO: 95)
ELASFKIELDIYFG,
(SEQ ID NO: 96)
ELASFKIELDIYFI,
(SEQ ID NO: 97)
ELEKFKYELDLYFI,
(SEQ ID NO: 98)
ELEKFKYELDLYFM,
(SEQ ID NO: 99)
ELGKFKYELDLYFM,
(SEQ ID NO: 100)
ELREFKIELDKYFA,
(SEQ ID NO: 101)
GLALFKVELDIYFL,
(SEQ ID NO: 102)
GLASFKIELDIYFI,
(SEQ ID NO: 103)
KLEEFKYELDIYFM,
(SEQ ID NO: 104)
NLDSFKEELDDYFK,
(SEQ ID NO: 105)
SLASFKIELDIYFF,
(SEQ ID NO: 106)
SLASFKYELDIYFG,
(SEQ ID NO: 107)
SLELFKFELDIYFL,
(SEQ ID NO: 108)
SLELFKFELDLYFL,
(SEQ ID NO: 109)
SLELFKWELDVYFL,
(SEQ ID NO: 110)
SLELFKYELDLYFL,
(SEQ ID NO: 111)
SLGKFKYELDLYFL,
(SEQ ID NO: 112)
SLSLFKYELDLYFL,
(SEQ ID NO: 159)
KLKLFKCELDLYFL,
(SEQ ID NO: 160)
KLKLFKCELDLYFL,
(SEQ ID NO: 161)
KLRLFKLELDLYFL,
(SEQ ID NO: 162)
RLKLFKCELDLYFL,
(SEQ ID NO: 163)
KLKLFKCELDLYFL,
(SEQ ID NO: 164)
KLKLFKCELDLYFL,
(SEQ ID NO: 165)
KLLLFKEELDLYFL,
(SEQ ID NO: 166)
SLKLFKCELDLYFL,
(SEQ ID NO: 167)
KLLLFKCELDLYFL,
(SEQ ID NO: 168)
ELKLFKCELDLYFL,
(SEQ ID NO: 169)
SLCGFKGELDLYFL,
(SEQ ID NO: 170)
KLKEFKYELDFYFI,
(SEQ ID NO: 171)
SLKEFKYELDKYFL,
(SEQ ID NO: 172)
SLPEFKYELDEYFI,
(SEQ ID NO: 173)
SLPEFKFELDRYFI,
(SEQ ID NO: 174)
KLPTFKYELDEYFI,
(SEQ ID NO: 175)
ELPEFKFELDKYFI,
(SEQ ID NO: 176)
SLPDFKYELDLYFI,
(SEQ ID NO: 177)
KLDSFKYELDEYFI,
(SEQ ID NO: 178)
NLKDFKFELDRYFI
or an amino acid sequence at least 90 identical thereto; and
the scaffold protein.

57. The amino acid epitope grafted into the scaffold protein of claim 56, wherein the scaffold protein is selected from the group consisting of:
a polypeptide from a Kai A protein from Anabaena sp PCC7120 (PDBid 1r5q), a T4 Lysozyme protein from Escherichia coli virus T4 (PDBid 1l23); a Conserved Hypothetical protein from Staphylococcus aureus (PDBid 1tsj), a Syntaxin-1A protein from Rattus norvegicus (PDBid 1ez3), a PG0816 protein from Porphyromonas gingivalis W83 (PDBid 2apl), a Response regulator receiver protein from Acetivibrio thermocellus ATCC 27405 (PDBid 3jte), a Phosphate Regulon protein from Escherichia coli (PDBid 2jb9), a CcmG protein from Escherichia coli (PDBid 2b1k), a ClpS 2 protein from Agrobacterium fabrum str. C58 (PDBid 4yjm), a t-SNARE VTI1 polypeptide from Saccharomyces cerevisiae (PDBid 3onj), a Catenin Alpha-protein from Mus musculus (PDBid 5y04), a PA2169 protein from Pseudomonas aeruginosa PA01 (PDBid 4etr), a Thioredoxin protein from Caulobacter vibrioides NA1000 (PDBid 6esx), a WA352 protein from Oryza sativa indica (PDBid 5zt3), and a B562RIL protein from Escherichia coli (PDBid 5yo4).

58. The amino acid epitope grafted into the scaffold protein of claim 56, wherein the scaffold protein is selected from the group consisting of:
a Ferredog-Diesel protein (synthetic construct) (PDBid 6nuk), a CRISPR complex subunit Csm2 protein from Staphylococcus epidermidis RP62A (PDBid 6nbu), a SarX protein from Staphylococcus aureus sp. NCTC 8325 (PDBid 5ywj), a B562RIL protein from Escherichia coli (PDBid 5ym7), a CB15 Pilus assembly protein CpaE from Caulobacter vibrioides (PDBid 4n0p), an AvtR protein from Acidianus filamentous virus 6 (PDBid 4hv0), an Acyl carrier RPA2022 protein from Rhodopseudomonas palustris (PDBid 31mo), a Response regulator protein from Hahella chejuensis KCTC 2396 (PDBid 3kht), a RBSTP2171 protein from Escherichia coli (PDBid 3fgx), a TM_1646 protein from Thermotoga maritima MSB8 (PDBid 2p61), a Diheme Cytochrome C protein from Rhodovulum sulfidophilum (PDBid 1h31), an ATP-dependent protease La 1 from Bacillus subtilis subsp. subtilis str. 168 (PDBid 3m65), a Mitochondrial antiviral signaling protein from Equus caballus (PDBid 4o9f), a Staphylococcal nuclease mutant T44V protein from Staphylococcus aureus (PDBid 2eyf), and a Putative periplasmic protein BACEGG_01429 protein from Bacteroides eggerthii DSM 20697 (PDBid 4hbr).

59. The amino acid epitope grafted into the scaffold protein of claim 56, wherein the scaffold protein is selected from the group consisting of:
a UPF0247 E protein from Staphylococcus aureus (PDBid 1vh0), a VPS54 protein from Mus musculus (PDBid 3n1b), a FFL_005 protein (Synthetic RSV epitope scaffold) (PDBid 4l8i), a Vtilb Habc domain from Mus musculus (PDBid 2qyw), a CDC37 protein from Homo sapiens (PDBid 2w0g), a SaeR protein from Staphylococcus aureus (PDBid 4wqw), a syntaxin 6 protein from Rattus norvegicus (PDBid 1lvf) and a Vtilb Habc domain from Saccharomyces cerevisiae (PDBid 3onj).

60. The amino acid epitope grafted into the scaffold protein of claim 56, wherein the RSX₁IEDX₂LF (SEQ ID NO: 179) comprises amino acids RSFIEDLLF (SEQ ID NO: 66), RSAIEDGLF (SEQ ID NO: 67), RSAIEDILF (SEQ ID NO: 68), RSAIEDLLF (SEQ ID NO: 69), RSGIEDFLF (SEQ ID NO: 70), RSHIEDVLF (SEQ ID NO: 71), RSIIEDLLF (SEQ ID NO: 72), RSLIEDYLF (SEQ ID NO: 73), RSQIEDLLF (SEQ ID NO: 74), RSTIEDFLF (SEQ ID NO: 75), RSVIEDTLF (SEQ ID NO: 76), RSYIEDVLF (SEQ ID NO: 77), RSYIEDWLF (SEQ ID NO: 78), or an amino acid sequence 90 or 95% identical thereto.

61. The amino acid epitope grafted into the scaffold protein of claim 56, wherein the FKX₁ELDX₂YF (SEQ ID NO: 180) comprises amino acids FKEELDKYF (SEQ ID NO: 79), FKDELDKYF (SEQ ID NO: 80), FKIELDKYF (SEQ ID NO: 81), FKLELDKYF (SEQ ID NO: 82), FKNELDKYF (SEQ ID NO: 83), FKRELDKYF (SEQ ID NO: 84), FKSELDKYF (SEQ ID NO: 85), FKVELDKYF (SEQ ID NO: 86), FKVELDLYF (SEQ ID NO: 87), FKYELDKYF (SEQ ID NO: 88) or an amino acid sequence 90 or 95% identical thereto.

62. A method for stimulating an immune response in an individual, comprising administering to the amino acid epitope grafted into the scaffold protein of claim 56.

63-64. (canceled)

65. A method for making the amino acid epitope grafted into a scaffold protein of claim 56, comprising:
selecting an epitope that can stimulate an immune response to an S2 region of a coronavirus spike protein;
identifying a scaffold protein with an exposed backbone region that matches the selected epitope (root-mean-square-deviation 0.5 Å); and
incorporating the epitope into the scaffold protein to obtain an epitope scaffold having an RMSD of less than about 1.0 Å as compared to the scaffold protein that has not incorporated the epitope.

66. An epitope scaffold, comprising a peptide/polypeptide having an amino acid sequence SEQ ID NO: 1-65 or SEQ ID NO: 139-158, or a peptide/polypeptide having at least 90% identity thereto.

67. An immunogenic composition, comprising the epitope scaffold of claim 66.

68. A composition comprising antibodies induced by the immunogenic composition of claim 67.

69-93. (canceled)

94. An antibody or antigen-binding fragment or variant thereof, comprising a heavy chain variable region (HCVR) and a light chain variable region (LCVR), wherein:
a) the amino acid sequence of the HCVR is QVQLQQSGPGLVKPSETLSLTCTVSGGSISSSSYYWGWIRQPPGKGLEWIGSIYYSG STYYNPSLKSRVTISVDTSKNQFSLKLSSVTAADTAVYYCARHGPLRFRLGYYDSSG YLFQHWGQGTLVTVSS (SEQ ID NO: 113) and the amino acid sequence of the LCVR is DIQLTQSPSTLSASVGDRVTITCRASQSISSWLAWYQQKPGKAPKLLIYDASSLESG VPSRFSGSGSGTEFTLTISSLQPDDFATYYCQQYNSYSRWTFGQGTKVEIK (SEQ ID NO: 126); or
b) the amino acid sequence of the HCVR is QVQLVQSGGGLVQPGGSLRLSCATSGFNFRAFAMSWVRQAPGKGLEWAAVMSGT DDGTYYVESVKGRFTIFRDNSENTVYLQMNSLRADDSAIYYCAKGTLGHCSGVDC YYLDYWGRGTLVTVSS (SEQ ID NO: 114) and the amino acid sequence of the LCVR is DIVLTQSPATLSLSPGERATLSCRASQSVGTYVAWYQHKVGQAPRLLIYDASTRAT DIPARFSGSGSGTDFTLTITSLEPEDVAIYYCQQRVNLVTFGGGTKLEIK (SEQ ID NO: 127); or
c) the amino acid sequence of the HCVR is QVQLVQSGAEVKKPGASVRLSCKASGDTFTNEYVQWVRQAPGQGLEWMGLINPS GSGTAFARNFQGRVSMTRDTSTRTVYMDLTSLRYEDTAVYYCARMSRAGTFDLW GQGTMVTVSS (SEQ ID NO: 115) and the amino acid sequence of the LCVR is EIVLTQSPDTLSLSLGERATLFCRASETIDSRYLAWYQQKPGRAPRLLMSGTSVRA TGIPDRFSGSGSGTDFTLTITRLEPEDFAVYYCQQYGSSPPRYTFGQGTKLAMK (SEQ ID NO: 128); or
d) the amino acid sequence of the HCVR is EVQLVESGGGLVQPGGSLRLSCAASGFTFDNYAMGWVRQPPGKGLEWVSSFSGRG VSTYYADSVKGRFTVSRDSSKNTLFLQMNYLRVEDTAVYYCATYPYDILTGYYGAF DYWGQGALVTVSS (SEQ ID NO: 116) and the amino acid sequence of the LCVR is DIQLTQSPSTLSASVGDRVTITCRASQTIDTWLAWYQQKPGKAPKLLIYGASSLQS GVPSRFSASGSGTEFTLTISSLQPDDFAIYYCQQYKDYSTFGQGSKVEFM (SEQ ID NO: 129); or
e) the amino acid sequence of the HCVR is QVQLQQWGAGLLKPSETLSLTCSFYGGSFSGYCWSWIRQSPGKGLEWIGEINHSGS TNYNPSLKTRVTILIDTSNNQFSLRLSSVTAADTAVYYCARERAIRRCTSTSCYRVG GVAGLDVWGQGTTVSVSS (SEQ ID NO: 117) and the amino acid sequence of the LCVR is DIVMTQSPSSLSASVGDRVTITCQASRDIYKYLNWYQQKPGQAPKLLISDASNLET GVPSRFSGSGSGTDFTFTISSLQPEDIAAYYCQQYDNPLITFGQGTRLEIK (SEQ ID NO: 130); or
f) the amino acid sequence of the HCVR is QVQLVQSGGGLAQPGESLRLSCAASGFTFSSYAMTWVRQAPGKGLEWVSSISGKG ENIEYAESVKGRFTISRDNAKNTVDLEMNSLRAEDTATYFCAKHIYGAFIVVPNTL YDALDVWGQATKVTVSS (SEQ ID NO: 118) and the amino acid sequence of the LCVR is EIVLTQSPGTLSLSPGERATLSCRASQSVSRNYLAWYQQKPGQAPRILVYDASNRAI GIPDRFSGSGSGTDFTLTISRLEPEDFAVYYCQQYGSSPGTFGQGTRLEIK (SEQ ID NO: 131); or
g) the amino acid sequence of the HCVR is QVQLQESGPGLVKPSETLSLTCAVSGGSVSSDTYYWSWIRQPPGKGLEWIGYIYNS GSTNYNPSLKSRVTISVDTSKSQFSLNLGSVTAADTAVYYCAREGAGTYPPKNDAF DIWGQGTLVTVSS (SEQ ID NO: 119) and the amino acid sequence of the LCVR is EIVLTQSPDSLAVSLGERATINCKSSQSVLFSSDNKNYLAWYQQKPGQPPKLLIYW ASTRESGVPDRFSGSGSGTDFTLTISSLQAEDVAVYYCQQYYNSPRTFGQGTKVEIK (SEQ ID NO: 132); or
h) the amino acid sequence of the HCVR is QVQLQESGGGLVKPGGSLRLSCAASGFTFSSYSMNWVRQAPGKGLEWVSSISSSSS YIYYADSVKGRFTISRDNAKNSLYLQMNSLRAEDTAVYYCARGIIDYGDYFDYWGQ GTLVTVSS (SEQ ID NO: 120) and the amino acid sequence of the LCVR is EIVLTQSPDTLSLSPGERATVSCRASQSVGAGYVAWYQQRPGQPPRLLIYGASVRA TGIPDRFSGSGSGTDFSLTINRVEPEDFAVYYCQNYASSPPRYTFGQGTKLEIK (SEQ ID NO: 133); or
i) the amino acid sequence of the HCVR is EVQLVESGAEVKKPGASVRVSCKASGFTFTDHYMHWVRQAPGQGLEWMGLINPT GVNTFYAQNFRGRVTMTRDTSTKTDYLEVRSLTSQDTAMYYCARMSRYGAFDIW GQGTMVTVSL (SEQ ID NO: 121) and the amino acid sequence of the LCVR is EIVLTQSPGTLSLSPGERATLSCRASQSVGSGYLAWYQQKPGQAPRLLIYGASVRAT GIPDRFSGSGSGTDFSLTINRVEPEDFAMYYCQNYGSSPPRYTFGQGTKLEIK (SEQ ID NO: 134); or
j) the amino acid sequence of the HCVR is EVQLVESGAEVKKPGASVRLSCKASGDTFNNKYVHWVRQAPGQGLEWMGLINPS GSGTAFAQKFQGRVSMTRDTSTRTVYLGLTSLTYEDTAVYYCARMSRAGTFDIWD QGTMVTVSS (SEQ ID NO: 122) and the amino acid sequence of the LCVR is EIVLTQSPGTLSLSPGDRATLSCRASENVGTSYLAWYQQKPGQAPRLLISGTSSRAT GVPDRFSGSGSGRDFTLTISRLEPEDFAVYYCQQYGSSPPRYTFGQGTKLDMK (SEQ ID NO: 135); or
k) the amino acid sequence of the HCVR is QVQLVQSGAEVKKPGASVKLSCRASGYPITSHYMHWVRQAPGQGLEWMGIINPSG TGTSFARNFQGRVTMTRDTATRTVYMELSSLKSEDSAVYYCAGGTMGPLFDYWG QGTLVTVSS (SEQ ID NO: 123) and the amino acid sequence of the LCVR is EIVLTQSPGTLSLSPGERATLSCRASQSVRRNFLAWYQQKPGQAPRLLIYEASTRA TGIPDRFSGSGSGTDFTLTISRLEPEDFAVYYCQQYDSSPPGVTFGPGTKVDIR (SEQ ID NO: 136); or
l) the amino acid sequence of the HCVR is QVQLVESGGGVVQPGKSLKLSCAASGFTFSTYSMHWVRQAPGKGLEWVAIISYDG RNKYYANSVKGRFTISRDNSKNTLYLEINNLRPQDTAVYYCATDASNVEVPGKFA PLDKWGQGTLVTVSS (SEQ ID NO: 124) and the amino acid sequence of the LCVR is EIVMTQSPATLSVSPGERATLSCRASQSVSSDLAWYQQKPGQAPRLLIYGATTRAT GVPARFSGSGSGAEFTLTISSLQSGDFAVYYCQQYNDWPLITFGQGTRLEIK (SEQ ID NO: 137); or
m) the amino acid sequence of the HCVR is QVQLVESGGGVVQPGRSLRLSCAASGFTLSSHGIHWVRQAPGKGLEWVAVTSFDG RNKKFGDSVKGRFTISRDNSKNTVYLQMNSLRTEDTAVYYCAKDWGDDYSRWYF DLWGRGTLVTVSS (SEQ ID NO: 125) and the amino acid sequence of the LCVR is DIQMTQSPSSLSASVGDRVTITCQASHDITKYLNWYQQKPGRAPKLLIYDASNLKT GVPSRFSGSGSGTDFTFTITSLQPEDLATYYCQQSDVLPITFGQGTRLEIK (SEQ ID NO: 138).

95. An antibody or antigen-binding fragment or variant thereof, comprising a heavy chain variable region (HCVR) and a light chain variable region (LCVR), wherein:
a) the HCVR region comprises an amino acid sequence that has at least 95% sequence identity to the amino acid sequence QVQLQQSGPGLVKPSETLSLTCTVSGGSISSSSYYWGWIRQPPGKGLEWIGSIYYSG STYYNPSLKSRVTISVDTSKNQFSLKLSSVTAADTAVYYCARHGPLRFRLGYYDSSG YLFQHWGQGTLVTVSS (SEQ ID NO: 113) and the LCVR region comprises an amino acid sequence that has at least 95% sequence identity to the amino acid sequence DIQLTQSPSTLSASVGDRVTITCRASQSISSWLAWYQQKPGKAPKLLIYDASSLESG VPSRFSGSGSGTEFTLTISSLQPDDFATYYCQQYNSYSRWTFGQGTKVEIK (SEQ ID NO: 126); or
b) the HCVR region comprises an amino acid sequence that has at least 95% sequence identity to the amino acid sequence QVQLVQSGGGLVQPGGSLRLSCATSGFNFRAFAMSWVRQAPGKGLEWAAVMSGT DDGTYYVESVKGRFTIFRDNSENTVYLQMNSLRADDSAIYYCAKGTLGHCSGVDC YYLDYWGRGTLVTVSS (SEQ ID NO: 114) and the LCVR region comprises an amino acid sequence that has at least 95% sequence identity to the amino acid sequence LCVR is DIVLTQSPATLSLSPGERATLSCRASQSVGTYVAWYQHKVGQAPRLLIYDASTRAT DIPARFSGSGSGTDFTLTITSLEPEDVAIYYCQQRVNLVTFGGGTKLEIK (SEQ ID NO: 127); or
c) the HCVR region comprises an amino acid sequence that has at least 95% sequence identity to the amino acid sequence QVQLVQSGAEVKKPGASVRLSCKASGDTFTNEYVQWVRQAPGQGLEWMGLINPS GSGTAFARNFQGRVSMTRDTSTRTVYMDLTSLRYEDTAVYYCARMSRAGTFDLW GQGTMVTVSS (SEQ ID NO: 115) and the LCVR region comprises an amino acid sequence that has at least 95% sequence identity to the amino acid sequence EIVLTQSPDTLSLSLGERATLFCRASETIDSRYLAWYQQKPGRAPRLLMSGTSVRA TGIPDRFSGSGSGTDFTLTITRLEPEDFAVYYCQQYGSSPPRYTFGQGTKLAMK (SEQ ID NO: 128); or
d) the HCVR region comprises an amino acid sequence that has at least 95% sequence identity to the amino acid sequence EVQLVESGGGLVQPGGSLRLSCAASGFTFDNYAMGWVRQPPGKGLEWVSSFSGRG VSTYYADSVKGRFTVSRDSSKNTLFLQMNYLRVEDTAVYYCATYPYDILTGYYGAF DYWGQGALVTVSS (SEQ ID NO: 116) and the LCVR region comprises an amino acid sequence that has at least 95% sequence identity to the amino acid sequence DIQLTQSPSTLSASVGDRVTITCRASQTIDTWLAWYQQKPGKAPKLLIYGASSLQS GVPSRFSASGSGTEFTLTISSLQPDDFAIYYCQQYKDYSTFGQGSKVEFM (SEQ ID NO: 129); or
e) the HCVR region comprises an amino acid sequence that has at least 95% sequence identity to the amino acid sequence QVQLQQWGAGLLKPSETLSLTCSFYGGSFSGYCWSWIRQSPGKGLEWIGEINHSGS TNYNPSLKTRVTILIDTSNNQFSLRLSSVTAADTAVYYCARERAIRRCTSTSCYRVG GVAGLDVWGQGTTVSVSS (SEQ ID NO: 117) and the LCVR region comprises an amino acid sequence that has at least 95% sequence identity to the amino acid sequence DIVMTQSPSSLSASVGDRVTITCQASRDIYKYLNWYQQKPGQAPKLLISDASNLET GVPSRFSGSGSGTDFTFTISSLQPEDIAAYYCQQYDNPLITFGQGTRLEIK (SEQ ID NO: 130); or
f) the HCVR region comprises an amino acid sequence that has at least 95% sequence identity to the amino acid sequence QVQLVQSGGGLAQPGESLRLSCAASGFTFSSYAMTWVRQAPGKGLEWVSSISGKG ENIEYAESVKGRFTISRDNAKNTVDLEMNSLRAEDTATYFCAKHIYGAFIVVPNTL YDALDVWGQATKVTVSS (SEQ ID NO: 118) and the LCVR region comprises an amino acid sequence that has at least 95% sequence identity to the amino acid sequence EIVLTQSPGTLSLSPGERATLSCRASQSVSRNYLAWYQQKPGQAPRILVYDASNRAI GIPDRFSGSGSGTDFTLTISRLEPEDFAVYYCQQYGSSPGTFGQGTRLEIK (SEQ ID NO: 131); or
g) the HCVR region comprises an amino acid sequence that has at least 95% sequence identity to the amino acid sequence QVQLQESGPGLVKPSETLSLTCAVSGGSVSSDTYYWSWIRQPPGKGLEWIGYIYNS GSTNYNPSLKSRVTISVDTSKSQFSLNLGSVTAADTAVYYCAREGAGTYPPKNDAF DIWGQGTLVTVSS (SEQ ID NO: 119) and the LCVR region comprises an amino acid sequence that has at least 95% sequence identity to the amino acid sequence EIVLTQSPDSLAVSLGERATINCKSSQSVLFSSDNKNYLAWYQQKPGQPPKLLIYW ASTRESGVPDRFSGSGSGTDFTLTISSLQAEDVAVYYCQQYYNSPRTFGQGTKVEIK (SEQ ID NO: 132); or
h) the HCVR region comprises an amino acid sequence that has at least 95% sequence identity to the amino acid sequence QVQLQESGGGLVKPGGSLRLSCAASGFTFSSYSMNWVRQAPGKGLEWVSSISSSSS YIYYADSVKGRFTISRDNAKNSLYLQMNSLRAEDTAVYYCARGIIDYGDYFDYWGQ GTLVTVSS (SEQ ID NO: 120) and the LCVR region comprises an amino acid sequence that has at least 95% sequence identity to the amino acid sequence EIVLTQSPDTLSLSPGERATVSCRASQSVGAGYVAWYQQRPGQPPRLLIYGASVRA TGIPDRFSGSGSGTDFSLTINRVEPEDFAVYYCQNYASSPPRYTFGQGTKLEIK (SEQ ID NO: 133); or
i) the HCVR region comprises an amino acid sequence that has at least 95% sequence identity to the amino acid sequence EVQLVESGAEVKKPGASVRVSCKASGFTFTDHYMHWVRQAPGQGLEWMGLINPT GVNTFYAQNFRGRVTMTRDTSTKTDYLEVRSLTSQDTAMYYCARMSRYGAFDIW GQGTMVTVSL (SEQ ID NO: 121) and the LCVR region comprises an amino acid sequence that has at least 95% sequence identity to the amino acid sequence EIVLTQSPGTLSLSPGERATLSCRASQSVGSGYLAWYQQKPGQAPRLLIYGASVRAT GIPDRFSGSGSGTDFSLTINRVEPEDFAMYYCQNYGSSPPRYTFGQGTKLEIK (SEQ ID NO: 134); or
j) the HCVR region comprises an amino acid sequence that has at least 95% sequence identity to the amino acid sequence EVQLVESGAEVKKPGASVRLSCKASGDTFNNKYVHWVRQAPGQGLEWMGLINPS GSGTAFAQKFQGRVSMTRDTSTRTVYLGLTSLTYEDTAVYYCARMSRAGTFDIWD QGTMVTVSS (SEQ ID NO: 122) and the LCVR region comprises an amino acid sequence that has at least 95% sequence identity to the amino acid sequence EIVLTQSPGTLSLSPGDRATLSCRASENVGTSYLAWYQQKPGQAPRLLISGTSSRAT GVPDRFSGSGSGRDFTLTISRLEPEDFAVYYCQQYGSSPPRYTFGQGTKLDMK (SEQ ID NO: 135); or
k) the HCVR region comprises an amino acid sequence that has at least 95% sequence identity to the amino acid sequence QVQLVQSGAEVKKPGASVKLSCRASGYPITSHYMHWVRQAPGQGLEWMGIINPSG TGTSFARNFQGRVTMTRDTATRTVYMELSSLKSEDSAVYYCAGGTMGPLFDYWG QGTLVTVSS (SEQ ID NO: 123) and the LCVR region comprises an amino acid sequence that has at least 95% sequence identity to the amino acid sequence EIVLTQSPGTLSLSPGERATLSCRASQSVRRNFLAWYQQKPGQAPRLLIYEASTRA TGIPDRFSGSGSGTDFTLTISRLEPEDFAVYYCQQYDSSPPGVTFGPGTKVDIR (SEQ ID NO: 136); or
l) the HCVR region comprises an amino acid sequence that has at least 95% sequence identity to the amino acid sequence QVQLVESGGGVVQPGKSLKLSCAASGFTFSTYSMHWVRQAPGKGLEWVAIISYDG RNKYYANSVKGRFTISRDNSKNTLYLEINNLRPQDTAVYYCATDASNVEVPGKFA PLDKWGQGTLVTVSS (SEQ ID NO: 124) and the LCVR region comprises an amino acid sequence that has at least 95% sequence identity to the amino acid sequence EIVMTQSPATLSVSPGERATLSCRASQSVSSDLAWYQQKPGQAPRLLIYGATTRAT GVPARFSGSGSGAEFTLTISSLQSGDFAVYYCQQYNDWPLITFGQGTRLEIK (SEQ ID NO: 137); or
m) the HCVR region comprises an amino acid sequence that has at least 95% sequence identity to the amino acid sequence QVQLVESGGGVVQPGRSLRLSCAASGFTLSSHGIHWVRQAPGKGLEWVAVTSFDG RNKKFGDSVKGRFTISRDNSKNTVYLQMNSLRTEDTAVYYCAKDWGDDYSRWYF DLWGRGTLVTVSS (SEQ ID NO: 125) and the LCVR region comprises an amino acid sequence that has at least 95% sequence identity to the amino acid sequence DIQMTQSPSSLSASVGDRVTITCQASHDITKYLNWYQQKPGRAPKLLIYDASNLKT GVPSRFSGSGSGTDFTFTITSLQPEDLATYYCQQSDVLPITFGQGTRLEIK (SEQ ID NO: 138).

96. The antibody or antigen-binding fragment or variant thereof of claim 95, wherein the fragment comprises an F(ab), an Fv, or an scFv.

97. The antibody or antigen-binding fragment or variant thereof of claim 95, wherein the fragment comprises a VhH.

98. A nanoparticle comprising the epitope scaffold of claim 66.

99-100. (canceled)
